(19) United States
(12) Patent Application Publication
(10) Pub. No.: US 2016/0193206 A1
(43) Pub. Date: Jul. 7, 2016

(54) BET-PROTEIN-INHIBITING DIHYDROPYRIDOPYRAZINONES

(71) Applicant: BAYER PHARMA AKTIENGESELLSCHAFT, Berlin (DE)

(72) Inventors: Norbert SCHMEES, Berlin (DE); Benjamin BADER, Berlin (DE); Bernard HAENDLER, Berlin (DE); Detlef STÖCKIGT, Potsdam (DE); Pascale LEJEUNE, Berlin (DE); Amaury Ernesto FERNANDEZ-MONTALVAN, Berlin (DE); Timo STELLFELD, Berlin (DE); Daniel GALLENKAMP, Wuppertal (DE)

(21) Appl. No.: 14/654,572

(22) PCT Filed: Dec. 17, 2013
(86) PCT No.: PCT/EP2013/076784

§ 371 Date: Jun. 22, 2015

(30) Foreign Application Priority Data
Dec. 20, 2012 (EP) 12198623.6
Aug. 29, 2013 (EP) 13182252.0
Nov. 7, 2013 (EP) 13191933.4

(51) Int. Cl. A61K 31/4985; C07D 493/10; C07D 495/10; C07D 471/04; A61K 31/5377

(52) U.S. Cl. CPC A61K 31/4985; C07D 471/04; A61K 31/5377; C07D 495/10; C07D 493/10

(57) ABSTRACT

The present invention relates to BET protein-inhibitory, especially BRD4-inhibitory, dihydropyridopyrazinones of the general formula (I)

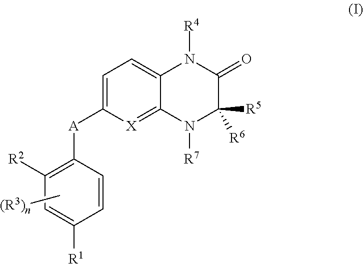

in which A, X, R¹, R², R³, R⁴, R⁴, R⁶, R⁷ and n have the meanings given in the description, to intermediates for preparation of the compounds according to the invention, to pharmaceutical compositions comprising the compounds according to the invention, and to the prophylactic and therapeutic use thereof in the case of hyperproliferative disorders, especially in the case of neoplastic disorders.

This invention further relates to the use of BET protein inhibitors in viral infections, in neurodegenerative disorders, in inflammation diseases, in atherosclerotic disorders and in male fertility control.

The present invention relates to BET protein-inhibitory, especially BRD4-inhibitory, dihydropyridopyrazinones, to intermediates for preparation of the compounds according to the invention, to pharmaceutical compositions comprising the compounds according to the invention, and to the prophylactic and therapeutic use thereof in the case of hyperproliferative disorders, especially in the case of neoplastic disorders. This invention further relates to the use of BET protein inhibitors in viral infections, in neurodegenerative disorders, in inflammation diseases, in atherosclerotic disorders and in male fertility control.

The human BET family (bromo domain and extra C-terminal domain family) has four members (BRD2, BRD3, BRD4 and BRDT) containing two related bromo domains and one extraterminal domain (Wu and Chiang, J. Biol. Chem., 2007, 282:13141-13145). The bromo domains are protein regions which recognize acetylated lysine residues. Such acetylated lysines are often found at the N-terminal end of histones (e.g. histone H3 or histone H4) and are features of an open chromatin structure and active gene transcription (Kuo and Allis, Bioessays, 1998, 20:615-626). In addition, bromo domains may recognize further acetylated proteins. For example, BRD4 binds to RelA, which leads to stimulation of NF-κB and transcriptional activity of inflammatory genes (Huang et al., Mol. Cell. Biol., 2009, 29:1375-1387). BRD4 also binds to cyclin T1 and forms an active complex which is important for transcription elongation (Schröder et al., J. Biol. Chem., 2012, 287:1090-1099). The extraterminal domain of BRD2, BRD3 and BRD4 interacts with several proteins involved in chromatin modulation and the regulation of gene expression (Rahman et al., Mol. Cell. Biol., 2011, 31:2641-2652).

In mechanistic terms, BET proteins play an important role in cell growth and in the cell cycle. They are associated with mitotic chromosomes, suggesting a role in epigenetic memory (Dey et al., Mol. Biol. Cell, 2009, 20:4899-4909; Yang et al., Mol. Cell. Biol., 2008, 28:967-976). Involvement of BRD4 in the post-mitotic reactivation of gene transcription has been demonstrated (Zhao et al., Nat. Cell. Biol., 2011, 13:1295-1304). BRD4 is essential for transcription elongation and recruits the elongation complex P-TEFb consisting of CDK9 and cyclin T1, which leads to activation of RNA polymerase II (Yang et al., Mol. Cell, 2005, 19:535-545; Schröder et al., J. Biol. Chem., 2012, 287:1090-1099). Consequently, the expression of genes involved in cell proliferation is stimulated, for example of c-Myc, cyclin D1 and aurora B (You et al., Mol. Cell. Biol., 2009, 29:5094-5103; Zuber et al., Nature, 2011, doi:10.1038). BRD2 is involved in the regulation of target genes of the androgen receptor (Draker et al., PLOS Genetics, 2012, 8, e1003047). BRD2 and BRD3 bind to transcribed genes in hyperacetylated chromatin regions and promote transcription by RNA polymerase II (LeRoy et al., Mol. Cell, 2008, 30:51-60).

Knock-down of BRD4 or the inhibition of the interaction with acetylated histones in various cell lines leads to G1 arrest (Mochizuki et al., J. Biol. Chem., 2008, 283:9040-9048; Mertz et al., Proc. Natl. Acad. Sci. USA, 2011, 108:16669-16674). It has also been shown that BRD4 binds to promoter regions of several genes which are activated in the G1 phase, for example cyclin D1 and D2 (Mochizuki et al., J. Biol. Chem., 2008, 283:9040-9048). In addition, inhibition of the expression of c-Myc, an essential factor in cell proliferation, after BRD4 inhibition has been demonstrated (Dawson et al., Nature, 2011, 478:529-533; Delmore et al., Cell, 2011, 146:1-14; Mertz et al., Proc. Natl. Acad. Sci. USA, 2011, 108:16669-16674). Inhibition of the expression of androgen-regulated genes and binding of BRD2 to corresponding regulatory regions has also been demonstrated (Draker et al., PLOS Genetics, 2012, 8, e1003047).

BRD2 and BRD4 knockout mice die early in embryogenesis (Gyuris et al., Biochim. Biophys. Acta, 2009, 1789:413-421; Houzelstein et al., Mol. Cell. Biol., 2002, 22:3794-3802). Heterozygotic BRD4 mice have various growth defects attributable to reduced cell proliferation (Houzelstein et al., Mol. Cell. Biol., 2002, 22:3794-3802).

BET proteins play an important role in various tumour types. Fusion between the BET proteins BRD3 or BRD4 and NUT, a protein which is normally expressed only in the testes, leads to an aggressive form of squamous cell carcinoma, called NUT midline carcinoma (French, Cancer Genet. Cytogenet., 2010, 203:16-20). The fusion protein prevents cell differentiation and promotes proliferation (Yan et al., J. Biol. Chem., 2011, 286:27663-27675). The growth of in vivo models derived therefrom is inhibited by a BRD4 inhibitor (Filippakopoulos et al., Nature, 2010, 468:1067-1073). Screening for therapeutic targets in an acute myeloid leukaemia cell line (AML) showed that BRD4 plays an important role in this tumour (Zuber et al., Nature, 2011, 478, 524-528). Reduction in BRD4 expression leads to a selective arrest of the cell cycle and to apoptosis. Treatment with a BRD4 inhibitor prevents the proliferation of an AML xenograft in vivo. Further experiments with a BRD4 inhibitor show that BRD4 is involved in various haematological tumours, for example multiple myeloma (Delmore et al., Cell, 2011, 146, 904-917) and Burkitt's lymphoma (Mertz et al., Proc. Natl. Acad. Sci. USA, 2011, 108, 16669-16674). In solid tumours too, for example lung cancer, BRD4 plays an important role (Lockwood et al., Proc. Natl. Acad. Sci. USA, 2012, 109, 19408-19413). Elevated expression of BRD4 has been detected in multiple myeloma, and amplification of the BRD4 gene has also been found in patients having multiple myeloma (Delmore et al., Cell, 2011, 146, 904-917). Amplification of the DNA region containing the BRD4 gene was detected in primary breast tumours (Kadota et al., Cancer Res, 2009, 69:7357-7365). For BRD2 too, there are data relating to a role in tumours. A transgenic mouse which overexpresses BRD2 selectively in B cells develops B cell lymphoma and leukaemia (Greenwall et al., Blood, 2005, 103:1475-1484).

BET proteins are also involved in viral infections. BRD4 binds to the E2 protein of various papillomaviruses and is important for the survival of the viruses in latently infected cells (Wu et al., Genes Dev., 2006, 20:2383-2396; Vosa et al., J. Virol., 2006, 80:8909-8919). The herpes virus, which is responsible for Kaposi's sarcoma, also interacts with various BET proteins, which is important for disease survival (Viejo-Borbolla et al., J. Virol., 2005, 79:13618-13629; You et al., J. Virol., 2006, 80:8909-8919). Through binding to P-TEFb, BRD4 also plays an important role in the replication of HIV-1 (Bisgrove et al., Proc. Natl. Acad. Sci. USA, 2007, 104:13690-13695). Treatment with a BRD4 inhibitor leads to stimulation of the dormant, untreatable reservoir of HIV-1 viruses in T cells (Banerjee et al., J. Leukoc. Biol., 2012, 92, 1147-1154). This reactivation could enable new therapeutic methods for AIDS treatment (Zinchenko et al., J. Leukoc. Biol., 2012, 92, 1127-1129). A critical role of BRD4 in DNA replication of polyomaviruses has also been reported (Wang et al., PLoS Pathog., 2012, 8, doi:10.1371).

BET proteins are additionally involved in inflammation processes. BRD2-hypomorphic mice show reduced inflammation in adipose tissue (Wang et al., Biochem. J., 2009, 425:71-83). Infiltration of macrophages in white adipose tissue is also reduced in BRD2-deficient mice (Wang et al., Biochem. J., 2009, 425:71-83). It has also been shown that BRD4 regulates a number of genes involved in inflammation. In LPS-stimulated macrophages, a BRD4 inhibitor prevents the expression of inflammatory genes, for example IL-1 or IL-6 (Nicodeme et al., Nature, 2010, 468:1119-1123).

BET proteins are also involved in the regulation of the ApoA1 gene (Mirguet et al., Bioorg. Med. Chem. Lett., 2012, 22:2963-2967). The corresponding protein is part of high-density lipoprotein (HDL), which plays an important role in atherosclerosis (Smith, Arterioscler. Thromb. Vasc. Biol., 2010, 30:151-155). Through the stimulation of ApoA1 expression, BET protein inhibitors can increase the concentrations of cholesterol HDL and hence may potentially be useful for the treatment of atherosclerosis (Mirguet et al., Bioorg. Med. Chem. Lett., 2012, 22:2963-2967). The BET protein BRDT plays an essential role in spermatogenesis through the regulation of the expression of several genes important during and after meiosis (Shang et al., Development, 2007, 134:3507-3515; Matzuk et al., Cell, 2012, 150:673-684). In addition, BRDT is involved in the post-meiotic organization of chromatin (Dhar et al., J. Biol. Chem., 2012, 287:6387-6405). In vivo experiments in mice show that treatment with a BET inhibitor which also inhibits BRDT leads to a decrease in sperm production and infertility (Matzuk et al., Cell, 2012, 150:673-684).

All these studies show that the BET proteins play an essential role in various pathologies, and also in male fertility. It would therefore be desirable to find potent and selective inhibitors which prevent the interaction between the BET proteins and acetylated proteins, in particular acetylated histone-H4 peptides. These novel inhibitors should also have suitable pharmacokinetic properties which allow inhibition of these interactions in vivo, i.e. in patients.

It has now been found that substituted dihydropyridopyrazinones have the desired properties, i.e. show BET protein-, in particular BRD4 protein-, inhibitory action. The compounds according to the invention are thus valuable active compounds for prophylactic and therapeutic use in the case of hyperproliferative disorders, especially in the case of neoplastic disorders. In addition, the compounds according to the invention can be employed in the case of viral infections, in the case of neurodegenerative disorders, in the case of inflammation disorders, in the case of atherosclerotic disorders and in male fertility control.

### PRIOR ART

The nomenclature applied in the assessment of the prior art (derived from the nomenclature software ACD Name batch, Version 12.01, from Advanced Chemical Development, Inc.) is illustrated by the following diagrams:

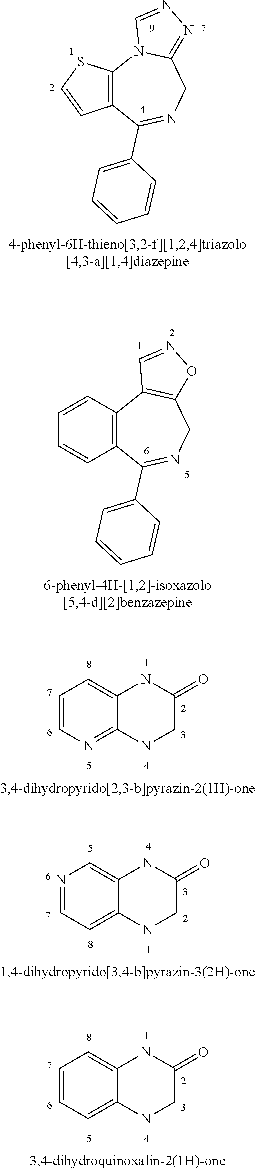 [SMILES: c1ccc(C2=NCc3nncn3-c3sccc32)cc1 ; c1ccc(C2=NCc3oncc3-c3ccccc32)cc1 ; O=C1CNc2ncccc2N1 ; O=C1CNc2ccncc2N1 ; O=C1CNc2ccccc2N1]

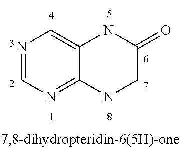 [SMILES: O=C1CNc2ncncc2N1]

Based on the chemical structure, only very few types of BRD4 inhibitors have been described to date (Chun-Wa Chung et al., Progress in Medicinal Chemistry 2012, 51, 1-55).

The first published BRD4 inhibitors were diazepines. For example, phenylthienotriazolo-1,4-diazepines (4-phenyl-6H-thieno[3,2-f][1,2,4]triazolo[4,3-a][1,4]diazepines) are described in WO2009/084693 (Mitsubishi Tanabe Pharma Corporation) and as compound JQ1 in WO2011/143669 (Dana Farber Cancer Institute). Replacement of the thieno moiety by a benzo moiety also leads to active inhibitors (J. Med. Chem. 2011, 54, 3827-3838; E. Nicodeme et al., Nature 2010, 468, 1119). Further 4-phenyl-6H-thieno[3,2-f][1,2,4]triazolo[4,3-a][1,4]diazepines and related compounds having alternative rings as a fusion partner rather than the benzo moiety are claimed generically or described explicitly in WO2012/075456 (Constellation Pharmaceuticals).

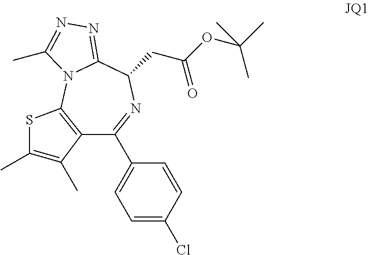 [SMILES: Cc1sc2c(c1C)C(c1ccc(Cl)cc1)=N[C@@H](CC(=O)OC(C)(C)C)c1nnc(C)n1-2]

Azepines as BRD-4 inhibitors have recently been described in WO2012/075383 (Constellation Pharmaceuticals). This application relates to 6-substituted 4H-isoxazolo[5,4-d][2]benzazepines and 4H-isoxazolo[3,4-d][2]benzazepines, including those compounds which have optionally substituted phenyl at position 6, and also to analogues with alternative heterocyclic fusion partners rather than the benzo moiety, for example thieno- or pyridoazepines. Another structural class of BRD4 inhibitors described is that of 7-isoxazoloquinolines and related quinolone derivatives (Bioorganic & Medicinal Chemistry Letters 22 (2012) 2963-2967). WO2011/054845 (GlaxoSmithKline) describes further benzodiazepines as BRD4 inhibitors.

The compounds according to the invention, in contrast, are substituted 3,4-dihydropyrido[2,3-b]pyrazin-2(1H)-one derivatives which differ structurally in various ways from the above-discussed chemotypes of BRD4 inhibitors. Because of the significant structural differences, it could not have been assumed that the compounds claimed here also have BRD4-inhibitory action. It is therefore surprising that the compounds according to the invention have good inhibitory action in spite of the considerable structural differences.

Some documents include compounds which are structurally similar but are aimed at completely different mechanisms of action, and in some cases also other indications.

Dihydropyridopyrazinones and related bicyclic systems have been described in a series of patent applications.

WO 2010/085570 (Takeda Pharmaceutical Company) describes inhibitors of poly-ADP-ribose polymerase (PARP) which are derived from a series of bi- and tricyclic skeletons, and which include 3,4-dihydropyrido[2,3-b]pyrazin-2(1H)-one derivatives, as medicaments for treatment of various diseases. The exemplary compounds disclosed therein differ from the compounds according to the invention for example by type and position of the substitution at the pyrido moiety of the dihydropyridopyrazinone skeleton.

WO 2006/005510 (Boehringer Ingelheim) describes 1,4-dihydropyrido[3,4-b]pyrazin-3(2H)-one derivatives as inhibitors of PLK-1 for treatment of hyperproliferative disorders. The substances disclosed in that publication differ from the compounds according to the invention in the position of the pyrido nitrogen.

WO 2008/117061 (Sterix Ltd) describes a number of bicyclic chemotypes as inhibitors of steroid sulphatase, inter alia for inhibiting the growth of tumours.

US 2006/0019961 (P. E. Mahaney et al.) describes substituted 3,4-dihydroquinoxalin-2(1H)-one derivatives as modulators of the oestrogen receptor for treatment of various inflammation disorders, cardiovascular disorders and autoimmune disorders.

WO 2006/050054, WO 2007/134169 and US 2009/0264384 (Nuada LLC) describe a series of bicyclic chemotypes as inhibitors of tumour necrosis factor alpha (TN-α) and various isoforms of phosphodiesterase for treatment of inflammation disorders among others.

WO 2012/088314 (Agios Pharmaceuticals) discloses a series of bicyclic chemotypes as modulators of pyruvate kinase M2.

WO 2003/020722 and WO 2004/076454 (Boehringer Ingelheim) disclose 7,8-dihydropteridin-6(5H)-ones as inhibitors of specific cell cycle kinases for the therapy of hyperproliferative disorders.

WO 2006/018182 (Boehringer Ingelheim) describes pharmaceutical preparations of 7,8-dihydropteridin-6(5H)-ones in combination inter alia with various cytostatics for the therapy of tumour disorders.

WO 2006/018185 (Boehringer Ingelheim) describes the use of 7,8-dihydropteridin-6(5H)-ones for the therapy of various tumour disorders.

WO 2011/101369 (Boehringer Ingelheim), WO 2011/113293 (Jiangsu Hengrui Medicine), WO 2009/141575 (Chroma Therapeutics), WO 2009/071480 (Nerviano Medical Sciences) and also WO 2006/021378, WO 2006/021379 and WO 2006/021548 (likewise Boehringer Ingelheim) disclose further 7,8-dihydropteridin-6(5H)-one derivatives as inhibitors of PLK-1 for treating hyperproliferative disorders.

U.S. Pat. No. 6,369,057 describes various quinoxaline and quinoxalinone derivatives as antivirally active compounds; EP 0657166 and EP 728481 describe combinations of such compounds with nucleosides or protease inhibitors having antiviral action.

WO 2007/022638 (Methylgene Inc.) discloses, in quite general terms, HDAC inhibitors of several chemotypes, but the structures of the example compounds disclosed differ distinctly from the compounds of the present invention.

WO 1999/050254 (Pfizer) describes a series of bicyclic chemotypes as inhibitors of serine proteases for antithrombotic therapy, but these compounds differ distinctly by the type and position of the substituents from the compounds according to the invention.

Some 3,4-dihydroquinoxalin-2(1H)-one derivatives substituted at C-6 by an aromatic amino group, in which the phenyl group is in turn substituted by a para-amide group (corresponding to 2-oxo-1,2,3,4-tetrahydroquinoxaline derivatives) are indexed by Chemical Abstracts as “Chemical Library” substances without a literature reference [see 4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide, CAS Registry No. 1026451-60-4, N-(1-benzylpiperidin-4-yl)-4-{[(3R)-4-cyclopentyl-1,3-dimethyl-2-oxo-1,2,3,4-tetrahydroquinoxalin-6-yl]amino}-3-methoxybenzamide, CAS Registry No. 1026961-36-3, 4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide, CAS Registry No. 1025882-57-8]. No therapeutic use for these compounds has been described to date.

Nevertheless, there is still a great need for selective active compounds for prophylaxis and therapy of disorders, in particular hyperproliferative disorders and especially neoplastic disorders.

It has now been found that compounds of the general formula (I)

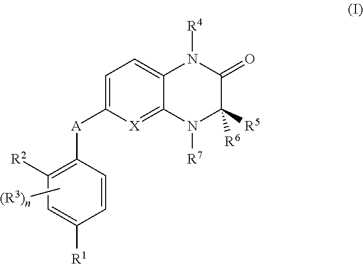

in which
A represents —NH— or —O—,
X represents —N—,
n represents 0 or 1,
R¹ represents —C(═O)NR⁸R⁹ or represents —S(═O)₂NR⁸R⁹,
   or represents oxazolin-2-yl which may optionally be mono- or disubstituted by identical or different C₁-C₃-alkyl substituents,
R² represents hydrogen, halogen, cyano, C₁-C₄-alkyl, C₂-C₄-alkenyl, C₂-C₄-alkynyl, halo-C₁-C₄-alkyl-, C₁-C₄-alkoxy-, C₁-C₄-alkoxy-C₁-C₄-alkyl-, halo-C₁-C₄-alkoxy-, C₁-C₄-alkylthio-, halo-C₁-C₄-alkylthio- or —NR¹⁰R¹¹,
R³ represents halogen, C₁-C₃-alkyl, C₁-C₃-alkoxy-, C₁-C₄-alkoxy-C₁-C₄-alkyl-, trifluoromethyl- or cyano and may be attached to any of the still-unoccupied positions in the aromatic system,
R⁴ represents methyl or ethyl,
R⁵ represents hydrogen or C₁-C₃-alkyl,
R⁶ represents hydrogen or C₁-C₃-alkyl,
   or
R⁵ and R⁶ together represent C₂-C₅-alkylene,
R⁷ represents C₁-C₆-alkyl, C₃-C₈-cycloalkyl, 4- to 8-membered heterocycloalkyl, phenyl or phenyl-C₁-C₃-alkyl,
   in which C₁-C₆-alkyl may optionally be mono-, di- or trisubstituted by identical or different substituents from the group consisting of fluorine, oxo, cyano, hydroxy, C₁-C₃-alkoxy- and —NR¹⁰R¹¹,
   and in which the phenyl radical may in each case optionally be mono-, di- or trisubstituted by identical or different substituents from the group consisting of halogen, cyano, C₁-C₄-alkyl, C₂-C₄-alkenyl, C₂-C₄-alkynyl, C₁-C₄-alkoxy-, halo-C₁-C₄-alkyl- and halo-C₁-C₄-alkoxy-,
   and in which 4- to 8-membered heterocycloalkyl may optionally be mono- or disubstituted by identical or different substituents from the group consisting of oxo, fluorine, cyano, C₁-C₄-alkyl, C₁-C₄-alkoxy-, C₁-C₄-alkylcarbonyl- and C₁-C₄-alkoxycarbonyl-,
R⁸ represents C₁-C₆-alkyl which may optionally be mono-, di- or trisubstituted by identical or different substituents from the group consisting of hydroxy, oxo, fluorine, cyano,
   C₁-C₄-alkoxy-, halo-C₁-C₄-alkoxy-, —NR¹⁰R¹¹, C₃-C₈-cycloalkyl, C₄-C₈-cycloalkenyl, 4- to 8-membered heterocycloalkyl, 4- to 8-membered heterocycloalkenyl, C₅-C₁₁-spirocycloalkyl, C₅-C₁₁-heterospirocycloalkyl, bridged C₆-C₁₂-cycloalkyl, bridged C₆-C₁₂-heterocycloalkyl, C₆-C₁₂-bicycloalkyl, C₆-C₁₂-heterobicycloalkyl, phenyl or 5- to 6-membered heteroaryl,
      in which C₃-C₈-cycloalkyl, C₄-C₈-cycloalkenyl, 4- to 8-membered heterocycloalkyl, 4- to 8-membered heterocycloalkenyl, C₅-C₁₁-spirocycloalkyl, C₅-C₁₁-heterospirocycloalkyl, bridged C₆-C₁₂-cycloalkyl, bridged C₆-C₁₂-heterocycloalkyl, C₆-C₁₂-bicycloalkyl, C₆-C₁₂-heterobicycloalkyl may in each case optionally be monosubstituted by oxo, C₁-C₄-alkyl or C₁-C₄-alkoxycarbonyl-,
      and in which phenyl and 5- to 6-membered heteroaryl may optionally be mono- or disubstituted by identical or different substituents from the group consisting of halogen, cyano, trifluoromethyl-, C₁-C₃-alkyl and C₁-C₃-alkoxy-,
   or represents C₃-C₆-alkenyl or C₃-C₆-alkynyl,
   or represents C₃-C₈-cycloalkyl, C₄-C₈-cycloalkenyl, C₅-C₁₁-spirocycloalkyl-, bridged C₆-C₁₂-cycloalkyl- or C₆-C₁₂-bicycloalkyl- which may optionally be mono- or disubstituted by identical or different substituents from the group consisting of hydroxy, oxo, cyano, fluorine, C₁-C₃-alkyl-, C₁-C₃-alkoxy-, trifluoromethyl-, —NR¹⁰R¹¹ and 4- to 8-membered heterocycloalkyl,
   or represents 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl which may optionally be mono- or disubstituted by identical or different substituents from the group consisting of hydroxy, oxo, cyano, fluorine, C₁-C₃-alkyl, C₁-C₃-alkoxy-, trifluoromethyl-, —NR¹⁰R¹¹, C₁-C₄-alkylcarbonyl- and C₁-C₄-alkoxycarbonyl-,
   or represents hydrogen,
R⁹ represents hydrogen or C₁-C₃-alkyl,
   or
R⁸ and R⁹ together with the nitrogen atom to which they are attached represent 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl which may optionally be mono- or disubstituted by identical or different substituents from the group consisting of hydroxy, oxo, cyano, fluorine, C₁-C₃-alkyl, C₃-C₆-cycloalkyl, C₁-C₃-alkoxy-, trifluoromethyl-, —NR¹⁰R¹¹, C₁-C₄-alkylcarbonyl- or C₁-C₄-alkoxycarbonyl-,
R¹⁰ and R¹¹ independently of one another represent hydrogen or represent C₁-C₆-alkyl which is optionally mono-, di- or trisubstituted by identical or different substituents from the group consisting of hydroxy, oxo and fluorine,
   or represent C₁-C₄-alkylcarbonyl- or C₁-C₄-alkoxycarbonyl-,
   or
R¹⁰ and R¹¹ together with the nitrogen atom to which they are attached represent 4- to 8-membered heterocycloalkyl which may optionally be mono- or disubstituted by identical or different substituents from the group consisting of hydroxy, oxo, cyano, fluorine, C₁-C₃-alkyl, halo-C₁-C₄-alkyl-, C₃-C₆-cycloalkyl-, C₃-C₆-cycloalkyl-C₁-C₃-alkyl-, benzyl or C₁-C₄-alkoxycarbonyl-,
   and diastereomers, racemates, polymorphs and physiologically acceptable salts thereof surprisingly inhibit the interaction between BRD4 and an acetylated histone 4 peptide and thus inhibit the growth of cancer and tumour cells.

Preference is given to those compounds of the general formula (I) in which
A represents —NH—,
X represents —N—,
n represents 0 or 1,
R¹ represents —C(═O)NR⁸R⁹ or represents —S(═O)₂NR⁸R⁹,
R² represents hydrogen, fluorine, chlorine, cyano, C₁-C₃-alkyl, fluoro-C₁-C₃-alkyl-, C₁-C₃-alkoxy-, fluoro-C₁-C₃-alkoxy-, C₁-C₃-alkylthio- or fluoro-C₁-C₃-alkylthio-,
R³ represents fluorine, chlorine, methoxy-, ethoxy- or cyano and may be attached to any of the still-unoccupied positions in the aromatic system,
R⁴ represents methyl or ethyl,
R⁵ represents C₁-C₃-alkyl,
R⁶ represents hydrogen,
R⁷ represents C₂-C₆-alkyl, C₃-C₇-cycloalkyl, 4- to 8-membered heterocycloalkyl, phenyl or phenyl-C₁-C₃-alkyl,
   in which C₂-C₆-alkyl may optionally be mono-, di- or trisubstituted by identical or different substituents from the group consisting of fluorine, C₁-C₃-alkoxy- and —NR¹⁰R¹¹,
   and in which the phenyl radical may in each case optionally be mono-, di- or trisubstituted by identical or different substituents from the group consisting of fluorine, chlorine, bromine, cyano, C₁-C₃-alkyl, C₁-C₃-alkoxy- and trifluoromethyl-,
   and in which 4- to 8-membered heterocycloalkyl may optionally be mono- or disubstituted by identical or different substituents from the group consisting of oxo, fluorine, C₁-C₄-alkyl, C₁-C₄-alkylcarbonyl- and C₁-C₄-alkoxycarbonyl-,
R⁸ represents C₁-C₆-alkyl which may optionally be mono-, di- or trisubstituted by identical or different substituents from the group consisting of hydroxy, oxo, fluorine, cyano, C₁-C₃-alkoxy, fluoro-C₁-C₃-alkoxy, —NR¹⁰R¹¹, 4- to 8-membered heterocycloalkyl, phenyl or 5- to 6-membered heteroaryl,
   in which the 4- to 8-membered heterocycloalkyl may optionally be monosubstituted by oxo, C₁-C₄-alkyl or C₁-C₄-alkoxycarbonyl-, and in which phenyl and 5- to 6-membered heteroaryl may optionally be mono- or disubstituted by identical or different substituents from the group consisting of fluorine, chlorine, cyano, trifluoromethyl-, methyl and methoxy-,
or represents C₃-C₈-cycloalkyl which may optionally be mono- or disubstituted by identical or different substituents from the group consisting of hydroxy, oxo, cyano, fluorine, —NR¹⁰R¹¹ and 4- to 8-membered heterocycloalkyl, or represents 4- to 8-membered heterocycloalkyl, C₆-C₈-heterospirocycloalkyl, bridged C₆-C₁₀-heterocycloalkyl or C₆-C₁₀-heterobicycloalkyl which may optionally be mono- or disubstituted by identical or different substituents from the group consisting of hydroxy, oxo, cyano, fluorine, C₁-C₃-alkyl, —NR¹⁰R¹¹, C₁-C₄-alkylcarbonyl- and C₁-C₄-alkoxycarbonyl-,
R⁹ represents hydrogen or C₁-C₃-alkyl,
   or
R⁸ and R⁹ together with the nitrogen atom to which they are attached represent 4- to 8-membered heterocycloalkyl, C₆-C₈-heterospirocycloalkyl, bridged C₆-C₁₀-heterocycloalkyl or C₆-C₁₀-heterobicycloalkyl which may optionally be mono- or disubstituted by identical or different substituents from the group consisting of oxo, cyano, fluorine, C₁-C₃-alkyl, C₃-C₆-cycloalkyl, —NR¹⁰R¹¹, C₁-C₄-alkylcarbonyl- and C₁-C₄-alkoxycarbonyl-,
R¹⁰ and R¹¹ independently of one another represent hydrogen or represent C₁-C₄-alkyl which is optionally mono-, di- or trisubstituted by identical or different substituents from the group consisting of hydroxy, oxo and fluorine,
   or represent C₁-C₄-alkylcarbonyl- or C₁-C₄-alkoxycarbonyl-,
   or
R¹⁰ and R¹¹ together with the nitrogen atom to which they are attached represent 4- to 7-membered heterocycloalkyl which may optionally be mono- or disubstituted by identical or different substituents from the group consisting of hydroxy, oxo, cyano, fluorine, C₁-C₃-alkyl, fluoro-C₁-C₃-alkyl-, C₃-C₆-cycloalkyl-, C₃-C₆-cycloalkylmethyl-, benzyl and C₁-C₄-alkoxycarbonyl-,
   and the diastereomers, racemates, polymorphs and physiologically acceptable salts thereof.

Particular preference is given to those compounds of the general formula I in which
A represents —NH—,
X represents —N—,
n represents 0 or 1,
R¹ represents —C(═O)NR⁸R⁹ or represents —S(═O)₂NR⁸R⁹,
R² represents hydrogen, fluorine, chlorine, methyl, ethyl, methoxy- or ethoxy-,
R³ represents methoxy- and may be attached to any of the still-unoccupied positions in the aromatic system,
R⁴ represents methyl,
R⁵ represents methyl or ethyl,
R⁶ represents hydrogen,
R⁷ represents C₂-C₅-alkyl, C₃-C₇-cycloalkyl, 5- to 6-membered heterocycloalkyl, phenyl or phenyl-C₁-C₃-alkyl,
   in which C₂-C₅-alkyl may optionally be monosubstituted by C₁-C₃-alkoxy, and in which 5- to 6-membered heterocycloalkyl may optionally be monosubstituted by C₁-C₄-alkoxycarbonyl-,
R⁸ represents C₁-C₄-alkyl which may optionally be monosubstituted by —NR¹⁰R¹¹, 4- to 8-membered heterocycloalkyl, phenyl or 5- to 6-membered heteroaryl,
   in which the 4- to 8-membered heterocycloalkyl may optionally be monosubstituted by oxo, C₁-C₄-alkyl or C₁-C₄-alkoxycarbonyl-, and in which phenyl and 5- to 6-membered heteroaryl may optionally be mono- or disubstituted by identical or different substituents from the group consisting of fluorine, chlorine, cyano, trifluoromethyl-, methyl and methoxy-,
or represents C₃-C₈-cycloalkyl which may optionally be mono- or disubstituted by identical or different substituents from the group consisting of hydroxy, oxo, —NR¹⁰R¹¹ and 5- to 6-membered heterocycloalkyl,
or represents 4- to 8-membered heterocycloalkyl which may optionally be mono- or disubstituted by identical or different substituents from the group consisting of oxo, C₁-C₃-alkyl, —NR¹⁰R¹¹, C₁-C₄-alkylcarbonyl- and C₁-C₄-alkoxycarbonyl-,
R⁹ represents hydrogen or methyl,
   or
R⁸ and R⁹ together with the nitrogen atom to which they are attached represent 5- to 6-membered heterocycloalkyl or C₆-C₈-heterospirocycloalkyl which may optionally be mono- or disubstituted by identical or different substituents from the group consisting of oxo, fluorine, C₁-C₃-alkyl, C₃-C₅-cycloalkyl, —NR¹⁰R¹¹, C₁-C₄-alkylcarbonyl- and C₁-C₄-alkoxycarbonyl-,
R¹⁰ and R¹¹ independently of one another represent hydrogen, C₁-C₄-alkyl or represent C₁-C₄-alkoxycarbonyl-,
   or
R¹⁰ and R¹¹ together with the nitrogen atom to which they are attached represent 5- to 6-membered heterocycloalkyl which may optionally be mono- or disubstituted by identical or different substituents from the group consisting of oxo, fluorine, C₁-C₃-alkyl, fluoro-C₁-C₃-alkyl-, C₃-C₅-cycloalkyl-, C₃-C₅-cycloalkylmethyl- and C₁-C₄-alkoxycarbonyl-,
   and the diastereomers, racemates, polymorphs and physiologically acceptable salts thereof.

Very particular preference is given to those compounds of the general formula I in which
A represents —NH—,
X represents —N—,
n represents 0 or 1,
R¹ represents —C(═O)NR⁸R⁹ or represents —S(═O)₂NR⁸R⁹,
R² represents hydrogen, fluorine, methyl or methoxy-,
R³ represents methoxy- and may be attached to any of the still-unoccupied positions in the aromatic system,
R⁴ represents methyl,
R⁵ represents methyl or ethyl,
R⁶ represents hydrogen,
R⁷ represents C₂-C₄-alkyl, C₅-C₇-cycloalkyl, pyrrolidinyl, piperidinyl, tetrahydropyranyl, phenyl or benzyl,
   in which C₂-C₄-alkyl may optionally be monosubstituted by methoxy-, and in which pyrrolidinyl and piperidinyl may optionally be monosubstituted by methoxycarbonyl, ethoxycarbonyl or tert-butoxycarbonyl-,
R⁸ represents C₁-C₂-alkyl which may optionally be monosubstituted by N,N-dimethylamino-, N-ethyl-N-methylamino-, N,N-diethylamino-, pyrrolidinyl, piperidinyl, piperazinyl, morpholinyl, phenyl or pyridinyl,
   in which pyrrolidinyl, piperidinyl, piperazinyl and morpholinyl may optionally be monosubstituted by methyl, ethyl or tert-butoxycarbonyl-, and in which phenyl and pyridinyl may optionally be monosubstituted by fluorine, chlorine, methyl or methoxy-,
or represents C₅-C₆-cycloalkyl which may optionally be monosubstituted by hydroxy, oxo, —NR¹⁰R¹¹, pyrrolidinyl, piperidinyl, piperazinyl, morpholinyl,
or represents oxetanyl, azetidinyl, pyrrolidinyl, tetrahydrofuranyl or piperidinyl which may optionally be monosubstituted by methyl, ethyl or acetyl-,
R⁹ represents hydrogen or methyl,
   or
R⁸ and R⁹ together with the nitrogen atom to which they are attached represent pyrrolidinyl, piperidinyl, piperazinyl, morpholinyl, 1-thia-6-azaspiro[3.3]hept-6-yl- or 2-oxa-6-azaspiro[3.3]hept-6-yl- which may optionally be mono- or disubstituted by identical or different substituents from the group consisting of oxo, fluorine, C₁-C₃-alkyl, cyclopropyl, piperidin-1-yl and tert-butoxycarbonyl-,
R¹⁰ and R¹¹ independently of one another represent hydrogen, C₁-C₃-alkyl or tert-butoxycarbonyl-,
   or
R¹⁰ and R¹¹ together with the nitrogen atom to which they are attached represent pyrrolidinyl, piperidinyl, piperazinyl or morpholinyl which may optionally be mono- or disubstituted by identical or different substituents from the group consisting of fluorine, 2,2,2-trifluoroethyl-, cyclopropyl, cyclopropylmethyl- and tert-butoxycarbonyl-,
   and the diastereomers, racemates, polymorphs and physiologically acceptable salts thereof.

Exceptional preference is given to those compounds of the general formula (I) in which
A represents —NH—,
X represents —N—,
n represents 0 or 1,
R¹ represents —C(═O)NR⁸R⁹ or represents —S(═O)₂NR⁸R⁹,
R² represents hydrogen, fluorine, methyl or methoxy-,
R³ represents methoxy- and may be attached to any of the still-unoccupied positions in the aromatic system,
R⁴ represents methyl,
R⁵ represents methyl,
R⁶ represents hydrogen,
R⁷ represents isopropyl, 2-methoxyethyl-, C₅-C₇-cycloalkyl, tetrahydropyran-4-yl, piperidin-4-yl, phenyl or benzyl,
   in which piperidin-4-yl may optionally be monosubstituted at its nitrogen atom by tert-butoxycarbonyl-,
R⁸ represents one of the groups below

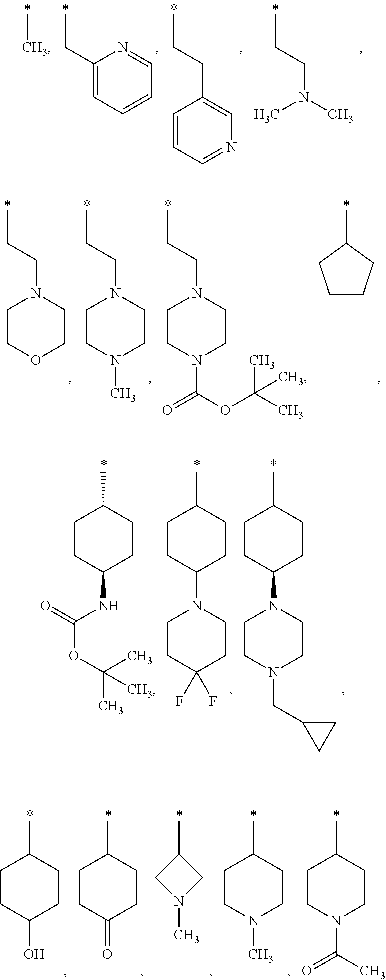

R⁹ represents hydrogen or methyl,
   or
R⁸ and R⁹ together with the nitrogen atom to which they are attached represent one of the groups below

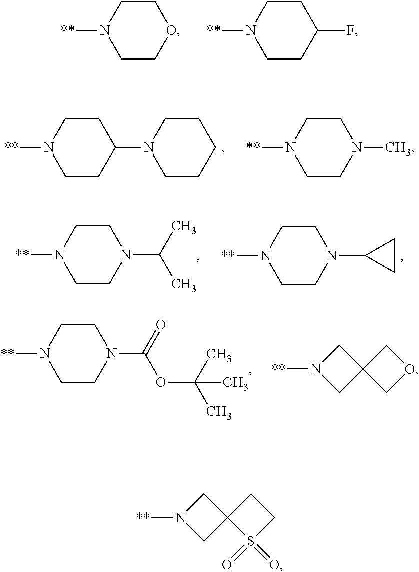

and the diastereomers, racemates, polymorphs and physiologically acceptable salts thereof.

In the definitions, “*” indicates the point of attachment to the nitrogen atom in —C(═O)NR⁸R⁹ and —S(═O)₂NR⁸R⁹, respectively.

In the definitions, “**” indicates the point of attachment to the carbonyl or sulphonyl group present in R¹.

Compounds which are furthermore of interest also include those compounds of the general formula (I) in which
A represents —NH— or —O—,
X represents —N—,
R¹ represents a group selected from
   a) —C(═O)NR⁸R⁹,
   b) —S(═O)₂NR⁸R⁹,
   c) oxazolin-2-yl, optionally substituted by one or two C₁-C₃-alkyl groups,
R² represents hydrogen, halogen, cyano, C₁-C₄-alkyl, C₂-C₄-alkenyl, C₂-C₄-alkynyl, halo-C₁-C₄-alkyl-, C₁-C₄-alkoxy-, halo-C₁-C₄-alkoxy-, C₁-C₄-alkylthio-, halo-C₁-C₄-alkylthio- or —NR¹⁰R¹¹,
R³ represents halogen, C₁-C₃-alkyl, C₁-C₃-alkoxy-, trifluoromethyl- or cyano and may be attached to any of the still-unoccupied positions in the aromatic system,
R⁴ represents methyl or ethyl,
R⁵ represents hydrogen or C₁-C₃-alkyl,
R⁶ represents hydrogen or C₁-C₃-alkyl,
   or
R⁵ and R⁶ together with the carbon atom to which they are attached represent C₃-C₆-cycloalkyl,
R⁷ represents C₁-C₆-alkyl, C₃-C₈-cycloalkyl, 4- to 8-membered heterocycloalkyl or phenyl-C₁-C₃-alkyl,
   in which the phenyl radical may optionally be mono-, di- or trisubstituted by identical or different substituents from the group consisting of halogen, cyano, C₁-C₄-alkyl, C₂-C₄-alkenyl, C₂-C₄-alkynyl, C₁-C₄-alkoxy-, halo-C₁-C₄-alkyl- and halo-C₁-C₄-alkoxy-,
R⁸ represents C₁-C₆-alkyl which may optionally and independently of the other be mono-, di- or trisubstituted by hydroxy, oxo, fluorine, cyano, C₁-C₄-alkoxy-, halo-C₁-C₄-alkoxy-, —NR¹⁰R¹¹, 4- to 8-membered heterocycloalkyl, 4- to 8-membered heterocycloalkenyl, C₅-C₁₁-heterospirocycloalkyl, bridged C₆-C₁₂-heterocycloalkyl, C₆-C₁₂-heterobicycloalkyl, phenyl or 5- to 6-membered heteroaryl, in which 4- to 8-membered heterocycloalkyl, 4- to 8-membered heterocycloalkenyl, C₅-C₁₁-heterospirocycloalkyl, bridged C₆-C₁₂-heterocycloalkyl, C₆-C₁₂-heterobicycloalkyl may each optionally contain one or more further heteroatoms and may optionally be monosubstituted by oxo, and in which phenyl and 5- to 6-membered heteroaryl may optionally be mono- or disubstituted by halogen, cyano, trifluoromethyl-, C₁-C₃-alkyl or C₁-C₃-alkoxy-, or represents C₃-C₆-alkenyl or C₃-C₆-alkynyl, or represents C₃-C₈-cycloalkyl or C₄-C₈-cycloalkenyl which may optionally be mono- or disubstituted by hydroxy, oxo, cyano, fluorine, C₁-C₃-alkyl, C₁-C₃-alkoxy-, trifluoromethyl-, —NR¹⁰R¹¹ or 4- to 8-membered heterocycloalkyl, or represents 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl, where the radicals mentioned may each optionally contain one or more further heteroatoms and where the radicals mentioned may optionally be mono- or disubstituted by hydroxy, oxo, cyano, fluorine, C₁-C₃-alkyl-, C₁-C₃-alkoxy-, trifluoromethyl- or —NR¹⁰R¹¹,
   or represents hydrogen,
R⁹ represents hydrogen or C₁-C₃-alkyl,
   or
R⁸ and R⁹ together with the nitrogen atom to which they are attached represent 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl, where the radicals mentioned may each optionally contain one or more further heteroatoms and where the radicals mentioned may optionally be mono- or disubstituted by hydroxy, oxo, cyano, fluorine, C₁-C₃-alkyl, C₁-C₃-alkoxy-, trifluoromethyl- or —NR¹⁰R¹¹,
n represents 0 or 1,
R¹⁰ and R¹¹ independently of one another represent hydrogen or C₁-C₆-alkyl which is optionally substituted by hydroxy, oxo or fluorine,
   or
R¹⁰ and R¹¹ together with the nitrogen atom to which they are attached represent 4- to 8-membered heterocycloalkyl which may optionally contain one or more further heteroatoms and may optionally be mono- or disubstituted by identical or different substituents from the group consisting of hydroxy, oxo, cyano, fluorine, C₁-C₃-alkyl, C₃-C₆-cycloalkyl, cyclopropylmethyl-, benzyl and C₁-C₄-alkoxycarbonyl-,
   and the diastereomers, racemates, polymorphs and physiologically acceptable salts thereof.

Of these, preference is given to those compounds of the general formula (I) in which
A represents —NH—,
X represents —N—,
R¹ represents a group selected from
   a) —C(═O)NR⁸R⁹,
   b) —S(═O)₂NR⁸R⁹,
   c) oxazolin-2-yl, optionally substituted by one or two C₁-C₃-alkyl groups,
R² represents hydrogen, fluorine, chlorine, cyano, C₁-C₃-alkyl, fluoro-C₁-C₃-alkyl-, C₁-C₃-alkoxy-, fluoro-C₁-C₃-alkoxy-, C₁-C₃-alkylthio- or fluoro-C₁-C₃-alkylthio-,
R³ represents fluorine, chlorine or cyano and may be attached to any of the still-unoccupied positions in the aromatic system,
R⁴ represents methyl or ethyl,
R⁵ represents C₁-C₃-alkyl,
R⁶ represents hydrogen,
R⁷ represents C₂-C₅-alkyl, C₃-C₆-cycloalkyl, 4- to 8-membered heterocycloalkyl or phenyl-C₁-C₃-alkyl,
   in which the phenyl radical may optionally be mono- or disubstituted by identical or different substituents from the group consisting of fluorine, chlorine, bromine, cyano, C₁-C₃-alkyl, C₁-C₃-alkoxy- and trifluoromethyl-,
R⁸ represents C₁-C₆-alkyl which may optionally and independently of the others be mono-, di- or trisubstituted by hydroxy, oxo, fluorine, cyano, C₁-C₃-alkoxy-, fluoro-C₁-C₃-alkoxy-, —NR¹⁰R¹¹, 4- to 8-membered heterocycloalkyl, phenyl or 5- to 6-membered heteroaryl,
   in which the 4- to 8-membered heterocycloalkyl may optionally contain one or more further heteroatoms and may optionally be monosubstituted by oxo,
   or represents C₃-C₆-cycloalkyl which may optionally be mono- or disubstituted by hydroxy, oxo, cyano, fluorine, —NR¹⁰R¹¹ and 4- to 8-membered heterocycloalkyl, or represents 4- to 8-membered heterocycloalkyl, C₆-C₈-heterospirocycloalkyl, bridged C₆-C₁₀-heterocycloalkyl or C₆-C₁₀-heterobicycloalkyl, where the radicals mentioned may each optionally contain one or more further heteroatoms and where the radicals mentioned may optionally be mono- or disubstituted by hydroxy, oxo, cyano, fluorine, C₁-C₃-alkyl or —NR¹⁰R¹¹,
R⁹ represents hydrogen or C₁-C₃-alkyl,
   or
R⁸ and R⁹ together with the nitrogen atom to which they are attached represent 4- or 8-membered heterocycloalkyl, C₆-C₈-heterospirocycloalkyl, bridged C₆-C₁₀-heterocycloalkyl or C₆-C₁₀-heterobicycloalkyl where the radicals mentioned may optionally contain one or more further heteroatoms and where the radicals mentioned may optionally be monosubstituted by oxo or C₁-C₃-alkyl,
n represents 0 or 1,
R¹⁰ and R¹¹ independently of one another represent hydrogen or C₁-C₄-alkyl which is optionally substituted by hydroxy, oxo or fluorine,
   or
R¹⁰ and R¹¹ together with the nitrogen atom to which they are attached represent 4- to 7-membered heterocycloalkyl which may optionally contain one or more further heteroatoms and may optionally be mono- or disubstituted by identical or different substituents from the group consisting of hydroxy, cyano, fluorine, C₁-C₃-alkyl, cyclopropyl, cyclopropylmethyl-, benzyl or C₁-C₄-alkoxycarbonyl-,
   and the diastereomers, racemates, polymorphs and physiologically acceptable salts thereof.

Of these, particular preference is given to those compounds of the general formula I in which
A represents —NH—,
X represents —N—,
R¹ represents a group selected from
   a) —C(═O)NR⁸R⁹,
   b) —S(═O)₂NR⁸R⁹,
R² represents hydrogen, fluorine, chlorine, methoxy- or ethoxy-,
R⁴ represents methyl,
R⁵ represents methyl or ethyl,
R⁶ represents hydrogen,
R⁷ represents C₃-C₅-alkyl, C₃-C₆-cycloalkyl, 5- to 6-membered heterocycloalkyl or phenyl-C₁-C₃-alkyl,
R⁸ represents C₁-C₄-alkyl or represents C₃-C₆-cycloalkyl which may optionally be monosubstituted by —NR¹⁰R¹¹ or 4- to 8-membered heterocycloalkyl,
   or represents 4- to 8-membered heterocycloalkyl,
   in which C₃-C₆-cycloalkyl or 4-8-membered heterocycloalkyl may optionally be monosubstituted by oxo, and in which the 4-8-membered heterocycloalkyl may optionally contain one or more further heteroatoms,
R⁹ represents hydrogen or methyl or
R⁸ and R⁹ together with the nitrogen atom to which they are attached represent 5- or 6-membered heterocycloalkyl or C₆-C₈-heterospirocycloalkyl, where the radicals mentioned may optionally contain one or more further heteroatoms and where the radicals mentioned may optionally be monosubstituted by oxo or C₁-C₃-alkyl,
n represents 0 and
R¹⁰ and R¹¹ independently of one another represent hydrogen or represent C₁-C₄-alkyl,
   or
R¹⁰ and R¹¹ together with the nitrogen atom to which they are attached represent 5- or 6-membered heterocycloalkyl which may optionally contain a further heteroatom and which may optionally be monosubstituted by C₁-C₃-alkyl, cyclopropyl, cyclopropylmethyl-, benzyl or tert-butoxycarbonyl-,
   and the diastereomers, racemates, polymorphs and physiologically acceptable salts thereof.

Of these, special preference is given to those compounds of the general formula I in which
A represents —NH—,
X represents —N—,
R¹ represents a group selected from
   a) —C(═O)NR⁸R⁹,
   b) —S(═O)₂NR⁸R⁹,
R² represents hydrogen or methoxy-,
R⁴ represents methyl,
R⁵ represents methyl,
R⁶ represents hydrogen,
R⁷ represents isopropyl, cyclopentyl, cyclohexyl, tetrahydropyran-4-yl or benzyl,
R⁸ represents

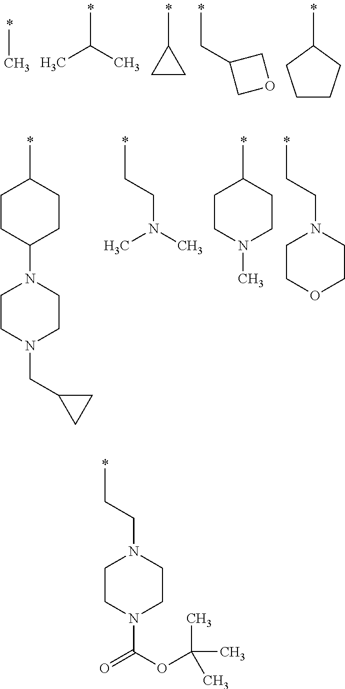

R⁹ represents hydrogen or methyl,
   or
R⁸ and R⁹ together with the nitrogen atom to which they are attached represent

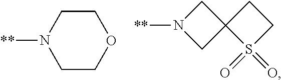

and
n represents 0,
   and the diastereomers, racemates, polymorphs and physiologically acceptable salts thereof.

Of these, special preferrence is furthermore given to those compounds of the general formula I in which
A represents —NH—,
X represents —N—,
R¹ represents a group selected from
   a) —C(═O)NR⁸R⁹,
   b) —S(═O)₂NR⁸R⁹,
R² represents hydrogen or methoxy-,
R⁴ represents methyl,
R⁵ represents methyl,
R⁶ represents hydrogen,
R⁷ represents isopropyl, cyclopentyl, cyclohexyl or tetrahydropyran-4-yl,
R⁸ represents

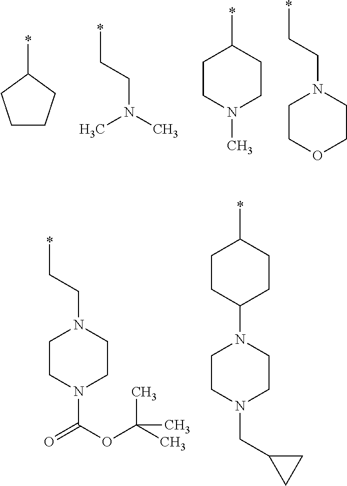

R⁹ represents hydrogen or methyl or
R⁸ and R⁹ together with the nitrogen atom to which they are attached represent

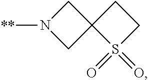

and
n represents 0,
   and the diastereomers, racemates, polymorphs and physiologically acceptable salts thereof.

In the definitions, “*” indicates the point of attachment to the nitrogen atom in —C(═O)NR⁸R⁹ and —S(═O)₂NR⁸R⁹, respectively.

In the definitions, “**” indicates the point of attachment to the carbonyl or sulphonyl group present in R¹.

Also furthermore of interest are those compounds of the general formula I in which
A represents —NH— or —O—,
X represents —N—,
R¹ represents a group selected from
   a) —C(═O)NR⁸R⁹,
   b) —S(═O)₂NR⁸R⁹,
   c) oxazolin-2-yl, optionally substituted by one or two C₁-C₃-alkyl groups,
R² represents hydrogen, halogen, cyano, C₁-C₄-alkyl, C₂-C₄-alkenyl, C₂-C₄-alkynyl, halo-C₁-C₄-alkyl-, C₁-C₄-alkoxy-, halo-C₁-C₄-alkoxy-, C₁-C₄-alkylthio-, halo-C₁-C₄-alkylthio- or —NR¹⁰R¹¹,
R³ represents halogen, C₁-C₃-alkyl, C₁-C₃-alkoxy-, trifluoromethyl- or cyano and may be attached to any of the still-unoccupied positions in the aromatic system,
R⁴ represents methyl or ethyl,
R⁵ represents C₁-C₃-alkyl,
R⁶ represents hydrogen or C₁-C₃-alkyl,
   or
R⁵ and R⁶ together with the carbon atom to which they are attached represent C₃-C₆-cycloalkyl,
R⁷ represents C₁-C₆-alkyl, C₃-C₈-cycloalkyl or phenyl-C₁-C₃-alkyl-, in which the phenyl radical may optionally be mono-, di- or trisubstituted by identical or different substituents from the group consisting of halogen, cyano, C₁-C₄-alkyl, C₂-C₄-alkenyl, C₂-C₄-alkynyl, C₁-C₄-alkoxy-, halo-C₁-C₄-alkyl- and halo-C₁-C₄-alkoxy-,
R⁸ represents C₁-C₆-alkyl which may optionally and independently of the other be mono-, di- or trisubstituted by hydroxy, oxo, fluorine, cyano, C₁-C₄-alkoxy-, halo-C₁-C₄-alkoxy-, —NR¹⁰R¹¹, 4- to 8-membered heterocycloalkyl, 4- to 8-membered heterocycloalkenyl, C₅-C₁₁-heterospirocycloalkyl, bridged C₆-C₁₂-heterocycloalkyl, C₆-C₁₂-heterobicycloalkyl, phenyl or 5- to 6-membered heteroaryl,
   in which 4- to 8-membered heterocycloalkyl, 4- to 8-membered heterocycloalkenyl, C₅-C₁₁-heterospirocycloalkyl, bridged C₆-C₁₂-heterocycloalkyl, C₆-C₁₂-heterobicycloalkyl may each optionally contain one or more further heteroatoms and may optionally be monosubstituted by oxo,
   and in which phenyl and 5- to 6-membered heteroaryl may optionally be mono- or disubstituted by halogen, cyano, trifluoromethyl-, C₁-C₃-alkyl or C₁-C₃-alkoxy-,
   or represents C₃-C₆-alkenyl or C₃-C₆-alkynyl,
   or represents C₃-C₈-cycloalkyl or C₄-C₈-cycloalkenyl which may optionally be mono- or disubstituted by hydroxy, oxo, cyano, fluorine, C₁-C₃-alkyl, C₁-C₃-alkoxy-, trifluoromethyl- or —NR¹⁰R¹¹,
   or represents 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl, where the radicals mentioned may each optionally contain one or more further heteroatoms and where the radicals mentioned may optionally be mono- or disubstituted by hydroxy, oxo, cyano, fluorine, C₁-C₃-alkyl, C₁-C₃-alkoxy-, trifluoromethyl- or —NR¹⁰R¹¹,
R⁹ represents hydrogen or C₁-C₃-alkyl,
   or
R⁸ and R⁹ together with the nitrogen atom to which they are attached represent 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl, where the radicals mentioned may each optionally contain one or more further heteroatoms and where the radicals mentioned may optionally be mono- or disubstituted by hydroxy, oxo, cyano, fluorine, C₁-C₃-alkyl, C₁-C₃-alkoxy-, trifluoromethyl- or —NR¹⁰R¹¹,
n represents 0 or 1,
R¹⁰ and R¹¹ independently of one another represent hydrogen or C₁-C₃-alkyl which is optionally substituted by hydroxy, oxo or fluorine,
   or
R¹⁰ and R¹¹ together with the nitrogen atom to which they are attached represent 4-8-membered heterocycloalkyl which may optionally contain one or more further heteroatoms and may optionally carry one or two substituents independently of one another selected from the group consisting of hydroxy, oxo, cyano, fluorine and C₁-C₃-alkyl,
   and the diastereomers, racemates, polymorphs and physiologically acceptable salts thereof.

Of these, compounds which are furthermore of interest are those compounds of the general formula I in which
A represents —NH— or —O—,
X represents —N—,
R¹ represents a group selected from
   a) —C(═O)NR⁸R⁹,
   b) —S(═O)₂NR⁸R⁹,
   c) oxazolin-2-yl, optionally substituted by one or two C₁-C₃-alkyl groups,
R² represents hydrogen, fluorine, chlorine, cyano, C₁-C₃-alkyl, fluoro-C₁-C₃-alkyl-, C₁-C₃-alkoxy-, fluoro-C₁-C₃-alkoxy-, C₁-C₃-alkylthio- or fluoro-C₁-C₃-alkylthio-,
R³ represents fluorine, chlorine or cyano and may be attached to any of the still-unoccupied positions in the aromatic system,
R⁴ represents methyl or ethyl,
R⁵ represents C₁-C₃-alkyl,
R⁶ represents hydrogen,
R⁷ represents C₂-C₅-alkyl, C₃-C₆-cycloalkyl or phenyl-C₁-C₃-alkyl-,
   in which the phenyl radical may optionally be mono- or disubstituted by identical or different substituents from the group consisting of fluorine, chlorine, bromine, cyano, C₁-C₃-alkyl, C₁-C₃-alkoxy- and trifluoromethyl-,
R⁸ represents C₁-C₆-alkyl which may optionally and independently of the others be mono-, di- or trisubstituted by hydroxy, oxo, fluorine, cyano, C₁-C₃-alkoxy-, fluoro-C₁-C₃-alkoxy-, —NR¹⁰R¹¹, 4- to 8-membered heterocycloalkyl, phenyl or 5- to 6-membered heteroaryl,
   in which the 4- to 8-membered heterocycloalkyl may optionally contain one or more further heteroatoms and may optionally be monosubstituted by oxo,
   or represents C₃-C₆-cycloalkyl which may optionally be mono- or disubstituted by hydroxy, oxo, cyano, fluorine or —NR¹⁰R¹¹,
   or represents 4- to 8-membered heterocycloalkyl, C₆-C₈-heterospirocycloalkyl, bridged C₆-C₁₀-heterocycloalkyl or C₆-C₁₀-heterobicycloalkyl, where the radicals mentioned may each optionally contain one or more further heteroatoms and where the radicals mentioned may optionally be mono- or disubstituted by hydroxy, oxo, cyano, fluorine, C₁-C₃-alkyl or —NR¹⁰R¹¹,
R⁹ represents hydrogen or C₁-C₃-alkyl,
   or
R⁸ and R⁹ together with the nitrogen atom to which they are attached represent 4- or 8-membered heterocycloalkyl, C₆-C₈-heterospirocycloalkyl, bridged C₆-C₁₀-heterocycloalkyl or C₆-C₁₀-heterobicycloalkyl, where the radicals mentioned may optionally contain one or more further heteroatoms and where the radicals mentioned may optionally be monosubstituted by oxo or C₁-C₃-alkyl,
n represents 0 or 1,
R¹⁰ and R¹¹ independently of one another represent hydrogen or C₁-C₃-alkyl which is optionally substituted by hydroxy, oxo or fluorine,
   or
R¹⁰ and R¹¹ together with the nitrogen atom to which they are attached represent 4-7-membered heterocycloalkyl which may optionally contain one or more further heteroatoms and may optionally carry one or two substituents independently of one another selected from the group consisting of hydroxy, cyano, fluorine and C₁-C₃-alkyl,
   and the diastereomers, racemates, polymorphs and physiologically acceptable salts thereof.

Of these, compounds which are furthermore of particular interest also include those compounds of the general formula I in which
A represents —NH— or —O—,
X represents —N—,
R¹ represents a group selected from
   a) —C(═O)NR⁸R⁹,
   b) —S(═O)₂NR⁸R⁹,
R² represents hydrogen, fluorine, chlorine, methoxy- or ethoxy-,
R⁴ represents methyl,
R⁵ represents methyl or ethyl,
R⁶ represents hydrogen,
R⁷ represents C₃-C₅-alkyl, C₃-C₆-cycloalkyl or phenyl-C₁-C₃-alkyl-,
R⁸ represents C₁-C₄-alkyl which may optionally be monosubstituted by —NR¹⁰R¹¹ or 4- to 8-membered heterocycloalkyl, or represents C₃-C₆-cycloalkyl, or represents 4- to 8-membered heterocycloalkyl,
   in which C₃-C₆-cycloalkyl or 4-8-membered heterocycloalkyl may optionally be monosubstituted by oxo, and in which the 4-8-membered heterocycloalkyl may optionally contain one or more further heteroatoms,
R⁹ represents hydrogen or methyl or
R⁸ and R⁹ together with the nitrogen atom to which they are attached represent 5- or 6-membered heterocycloalkyl or C₆-C₈-heterospirocycloalkyl, where the radicals mentioned may optionally contain one or more further heteroatoms and where the radicals mentioned may optionally be monosubstituted by oxo or C₁-C₃-alkyl,
n represents 0 and
R¹⁰ and R¹¹ independently of one another represent hydrogen, methyl or ethyl,
   and the diastereomers, racemates, polymorphs and physiologically acceptable salts thereof.

Of these, compounds which are furthermore of more interest are those compounds of the general formula I in which
A represents —NH— or —O—,
X represents —N—,
R¹ represents a group selected from
   a) —C(═O)NR⁸R⁹,
   b) —S(═O)₂NR⁸R⁹,
R² represents hydrogen or methoxy-,
R⁴ represents methyl,
R⁵ represents methyl,
R⁶ represents hydrogen,
R⁷ represents isopropyl, cyclopentyl, cyclohexyl or benzyl,
R⁸ represents

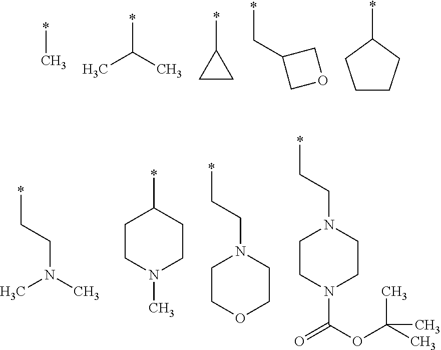

R⁹ represents hydrogen or methyl,
   or
R⁸ and R⁹ together with the nitrogen atom to which they are attached represent

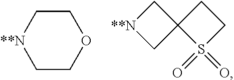

and
n represents 0,
   and the diastereomers, racemates, polymorphs and physiologically acceptable salts thereof.

Of these, compounds which are furthermore of particular interest are those compounds of the general formula I in which
A represents —NH—,
X represents —N—,
R¹ represents a group selected from
   a) —C(═O)NR⁸R⁹,
   b) —S(═O)₂NR⁸R⁹,
R² represents hydrogen or methoxy-,
R⁴ represents methyl,
R⁵ represents methyl,
R⁶ represents hydrogen,
R⁷ represents isopropyl, cyclopentyl, cyclohexyl or benzyl,
R⁸ represents

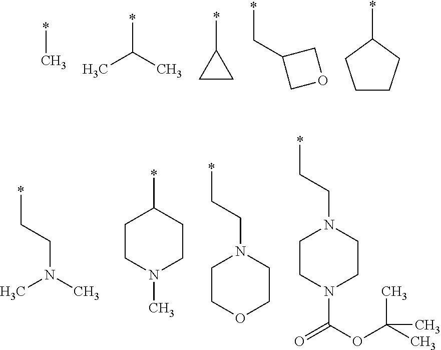

R⁹ represents hydrogen or methyl or
R⁸ and R⁹ together with the nitrogen atom to which they are attached represent

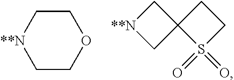

and
n represents 0,
   and the diastereomers, racemates, polymorphs and physiologically acceptable salts thereof.

Of these, compounds which are furthermore likewise especially preferred are compounds of the general formula I in which
A represents —O—,
X represents —N—,
R¹ represents a group selected from
   a) —C(═O)NR⁸R⁹,
   b) —S(═O)₂NR⁸R⁹,
R² represents hydrogen or methoxy-,
R⁴ represents methyl,
R⁵ represents methyl,
R⁶ represents hydrogen,
R⁷ represents isopropyl, cyclopentyl, cyclohexyl or benzyl,
R⁸ represents

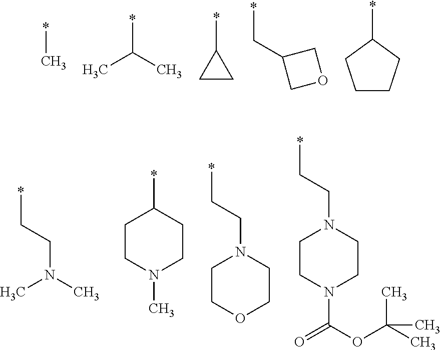

R⁹ represents hydrogen or methyl or
R⁸ and R⁹ together with the nitrogen atom to which they are attached represent

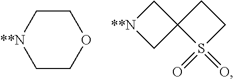

and
n represents 0,
   and the diastereomers, racemates, polymorphs and physiologically acceptable salts thereof.

In the definitions, “*” indicates the point of attachment to the nitrogen atom in —C(═O)NR⁸R⁹ and —S(═O)₂NR⁸R⁹, respectively.

In the definitions, “**” indicates the point of attachment to the carbonyl or sulphonyl group present in R¹.

Preference is additionally given to compounds of the general formula (I) in which A represents —NH—.

Preference is given to compounds of the general formula (I) in which R¹ represents —C(═O)NR⁸R⁹.

Preference is given to compounds of the general formula (I) in which R¹ represents —S(═O)₂NR⁸R⁹.

Preference is given to compounds of the general formula (I) in which R² represents hydrogen, fluorine, chlorine, cyano, C₁-C₃-alkyl, fluoro-C₁-C₃-alkyl-, C₁-C₃-alkoxy- or fluoro-C₁-C₃-alkoxy-.

Preference is given to compounds of the general formula (I) in which R² represents hydrogen, fluorine, chlorine, C₁-C₃-alkyl or C₁-C₃-alkoxy-.

Preference is given to compounds of the general formula (I) in which R² represents hydrogen, fluorine, chlorine, C₁-C₃-alkyl or C₁-C₃-alkoxy- and in which n represents the number 0.

Preference is given to compounds of the general formula (I) in which R² represents hydrogen, fluorine, chlorine, methyl or methoxy-.

Preference is given to compounds of the general formula (I) in which R² represents hydrogen, fluorine, chlorine, methyl or methoxy- and in which n represents the number 0.

Preference is given to compounds of the general formula (I) in which R² represents C₁-C₃-alkoxy-.

Preference is given to compounds of the general formula (I) in which R² represents C₁-C₃-alkyl.

Preference is given to compounds of the general formula (I) in which R² represents ethoxy-.

Preference is given to compounds of the general formula (I) in which R² represents fluorine.

Preference is given to compounds of the general formula (I) in which R² represents chlorine.

Particular preference is given to compounds of the general formula (I) in which R² represents methoxy-.

Particular preference is given to compounds of the general formula (I) in which R² represents methyl.

Particular preference is given to compounds of the general formula (I) in which R² represents hydrogen.

Particular preference is given to compounds of the general formula (I) in which R² represents methoxy- and in which n represents the number 0.

Particular preference is given to compounds of the general formula (I) in which R² represents methyl and in which n represents the number 0.

Particular preference is given to compounds of the general formula (I) in which R² represents hydrogen and in which n represents the number 0.

Particular preference is given to compounds of the general formula (I) in which R² represents hydrogen, fluorine, methyl or methoxy-, R⁴ and R⁵ each represent methyl, R⁶ represents hydrogen and in which n represents the number 0.

Particular preference is given to compounds of the general formula (I) in which R² represents hydrogen, methyl or methoxy-, R⁴ and R⁵ each represent methyl, R⁶ represents hydrogen and in which n represents the number 0.

Particular preference is given to compounds of the general formula (I) in which R² represents methoxy-, R⁴ and R⁵ each represent methyl, R⁶ represents hydrogen and in which n represents the number 0.

Particular preference is given to compounds of the general formula (I) in which R² represents methyl, R⁴ and R⁵ each represent methyl, R⁶ represents hydrogen and in which n represents the number 0.

Particular preference is given to compounds of the general formula (I) in which R² represents hydrogen, R⁴ and R⁵ each represent methyl, R⁶ represents hydrogen and in which n represents the number 0.

Preference is given to compounds of the general formula (I) in which R³ represents C₁-C₃-alkoxy-.

Preference is given to compounds of the general formula (I) in which R⁴ represents methoxy-.

Preference is given to compounds of the general formula (I) in which R⁴ represents methyl or ethyl.

Preference is given to compounds of the general formula (I) in which R⁴ represents ethyl.

Particular preference is given to compounds of the general formula (I) in which R⁴ represents methyl.

Particular preference is given to compounds of the general formula (I) in which R⁴ and R⁵ each represent methyl.

Particular preference is given to compounds of the general formula (I) in which R⁴ and R⁵ each represent methyl and in which n represents the number 0.

Particular preference is given to compounds of the general formula (I) in which R⁴ represents methyl and R⁶ represents hydrogen.

Particular preference is given to compounds of the general formula (I) in which R⁴ and R⁵ each represent methyl and R⁶ represents hydrogen.

Particular preference is given to compounds of the general formula (I) in which R⁴ and R⁵ each represent methyl, R⁶ represents hydrogen and in which n represents the number 0.

Preference is given to compounds of the general formula (I) in which R⁵ represents methyl or ethyl.

Preference is given to compounds of the general formula (I) in which R⁵ represents ethyl.

Particular preference is given to compounds of the general formula (I) in which R⁵ represents methyl.

Particular preference is given to compounds of the general formula (I) in which R⁵ represents methyl and in which R⁶ represents hydrogen.

Preference is given to compounds of the general formula (I) in which R⁶ represents hydrogen.

Preference is given to compounds of the general formula (I) in which R⁷ represents C₃-C₅-alkyl, C₃-C₆-cycloalkyl, 5- to 6-membered heterocycloalkyl or phenyl-C₁-C₃-alkyl-.

Preference is given to compounds of the general formula (I) in which R⁷ represents C₃-C₅-alkyl.

Preference is given to compounds of the general formula (I) in which R⁷ represents C₃-C₆-cycloalkyl.

Preference is given to compounds of the general formula (I) in which R⁷ represents 5- to 6-membered heterocycloalkyl.

Preference is given to compounds of the general formula (I) in which R⁷ represents phenyl-C₁-C₃-alkyl-.

Preference is given to compounds of the general formula (I) in which R⁷ represents C₂-C₅-alkyl, C₃-C₇-cycloalkyl, 5- to 6-membered heterocycloalkyl, phenyl or phenyl-C₁-C₃-alkyl-

in which C₂-C₅-alkyl may optionally be monosubstituted by C₁-C₃-alkoxy,
and in which 5- to 6-membered heterocycloalkyl may optionally be monosubstituted by C₁-C₄-alkoxycarbonyl-.

Preference is given to compounds of the general formula (I) in which R⁷ represents C₂-C₅-alkyl in which C₂-C₅-alkyl may optionally be monosubstituted by C₁-C₃-alkoxy-.

Preference is given to compounds of the general formula (I) in which R⁷ represents C₃-C₇-cycloalkyl.

Preference is given to compounds of the general formula (I) in which R⁷ represents 5- to 6-membered heterocycloalkyl in which 5- to 6-membered heterocycloalkyl may optionally be monosubstituted by C₁-C₄-alkoxycarbonyl-.

Preference is given to compounds of the general formula (I) in which R⁷ represents phenyl

Preference is given to compounds of the general formula (I) in which R⁷ represents C₂-C₄-alkyl, C₅-C₇-cycloalkyl, pyrrolidinyl, piperidinyl, tetrahydropyranyl, phenyl or benzyl,

in which C₂-C₄-alkyl may optionally be monosubstituted by methoxy-,
and in which pyrrolidinyl and piperidinyl may optionally be monosubstituted by methoxycarbonyl, ethoxycarbonyl or tert-butoxycarbonyl-.

Preference is given to compounds of the general formula (I) in which R⁷ represents C₂-C₄-alkyl in which C₂-C₄-alkyl may optionally be monosubstituted by methoxy-.

Preference is given to compounds of the general formula (I) in which R⁷ represents C₅-C₇-cycloalkyl.

Preference is given to compounds of the general formula (I) in which R⁷ represents pyrrolidinyl, piperidinyl or tetrahydropyranyl,

in which pyrrolidinyl and piperidinyl may optionally be monosubstituted by methoxycarbonyl, ethoxycarbonyl or tert-butoxycarbonyl-.

Preference is given to compounds of the general formula (I) in which R⁷ represents pyrrolidinyl, piperidinyl or tetrahydropyranyl,

in which pyrrolidinyl and piperidinyl may optionally be monosubstituted by tert-butoxycarbonyl-.

Preference is given to compounds of the general formula (I) in which R⁷ represents pyrrolidinyl or piperidinyl,

in which pyrrolidinyl and piperidinyl may optionally be monosubstituted by methoxycarbonyl, ethoxycarbonyl or tert-butoxycarbonyl-.

Preference is given to compounds of the general formula (I) in which R⁷ represents pyrrolidinyl or piperidinyl,

in which pyrrolidinyl and piperidinyl may optionally be monosubstituted by tert-butoxycarbonyl-.

Preference is given to compounds of the general formula (I) in which R⁷ represents tetrahydropyranyl.

Preference is given to compounds of the general formula (I) in which R⁷ represents phenyl or benzyl.

Preference is given to compounds of the general formula (I) in which R⁷ represents phenyl.

Preference is given to compounds of the general formula (I) in which R⁷ represents benzyl.

Preference is given to compounds of the general formula (I) in which R⁷ represents isopropyl, cyclopentyl, cyclohexyl, tetrahydropyran-4-yl or benzyl.

Particular preference is given to compounds of the general formula (I) in which R⁷ represents isopropyl, cyclopentyl, cyclohexyl or tetrahydropyran-4-yl.

Particular preference is given to compounds of the general formula (I) in which R⁷ represents isopropyl.

Particular preference is given to compounds of the general formula (I) in which R⁷ represents 2-methoxyethyl.

Particular preference is given to compounds of the general formula (I) in which R⁷ represents cyclopentyl.

Particular preference is given to compounds of the general formula (I) in which R⁷ represents cyclohexyl.

Particular preference is given to compounds of the general formula (I) in which R⁷ represents cycloheptyl.

Particular preference is given to compounds of the general formula (I) in which R⁷ represents tetrahydropyran-4-yl.

Particular preference is given to compounds of the general formula (I) in which R⁷ represents piperidin-4-yl, in which piperidin-4-yl may optionally be monosubstituted at its nitrogen atom by methoxycarbonyl, ethoxycarbonyl or tert-butoxycarbonyl-.

Particular preference is given to compounds of the general formula (I) in which R⁷ represents piperidin-4-yl, in which piperidin-4-yl may optionally be monosubstituted at its nitrogen atom by tert-butoxycarbonyl-.

Preference is given to compounds of the general formula (I) in which R⁸ represents C₁-C₄-alkyl which may optionally be monosubstituted by —NR¹⁰R¹¹, 4- to 8-membered heterocycloalkyl, phenyl or 5- to 6-membered heteroaryl,

in which the 4- to 8-membered heterocycloalkyl may optionally be monosubstituted by oxo, C₁-C₄-alkyl or C₁-C₄-alkoxycarbonyl-,
and in which phenyl and 5- to 6-membered heteroaryl may optionally be mono- or disubstituted by identical or different substituents from the group consisting of fluorine, chlorine, cyano, trifluoromethyl-, methyl or methoxy-,
or represents C₃-C₈-cycloalkyl which may optionally be mono- or disubstituted by identical or different substituents from the group consisting of hydroxy, oxo, —NR¹⁰R¹¹ and 5- to 6-membered heterocycloalkyl,
or represents 4- to 8-membered heterocycloalkyl which may optionally be mono- or disubstituted by identical or different substituents from the group consisting of oxo, C₁-C₃-alkyl, —NR¹⁰R¹¹, C₁-C₄-alkylcarbonyl- and C₁-C₄-alkoxycarbonyl-.

Preference is given to compounds of the general formula (I) in which R⁸ represents C₁-C₄-alkyl which may optionally be monosubstituted by —NR¹⁰R¹¹, 4- to 8-membered heterocycloalkyl, phenyl or 5- to 6-membered heteroaryl,

in which the 4- to 8-membered heterocycloalkyl may optionally be monosubstituted by oxo, C₁-C₄-alkyl or C₁-C₄-alkoxycarbonyl-,
and in which phenyl and 5- to 6-membered heteroaryl may optionally be mono- or disubstituted by identical or different substituents from the group consisting of fluorine, chlorine, cyano, trifluoromethyl-, methyl and methoxy-.

Preference is given to compounds of the general formula (I) in which R⁸ represents C₃-C₈-cycloalkyl which may optionally be mono- or disubstituted by identical or different substituents from the group consisting of hydroxy, oxo, —NR¹⁰R¹¹ and 5- to 6-membered heterocycloalkyl.

Preference is given to compounds of the general formula (I) in which R⁸ represents 4- to 8-membered heterocycloalkyl which may optionally be mono- or disubstituted by identical or different substituents from the group consisting of oxo, C₁-C₃-alkyl, —NR¹⁰R¹¹, C₁-C₄-alkylcarbonyl- and C₁-C₄-alkoxycarbonyl-.

Preference is given to compounds of the general formula (I) in which R⁸ represents C₁-C₄-alkyl or represents C₃-C₆-cycloalkyl which may optionally be monosubstituted by —NR¹⁰R¹¹ or 4- to 8-membered heterocycloalkyl,

or represents 4- to 8-membered heterocycloalkyl,
in which C₃-C₆-cycloalkyl or 4-8-membered heterocycloalkyl may optionally be monosubstituted by oxo, and in which the 4-8-membered heterocycloalkyl may optionally contain one or more further heteroatoms.

Preference is given to compounds of the general formula (I) in which R⁸ represents a C₁-C₄-alkyl group which may optionally be monosubstituted by —NR¹⁰R¹¹ or a 4-8-membered heterocycloalkyl group which may optionally contain one or more further heteroatoms and may optionally be substituted by oxo.

Preference is given to compounds of the general formula (I) in which R⁸ represents a C₃-C₆-cycloalkyl group which may optionally be monosubstituted by —NR¹⁰R¹¹ or oxo.

Preference is given to compounds of the general formula (I) in which R⁸ represents a C₃-C₆-cycloalkyl group which may optionally be monosubstituted by —NR¹⁰R¹¹.

Preference is given to compounds of the general formula (I) in which R⁸ represents a C₃-C₆-cycloalkyl group which may optionally be monosubstituted by oxo.

Preference is given to compounds of the general formula (I) in which R⁸ represents a C₃-C₆-cycloalkyl group.

Preference is given to compounds of the general formula (I) in which R⁸ represents a 4- to 8-membered heterocycloalkyl group which may optionally contain one or more further heteroatoms and may optionally be monosubstituted by oxo.

Preference is given to compounds of the general formula (I) in which R⁸ represents a 4- to 7-membered heterocycloalkyl group which may optionally contain one or more further heteroatoms and may optionally be monosubstituted by oxo.

Preference is given to compounds of the general formula (I) in which R⁸ represents a 5- to 6-membered heterocycloalkyl group which may optionally contain one or more further heteroatoms and may optionally be monosubstituted by oxo.

Preference is given to compounds of the general formula (I) in which R⁸ represents a C₆-C₈-heterospirocycloalkyl group which may optionally contain one or more further heteroatoms and may optionally be monosubstituted by oxo.

Preference is given to compounds of the general formula (I) in which R⁸ represents a C₆-C₁₀-heterobicycloalkyl group which may optionally contain one or more further heteroatoms and may optionally be monosubstituted by oxo.

Preference is given to compounds of the general formula (I) in which R⁸ represents a bridged C₆-C₁₀-heterocycloalkyl group which may optionally contain one or more further heteroatoms and may optionally be monosubstituted by oxo.

Preference is given to compounds of the general formula (I) in which R⁸ represents C₁-C₂-alkyl which may optionally be monosubstituted by N,N-dimethylamino-, N-ethyl-N-methylamino-, N,N-diethylamino-, pyrrolidinyl, piperidinyl, piperazinyl, morpholinyl, phenyl or pyridinyl,

in which pyrrolidinyl, piperidinyl, piperazinyl and morpholinyl may optionally be monosubstituted by methyl, ethyl or tert-butoxycarbonyl-,
and in which phenyl and pyridinyl may optionally be monosubstituted by fluorine, chlorine, methyl or methoxy-,
or represents C₅-C₆-cycloalkyl which may optionally be monosubstituted by hydroxy, oxo, —NR¹⁰R¹¹, pyrrolidinyl, piperidinyl, piperazinyl, morpholinyl,
or represents oxetanyl, azetidinyl, pyrrolidinyl, tetrahydrofuranyl or piperidinyl which may optionally be monosubstituted by methyl, ethyl or acetyl-.

Preference is given to compounds of the general formula (I) in which R⁸ represents C₁-C₂-alkyl which may optionally be monosubstituted by N,N-dimethylamino-, N-ethyl-N-methylamino-, N,N-diethylamino-, pyrrolidinyl, piperidinyl, piperazinyl, morpholinyl, phenyl or pyridinyl,

in which pyrrolidinyl, piperidinyl, piperazinyl and morpholinyl may optionally be monosubstituted by methyl, ethyl or tert-butoxycarbonyl-
and in which phenyl and pyridinyl may optionally be monosubstituted by fluorine, chlorine, methyl or methoxy.

Preference is given to compounds of the general formula (I) in which R⁸ represents C₁-C₂-alkyl which may optionally be monosubstituted by N,N-dimethylamino-, piperazinyl, morpholinyl, phenyl or pyridinyl,

in which piperazinyl and morpholinyl may optionally be monosubstituted by methyl or tert-butoxycarbonyl-.

Preference is given to compounds of the general formula (I) in which R⁸ represents C₅-C₆-cycloalkyl which may optionally be monosubstituted by hydroxy, oxo, —NR¹⁰R¹¹, pyrrolidinyl, piperidinyl, piperazinyl, morpholinyl.

Preference is given to compounds of the general formula (I) in which R⁸ represents oxetanyl, azetidinyl, pyrrolidinyl, tetrahydrofuranyl or piperidinyl which may optionally be monosubstituted by methyl, ethyl or acetyl-.

Particular preference is given to compounds of the general formula (I) in which R⁸ represents a groups selected from

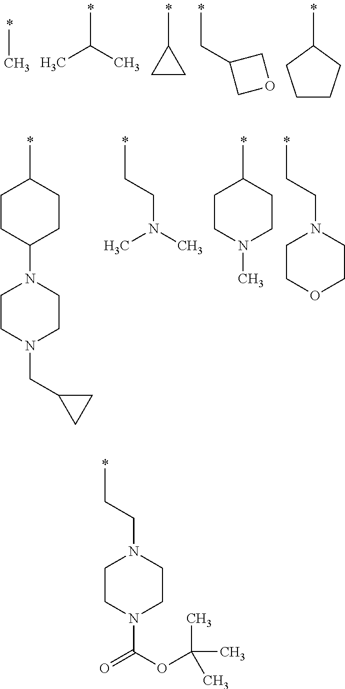

in which “*” indicates the point of attachment to the nitrogen atom in —C(═O)NR⁸R⁹ and —S(═O)₂NR⁸R⁹, respectively.

Particular preference is given to compounds of the general formula (I) in which R⁸ represents a groups selected from

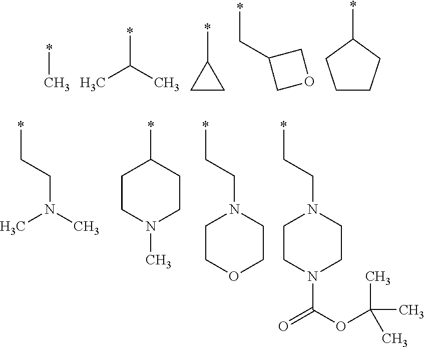

in which “*” indicates the point of attachment to the nitrogen atom in —C(═O)NR⁸R⁹ and —S(═O)₂NR⁸R⁹, respectively.

Particular preference is given to compounds of the general formula (I) in which R⁸ represents a groups selected from

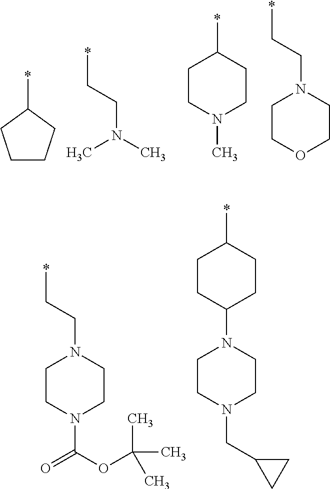

in which “*” indicates the point of attachment to the nitrogen atom in —C(═O)NR⁸R⁹ and —S(═O)₂NR⁸R⁹, respectively.

Particular preference is given to compounds of the general formula (I) in which R⁸ represents one of the groups below

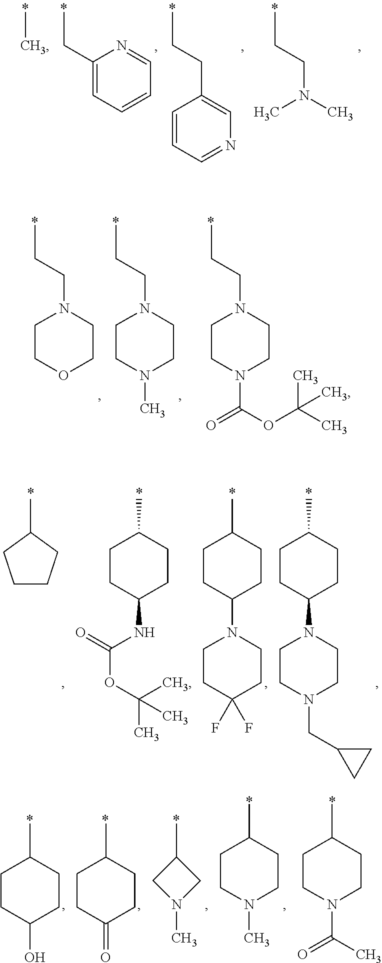

in which “*” indicates the point of attachment to the nitrogen atom in —C(═O)NR⁸R⁹ and —S(═O)₂NR⁸R⁹, respectively.

Preference is given to compounds of the general formula (I) in which R⁹ represents hydrogen or methyl.

Preference is given to compounds of the general formula (I) in which R⁹ represents hydrogen.

Preference is given to compounds of the general formula (I) in which R⁹ represents methyl.

Preference is given to compounds of the general formula (I) in which R⁸ and R⁹ together with the nitrogen atom to which they are attached represent 5- to 6-membered heterocycloalkyl or C₆-C₈-heterospirocycloalkyl which may optionally be mono- or disubstituted by identical or different substituents from the group consisting of oxo, fluorine, C₁-C₃-alkyl, C₃-C₅-cycloalkyl, —NR¹⁰R¹¹, C₁-C₄-alkylcarbonyl- and C₁-C₄-alkoxycarbonyl-.

Preference is given to compounds of the general formula (I) in which R⁸ and R⁹ together with the nitrogen atom to which they are attached represent 5- to 6-membered heterocycloalkyl which may optionally be mono- or disubstituted by identical or different substituents from the group consisting of oxo, fluorine, C₁-C₃-alkyl, C₃-C₅-cycloalkyl, —NR¹⁰R¹¹, C₁-C₄-alkylcarbonyl- and C₁-C₄-alkoxycarbonyl-.

Preference is given to compounds of the general formula (I) in which R⁸ and R⁹ together with the nitrogen atom to which they are attached represent C₆-C₈-heterospirocycloalkyl which may optionally be mono- or disubstituted by identical or different substituents from the group consisting of oxo, fluorine, C₁-C₃-alkyl, C₃-C₅-cycloalkyl, —NR¹⁰R¹¹, C₁-C₄-alkylcarbonyl- and C₁-C₄-alkoxycarbonyl-.

Preference is given to compounds of the general formula (I) in which R⁸ and R⁹ together with the nitrogen atom to which they are attached represent pyrrolidinyl, piperidinyl, piperazinyl, morpholinyl, 1-thia-6-azaspiro[3.3]hept-6-yl- or 2-oxa-6-azaspiro[3.3]hept-6-yl- which may optionally be mono- or disubstituted by identical or different substituents from the group consisting of oxo, fluorine, C₁-C₃-alkyl, cyclopropyl, piperidin-1-yl and tert-butoxycarbonyl-.

Preference is given to compounds of the general formula (I) in which R⁸ and R⁹ together with the nitrogen atom to which they are attached represent 4- to 7-membered heterocycloalkyl which may optionally contain one or more further heteroatoms and may optionally be monosubstituted by oxo or C₁-C₃-alkyl.

Preference is given to compounds of the general formula (I) in which R⁸ and R⁹ together with the nitrogen atom to which they are attached represent 5- or 6-membered heterocycloalkyl which may optionally contain one or more further heteroatoms and may optionally be monosubstituted by oxo or C₁-C₃-alkyl.

Preference is given to compounds of the general formula (I) in which NR⁸R⁹ represents 6- to 8-membered heterospirocycloalkyl which may optionally contain one or more further heteroatoms and may optionally be monosubstituted by oxo or C₁-C₃-alkyl.

Particular preference is given to compounds of the general formula (I) in which R⁸ and R⁹ together with the nitrogen atom to which they are attached represent a group selected from

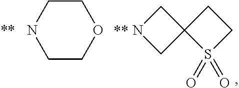

in which “**” indicates the point of attachment to the carbonyl or sulphonyl group present in R¹.

Particular preference is given to compounds of the general formula (I) in which R⁸ and R⁹ together with the nitrogen atom to which they are attached represent a group selected from

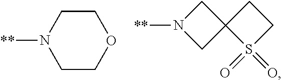

in which “**” indicates the point of attachment to the carbonyl or sulphonyl group present in R¹.

Particular preference is given to compounds of the general formula (I) in which R⁸ and R⁹ together with the nitrogen atom to which they are attached represent a group

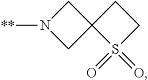

in which “**” indicates the point of attachment to the carbonyl or sulphonyl group present in R¹.

Particular preference is given to compounds of the general formula (I) in which R⁸ represents one of the groups below

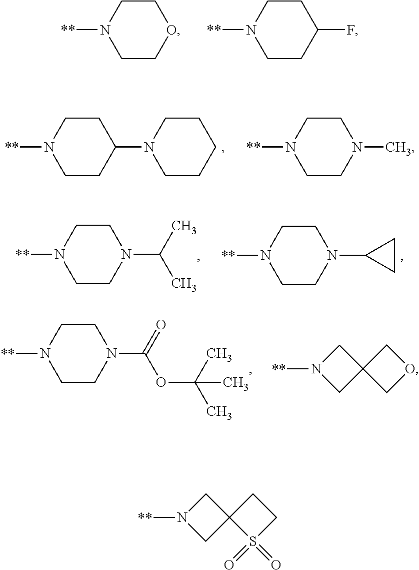

in which “**” indicates the point of attachment to the carbonyl or sulphonyl group present in R¹.

Preference is given to compounds of the general formula (I) in which n represents the number 0.

Preference is given to compounds of the general formula (I) in which n represents the number 1.

Preference is given to compounds of the general formula (I) in which R¹⁰ and R¹¹ independently of one another represent hydrogen or C₁-C₄-alkyl which is optionally substituted by hydroxy or fluorine.

Preference is given to compounds of the general formula (I) in which R¹⁰ and R¹¹ independently of one another represent hydrogen or C₁-C₃-alkyl which is optionally substituted by hydroxy or fluorine.

Preference is given to compounds of the general formula (I) in which R¹⁰ and R¹¹ independently of one another represent hydrogen or represent C₁-C₄-alkyl which is optionally mono-, di- or trisubstituted by identical or different substituents from the group consisting of hydroxy, oxo and fluorine,

or represent C₁-C₄-alkylcarbonyl- or C₁-C₄-alkoxycarbonyl-.

Preference is given to compounds of the general formula (I) in which R¹⁰ and R¹¹ independently of one another represent hydrogen or represent C₁-C₄-alkyl which is optionally mono-, di- or trisubstituted by identical or different substituents from the group consisting of hydroxy, oxo and fluorine.

Preference is given to compounds of the general formula (I) in which R¹⁰ and R¹¹ independently of one another represent hydrogen or represent C₁-C₄-alkylcarbonyl- or C₁-C₄-alkoxycarbonyl-.

Preference is given to compounds of the general formula (I) in which R¹⁰ and R¹¹ independently of one another represent hydrogen, C₁-C₄-alkyl or represent C₁-C₄-alkoxycarbonyl-.

Preference is given to compounds of the general formula (I) in which R¹⁰ and R¹¹ independently of one another represent hydrogen or C₁-C₄-alkyl.

Preference is given to compounds of the general formula (I) in which R¹⁰ and R¹¹ independently of one another represent hydrogen or C₁-C₄-alkoxycarbonyl-.

Preference is given to compounds of the general formula (I) in which R¹⁰ and R¹¹ independently of one another represent hydrogen, C₁-C₃-alkyl or tert-butoxycarbonyl-.

Preference is given to compounds of the general formula (I) in which R¹⁰ and R¹¹ independently of one another represent hydrogen or C₁-C₃-alkyl.

Preference is given to compounds of the general formula (I) in which R¹⁰ and R¹¹ independently of one another represent hydrogen or tert-butoxycarbonyl-.

Particular preference is given to compounds of the general formula (I) in which R¹⁰ represents hydrogen or C₁-C₄-alkyl.

Particular preference is given to compounds of the general formula (I) in which R¹¹ represents hydrogen or C₁-C₄-alkyl.

Particular preference is given to compounds of the general formula (I) in which R¹⁰ represents hydrogen, methyl or ethyl.

Particular preference is given to compounds of the general formula (I) in which R¹¹ represents hydrogen, methyl or ethyl.

Preference is given to compounds of the general formula (I) in which R¹⁰ represents C₁-C₄-alkoxycarbonyl- and R¹¹ represents hydrogen.

Particular preference is given to compounds of the general formula (I) in which R¹⁰ and R¹¹ independently of one another represent hydrogen, C₁-C₃-alkyl or tert-butoxycarbonyl-.

Preference is given to compounds of the general formula (I) in which R¹⁰ represents C₁-C₄-alkyl and R¹¹ represents hydrogen.

Preference is given to compounds of the general formula (I) in which R¹⁰ represents C₁-C₂-alkyl and R¹¹ represents hydrogen.

Preference is given to compounds of the general formula (I) in which R¹⁰ represents methyl and R¹¹ represents hydrogen.

Preference is given to compounds of the general formula (I) in which R¹⁰ represents tert-butoxycarbonyl- and R¹¹ represents hydrogen.

Preference is given to compounds of the general formula (I) in which R¹⁰ represents C₁-C₃-alkyl and R¹¹ represents C₁-C₃-alkyl.

Particular preference is given to compounds of the general formula (I) in which R¹⁰ represents C₁-C₂-alkyl and R¹¹ represents C₁-C₂-alkyl.

Particular preference is given to compounds of the general formula (I) in which R¹⁰ represents methyl and R¹¹ represents methyl.

Preference is given to compounds of the general formula (I) in which R¹⁰ and R¹¹ together with the nitrogen atom to which they are attached represent 4- to 7-membered heterocycloalkyl which may optionally be mono- or disubstituted by identical or different substituents from the group consisting of hydroxy, oxo, cyano, fluorine, C₁-C₃-alkyl, fluoro-C₁-C₃-alkyl-, C₃-C₆-cycloalkyl, C₃-C₆-cycloalkylmethyl-, benzyl and C₁-C₄-alkoxycarbonyl-.

Preference is given to compounds of the general formula (I) in which R¹⁰ and R¹¹ together with the nitrogen atom to which they are attached represent 4- to 7-membered heterocycloalkyl which may optionally contain one or more further heteroatoms and may optionally be mono- or disubstituted by identical or different substituents from the group consisting of hydroxy, cyano, fluorine, C₁-C₃-alkyl, cyclopropyl, cyclopropylmethyl-, benzyl or C₁-C₄-alkoxycarbonyl-.

Preference is given to compounds of the general formula (I) in which R¹⁰ and R¹¹ together with the nitrogen atom to which they are attached represent 4- to 7-membered heterocycloalkyl which may optionally contain one or more further heteroatoms and may optionally carry one or two substituents independently of one another selected from the group consisting of hydroxy, oxo, cyano, fluorine and C₁-C₃-alkyl.

Preference is given to compounds of the general formula (I) in which R¹⁰ and R¹¹ together with the nitrogen atom to which they are attached represent 5- to 6-membered heterocycloalkyl which may optionally be mono- or disubstituted by identical or different substituents from the group consisting of oxo, fluorine, C₁-C₃-alkyl, fluoro-C₁-C₃-alkyl-, C₃-C₅-cycloalkyl, C₃-C₅-cycloalkylmethyl- and C₁-C₄-alkoxycarbonyl-.

Particular preference is given to compounds of the general formula (I) in which R¹⁰ and R¹¹ together with the nitrogen atom to which they are attached represent 5- to 6-membered heterocycloalkyl which may optionally be monosubstituted by C₁-C₃-alkyl, cyclopropyl, cyclopropylmethyl-, benzyl or tert-butoxycarbonyl-.

Particular preference is given to compounds of the general formula (I) in which R¹⁰ and R¹¹ together with the nitrogen atom to which they are attached represent pyrrolidinyl, piperidinyl, piperazinyl or morpholinyl which may optionally be mono- or disubstituted by identical or different substituents from the group consisting of fluorine, 2,2,2-trifluoroethyl-, cyclopropyl, cyclopropylmethyl- and tert-butoxycarbonyl-.

The specific radical definitions given in the particular combinations or preferred combinations of radicals are, irrespective of the particular combinations of radicals specified, also replaced as desired by radical definitions of other combinations.

Very particular preference is given to combinations of two or more of the abovementioned preferred ranges.

Very particular preference is given to the following compounds of the general formula (I):
4-{[(3R)-4-cyclopentyl-1,3-dimethyl-2-oxo-1,2,3,4-tetrahydropyrido[2,3-b]pyrazin-6-yl]amino}-3-methoxy-N-(1-methylpiperidin-4-yl)benzamide;
4-{[(3R)-1,3-dimethyl-2-oxo-4-(propan-2-yl)-1,2,3,4-tetrahydropyrido[2,3-b]pyrazin-6-yl]amino}-3-methoxy-N-(1-methylpiperidin-4-yl)benzamide;
4-{[(3R)-1,3-dimethyl-2-oxo-4-(propan-2-yl)-1,2,3,4-tetrahydropyrido[2,3-b]pyrazin-6-yl]amino}-3-methoxy-N-[2-(morpholin-4-yl)ethyl]benzamide;
1-tert-butyl 4-{2-[(4-{[(3R)-1,3-dimethyl-2-oxo-4-(propan-2-yl)-1,2,3,4-tetrahydropyrido[2,3-b]pyrazin-6-yl]amino}-3-methoxybenzoyl)amino]ethyl}piperazinecarboxylate;
N-[2-(dimethylamino)ethyl]-4-{[(3R)-1,3-dimethyl-2-oxo-4-(propan-2-yl)-1,2,3,4-tetrahydropyrido[2,3-b]pyrazin-6-yl]amino}-3-methoxybenzamide;
N-cyclopentyl-4-{[(3R)-1,3-dimethyl-2-oxo-4-(propan-2-yl)-1,2,3,4-tetrahydropyrido[2,3-b]pyrazin-6-yl]amino}-3-methoxybenzamide;
(3R)-4-cyclopentyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one;
(3R)-6-({4-[(1,1-dioxido-1-thia-6-azaspiro[3.3]hept-6-yl)carbonyl]-2-methoxyphenyl}amino)-4-isopropyl-1,3-dimethyl-3,4-dihydropyrido[2,3-b]pyrazin-2(1H)-one;
(3R)-6-({4-[(1,1-dioxido-1-thia-6-azaspiro[3.3]hept-6-yl)carbonyl]phenyl}amino)-4-isopropyl-1,3-dimethyl-3,4-dihydropyrido[2,3-b]pyrazin-2(1H)-one;
(3R)-4-cyclopentyl-6-({4-[(1,1-dioxido-1-thia-6-azaspiro[3.3]hept-6-yl)carbonyl]-2-methoxyphenyl}amino)-1,3-dimethyl-3,4-dihydropyrido[2,3-b]pyrazin-2(1H)-one;
4-{[(3R)-4-cyclopentyl-1,3-dimethyl-2-oxo-1,2,3,4-tetrahydropyrido[2,3-b]pyrazin-6-yl]amino}-N-(1-methylpiperidin-4-yl)benzamide;
4-{[(3R)-4-cyclohexyl-1,3-dimethyl-2-oxo-1,2,3,4-tetrahydropyrido[2,3-b]pyrazin-6-yl]amino}-3-methoxy-N-(1-methylpiperidin-4-yl)benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(1-methylpiperidin-4-yl)benzamide;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzamide;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
(3R)-1,3-dimethyl-6-[(4-{[4-(propan-2-yl)piperazin-1-yl]sulphonyl}phenyl)amino]-4-(tetrahydro-2H-pyran-4-yl)-3,4-dihydropyrido[2,3-b]pyrazin-2(1H)-one;
4-{[(3R)-4-cyclohexyl-1,3-dimethyl-2-oxo-1,2,3,4-tetrahydropyrido[2,3-b]pyrazin-6-yl]amino}-N,N-dimethylbenzenesulphonamide;
(3R)-1,3-dimethyl-6-{[4-(morpholin-4-ylsulphonyl)phenyl]amino}-4-(tetrahydro-2H-pyran-4-yl)-3,4-dihydropyrido[2,3-b]pyrazin-2(1H)-one;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-[2-(pyridin-3-yl)ethyl]benzenesulphonamide;
(3R)-1,3-dimethyl-6-({4-[(4-methylpiperazin-1-yl)sulphonyl]phenyl}amino)-4-(tetrahydro-2H-pyran-4-yl)-3,4-dihydropyrido[2,3-b]pyrazin-2(1H)-one;
(3R)-6-({2-fluoro-4-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzenesulphonamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-[2-(4-methylpiperazin-1-yl)ethyl]benzenesulphonamide;
N-[2-(dimethylamino)ethyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(pyridin-2-ylmethyl)benzenesulphonamide;
(3R)-6-({3-methoxy-4-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N,N-dimethylbenzenesulphonamide;
(3R)-4-cyclohexyl-6-[(2-methoxy-4-{[4-(propan-2-yl)piperazin-1-yl]carbonyl}phenyl)amino]-1,3-dimethyl-3,4-dihydropyrido[2,3-b]pyrazin-2(1H)-one;
(3R)-4-cyclohexyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one;
4-{[(3R)-4-cyclohexyl-1,3-dimethyl-2-oxo-1,2,3,4-tetrahydropyrido[2,3-b]pyrazin-6-yl]amino}-N-(1-methylazetidin-3-yl)benzamide;
(3R)-4-cyclohexyl-1,3-dimethyl-6-[(4-{[4-(propan-2-yl)piperazin-1-yl]carbonyl}phenyl)amino]-3,4-dihydropyrido[2,3-b]pyrazin-2(1H)-one;
(3R)-4-cycloheptyl-6-{[2-methoxy-4-(2-oxa-6-azaspiro[3.3]hept-6-ylcarbonyl)phenyl]amino}-1,3-dimethyl-3,4-dihydropyrido[2,3-b]pyrazin-2(1H)-one;
(3R)-4-cycloheptyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one;
4-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}-3-methoxybenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(pyridin-2-ylmethyl)benzamide;
(3R)-1,3-dimethyl-6-({2-methyl-4-[(4-methylpiperazin-1-yl)carbonyl]phenyl}amino)-4-(tetrahydro-2H-pyran-4-yl)-3,4-dihydropyrido[2,3-b]pyrazin-2(1H)-one;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(4-oxocyclohexyl)benzamide;
N-(1-acetylpiperidin-4-yl)-4-{[(3R)-4-cyclohexyl-1,3-dimethyl-2-oxo-1,2,3,4-tetrahydropyrido[2,3-b]pyrazin-6-yl]amino}-3-methoxybenzamide;
(3R)-4-cycloheptyl-6-[(2-methoxy-4-{[4-(propan-2-yl)piperazin-1-yl]carbonyl}phenyl)amino]-1,3-dimethyl-3,4-dihydropyrido[2,3-b]pyrazin-2(1H)-one;
(3R)-4-benzyl-1,3-dimethyl-6-{[4-(2-oxa-6-azaspiro[3.3]hept-6-ylcarbonyl)phenyl]amino}-3,4-dihydropyrido[2,3-b]pyrazin-2(1H)-one;
4-{[(3R)-4-benzyl-1,3-dimethyl-2-oxo-1,2,3,4-tetrahydropyrido[2,3-b]pyrazin-6-yl]amino}-N-(4-hydroxycyclohexyl)benzamide;
(3R)-4-benzyl-6-({4-[(4-fluoropiperidin-1-yl)carbonyl]-2-methoxyphenyl}amino)-1,3-dimethyl-3,4-dihydropyrido[2,3-b]pyrazin-2(1H)-one;
4-{[(3R)-4-cycloheptyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide;
(3R)-6-({2-methoxy-4-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one;
N-[2-(dimethylamino)ethyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(pyridin-2-ylmethyl)benzamide;
N-[2-(dimethylamino)ethyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-[2-(pyridin-3-yl)ethyl]benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylazetidin-3-yl)benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-[2-(4-methylpiperazin-1-yl)ethyl]benzamide;
N-[4-(4,4-difluoropiperidin-1-yl)cyclohexyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}benzamide;
(3R)-6-{[4-(1,4′-bipiperidin-1′-ylcarbonyl)-2-methoxyphenyl]amino}-1,3-dimethyl-4-(tetrahydro-2H-pyran-4-yl)-3,4-dihydropyrido[2,3-b]pyrazin-2(1H)-one;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methyl-N-(1-methylpiperidin-4-yl)benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(1-methylazetidin-3-yl)benzamide;
(3R)-1,3-dimethyl-6-({4-[(4-methylpiperazin-1-yl)carbonyl]phenyl}amino)-4-(tetrahydro-2H-pyran-4-yl)-3,4-dihydropyrido[2,3-b]pyrazin-2(1H)-one;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzamide;
tert-butyl {trans-4-[(4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzoyl)amino]cyclohexyl}carbamate;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(4-hydroxycyclohexyl)-3-methoxybenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-[2-(pyridin-3-yl)ethyl]benzamide;
N-[4-(4,4-difluoropiperidin-1-yl)cyclohexyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzamide;
N-[2-(dimethylamino)ethyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-[2-(4-methylpiperazin-1-yl)ethyl]benzamide;
(3R)-6-({trans-4-[(4-cyclopropylpiperazin-1-yl)carbonyl]-2-methoxyphenyl}amino)-1,3-dimethyl-4-(tetrahydro-2H-pyran-4-yl)-3,4-dihydropyrido[2,3-b]pyrazin-2(1H)-one;
4-{[(3R)-4-cyclohexyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
4-{[(3R)-4-cyclohexyl-1,3-dimethyl-2-oxo-1,2,3,4-tetrahydropyrido[2,3-b]pyrazin-6-yl]amino}-3-methoxy-N-[2-(4-methylpiperazin-1-yl)ethyl]benzamide;
4-{[(3R)-4-cyclohexyl-1,3-dimethyl-2-oxo-1,2,3,4-tetrahydropyrido[2,3-b]pyrazin-6-yl]amino}-N-{trans-4-[4-(cyclopropylmethyl) piperazin-1-yl]cyclohexyl}benzamide;
(3R)-4-cyclohexyl-1,3-dimethyl-6-{[4-(2-oxa-6-azaspiro[3.3]hept-6-ylcarbonyl)phenyl]amino}-3,4-dihydropyrido[2,3-b]pyrazin-2(1H)-one;
4-{[(3R)-4-cyclohexyl-1,3-dimethyl-2-oxo-1,2,3,4-tetrahydropyrido[2,3-b]pyrazin-6-yl]amino}-N-{trans-4-[4-(cyclopropylmethyl) piperazin-1-yl]cyclohexyl}-3-methoxybenzamide;
4-{[(3R)-4-cyclohexyl-1,3-dimethyl-2-oxo-1,2,3,4-tetrahydropyrido[2,3-b]pyrazin-6-yl]amino}-N-[4-(4,4-difluoropiperidin-1-yl)cyclohexyl]benzamide;
4-{[(3R)-4-cyclohexyl-1,3-dimethyl-2-oxo-1,2,3,4-tetrahydropyrido[2,3-b]pyrazin-6-yl]amino}-3-methoxy-N-(1-methylazetidin-3-yl)benzamide;
(3R)-4-cyclohexyl-6-{[2-methoxy-4-(2-oxa-6-azaspiro[3.3]hept-6-ylcarbonyl)phenyl]amino}-1,3-dimethyl-3,4-dihydropyrido[2,3-b]pyrazin-2(1H)-one;
(3R)-4-cyclohexyl-6-({4-[(1,1-dioxido-1-thia-6-azaspiro[3.3]hept-6-yl)carbonyl]-2-methoxyphenyl}amino)-1,3-dimethyl-3,4-dihydropyrido[2,3-b]pyrazin-2(1H)-one;
(3R)-4-benzyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one;
tert-butyl 4-(4-{[(3R)-4-benzyl-1,3-dimethyl-2-oxo-1,2,3,4-tetrahydropyrido[2,3-b]pyrazin-6-yl]amino}benzoyl)piperazine-1-carboxylate;
N-(1-acetylpiperidin-4-yl)-4-{[(3R)-4-benzyl-1,3-dimethyl-2-oxo-1,2,3,4-tetrahydropyrido[2,3-b]pyrazin-6-yl]amino}-3-methoxybenzamide;
N-(1-acetylpiperidin-4-yl)-4-{[(3R)-4-benzyl-1,3-dimethyl-2-oxo-1,2,3,4-tetrahydropyrido[2,3-b]pyrazin-6-yl]amino}benzamide;
4-{[(3R)-4-benzyl-1,3-dimethyl-2-oxo-1,2,3,4-tetrahydropyrido[2,3-b]pyrazin-6-yl]amino}-N-(4-hydroxycyclohexyl)-3-methoxybenzamide;
(3R)-4-benzyl-6-[(2-methoxy-4-{[4-(propan-2-yl)piperazin-1-yl]carbonyl}phenyl)amino]-1,3-dimethyl-3,4-dihydro pyrido[2,3-b]pyrazin-2(1H)-one;
4-{[(3R)-4-benzyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
4-{[(3R)-4-benzyl-1,3-dimethyl-2-oxo-1,2,3,4-tetrahydropyrido[2,3-b]pyrazin-6-yl]amino}-3-methoxy-N-[2-(4-methylpiperazin-1-yl)ethyl]benzamide;
(3R)-4-benzyl-6-{[2-methoxy-4-(2-oxa-6-azaspiro[3.3]hept-6-ylcarbonyl)phenyl]amino}-1,3-dimethyl-3,4-dihydropyrido[2,3-b]pyrazin-2(1H)-one;
(3R)-4-cycloheptyl-6-({4-[(1,1-dioxido-1-thia-6-azaspiro[3.3]hept-6-yl)carbonyl]-2-methoxyphenyl}amino)-1,3-dimethyl-3,4-dihydropyrido[2,3-b]pyrazin-2(1H)-one;
4-{[(3R)-4-cycloheptyl-1,3-dimethyl-2-oxo-1,2,3,4-tetrahydropyrido[2,3-b]pyrazin-6-yl]amino}-N-(1-methylazetidin-3-yl)benzamide;
N-(1-acetylpiperidin-4-yl)-4-{[(3R)-4-cycloheptyl-1,3-dimethyl-2-oxo-1,2,3,4-tetrahydropyrido[2,3-b]pyrazin-6-yl]amino}benzamide;
4-{[(3R)-4-cycloheptyl-1,3-dimethyl-2-oxo-1,2,3,4-tetrahydropyrido[2,3-b]pyrazin-6-yl]amino}-3-methoxy-N-(1-methylazetidin-3-yl)benzamide;
N-(1-acetylpiperidin-4-yl)-4-{[(3R)-4-cycloheptyl-1,3-dimethyl-2-oxo-1,2,3,4-tetrahydropyrido[2,3-b]pyrazin-6-yl]amino}-3-methoxybenzamide;
4-{[(3R)-4-cycloheptyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
4-{[(3R)-4-cycloheptyl-1,3-dimethyl-2-oxo-1,2,3,4-tetrahydropyrido[2,3-b]pyrazin-6-yl]amino}-N-(4-hydroxycyclohexyl)benzamide;
4-{[(3R)-4-cycloheptyl-1,3-dimethyl-2-oxo-1,2,3,4-tetrahydropyrido[2,3-b]pyrazin-6-yl]amino}-3-methoxy-N-[2-(4-methylpiperazin-1-yl)ethyl]benzamide;
(3R)-4-cycloheptyl-1,3-dimethyl-6-[(4-{[4-(propan-2-yl)piperazin-1-yl]carbonyl}phenyl)amino]-3,4-dihydropyrido[2,3-b]pyrazin-2(1H)-one;
(3R)-4-cycloheptyl-1,3-dimethyl-6-{[4-(2-oxa-6-azaspiro[3.3]hept-6-ylcarbonyl)phenyl]amino}-3,4-dihydropyrido[2,3-b]pyrazin-2(1H)-one;
4-{[(3R)-4-benzyl-1,3-dimethyl-2-oxo-1,2,3,4-tetrahydropyrido[2,3-b]pyrazin-6-yl]amino}-N,N-dimethylbenzenesulphonamide;
4-{[(3R)-4-cycloheptyl-1,3-dimethyl-2-oxo-1,2,3,4-tetrahydropyrido[2,3-b]pyrazin-6-yl]amino}-N,N-dimethylbenzenesulphonamide;
N-{trans-4-[4-(cyclopropylmethyl)piperazin-1-yl]cyclohexyl}-4-{[(3R)-1,3-dimethyl-2-oxo-4-(propan-2-yl)-1,2,3,4-tetrahydropyrido[2,3-b]pyrazin-6-yl]amino)}benzamide;
4-{[(3R)-1,3-dimethyl-2-oxo-4-(propan-2-yl)-1,2,3,4-tetrahydropyrido[2,3-b]pyrazin-6-yl]amino}-N-(4-hydroxycyclohexyl)-3-methoxybenzamide;
4-{[(3R)-1,3-dimethyl-2-oxo-4-(propan-2-yl)-1,2,3,4-tetrahydropyrido[2,3-b]pyrazin-6-yl]amino}-N-(1-methylpiperidin-4-yl)benzamide;
(3R)-1,3-dimethyl-6-({4-[(4-methylpiperazin-1-yl) sulphonyl]phenyl}amino)-4-(propan-2-yl)-3,4-dihydropyrido[2,3-b]pyrazin-2(1H)-one;
4-{[(3R)-1,3-dimethyl-2-oxo-4-(propan-2-yl)-1,2,3,4-tetrahydropyrido[2,3-b]pyrazin-6-yl]amino}-N,N-dimethylbenzenesulphonamide;
(3R)-1,3-dimethyl-6-{[4-(morpholin-4-ylsulphonyl)phenyl]amino}-4-(propan-2-yl)-3,4-dihydropyrido[2,3-b]pyrazin-2(1H)-one;
(3R)-4-cyclopentyl-1,3-dimethyl-6-[(4-{[4-(propan-2-yl)piperazin-1-yl]sulphonyl}phenyl)amino]-3,4-dihydropyrido[2,3-b]pyrazin-2(1H)-one;
4-{[(3R)-4-cyclopentyl-1,3-dimethyl-2-oxo-1,2,3,4-tetrahydropyrido[2,3-b]pyrazin-6-yl]amino}-N,N-dimethylbenzenesulphonamide;
4-{[4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide;
N-{trans-4-[4-(cyclopropylmethyl)piperazin-1-yl]cyclohexyl}-4-{[4-(2-methoxyethyl)-1,3-dimethyl-2-oxo-1,2,3,4-tetrahydropyrido[2,3-b]pyrazin-6-yl]amino}benzamide;
tert-butyl 4-[(3R)-6-{[4-(dimethylsulphamoyl)phenyl]amino}-1,3-dimethyl-2-oxo-2,3-dihydropyrido[2,3-b]pyrazin-4(1H)-yl]piperidine-1-carbonate;
4-[(1,3-dimethyl-2-oxo-4-phenyl-1,2,3,4-tetrahydropyrido[2,3-b]pyrazin-6-yl)amino]-N-(1-methylpiperidin-4-yl)benzenesulphonamide;
4-{[(3R)-1,3-dimethyl-2-oxo-4-phenyl-1,2,3,4-tetrahydropyrido[2,3-b]pyrazin-6-yl]amino}-N-(1-methylpiperidin-4-yl)benzenesulphonamide;
4-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzenesulphonamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(1-methylpiperidin-4-yl)benzenesulphonamide;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzenesulphonamide and
(3R)-6-({2-methoxy-4-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one,
   and the diastereomers, racemates, polymorphs and physiologically acceptable salts thereof.

### DEFINITIONS

C₁-C₆-Alkyl, or a C₁-C₆-alkyl group, is understood to mean a straight-chain or branched, saturated monovalent hydrocarbon radical, for example a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl or 1,2-dimethylbutyl radical. Preferably, C₁-C₆-alkyl, or a C₁-C₆-alkyl group, is understood to mean C₁-C₄-alkyl, C₂-C₄-alkyl or C₂-C₅-alkyl, particularly preferably C₁-C₃-alkyl or a methyl, ethyl, propyl or isopropyl radical.

C₂-C₅-Alkylene, or a C₂-C₅-alkylene group, is understood to mean a straight-chain or branched, saturated, bivalent hydrocarbon radical, for example an ethylene, propylene, butylene, pentylene, isopropylene, isobutylene, sec-butylene, tert-butylene, isopentylene, 2-methylbutylene, 1-methylbutylene, 1-ethylpropylene, 1,2-dimethylpropylene, neo-pentylene or 1,1-dimethylpropylene radical.

C₂-C₆-Alkenyl, or a C₂-C₆-alkenyl group, is understood to mean a straight-chain or branched, monovalent hydrocarbon radical having one or two C═C double bonds, for example an ethenyl, (E)-prop-2-enyl, (Z)-prop-2-enyl, allyl (prop-1-enyl), allenyl, buten-1-yl or buta-1,3-dienyl radical. Preference is given to C₃-C₆-alkenyl or C₂-C₄-alkenyl, particular preference to ethenyl and allyl.

C₂-C₆-Alkynyl, or a C₂-C₆-alkynyl group, is understood to mean a straight-chain or branched, monovalent hydrocarbon radical having one C═C triple bond, for example an ethynyl, propargyl (prop-1-ynyl) or butyn-1-yl radical. Preference is given to C₃-C₆-alkynyl or C₂-C₄-alkynyl, particular preference to ethynyl and propargyl.

C₁-C₄-Alkoxy, or a C₁-C₄-alkoxy group, is understood to mean a straight-chain or branched, saturated alkyl ether radical —O-alkyl, for example a methoxy, ethoxy, n-propoxy, isopropoxy or tert-butoxy radical.

Preferably, C₁-C₄-alkoxy, or a C₁-C₄-alkoxy group, is understood to mean C₁-C₃-alkoxy-, particularly preferably a methoxy or ethoxy radical.

C₁-C₄-Alkylthio, or a C₁-C₄-alkylthio group, is understood to mean a straight-chain or branched, saturated alkyl thioether radical —S-alkyl, for example a methylthio, ethylthio, n-propylthio, isopropylthio or tert-butylthio radical.

Preferably, C₁-C₄-alkylthio, or a C₁-C₄-alkylthio group, is understood to mean C₁-C₃-alkylthio-, particularly preferably a methylthio or ethylthio radical.

A heteroatom is understood to mean —O—, NH—, ═N— or —S—, including the oxidized forms thereof —S(═O)— and —S(═O)₂— and a sulphoximine —S(═O)(═NH)— derived from —S(═O)₂—. The heteroatom —NH— may optionally be substituted by C₁-C₃-alkyl, C₁-C₃-alkylcarbonyl-, C₁-C₄-alkoxycarbonyl- or —S(═O)₂—C₁-C₃-alkyl. The ═NH of the abovementioned sulphoximine may optionally be substituted by C₁-C₃-alkyl, C₁-C₃-alkylcarbonyl-, C₁-C₄-alkoxycarbonyl-.

Preference is given to an oxygen or nitrogen atom.

Oxo, or an oxo substituent, is understood to mean a double-bonded oxygen atom ═O. Oxo may be bonded to atoms of suitable valency, for example to a saturated carbon atom or to sulphur.

Preference is given to the bond to carbon to form a carbonyl group.

Preference is furthermore given to two doubly attached oxygen atoms being bonded to sulphur with formation of a sulphonyl group —(S═O)₂—.

Halogen is understood to mean fluorine, chlorine bromine or iodine.

Fluorine, chlorine bromine or iodine which is an optional substituent on the phenyl ring may be in the ortho, meta or para position. Preference is given to fluorine or chlorine.

The preferred position is the meta or para position.

A halo-C₁-C₄-alkyl radical is understood to mean a C₁-C₄-alkyl radical having at least one halogen substituent, preferably having at least one fluorine substituent.

Preference is given to fluoro-C₁-C₃-alkyl radicals, for example difluoromethyl-, trifluoromethyl-, 2,2,2-trifluoroethyl- or pentafluoroethyl-.

Particular preference is given to perfluorinated alkyl radicals such as trifluoromethyl- or pentafluoroethyl-.

Phenyl-C₁-C₃-alkyl is understood to mean a group composed of an optionally substituted phenyl radical and a C₁-C₃-alkyl group, and which is attached to the rest of the molecule via the C₁-C₃-alkyl group. Preference is given to benzyl.

C₃-C₆-Cycloalkyl-C₁-C₃-alkyl, or a C₃-C₆-cycloalkyl-C₁-C₃-alkyl group, is understood to mean a group which is composed of C₃-C₆-cycloalkyl as defined below and a C₁-C₃-alkyl group, and which is attached to the rest of the molecule via the C₁-C₃-alkyl group. Preference is given to C₃-C₆-cycloalkylmethyl-, particular preference to cyclopropylmethyl-.

A halo-C₁-C₄-alkoxy radical is understood to mean a C₁-C₄-alkoxy radical having at least one halogen substituent, preferably having at least one fluorine substituent.

Preference is given to fluoro-C₁-C₃-alkoxy radicals, for example difluoromethoxy, trifluoromethoxy or 2,2,2-trifluoroethoxy radicals.

A halo-C₁-C₄-alkylthio radical is understood to mean a C₁-C₄-alkylthio radical having at least one halogen substituent, preferably having at least one fluorine substituent.

Preference is given to fluoro-C₁-C₃-alkylthio radicals, in particular trifluoromethylthio-.

A C₁-C₄-alkylcarbonyl radical is understood to mean a C₁-C₄-alkyl-C(═O) group. Preference is given to acetyl or propanoyl.

A C₁-C₄-alkylcarbonyl radical is understood to mean a C₁-C₄-alkyl-C(═O) group. Preference is given to methoxycarbonyl, ethoxycarbonyl or tert-butoxycarbonyl.

A C₁-C₄-alkoxy-C₁-C₄-alkyl radical is understood to mean a C₁-C₄-alkoxy-substituted C₁-C₄-alkyl radical, for example methoxymethyl-, methoxyethyl-, ethoxymethyl- and ethoxyethyl-.

Aryl is understood to mean an unsaturated, fully conjugated system which is formed from carbon atoms and has 3, 5 or 7 conjugated double bonds, for example phenyl, naphthyl or phenanthryl. Preference is given to phenyl.

Heteroaryl is understood to mean ring systems which have an aromatically conjugated ring system and contain at least one and up to five heteroatoms as defined above. These ring systems may have 5, 6 or 7 ring atoms, or else, in the case of fused or benzofused ring systems, combinations of 5- and 6-membered ring systems, 5- and 5-membered ring systems, or else 6- and 6-membered ring systems. Examples which may be mentioned are ring systems such as pyrrolyl, pyrazolyl, imidazolyl, triazolyl, tetrazolyl, furyl, thienyl, oxazolyl, thiazolyl, isoxazolyl, oxadiazolyl, thiadiazolyl, pyridinyl, pyrimidinyl, pyrazinyl, triazinyl, oxazinyl, indolyl, benzimidazolyl, indazolyl, benzotriazolyl, benzothiazolyl, benzoxazolyl, benzofuryl, benzothienyl, quinolinyl, isoquinolinyl, cinnolinyl, quinazolinyl, quinoxalinyl, imidazopyridinyl or else benzoxazinyl. Preference is given to 5- to 6-membered, monocyclic heteroaryl, for example pyrrolyl, pyrazolyl, imidazolyl, triazolyl, tetrazolyl, furyl, thienyl, oxazolyl, thiazolyl, isoxazolyl, oxadiazolyl, thiadiazolyl, pyridinyl, pyrimidinyl, pyrazinyl, triazinyl.

C₃-C₆-Cycloalkenyl, C₃-C₈-cycloalkenyl and C₅-C₈-cycloalkenyl are understood to mean a monocyclic, saturated ring system formed exclusively from carbon atoms and having, respectively, 3 to 6, 3 to 8, and 5 to 8 atoms. Examples are cyclopropyl, cyclobutyl, cyclopentyl, cyclohexyl, cycloheptyl or cyclooctyl.

C₄-C₆-Cycloalkenyl, C₄-C₈-cycloalkenyl, and C₅-C₈-cycloalkenyl are understood to mean a monocyclic, mono- or polyunsaturated, non-aromatic ring system formed exclusively from carbon atoms and having, respectively, 4 to 6, 4 to 8, and 5 to 8 atoms. Examples are cyclobuten-1-yl, cyclopenten-1-yl, cyclohexen-2-yl, cyclohexen-1-yl or cycloocta-2,5-dienyl.

Heterocycloalkyl is understood to mean a 4- to 8-membered monocyclic, saturated ring system having 1 to 3 heteroatoms as defined above in any combination. Preference is given to 4- to 7-membered heterocycloalkyl groups, particular preference to 5- to 6-membered heterocycloalkyl groups. Examples which may be mentioned are pyrrolidinyl, piperidinyl, tetrahydrofuranyl, tetrahydropyranyl, oxetanyl, azetidinyl, azepanyl, morpholinyl, thiomorpholinyl or piperazinyl.

Heterocycloalkenyl is understood to mean a 4- to 8-membered monocyclic, mono- or polyunsaturated, nonaromatic ring system having 1 to 3 heteroatoms as defined above in any combination. Preference is given to 4- to 7-membered heterocycloalkyl groups, particular preference to 5- to 6-membered heterocycloalkyl groups. Examples which may be mentioned are 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl.

C₅-C₁₁-Spirocycloalkyl or C₅-C₁₁-heterospirocycloalkyl having a replacement of 1-4 carbon atoms by heteroatoms as defined above in any combination is understood to mean a fusion of two saturated ring systems which share a common atom. Examples are spiro[2.2]pentyl, spiro[2.3]hexyl, azaspiro[2.3]hexyl, spiro[3.3]heptyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, oxazaspiro[5.5]undecyl, diazaspiro[3.3]heptyl, thiazaspiro[3.3]heptyl, thiazaspiro[4.3]octyl, azaspiro[5.5]decyl, and the further homologous spiro[3.4], spiro[4.4], spiro[5.5], spiro[6.6], spiro[2.4], spiro[2.5], spiro[2.6], spiro[3.5], spiro[3.6], spiro[4.5], spiro[4.6] and spiro[5.6] systems including the variants modified by heteroatoms as per the definition. Preference is given to C₆-C₈-heterospirocycloalkyl.

C₆-C₁₂-Bicycloalkyl or C₆-C₁₂-heterobicycloalkyl having a replacement of 1-4 carbon atoms by heteroatoms as defined above in any combination is understood to mean a fusion of two saturated ring systems which share two directly adjacent atoms. Examples are bicyclo[2.2.0]hexyl, bicyclo[3.3.0]octyl, bicyclo[4.4.0]decyl, bicyclo[5.4.0]undecyl, bicyclo[3.2.0]heptyl, bicyclo[4.2.0]octyl, bicyclo[5.2.0]nonyl, bicyclo[6.2.0]decyl, bicyclo[4.3.0]nonyl, bicyclo[5.3.0]decyl, bicyclo[6.3.0]undecyl and bicyclo[5.4.0]undecyl, including the variants modified by heteroatoms, for example azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl, and the further possible combinations as per the definition. Preference is given to C₆-C₁₀-heterobicycloalkyl.

A bridged C₆-C₁₂ ring system such as bridged C₆-C₁₂-cycloalkyl or bridged C₆-C₁₂-heterocycloalkyl is understood to mean a fusion of at least two saturated rings which share two atoms that are not directly adjacent. This may give rise either to a bridged carbocycle (bridged cycloalkyl) or to a bridged heterocycle (bridged heterocycloalkyl) having a replacement of 1-4 carbon atoms by heteroatoms as defined above in any combination. Examples are bicyclo[2.2.1]heptyl, azabicyclo[2.2.1]heptyl, oxazabicyclo[2.2.1]heptyl, thiazabicyclo[2.2.1]heptyl, diazabicyclo[2.2.1]heptyl, bicyclo[2.2.2]octyl, azabicyclo[2.2.2]octyl, diazabicyclo[2.2.2]octyl, oxazabicyclo[2.2.2]octyl, thiazabicyclo[2.2.2]octyl, bicyclo[3.2.1]octyl, azabicyclo[3.2.1]octyl, diazabicyclo[3.2.1]octyl, oxazabicyclo[3.2.1]octyl, thiazabicyclo[3.2.1]octyl, bicyclo[3.3.1]nonyl, azabicyclo[3.3.1]nonyl, diazabicyclo[3.3.1]nonyl, oxazabicyclo[3.3.1]nonyl, thiazabicyclo[3.3.1]nonyl, bicyclo[4.2.1]nonyl, azabicyclo[4.2.1]nonyl, diazabicyclo[4.2.1]nonyl, oxazabicyclo[4.2.1]nonyl, thiazabicyclo[4.2.1]nonyl, bicyclo[3.3.2]decyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl and the further possible combinations according to the definition. Preference is given to bridged C₆-C₁₀-heterocycloalkyl.

Inventive compounds are the compounds of the general formula (I) and the salts, solvates and solvates of the salts thereof, the compounds encompassed by the general formula (I) of the formulae specified hereinafter and the salts, solvates and solvates of the salts thereof, and the compounds encompassed by the general formula (I) and specified hereinafter as working examples and the salts, solvates and solvates of the salts thereof, to the extent that the compounds encompassed by the general formula (I) and specified hereinafter are not already salts, solvates and solvates of the salts.

The present invention is likewise considered to encompass the use of the salts of the compounds according to the invention.

In the context of the present invention, preferred salts are physiologically acceptable salts of the compounds according to the invention. Also included, however, are salts which are themselves unsuitable for pharmaceutical applications but can be used, for example, for the isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

The present invention further provides all the possible crystalline and polymorphous forms of the compounds according to the invention, where the polymorphs may be present either as single polymorphs or as a mixture of a plurality of polymorphs in all concentration ranges.

The present invention also relates to medicaments comprising the compounds according to the invention together with at least one or more than one further active ingredient, especially for prophylaxis and/or therapy of neoplastic disorders.

In the context of the invention, solvates refer to those forms of the compounds according to the invention which, in the solid or liquid state, form a complex by coordination with solvent molecules. Hydrates are a specific form of the solvates in which the coordination is with water. Preferred solvates in the context of the present invention are hydrates.

Depending on their structure, the compounds according to the invention may exist in different stereoisomeric forms, i.e. in the form of configurational isomers or if appropriate also as conformational isomers. The compounds according to the invention may have a centre of asymmetry at the carbon atom to which R⁵ and R⁶ are attached (C-3). They may therefore take the form of pure enantiomers, racemates, or else of diastereomers or mixtures thereof when one or more of the substituents described in the formula (I) contains a further element of asymmetry, for example a chiral carbon atom. The present invention therefore also encompasses diastereomers and the respective mixtures thereof. The pure stereoisomers can be isolated from such mixtures in a known manner; chromatography processes are preferably used for this, in particular HPLC chromatography on a chiral or achiral phase.

In general, the enantiomers according to the invention inhibit the target proteins to different degrees and have different activity in the cancer cell lines studied. The more active enantiomer is preferred, which is often that in which the centre of asymmetry represented by the carbon atom bonded to R⁵ and R⁶ has (R) configuration.

The present invention further provides enantiomer mixtures of the (3R)-configured compounds according to the invention with their (3S) enantiomers, especially the corresponding racemates and enantiomer mixtures in which the (3R) form predominates.

Where the compounds according to the invention can occur in tautomeric forms, the present invention encompasses all the tautomeric forms.

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. Furthermore, the incorporation of isotopes, for example of deuterium, can lead to particular therapeutic advantages as a consequence of greater metabolic stability of the compound, for example an extension of the half-life in the body or a reduction in the active dose required; such modifications of the compounds according to the invention may therefore, in some cases, also constitute a preferred embodiment of the present invention. Isotopic variants of the compounds according to the invention can be prepared by the processes known to those skilled in the art, for example by the methods described below and the instructions reproduced in the working examples, by using corresponding isotopic modifications of the particular reagents and/or starting compounds therein.

The compounds according to the invention can act systemically and/or locally. For this purpose, they can be administered in a suitable manner, for example by the oral, parenteral, pulmonary, nasal, sublingual, lingual, buccal, rectal, dermal, transdermal, conjunctival, otic route, or as an implant or stent.

The compounds according to the invention can be administered in administration forms suitable for these administration routes.

Suitable administration forms for oral administration are all administration forms capable of releasing the compounds according to the invention rapidly. Here, the compounds according to the invention can be present in crystalline, amorphous and/or dissolved form, for example in tablets (non-coated or coated tablets, for example coated with enteric, slowly dissolving or insoluble coats which control the release of the compound according to the invention), in tablets which decompose rapidly in the oral cavity, in films/wafers, in films/lyophylizates, in capsules (for example hard gelatin capsules or soft gelatin capsules), in sugar-coated tablets, in granules, in pellets, in powders, in emulsions, in suspensions, in aerosols or in solutions.

Parenteral administration can bypass an absorption step (for example intravenously, intraarterially, intracardially, intraspinally or intralumbarly) or include an absorption (for example intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds according to the invention can be converted to the administration forms mentioned. This can be accomplished in a manner known per se to the person skilled in the art, by mixing with inert nontoxic pharmaceutically suitable auxiliaries. These auxiliaries include carriers (for example microcrystalline cellulose, lactose, mannitol), solvents (for example liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (for example antioxidants, for example ascorbic acid), dyes (for example inorganic pigments such as iron oxides) and flavour and/or odour correctors.

The present invention furthermore provides medicaments which comprise the compounds according to the invention, typically together with one or more inert, nontoxic, pharmaceutically suitable auxiliaries, and the use thereof for the aforementioned purposes.

The formulation of the compounds according to the invention to give pharmaceutical preparations is effected in a manner known per se, by converting the active ingredient(s) to the desired administration form with the auxiliaries customary in pharmaceutical formulation.

The auxiliaries used may, for example, be carrier substances, fillers, disintegrants, binders, humectants, glidants, absorbents and adsorbents, diluents, solvents, cosolvents, emulsifiers, solubilizers, taste correctants, colorants, preservatives, stabilizers, wetting agents, salts for modifying osmotic pressure or buffers. Reference should be made to Remington's Pharmaceutical Science, 15th ed. Mack Publishing Company, East Pennsylvania (1980).

The pharmaceutical formulations may be in solid form, for example in the form of tablets, coated tablets, pills, suppositories, capsules, transdermal systems, or in semisolid form, for example as ointments, creams, gels, suppositories, emulsions, or in liquid form, for example as solutions, tinctures, suspensions or emulsions.

The auxiliaries used in the context of the invention may, for example, be salts, saccharides (mono-, di-, tri-, oligo- and/or polysaccharides), proteins, amino acids, peptides, fats, waxes, oils, hydrocarbons and derivatives thereof, and the auxiliaries may be of natural origin or synthetic or partially synthetic.

Useful forms for oral or peroral administration are especially tablets, coated tablets, capsules, pills, powders, granules, pastilles, suspensions, emulsions or solutions.

Useful forms for parenteral administration are especially suspensions, emulsions, and particularly solutions.

The compounds according to the invention are suitable for prophylaxis and/or therapy of hyperproliferative disorders, for example psoriasis, keloids and other hyperplasias which affect the skin, and for prophylaxis and/or therapy of benign prostate hyperplasias (BPH), solid tumours and haematological tumours.

Solid tumours that can be treated in accordance with the invention are, for example, tumours of the breast, the respiratory tract, the brain, the reproductive organs, the gastrointestinal tract, the urogenital tract, the eye, the liver, the skin, the head and the neck, the thyroid gland, the parathyroid gland, the bones, and the connective tissue and metastases of these tumours.

Haematological tumours that can be treated are, for example, multiple myeloma, lymphoma or leukaemia.

Breast tumours that can be treated are, for example, mammary carcinoma with positive hormone receptor status, mammary carcinoma with negative hormone receptor status, Her2-positive mammary carcinoma, hormone receptor- and Her2-negative mammary carcinoma, BRCA-associated mammary carcinoma and inflammatory mammary carcinoma.

Tumours of the respiratory tract that can be treated are, for example, non-small-cell bronchial carcinoma and small-cell bronchial carcinoma.

Brain tumours that can be treated are, for example, glioma, glioblastoma, astrocytoma, meningioma and medulloblastoma.

Tumours of the male reproductive organs that can be treated are, for example, prostate carcinoma, malignant epididymal tumours, malignant testicular tumours and penile carcinoma.

Tumours of the female reproductive organs that can be treated are, for example, endometrial carcinoma, cervical carcinoma, ovarian carcinoma, vaginal carcinoma and vulvar carcinoma.

Tumours of the gastrointestinal tract that can be treated are, for example, colorectal carcinoma, anal carcinoma, gastric carcinoma, pancreatic carcinoma, oesophageal carcinoma, gallbladder carcinoma, small-intestinal carcinoma, salivary gland carcinoma, neuroendocrine tumours and gastrointestinal stromal tumours.

Tumours of the urogenital tract that can be treated are, for example, urinary bladder carcinoma, renal cell carcinoma, and carcinoma of the renal pelvis and of the urinary tract.

Tumours of the eye that can be treated are, for example, retinoblastoma and intraocular melanoma.

Tumours of the liver that can be treated are, for example, hepatocellular carcinoma and cholangiocellular carcinoma.

Tumours of the skin that can be treated are, for example, malignant melanoma, basalioma, spinalioma, Kaposi's sarcoma and Merkel cell carcinoma.

Tumours of the head and neck that can be treated are, for example, laryngeal carcinoma and carcinoma of the pharynx and of the oral cavity.

Sarcomas that can be treated are, for example, soft tissue sarcoma and osteosarcoma.

Lymphomas that can be treated are, for example, non-Hodgkin's lymphoma, Hodgkin's lymphoma, cutaneous lymphoma, lymphoma of the central nervous system and AIDS-associated lymphoma.

Leukaemias that can be treated are, for example, acute myeloid leukaemia, chronic myeloid leukaemia, acute lymphatic leukaemia, chronic lymphatic leukaemia and hair cell leukaemia.

Advantageously, the compounds according to the invention can be used for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

Particularly advantageously, the compounds according to the invention can be used for prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The compounds according to the invention are also suitable for prophylaxis and/or therapy of benign hyperproliferative diseases, for example endometriosis, leiomyoma and benign prostate hyperplasia.

The compounds according to the invention are also suitable for prophylaxis and/or therapy of systemic inflammatory diseases, especially LPS-induced endotoxic shock and/or bacteria-induced sepsis.

The compounds according to the invention are also suitable for prophylaxis and/or therapy of inflammatory or autoimmune disorders, for example:
pulmonary disorders associated with inflammatory, allergic and/or proliferative processes: chronic obstructive pulmonary disorders of any origin, particularly bronchial asthma; bronchitis of different origin; all forms of restrictive pulmonary disorders, particularly allergic alveolitis; all forms of pulmonary oedema, particularly toxic pulmonary oedema; sarcoidoses and granulomatoses, particularly Boeck's disease,
rheumatic disorders/autoimmune disorders/joint disorders associated with inflammatory, allergic and/or proliferative processes: all forms of rheumatic disorders, especially rheumatoid arthritis, acute rheumatic fever, polymyalgia rheumatica; reactive arthritis; inflammatory soft-tissue disorders of other origin; arthritic symptoms in the case of degenerative joint disorders (arthroses); traumatic arthritis; collagenoses of any origin, for example systemic lupus erythematosus, sclerodermia, polymyositis, dermatomyositis, Sjögren's syndrome, Still's syndrome, Felty's syndrome,
allergies associated with inflammatory and/or proliferative processes: all forms of allergic reactions, for example angiooedema, hay fever, insect bites, allergic reactions to medicaments, blood derivatives, contrast agents, etc., anaphylactic shock, urticaria, contact dermatitis,
vascular inflammation (vasculitis): panarteritis nodosa, temporal arteritis, erythema nodosum,
dermatological disorders associated with inflammatory, allergic and/or proliferative processes: atopic dermatitis; psoriasis; pityriasis rubra pilaris; erythematous disorders triggered by different noxae, for example radiation, chemicals, burns, etc.; bullous dermatoses; lichenoid disorders; pruritus; seborrhoeic eczema; rosacea; pemphigus vulgaris; erythema exsudativum multiforme; balanitis; vulvitis; hair loss, such as alopecia areata; cutaneous T-cell lymphoma,
renal disorders associated with inflammatory, allergic and/or proliferative processes: nephrotic syndrome; all nephritides,
hepatic disorders associated with inflammatory, allergic and/or proliferative processes: acute hepatic disintegration; acute hepatitis of different origin, for example viral, toxic, medicament-induced; chronic aggressive and/or chronic intermittent hepatitis,
gastrointestinal disorders associated with inflammatory, allergic and/or proliferative processes: regional enteritis (Crohn's disease); ulcerative colitis; gastritis; reflux oesophagitis; gastroenteritides of other origin, e.g. indigenous sprue,
proctological disorders associated with inflammatory, allergic and/or proliferative processes: anal eczema; fissures; haemorrhoids; idiopathic proctitis,
ocular disorders associated with inflammatory, allergic and/or proliferative processes: allergic keratitis, uveitis, iritis; conjunctivitis; blepharitis; optic neuritis; chlorioditis; sympathetic ophthalmia,
disorders of the ear-nose-throat region associated with inflammatory, allergic and/or proliferative processes: allergic rhinitis, hay fever; otitis externa, for example caused by contact eczema, infection, etc.; otitis media,
neurological disorders associated with inflammatory, allergic and/or proliferative processes: cerebral oedema, particularly tumour-related cerebral oedema; multiple sclerosis; acute encephalomyelitis; meningitis; various forms of seizure, for example West's syndrome,
haematological disorders associated with inflammatory, allergic and/or proliferative processes: congenital haemolytic anaemia; idiopathic thrombocytopenia,
neoplastic disorders associated with inflammatory, allergic and/or proliferative processes: acute lymphatic leukaemia; malignant lymphoma; lymphogranulomatoses; lymphosarcoma; extensive metastases, particularly in the case of mammary, bronchial and prostate carcinoma,
endocrine disorders associated with inflammatory, allergic and/or proliferative processes: endocrine orbitopathy; thyrotoxic crisis; de Quervain's thyroiditis; Hashimoto's thyroiditis; Basedow's disease,
organ and tissue transplants, graft-versus-host disease,
severe states of shock, for example anaphylactic shock, systemic inflammatory response syndrome (SIRS),
substitution therapy in the case of: congenital primary renal insufficiency, for example congenital adrenogenital syndrome; acquired primary renal insufficiency, for example Addison's disease, autoimmune adrenalitis, for example postinfectious, tumours, metastases, etc; congenital secondary renal insufficiency, for example congenital hypopituitarism; acquired secondary renal insufficiency, for example postinfectious, tumours, etc.,
emesis associated with inflammatory, allergic and/or proliferative processes, for example in combination with a 5-HT3 antagonist in the case of cytostatic-induced vomiting,
pain of inflammatory origin, for example lumbago.

The compounds according to the invention are also suitable for the treatment of viral disorders, for example infections caused by papillomaviruses, herpesviruses, Epstein-Barr viruses, hepatitis B or C viruses, and human immunodeficiency viruses.

The compounds according to the invention are also suitable for the treatment of atherosclerosis, dyslipidaemia, hypercholesterolaemia, hypertriglyceridaemia, peripheral vascular disorders, cardiovascular disorders, angina pectoris, ischaemia, stroke, myocardial infarction, angioplastic restenosis, hypertension, thrombosis, obesity, endotoxaemia.

The compounds according to the invention are also suitable for the treatment of neurodegenerative diseases, for example multiple sclerosis, Alzheimer's disease and Parkinson's disease.

These disorders are well-characterized in man, but also exist in other mammals.

The present invention further provides for the use of the compounds according to the invention as a medicament, in particular for prophylaxis and/or therapy of neoplastic disorders.

The present invention further provides the use of the compounds according to the invention for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present invention furthermore provides for the use of the compounds according to the invention for prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention furthermore provides for the use of the compounds according to the invention for production of a medicament.

The present invention furthermore provides for the use of the compounds according to the invention for production of a medicament for prophylaxis and/or therapy of neoplastic disorders.

The present application furthermore provides for the use of the compounds according to the invention for production of a medicament for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present invention furthermore provides for the use of the compounds according to the invention for producing a medicament for the prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present invention furthermore provides for the use of the compounds according to the invention for prophylaxis and/or therapy of neoplastic disorders.

The present invention furthermore provides for the use of the compounds according to the invention for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present invention furthermore provides for the use of the compounds according to the invention for prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present invention furthermore provides pharmaceutical formulations in the form of tablets comprising one of the compounds according to the invention for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application furthermore provides pharmaceutical formulations in the form of tablets comprising one of the compounds according to the invention for prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention furthermore provides for the use of the compounds according to the invention for treatment of disorders associated with proliferative processes.

The invention further provides for the use of the compounds according to the invention for treatment of benign hyperplasias, inflammation disorders, autoimmune disorders, sepsis, viral infections, vascular disorders and neurodegenerative disorders.

The compounds according to the invention can be used alone or, if required, in combination with one or more further pharmacologically active substances, provided that this combination does not lead to undesirable and unacceptable side effects. The present invention therefore further provides medicaments comprising a compound according to the invention and one or more further active ingredients, especially for prophylaxis and/or therapy of the aforementioned disorders.

For example, the compounds according to the invention can be combined with known antihyperproliferative, cytostatic or cytotoxic chemical and biological substances for treatment of cancer. The combination of the compounds according to the invention with other substances commonly used for cancer treatment, or else with radiotherapy, is particularly appropriate.

An illustrative but nonexhaustive list of suitable combination active ingredients is as follows: abiraterone acetate, abraxane, acolbifene, Actimmune, actinomycin D (dactinomycin), afatinib, affinitak, Afinitor, aldesleukin, alendronic acid, alfaferone, alitretinoin, allopurinol, Aloprim, Aloxi, alpharadin, altretamine, aminoglutethimide, aminopterin, amifostine, amrubicin, amsacrine, anastrozole, anzmet, apatinib, Aranesp, arglabin, arsenic trioxide, Aromasin, arzoxifen, asoprisnil, L-asparaginase, atamestane, atrasentane, avastin, axitinib, 5-azacytidine, azathioprine, BCG or Tice BCG, bendamustine, bestatin, beta-methasone acetate, betamethasone sodium phosphate, bexarotene, bicalutamide, bleomycin sulphate, broxuridine, bortezomib, bosutinib, busulfan, cabazitaxel, calcitonin, campath, camptothecin, capecitabine, carboplatin, carfilzomib, carmustine, casodex, CCI-779, CDC-501, cediranib, cefesone, celebrex, celmoleukin, cerubidine, cediranib, chlorambucil, cisplatin, cladribine, clodronic acid, clofarabine, colaspase, corixa, crisnatol, crizotinib, cyclophosphamide, cyproterone acetate, cytarabine, dacarbazine, dactinomycin, dasatinib, daunorubicin, DaunoXome, Decadron, Decadron Phosphate, decitabine, degarelix, delestrogen, denileukin diftitox, depomedrol, deslorelin, dexrazoxane, diethylstilbestrol, diflucan, 2′,2′-difluorodeoxycytidine, DN-101, docetaxel, doxifluridine, doxorubicin (Adriamycin), dronabinol, dSLIM, dutasteride, DW-166HC, edotecarin, eflornithine, Eligard, Elitek, Ellence, Emend, enzalutamide, epirubicin, epoetin-alfa, Epogen, epothilone and derivatives thereof, eptaplatin, ergamisol, erlotinib, erythro-hydroxynonyladenine, estrace, oestradiol, oestramustine sodium phosphate, ethinyloestradiol, Ethyol, etidronic acid, etopophos, etoposide, everolimus, exatecan, exemestane, fadrozole, farston, fenretinide, filgrastim, finasteride, fligrastim, floxuridine, fluconazole, fludarabine, 5-fluorodeoxyuridine monophosphate, 5-fluorouracil (5-FU), fluoxymesterone, flutamide, folotin, formestane, fosteabine, fotemustine, fulvestrant, Gammagard, gefitinib, gemcitabine, gemtuzumab, Gleevec, Gliadel, goserelin, gossypol, granisetrone hydrochloride, hexamethylmelamine, histamine dihydrochloride, histrelin, holmium-166-DOTPM, hycamtin, hydrocortone, erythro-hydroxynonyladenine, hydroxyurea, hydroxyprogesterone caproate, ibandronic acid, ibritumomab tiuxetan, idarubicin, ifosfamide, imatinib, iniparib, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, ixabepilone, keyhole limpet haemocyanin, kytril, lanreotide, lapatinib, lasofoxifene, lenalidomide, lentinan sulphate, lestaurtinib, letrozole, leucovorin, leuprolide, leuprolide acetate, levamisole, levofolic acid calcium salt, levothroid, levoxyl, Libra, liposomal MTP-PE, lomustine, lonafarnib, lonidamine, marinol, mechlorethamine, mecobalamine, medroxyprogesterone acetate, megestrol acetate, melphalan, Menest, 6-mercaptopurine, mesna, methotrexate, metvix, miltefosine, minocycline, minodronate, miproxifen, mitomycin C, mitotan, mitoxantrone, modrenal, MS-209, MX-6, myocet, nafarelin, nedaplatin, nelarabine, nemorubicin, neovastat, neratinib, neulasta, neumega, neupogen, nilotimib, nilutamide, nimustine, nolatrexed, nolvadex, NSC-631570, obatoclax, oblimersen, OCT-43, octreotide, olaparib, ondansetron hydrochloride, Onco-TCS, Orapred, osidem, oxaliplatin, paclitaxel, pamidronate disodium, pazopanib, pediapred, pegaspargase, pegasys, pemetrexed, pentostatin, N-phosphonoacetyl-L-aspartate, picibanil, pilocarpine hydrochloride, pirarubicin, plerixafor, plicamycin, PN-401, porfimer sodium, prednimustine, prednisolone, prednisone, Premarin, procarbazine, Procrit, QS-21, quazepam, R-1589, raloxifene, raltitrexed, ranpirnas, RDEA119, Rebif, regorafenib, 13-cis-retinoic acid, rhenium-186 etidronate, rituximab, roferon-A, romidepsin, romurtide, ruxolitinib, salagen, salinomycin, sandostatin, sargramostim, satraplatin, semaxatinib, semustine, seocalcitol, sipuleucel-T, sizofiran, sobuzoxan, Solu-Medrol, sorafenib, streptozocin, strontium-89 chloride, sunitinib, Synthroid, T-138067, tamoxifen, tamsulosin, Tarceva, tasonermin, tastolactone, Taxoprexin, Taxoter, teceleukin, temozolomide, temsirolimus, teniposide, testosterone propionate, Testred, thalidomide, thymosin alpha-1, thioguanine, thiotepa, thyrotropin, tiazorufin, tiludronic acid, tipifarnib, tirapazamine, TLK-286, toceranib, topotecan, toremifen, tositumomab, tastuzumab, teosulfan, transMID-107R, tretinoin, Trexall, trimethylmelamine trimetrexate, triptorelin acetate, triptorelin pamoate, trofosfamide, UFT, uridine, valrubicin, valspodar, vandetanib, vapreotide, vatalanib, vemurafinib, verte-porfin, vesnarinone, vinblastine, vincristine, vindesine, vinflumine, vinorelbine, virulizin, vismodegib, Xeloda, Z-100, Zinecard, zinostatin stimalamer, zofran, zoledronic acid.

More particularly, the compounds according to the invention can be combined with antibodies, for example aflibercept, alemtuzumab, bevacizumab, brentuximumab, catumaxomab, cetuximab, denosumab, edrecolomab, gemtuzumab, ibritumomab, ipilimumab, ofatumumab, panitumumab, pertuzumab, rituximab, tositumumab or trastuzumab, and also with recombinant proteins.

More particularly, the compounds according to the invention can be used in combination with treatments directed against angiogenesis, for example bevacizumab, axitinib, regorafenib, cediranib, sorafenib, sunitinib, lenalidomide or thalidomide.

Combinations with antihormones and steroidal metabolic enzyme inhibitors are particularly suitable because of their favourable profile of side effects.

Combinations with P-TEFb and/or CDK9 inhibitors are likewise particularly suitable because of the possible synergistic effects.

Generally, the following aims can be pursued with the combination of the compounds according to the invention with other cytostatically or cytotoxically active agents:
improved efficacy in slowing the growth of a tumour, in reducing its size or even in the complete elimination thereof, compared with treatment with an individual active compound;
the possibility of using the chemotherapeutics used in a lower dosage than in the case of monotherapy;
the possibility of a more tolerable therapy with fewer side effects compared with individual administration;
the possibility of treatment of a broader spectrum of tumours;
the achievement of a higher rate of response to the therapy;
a longer survival time of the patient compared with present-day standard therapy.

In addition, the compounds according to the invention can also be used in conjunction with radiotherapy and/or surgical intervention.

### Preparation of the Compounds According to the Invention:

In the present description:

NMR signals are reported with their respectively apparent multiplicities or combinations thereof. In this context, s=singlet, d=doublet, t=triplet, q=quartet, qi=quintet, sp=septet, m=multiplet, b=broad signal. Signals having combined multiplicities are reported, for example, as dd=doublet of doublets.
ACN acetonitrile
sel. selected
Ex Example
(+)-BINAP (R)-(+)-2,2′-bis(diphenylphosphino)-1,1′-binaphthyl
(+)-BINAP 2,2′-bis(diphenylphosphino)-1,1′-binaphthyl (racemic)
Boc tert-butoxycarbonyl
Cbz carbamazepin
CDCl₃ deuterochloroform
CHAPS 3-{dimethyl[3-(4-{5,9,16-trihydroxy-2,15-dimethyltetracyclo-[8.7.0.0^{2,7}.0^{11,15}]heptadecan-14-yl}pentanamido)propyl]-azaniumyl}propane-1-sulphonate
DAD dioden array detector
dba dibenzylideneacetone
DCC dicyclohexylcarbodiimide
DMF N,N-dimethylformamide
DMSO-d6 deuterated dimethyl sulphoxide
DMSO dimethyl sulphoxide
EA ethyl acetate
Fmoc fluorenylmethoxycarbonyl
HATU (7-aza-1H-benzotriazol-1-yl)-1,1,3,3-tetramethyluronium hexafluorophosphate
HBTU O-benzotriazol-N,N,N′,N′-tetramethyluronium hexafluorophosphate
KOtBu potassium tert-butoxide
KHMDS potassium bis(trimethylsilyl)amide
LCMS liquid chromatography coupled with mass spectrometry
LiHMDS lithium bis(trimethylsilyl)amide
PyBOB (benzotriazol-1-yl)oxytripyrrolidinophosphonium hexafluorophosphate
RP-HPLC reverse-phase high-pressure liquid chromatography
RT room temperature
THF tetrahydrofuran
T3P 2,4,6-tripropyl-1,3,5,2,4,6-trioxatriphosphorinane 2,4,6-trioxide
TFA trifluoroacetic acid
TBTU (benzotriazol-1-yloxy)bisdimethylaminomethylium fluoroborate
UPLC ultra high performance chromatography
Xanthphos 4,5-bis(diphenylphosphino)-9,9-dimethylxanthene

### General Description of the Preparation of the Compounds of the General Formula (I) According to the Invention:

The compounds of the formulae (Ia) and (Ib) according to the invention shown in Scheme 1 can be prepared via synthesis routes described hereinafter. The formulae specified represent different portions of the general formula (I) in which A, R², R³, R⁴, R⁵, R⁶, R⁷, R⁸, R⁹ and n are each as defined for the general formula (I). In compounds of the formula (Ia) there is a group —C(═O)NR⁸R⁹ located in the position of R¹; in compounds of the formula (Ib) there is a group —S(═O)₂NR⁸R⁹ located in the position of R¹.

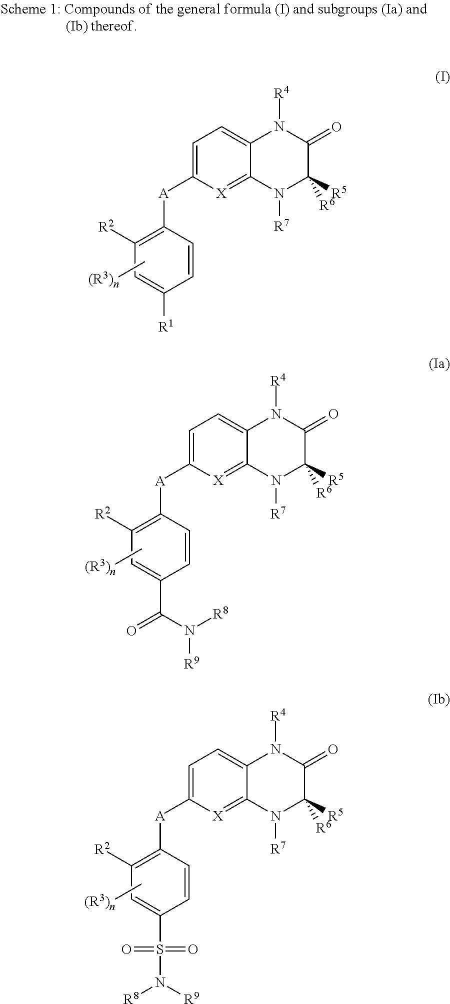

In addition to the synthesis sequences discussed hereinafter, it is also possible, in accordance with the general knowledge of the person skilled in the art in organic chemistry, to take further synthesis routes for the synthesis of compounds of the general formula (I) according to the invention. The sequence of the synthesis steps shown in the schemes which follow is not binding, and synthesis steps from various of the schemes shown hereinafter may optionally be combined to form new sequences. In addition, interconversions of the substituents R², R³, R⁴, R⁵, R⁶, R⁷, R⁸, R⁹ can be performed before or after the synthesis stages shown. Examples of such conversions are the introduction or elimination of protecting groups, reduction or oxidation of functional groups, halogenation, metallation, metal-catalysed coupling reactions, substitution reactions or further reactions known to the person skilled in the art. These reactions include conversions which introduce a functional group which enables the further conversion of substituents. Suitable protective groups and methods for their introduction and removal are known to the person skilled in the art (see, for example, T. W. Greene and P. G. M. Wuts in: Protective Groups in Organic Synthesis, 3. Edition, Wiley 1999). In addition, it is possible to combine two or more reaction steps without intermediate workup in a manner known to the person skilled in the art (for example in what are called “one-pot” reactions).

Compounds of the general formula (I) and the precursors thereof described hereinafter, in which mutually different substituents R⁵ and R⁶ are present are chiral and may occur as enantiomer mixtures, for example racemates, or as pure enantiomers. The enantiomer mixtures mentioned can be separated into the enantiomers by separation methods familiar to the person skilled in the art, for example preparative HPLC on a chiral stationary phase.

Scheme 2 illustrates the construction of amides of the formula (V) from simple pyridine derivatives such as 3-amino-2,6-dichloropyridine ((II), CAS-No. 62476-56-6). For the preparation of compounds of the formula (III) from (II), a large number of methods for preparing amides from the azidocarboxylic acids of the formula (IIa) in which R⁵ and R⁶ are as defined for the general formula (I) may be employed. Thus, it is possible to make use of coupling reagents known to the person skilled in the art, such as TBTU, HATU or DCC. Also suitable is the reaction of the azidocarboxylic acids employed with an inorganic acid chloride such as thionyl chloride, phosphorus oxychloride or oxalyl chloride, followed by addition of the pyridineamine. The preparation of the azidocarboxylic acids required is described in the literaturen (Chem Eur J (2010), 16, p 7572 ff, D. Tietze et al.; J Org Chem (2010), 75, p 6532ff, Katritzky et al.). The carboxylic acid azides have to be handled very carefully as they may decompose explosively. Also, storage of the reagents required for introducing the azide should be dispensed with. These aspects are discussed in Katritzky et al.

To reduce the azido group in (III), which leads to amines of the formula (IV), the reaction with trialkyl- or triarylphosphines according to Staudinger (Tetrahedron (2012), 68, p 697ff, Laschat et al.) may be performed. An example of a suitable phosphine is trimethylphosphine. The amines (IV) can be isolated as free base or, advantageously, in salt form, for example as hydrochloride. To this end, the crude amine of the formula (IV) is dissolved in a non-polar solvent, for example diethyl ether, and precipitated as salt by addition of an acid, for example hydrogen chloride. Further conversion into compounds of the formula (V) with introduction of the radical R⁷, which is defined as for the general formula (I), can preferably take place via the reductive amination known to the person skilled in the art (for representative procedures see, for example, US2010/105906 A1). Here, the primary amine (IV), as free base or in salt form, is reacted in situ with an aldehyde or ketone suitable for introducing R⁷ to afford an imine, and the latter is then transformed by addition of a suitable reducing agent such as, for example, sodium triacetoxyborohydride into the secondary amine of the formula (V).

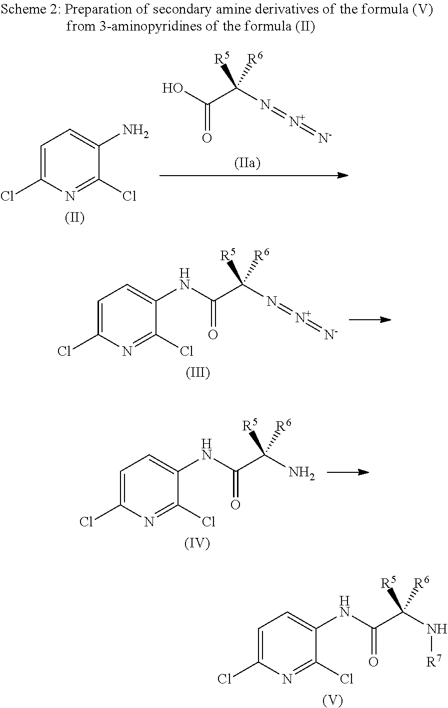

An alternative route to compounds of the formula (IV) is described in Scheme 3. To this end, nitrogen-atom-protected amino acids of the formula (IIb) in which R⁵ and R⁶ are as defined in the general formula (I) and in which PG represents a protective group such as, for example, Boc, Cbz or else Fmoc are reacted with suitable aminopyridine derivatives, for example 3-amino-2,6-dichloropyridine ((II), CAS-No. 62476-56-6). Here, use is made of coupling reagents known to the person skilled in the art, such as TBTU, HATU or DCC. The conversion of the carboxylic acids to their amides is described in general terms in reference books such as “Compendium of Organic Synthetic Methods”, volume I-VI (Wiley Interscience) or “The Practice of Peptide Synthesis”, Bodansky (Springer Verlag). Compounds of the formula (IIb) are known to those skilled in the art and commercially available. The resulting compounds of the formula (IIIa) are then converted into the compounds of the formula (IV) by removing the protective group PG at the amine by suitable methods. A large number of methods suitable for this pursose is known; these can be found in standard references (see, for example, T. W. Greene and P. G. M. Wuts in: Protective Groups in Organic Synthesis, 3. Edition, Wiley 1999).

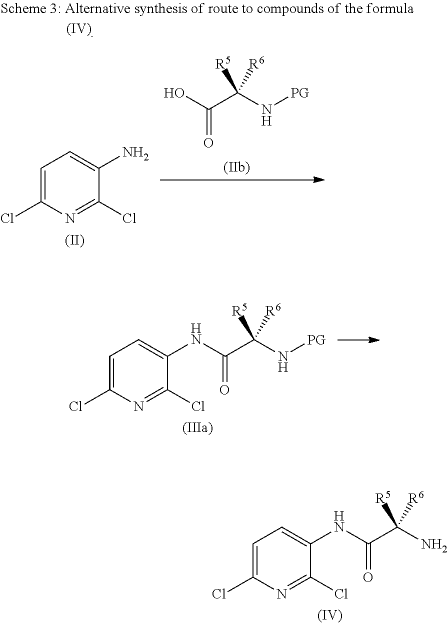

As shown in Scheme 4, the secondary amines of the formula (V) can be converted by cyclization into dihydropyridopyrazinones of the formula (VI). To this end, compounds of the formula (V) can be reacted in the presence of a suitable base, for example a trialkylamine such as triethylamine or N,N-diisopropylethylamine, at elevated temperature (see also WO2010/96426 A2, Example 16). The subsequent alkylation to give compounds (VII) can be effected by reaction with R⁴-LG in which R⁴ is as defined in the general formula (I) and LG is a leaving group, preferably iodide, in the presence of a suitable base such as sodium hydride, under conditions known to the person skilled in the art. Further reaction of the resulting compounds of the formula (VII) to the ester derivatives (VIII) can be performed by reaction with compounds of the formula (VIIa) in which A, R², R³ and n are as defined in the general formula I and in which R^{E} represents C₁-C₆-alkyl, in a palladium-catalysed coupling reaction according to Buchwald and Hartwig (see, for example, J. Organomet. Chem. (1999), 576, p 125ff). Examples of palladium sources suitable here are palladium(II) acetate or palladium-dba complexes, for example Pd₂(dba)₃ (CAS Nos. 51364-51-3 and 52409-22-0). The conversion depends strongly on the ligands used. In this manner, the examples given in the experimental part were obtained, for example, by using (+)-BINAP or xanthphos (cf. also US2006/009457 A1).

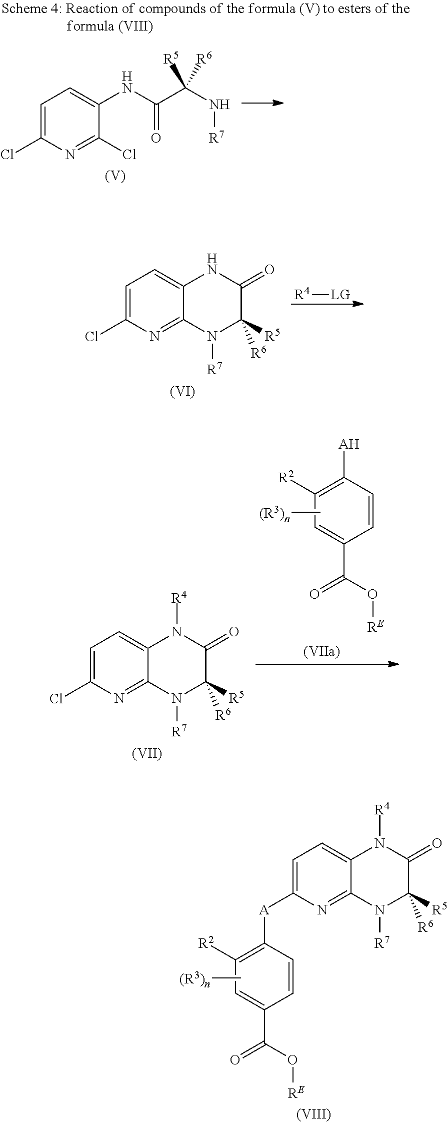

The preparation of carboxamides of the general formula (Ia) can be effected in accordance with Scheme 5 by means of hydrolysis of the respective esters of the formula (VIII) to give the corresponding carboxylic acids of the formula (IX) by methods known to the person skilled in the art. These reactions are preferably carried out using alkali metal hydroxides such as lithium hydroxide, sodium hydroxide or potassium hydroxide in aqueous alcoholic solutions, if appropriate with addition of a cyclic ether such as tetrahydrofuran.

The carboxylic acids (IX) obtained in this manner can be converted to the carboxamides of the general formula (Ia) according to the invention by reaction, for example, with the generally commercially available amines, specified in the working examples, of the formula R⁸R⁹NH in which R⁸ and R⁹ are as defined for the general formula (I), with additional activation by a method as commonly known to the person skilled in the art. Possible methods which should be mentioned here include the use of TBTU, HATU, HBTU, PyBOB or T3P with the addition of a suitable base. The conversion of the carboxylic acids to their amides is described in general terms in reference books such as “Compendium of Organic Synthetic Methods”, volume I-VI (Wiley Interscience) or “The Practice of Peptide Synthesis”, Bodansky (Springer Verlag).

The reaction routes described above allow, in the case of the use of an enantiomerically pure azidocarboxylic acid of the formula (IIa) or of an enantiomerically pure nitrogen-protected amino acid of the formula (IIb) at the start of the sequence, very substantial suppression of epimerization or racemization of the stereogenic site at the carbon atom attached to R⁵ and R⁶.

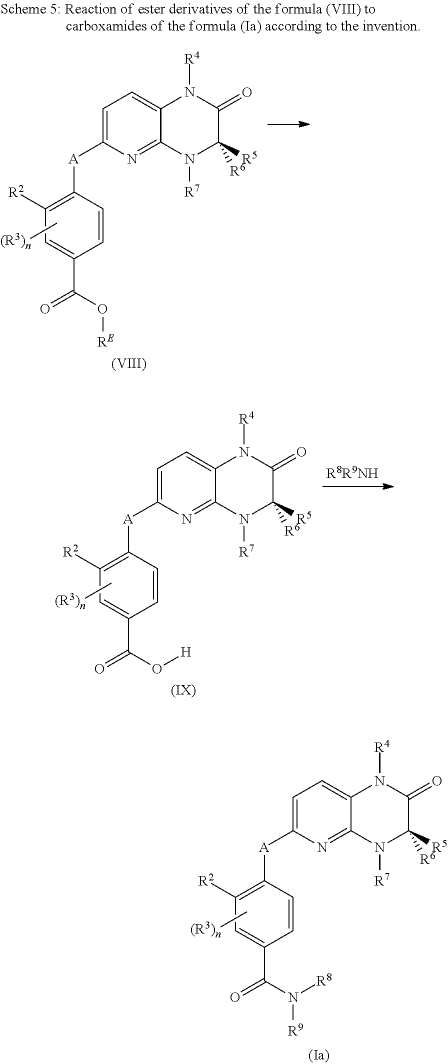

The preparation of the compounds of the formula (Ib) according to the invention having a sulphonamide group in the position of R¹ can be effected according to Scheme 6. In this context, compounds of the formula (VII) can be reacted directly, in a manner analogous to that discussed in Scheme 4 for the conversion of (VII) to (VIII), with compounds of the formula (X) in which A, R², R³, R⁸, R⁹ and n are each as defined in the general formula (I) in a Palladium-catalysed coupling reaction according to Buchwald and Hartwig to give the compounds of the formula (Ib) according to the invention.

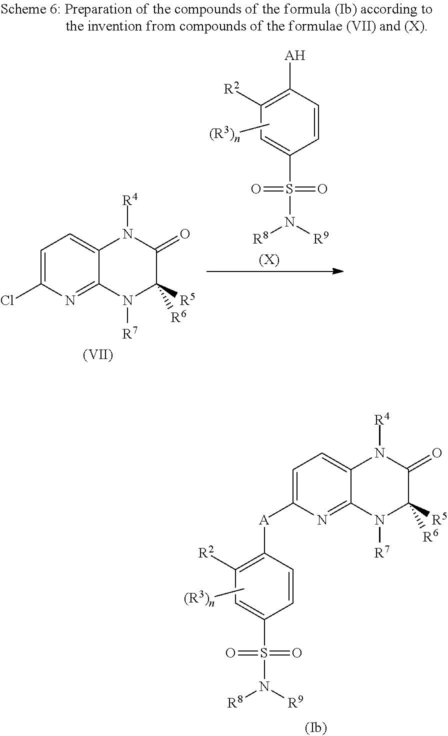

The preparation of intermediates of the formula (VIa) in which R⁷ is optionally substituted phenyl as per the definition of the general formula (I) is described in Scheme 7.

3-Amino-2,6-dichloropyridine ((II), CAS-No. 62476-56-6) is reacted with compounds of the formula (XI) in which R⁵ and R⁶ are as defined for the general formula (I), and in which LG and LG′ are each independently of one another a leaving group, preferably chlorine or bromine, for example 2-bromopropionyl bromide (CAS 563-76-8). This is done by conversion, under conditions known to the person skilled in the art, with a suitable solvent such as dichloromethane or THF and with addition of a base such as triethylamine, diisopropylethylamine or pyridine. The base can also be used as the solvent. This gives compounds of the formula (XII). These intermediates (XII) are reacted with anilines of the formula R⁷—NH₂ in which R⁷ is optionally substituted phenyl as per the definition of the general formula (I) to give compounds of the formula (XIII). This reaction can be carried out in various solvents such as toluene or acetonitrile and with addition of a base such as, for example, potassium carbonate, diisopropylethylamine or triethylamine at elevated temperature (Org. Lett. (2008), 10, S. 2905 ff, S. P. Marsden et al.). Dihydropyridopyrazinones of the formula (VIa) in which R⁷ is optionally substituted phenyl as per the definition of the general formula (I) are obtained by cyclizing the compounds of the formula (XIII) in the presence of a suitable base such as triethylamine, diisopropylethylamine or potassium carbonate under elevated temperature in solvents such as, for example, N,N-dimethylformamide, N,N-dimethylacetamide, N-methylpyrrolidone or else dimethyl sulphoxide (in this regard, see also WO2010/96426 A2, Example 16). From these intermediates of the formula (VIa), it is possible according to Schemes 4, 5 and 6 to prepare the corresponding compounds of the formula (I) according to the invention in which R⁷ is optionally substituted phenyl as per the definition of the general formula (I). This gives the compounds of the formula (I) as racemates if R⁵ and R⁶ are different from one another. These can optionally be separated into the enantiomers by separation methods familiar to the person skilled in the art, for example preparative HPLC on a chiral stationary phase.

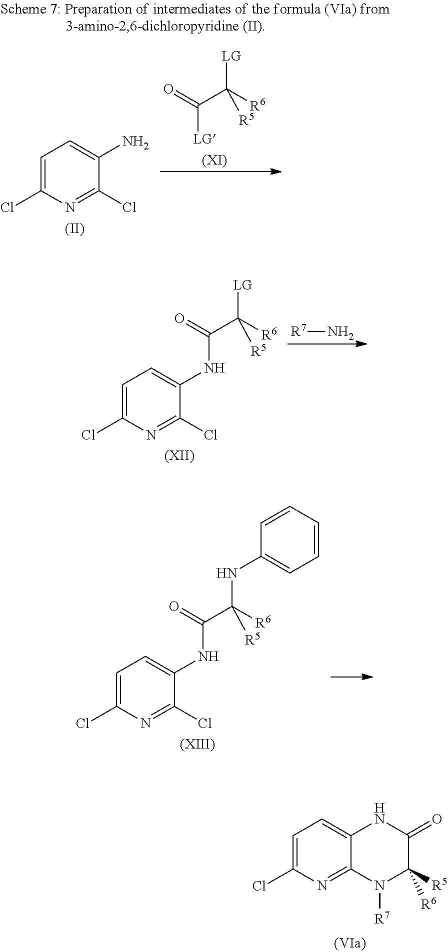

The present invention likewise provides the intermediates of the compounds of the general formula (VIII)

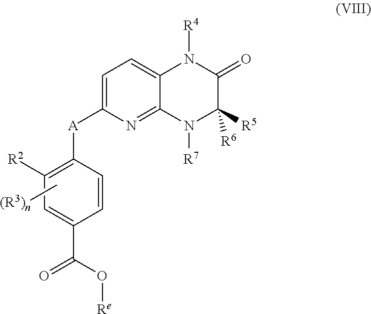

in which A, R², R³, R⁴, R⁵, R⁶, R⁷ and n are each as defined in the general formula (I) and R^{E} represents C₁-C₆-alkyl, which can preferably be used for preparation of the compounds of the general formula (I) according to the invention.

The present invention furthermore provides the intermediates of the compounds of the general formula (IX)

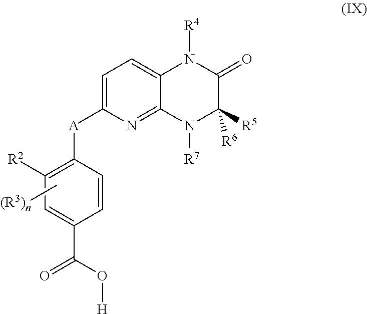

in which A, R², R³, R⁴, R⁵, R⁶, R⁷ and n are each as defined in the general formula (I), and which can likewise preferably be used for preparation of the compounds of the general formula (I) according to the invention.

Especially valuable intermediates for preparation of the compounds according to the invention are the following compounds:
methyl 4-{[(3R)-4-cyclopentyl-1,3-dimethyl-2-oxo-1,2,3,4-tetrahydropyrido[2,3-b]pyrazin-6-yl]amino}-3-methoxybenzoate;
methyl 4-{[(3R)-4-cyclohexyl-1,3-dimethyl-2-oxo-1,2,3,4-tetrahydropyrido[2,3-b]pyrazin-6-yl]amino}-3-methoxybenzoate;
methyl 4-{[(3R)-1,3-dimethyl-2-oxo-4-(propan-2-yl)-1,2,3,4-tetrahydropyrido[2,3-b]pyrazin-6-yl]amino}-3-methoxybenzoate;
methyl 4-{[(3R)-4-cyclopentyl-1,3-dimethyl-2-oxo-1,2,3,4-tetrahydropyrido[2,3-b]pyrazin-6-yl]amino}benzoate;
methyl 4-{[(3R)-4-isopropyl-1,3-dimethyl-2-oxo-1,2,3,4-tetrahydropyrido[2,3-b]pyrazin-6-yl]amino}benzoate;
methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzoate;
methyl 4-{[(3R)-4-cyclohexyl-1,3-dimethyl-2-oxo-1,2,3,4-tetrahydropyrido[2,3-b]pyrazin-6-yl]amino}benzoate;
methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}benzoate;
methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzoate;
methyl 4-{[(3R)-4-cycloheptyl-1,3-dimethyl-2-oxo-1,2,3,4-tetrahydropyrido[2,3-b]pyrazin-6-yl]amino}-3-methoxybenzoate;
methyl 4-{[(3R)-4-cycloheptyl-1,3-dimethyl-2-oxo-1,2,3,4-tetrahydropyrido[2,3-b]pyrazin-6-yl]amino}benzoate;
methyl 4-{[(3R)-4-benzyl-1,3-dimethyl-2-oxo-1,2,3,4-tetrahydropyrido[2,3-b]pyrazin-6-yl]amino}-3-methoxybenzoate;
methyl 4-{[(3R)-4-benzyl-1,3-dimethyl-2-oxo-1,2,3,4-tetrahydropyrido[2,3-b]pyrazin-6-yl]amino}benzoate;
ethyl 4-{[4-(2-methoxyethyl)-1,3-dimethyl-2-oxo-1,2,3,4-tetrahydropyrido[2,3-b]pyrazin-6-yl]amino}benzoate;
4-{[(3R)-4-cyclopentyl-1,3-dimethyl-2-oxo-1,2,3,4-tetrahydropyrido[2,3-b]pyrazin-6-yl]amino}-3-methoxybenzoic acid;
4-{[(3R)-4-cyclohexyl-1,3-dimethyl-2-oxo-1,2,3,4-tetrahydropyrido[2,3-b]pyrazin-6-yl]amino}-3-methoxybenzoic acid;
4-{[(3R)-1,3-dimethyl-2-oxo-4-(propan-2-yl)-1,2,3,4-tetrahydropyrido[2,3-b]pyrazin-6-yl]amino}-3-methoxybenzoic acid;
4-{[(3R)-4-cyclopentyl-1,3-dimethyl-2-oxo-1,2,3,4-tetrahydropyrido[2,3-b]pyrazin-6-yl]amino}benzoic acid;
4-{[(3R)-4-isopropyl-1,3-dimethyl-2-oxo-1,2,3,4-tetrahydropyrido[2,3-b]pyrazin-6-yl]amino}benzoic acid;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzoic acid;
4-{[(3R)-4-cyclohexyl-1,3-dimethyl-2-oxo-1,2,3,4-tetrahydropyrido[2,3-b]pyrazin-6-yl]amino}benzoic acid;
4-{[(3R)-1,3-dimethyl-2-oxo-4-(tetrahydro-2H-pyran-4-yl)-1,2,3,4-tetrahydropyrido[2,3-b]pyrazin-6-yl]amino}benzoic acid;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzoic acid;
4-{[(3R)-4-cycloheptyl-1,3-dimethyl-2-oxo-1,2,3,4-tetrahydropyrido[2,3-b]pyrazin-6-yl]amino}-3-methoxybenzoic acid;
4-{[(3R)-4-cycloheptyl-1,3-dimethyl-2-oxo-1,2,3,4-tetrahydropyrido[2,3-b]pyrazin-6-yl]amino}benzoic acid;
4-{[(3R)-4-benzyl-1,3-dimethyl-2-oxo-1,2,3,4-tetrahydropyrido[2,3-b]pyrazin-6-yl]amino}-3-methoxybenzoic acid;
4-{[(3R)-4-benzyl-1,3-dimethyl-2-oxo-1,2,3,4-tetrahydropyrido[2,3-b]pyrazin-6-yl]amino}benzoic acid and
4-{[4-(2-methoxyethyl)-1,3-dimethyl-2-oxo-1,2,3,4-tetrahydropyrido[2,3-b]pyrazin-6-yl]amino}benzoic acid.

### WORKING EXAMPLES

The examples which follow illustrate the preparation of the compounds according to the invention, without restricting the invention to these examples.

Firstly, the preparation of the intermediates is described, which are preferably used ultimately for preparation of the compounds according to the invention.

IUPAC names were created with the aid of the nomenclature software ACD Name batch, Version 12.01, from Advanced Chemical Development, Inc., and adapted if required, for example to German-language nomenclature.

### Preparation of the Intermediates

### Intermediate 1

### (2R)-2-Azido-N-(2,6-dichloropyridin-3-yl)propanamide

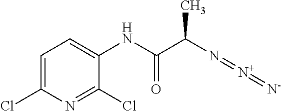 [SMILES: C[C@@H](N=[N+]=[N-])C(=O)Nc1ccc(Cl)nc1Cl]

At −10° C., 5.02 ml of thionyl chloride were added dropwise to a solution of 6.6 g of (2R)-2-azidopropanoic acid (Chem. Eur. J. (2010), 16, pp. 7572-7578) in 250 ml of N,N-dimethylacetamide. The mixture was stirred for 30 min at −10° C., and 10.6 g of 3-amino-2,6-dichloropyridine (CAS 2013-03-13) were then added. The mixture was slowly warmed to RT and stirred for a further 3 hours. Water was added, and the reaction solution was extracted three times with ethyl acetate. The combined organic phases were washed with water and brine, dried over sodium sulphate and concentrated under reduced pressure. The residue was purified by chromatography on silica gel (hexane/ethyl acetate gradient). This gave 10.6 g of (2R)-2-azido-N-(2,6-dichloropyridin-3-yl)propanamide.

¹H NMR (400 MHz, DMSO-d6): δ=1.47 (d, 3H); 4.27 (q, 1H); 7.61 (d, 1H); 8.22 (d, 1H); 10.08 (bs, 1H).

### Intermediate 2

### N-(2,6-Dichloropyridin-3-yl)-D-alaninamide hydrochloride

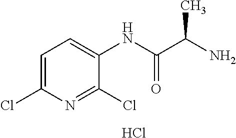

Under argon and at RT, 50 ml of a solution of trimethylphosphine (1M in THF) were added slowly to a solution of 10.0 g of Intermediate 1 in 150 ml THF. The mixture was stirred at RT for 14 hours, and water was then added. The reaction was then evaporated to dryness and the residue was taken up in water. The aqueous solution was extracted twice with dichloromethane and the combined organic phases were dried over sodium sulphate and evaporated to dryness. The residue was taken up in diethyl ether, and HCl (solution in diethyl ether) was added. The resulting crystals were filtered off with suction and dried in a drying cabinet under reduced pressure. This gave 11.4 g of N-(2,6-dichloropyridin-3-yl)-D-alaninamide hydrochloride. The product was pure enough for further reactions.

### Alternative Preparation of Intermediate 2:

At 0° C., 886 ml of a 50% strength solution of T3P (in ethyl acetate) were added slowly to a solution of 50 g of 3-amino-2,6-dichloropyridine (CAS 2013-03-13) and 56.3 g of D-Boc-alanine in 400 ml of pyridine. The mixture was left stirring at 0° C. for a further 4 hours and at RT for 16 hours. The mixture was added to ice-water, and potassium carbonate was added carefully until the solution was alkaline. The reaction was extracted with ethyl acetate and the organic phase was washed with saturated sodium chloride solution, dried over sodium sulphate and evaporated to dryness. This gave 73 g of tert-butyl {(2R)-1-[(2,6-dichloropyridin-3-yl)amino]-1-oxopropan-2-yl}carbamate. These were taken up in 370 ml of dioxane, and 89 ml of conc. hydrochloric acid were added at RT. The mixture was stirred at RT for 90 min, 1000 ml of ethyl acetate were added and the pH was adjusted to alkaline using sodium hydroxide. The suspension was decanted, the phases were separated and the organic phase was evaporated to dryness. The residue was taken up in diethyl ether, and 260 ml of 1N HCl (solution in diethyl ether) were added. The mixture was cooled to 0° C. and the precipitate was filtered off with suction. The precipitate was washed with a little diethyl ether and dried in a drying cabinet. This gave 45.6 g of N-(2,6-dichloropyridin-3-yl)-D-alaninamide hydrochloride.

¹H NMR (400 MHz, DMSO-d6): δ=1.50 (d, 3H); 4.23 (bq, 1H); 7.63 (d, 1H); 8.15 (d, 1H); 8.42 bs, 1H); 10.58 (s, 1H).

### Intermediate 3

### N2-Cyclopentyl-N1-(2,6-dichloropyridin-3-yl)-D-alaninamide

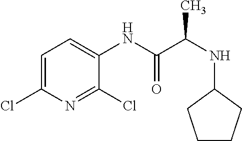 [SMILES: C[C@@H](NC1CCCC1)C(=O)Nc1ccc(Cl)nc1Cl]

At 0° C. and under an argon atmosphere, 23.5 g of sodium triacetoxyborohydride were added to a solution of 10 g of Intermediate 2, 4.04 g of cyclopentanone and 6.06 g of sodium acetate in 400 ml of dichloromethane. After 24 hours, the mixture was carefully poured into saturated sodium bicarbonate solution, the phases were separated and the aqueous phase was extracted once more with dichloromethane. The combined organic phases were dried over sodium sulphate and concentrated under reduced pressure. The residue was purified by chromatography on silica gel (hexane/ethyl acetate gradient). This gave 8.4 g of N2-cyclopentyl-N1-(2,6-dichloropyridin-3-yl)-D-alaninamide

¹H NMR (400 MHz, DMSO-d6): δ=1.27 (d, 3H); 1.31-1.41 (m, 2H); 1.42-1.55 (m, 2H); 1.59-1.73 (m, 3H); 1.73-1.83 (m, 1H); 3.06 (qi, 1H); 3.27 (q, 1H); 7.58 (d, 1H); 8.67 (d, 1H).

### Intermediate 4

### (3R)-6-Chloro-4-cyclopentyl-3-methyl-3,4-dihydropyrido[2,3-b]pyrazin-2(1H)-one

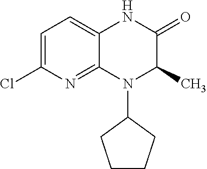 [SMILES: C[C@@H]1C(=O)Nc2ccc(Cl)nc2N1C1CCCC1]

A solution of 8.4 g of Intermediate 3 and 37.8 ml of N,N-diisopropylethylamine in 200 ml of THF was stirred at 170° C. bath temperature for 96 hours. The reaction was diluted with water and extracted three times with dichloromethane. The combined organic phases were concentrated under reduced pressure. Toluene was added, and the mixture was once more evaporated to dryness. The residue was purified by chromatography on silica gel (hexane/ethyl acetate gradient). This gave 6.7 g of (3R)-6-chloro-4-cyclopentyl-3-methyl-3,4-dihydropyrido[2,3-b]pyrazin-2(1H)-one.

¹H NMR (400 MHz, DMSO-d6): δ=1.15 (d, 3H); 1.47-1.83 (sm, 6H); 1.84-1.98 (m, 2H); 4.12 (q, 1H); 4.19 (qi, 1H); 6.67 (d, 1H); 7.00 (d, 1H); 10.61 (s, 1H).

### Intermediate 5

### (3R)-6-Chloro-4-cyclopentyl-1,3-dimethyl-3,4-dihydropyrido[2,3-b]pyrazin-2(1H)-one

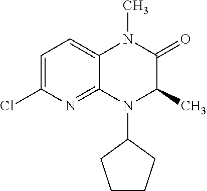 [SMILES: C[C@@H]1C(=O)N(C)c2ccc(Cl)nc2N1C1CCCC1]

At 0° C., 1.51 g of sodium hydride (60% in white oil) were added a little at a time to a solution of 6.7 g of Intermediate 4 and 2.35 ml of methyl iodide in 180 ml of DMF. After 1 hour of stirring at 0° C., the reaction was poured into ice-water and neutralized with saturated aqueous ammonium chloride solution. The mixture was extracted three times with ethyl acetate and the combined organic phases were washed with water, dried over sodium sulphate and evaporated to dryness. The residue was purified by chromatography on silica gel (hexane/ethyl acetate 2:1). This gave 7.1 g of (3R)-6-chloro-4-cyclopentyl-1,3-dimethyl-3,4-dihydropyrido[2,3-b]pyrazin-2(1H)-one.

¹H NMR (400 MHz, DMSO-d6): δ=1.11 (d, 3H); 1.48-1.62 (m, 2H); 1.63-1.82 (m, 4H); 1.87-1.98 (m, 2H); 3.23 (s, 3H); 4.21 (qi, 1H); 4.27 (q, 1H); 6.78 (d, 1H); 7.31 (d, 1H).

### Intermediate 6

### Methyl 4-{[(3R)-4-cyclopentyl-1,3-dimethyl-2-oxo-1,2,3,4-tetrahydropyrido[2,3-b]pyrazin-6-yl]amino}-3-methoxybenzoate

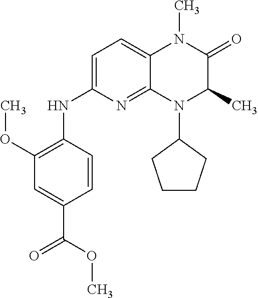 [SMILES: COC(=O)c1ccc(Nc2ccc3c(n2)N(C2CCCC2)[C@H](C)C(=O)N3C)c(OC)c1]

A suspension of 1.5 g of Intermediate 5, 1.94 g of methyl 4-amino-3-methoxybenzoate (CAS 41608-64-4), 0.24 g of palladium(II) acetate, 8.7 g of caesium carbonate and 0.67 g of (+)-BINAP in 120 ml of toluene was stirred at 110° C. under an argon atmosphere for 2.5 hours. The reaction solution was filtered off, the residue was washed with ethyl acetate and the combined organic phases were evaporated to dryness. The residue was purified by RP-HPLC chromatography (column: X-Bridge C18 5 μm 100×30 mm, mobile phase: acetonitrile/water (0.1% by volume formic acid) gradient). This gave 1.08 g of methyl 4-{[(3R)-4-cyclopentyl-1,3-dimethyl-2-oxo-1,2,3,4-tetrahydropyrido[2,3-b]pyrazin-6-yl]amino}-3-methoxybenzoate.

¹H NMR (400 MHz, DMSO-d6): δ=1.08 (d, 3H); 1.58-1.77 (m, 6H); 1.92-2.06 (m, 2H); 3.22 (s, 3H); 3.81 (s, 3H); 3.93 (s, 3H); 4.21 (q, 1H); 4.37 (qi, 1H); 6.65 (d, 1H); 7.28 (d, 1H); 7.45 (d, 1H); 7.52 (dd, 1H); 8.25 (s, 1H); 8.45 (d, 1H).

### Intermediate 7

### 4-{[(3R)-4-Cyclopentyl-1,3-dimethyl-2-oxo-1,2,3,4-tetrahydropyrido[2,3-b]pyrazin-6-yl]amino}-3-methoxybenzoic acid

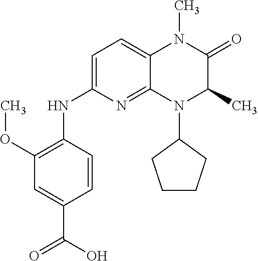 [SMILES: COc1cc(C(=O)O)ccc1Nc1ccc2c(n1)N(C1CCCC1)[C@H](C)C(=O)N2C]

At RT, 25 ml of a 1N lithium hydroxide solution were added to a solution of 1.08 g of Intermediate 6 in 8 ml of THF and 60 ml of methanol, and the mixture was stirred at 50° C. for 14 hours. The mixture was adjusted to pH=7 using 1 N hydrochloric acid and extracted twice with chloroform/methanol (9:1). The combined organic phases were dried over sodium sulphate and the solvent was removed completely under reduced pressure. This gave 1.1 g of 4-{[(3R)-4-cyclopentyl-1,3-dimethyl-2-oxo-1,2,3,4-tetrahydropyrido[2,3-b]pyrazin-6-yl]amino)}-3-methoxybenzoic acid.

UPLC-MS: Rt=1.19 min (M⁺+1=411)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 8

### N2-Cyclohexyl-N1-(2,6-dichloropyridin-3-yl)-D-alaninamide

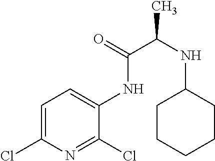 [SMILES: C[C@@H](NC1CCCCC1)C(=O)Nc1ccc(Cl)nc1Cl]

Analogously to the preparation of Intermediate 3, N2-cyclohexyl-N1-(2,6-dichloropyridin-3-yl)-D-alaninamide was prepared from 1.5 g of Intermediate 2, 707 mg of cyclohexanone, 909 mg of sodium acetate and 3.5 g of sodium triacetoxyborohydride in 80 ml of dichloromethane at 0° C. This gave 1.3 g of N2-cyclohexyl-N1-(2,6-dichloropyridin-3-yl)-D-alaninamide as a crude product which could be used without further purification for the next step.

UPLC-MS: Rt=1.49 min (M⁺+1=316, 318, 320)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 9

### (3R)-6-Chloro-4-cyclohexyl-3-methyl-3,4-dihydropyrido[2,3-b]pyrazin-2(1H)-one

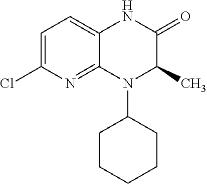 [SMILES: C[C@@H]1C(=O)Nc2ccc(Cl)nc2N1C1CCCCC1]

Analogously to the synthesis of Intermediate 4, (3R)-6-chloro-4-cyclohexyl-3-methyl-3,4-dihydropyrido[2,3-b]pyrazin-2(1H)-one was prepared from 1.3 g of Intermediate 8 and 5.59 ml of N,N-diisopropylethylamine in 100 ml of DMF by heating for 120 hours at a bath temperature of 170° C. This gave 1.08 g of (3R)-6-chloro-4-cycloxyl-3-methyl-3,4-dihydropyrido[2,3-b]pyrazin-2(1H)-one.

¹H NMR (300 MHz, DMSO-d6): δ=1.14 (d, 3H); 1.15-1.97 (5m, 10H); 4.03-4.13 (m, 1H); 4.15 (q, 1H); 6.65 (d, 1H); 7.00 (d, 1H); 10.58 (s, 1H).

### Intermediate 10

### (3R)-6-Chloro-4-cyclohexyl-1,3-dimethyl-3,4-dihydropyrido[2,3-b]pyrazin-2(1H)-one

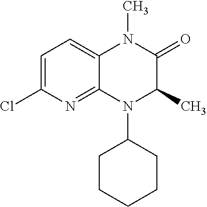 [SMILES: C[C@@H]1C(=O)N(C)c2ccc(Cl)nc2N1C1CCCCC1]

Analogously to the preparation of Intermediate 5, (3R)-6-chloro-4-cyclohexyl-1,3-dimethyl-3,4-dihydropyrido[2,3-b]pyrazin-2(1H)-one was prepared from 1.08 g of Intermediate 9, 232 mg of sodium hydride (60% in white oil) and 0.36 ml of methyl iodide in 50 ml of DMF. Purification by chromatography on silica gel (hexane/ethyl acetate 3:1) gave 1.06 g of (3R)-6-chloro-4-cyclohexyl-1,3-dimethyl-3,4-dihydropyrido[2,3-b]pyrazin-2(1H)-one.

¹H NMR (400 MHz, DMSO-d6): δ=1.10 (d, 3H); 1.17 (tt, 1H); 1.24-1.43 (m, 2H); 1.45-1.85 (m, 6H); 1.94 (bd, 1H); 3.22 (s, 3H); 4.11 (tt, 1H); 4.31 (q, 1H); 6.76 (d, 1H); 7.31 (d, 1H).

### Intermediate 11

### Methyl 4-{[(3R)-4-cyclohexyl-1,3-dimethyl-2-oxo-1,2,3,4-tetrahydropyrido[2,3-b]pyrazin-6-yl]amino}-3-methoxybenzoate

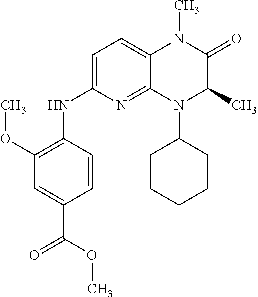 [SMILES: COC(=O)c1ccc(Nc2ccc3c(n2)N(C2CCCCC2)[C@H](C)C(=O)N3C)c(OC)c1]

Analogously to the preparation of Intermediate 6, methyl 4-{[(3R)-4-cyclohexyl-1,3-dimethyl-2-oxo-1,2,3,4-tetrahydropyrido[2,3-b]pyrazin-6-yl]amino}-3-methoxybenzoate was prepared from 450 mg of Intermediate 10, 555 mg of methyl 4-amino-3-methoxybenzoate, 69 mg of palladium(II) acetate, 2.5 g of caesium carbonate and 0.19 g of (+)-BINAP in 15 ml of toluene by stirring for 2.5 hours at 110° C. under an argon atmosphere. Chromatography on silica gel (hexane/ethyl acetate gradient) gave 620 mg of methyl 4-{[(3R)-4-cyclohexyl-1,3-dimethyl-2-oxo-1,2,3,4-tetrahydropyrido[2,3-b]pyrazin-6-yl]amino}-3-methoxybenzoate.

¹H NMR (400 MHz, DMSO-d6): δ=1.08 (d, 3H); 1.14-1.56 (m, 4H); 1.58-1.74 (m, 3H); 1.76-1.94 (m, 2H); 2.09 (bd, 1H); 3.20 (s, 3H); 3.81 (s, 3H); 3.93 (s, 3H); 4.11-4.29 (m, 2H); 6.63 (d, 1H); 7.27 (d, 1H); 7.45 (d, 1H); 7.50 (dd, 1H); 8.31 (s, 1H); 8.59 (d, 1H).

### Intermediate 12

### 4-{[(3R)-4-Cyclohexyl-1,3-dimethyl-2-oxo-1,2,3,4-tetrahydropyrido[2,3-b]pyrazin-6-yl]amino}-3-methoxybenzoic acid

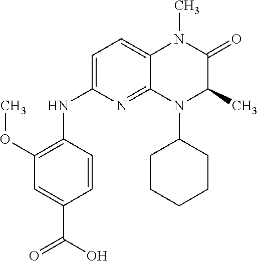 [SMILES: COc1cc(C(=O)O)ccc1Nc1ccc2c(n1)N(C1CCCCC1)[C@H](C)C(=O)N2C]

Analogously to the preparation of Intermediate 7, 4-{[(3R)-4-cyclohexyl-1,3-dimethyl-2-oxo-1,2,3,4-tetrahydropyrido[2,3-b]pyrazin-6-yl]amino}-3-methoxybenzoic acid was prepared from 620 mg of Intermediate 11 and 14 ml of 1N aqueous lithium hydroxide solution in 5 ml of THF and 50 ml of methanol. This gave 710 mg of 4-{[(3R)-4-cyclohexyl-1,3-dimethyl-2-oxo-1,2,3,4-tetrahydropyrido[2,3-b]pyrazin-6-yl]amino}-3-methoxybenzoic acid, which was used in the next stage without further purification.

UPLC-MS: Rt=1.22 min (M⁺+1=425)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 13

### N2-(1-Methylethyl)-N1-(2,6-dichloropyridin-3-yl)-D-alaninamide

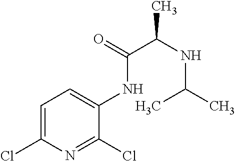 [SMILES: CC(C)N[C@H](C)C(=O)Nc1ccc(Cl)nc1Cl]

Analogously to the preparation of Intermediate 3, N2-(1-methylethyl)-N1-(2,6-dichloropyridin-3-yl)-D-alaninamide was prepared from 0.5 g of Intermediate 2, 0.27 ml of acetone, 303 mg of sodium acetate and 1.18 g of sodium triacetoxyborohydride in 40 ml of dichloromethane at 0° C. This gave 420 mg of N2-(1-methylethyl)-N1-(2,6-dichloropyridin-3-yl)-D-alaninamide. This was used directly in the synthesis of the next stage.

¹H NMR (400 MHz, DMSO-d6): δ=1.02 (d, 3H); 1.05 (d, 3H); 1.27 (d, 3H); 2.77 (sp, 1H); 3.30 (q, 1H); 7.58 (d, 1H); 8.67 (d, 1H).

### Intermediate 14

### (3R)-6-Chloro-3-methyl-4-(propan-2-yl)-3,4-dihydropyrido[2,3-b]pyrazin-2(1H)-one

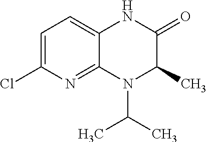 [SMILES: CC(C)N1c2nc(Cl)ccc2NC(=O)[C@H]1C]

Analogously to the synthesis of Intermediate 4, (3R)-6-chloro-3-methyl-4-(propan-2-yl)-3,4-dihydropyrido[2,3-b]pyrazin-2(1H)-one was prepared from 420 mg of Intermediate 13 and 2.1 ml of N,N-diisopropylethylamine in 40 ml of DMF by heating for 72 hours at a bath temperature of 170° C. This gave 320 mg of (3R)-6-chloro-3-methyl-4-(propan-2-yl)-3,4-dihydropyrido[2,3-b]pyrazin-2(1H)-one.

¹H NMR (300 MHz, DMSO-d6): δ=1.16 (d, 3H); 1.24 (d, 3H); 1.27 (d, 3H); 4.16 (q, 1H); 4.43 (sp, 1H); 6.65 (d, 1H); 7.00 (d, 1H); 10.56 (s, 1H).

### Intermediate 15

### (3R)-6-Chloro-1,3-dimethyl-4-(propan-2-yl)-3,4-dihydropyrido[2,3-b]pyrazin-2(1H)-one

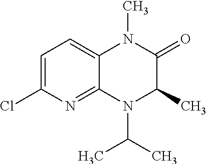 [SMILES: CC(C)N1c2nc(Cl)ccc2N(C)C(=O)[C@H]1C]

Analogously to the preparation of Intermediate 5, (3R)-6-chloro-1,3-dimethyl-4-(propan-2-yl)-3,4-dihydropyrido[2,3-b]pyrazin-2(1H)-one was prepared from 320 mg of Intermediate 14, 80 mg of sodium hydride (60% in white oil) and 0.13 ml of methyl iodide in 20 ml of DMF. Purification by chromatography on silica gel (hexane/ethyl acetate 2:1) gave 280 mg of (3R)-6-chloro-1,3-dimethyl-4-(propan-2-yl)-3,4-dihydropyrido[2,3-b]pyrazin-2(1H)-one.

¹H NMR (400 MHz, DMSO-d6): δ=1.12 (d, 3H); 1.23 (d, 3H); 1.27 (d, 3H); 3.22 (s, 3H); 4.32 (q, 1H); 4.47 (sp, 1H); 6.76 (d, 1H); 7.31 (d, 1H).

### Intermediate 16

### Methyl 4-{[(3R)-1,3-dimethyl-2-oxo-4-(propan-2-yl)-1,2,3,4-tetrahydropyrido[2,3-b]pyrazin-6-yl]amino}-3-methoxybenzoate

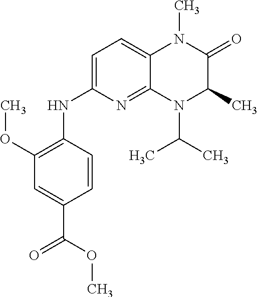 [SMILES: COC(=O)c1ccc(Nc2ccc3c(n2)N(C(C)C)[C@H](C)C(=O)N3C)c(OC)c1]

Analogously to the preparation of Intermediate 6, methyl 4-{[(3R)-1,3-dimethyl-2-oxo-4-(propan-2-yl)-1,2,3,4-tetrahydropyrido[2,3-b]pyrazin-6-yl]amino}-3-methoxybenzoate was prepared from 725 mg of Intermediate 15, 1.04 g of methyl 4-amino-3-methoxybenzoate, 128 mg of palladium(II) acetate, 4.65 g of caesium carbonate and 356 mg of (+)-BINAP in 40 ml of toluene under an argon atmosphere. Purification by chromatography on silica gel (hexane/ethyl acetate gradient) gave 442 mg of methyl 4-{[(3R)-1,3-dimethyl-2-oxo-4-(propan-2-yl)-1,2,3,4-tetrahydropyrido[2,3-b]pyrazin-6-yl]amino}-3-methoxybenzoate.

¹H NMR (400 MHz, CDCl₃): δ=1.10 (d, 3H); 1.26 (d, 3H); 1.33 (d, 3H); 3.21 (s, 3H); 3.81 (s, 3H); 3.93 (s, 3H); 4.26 (q, 1H); 4.58 (sp, 1H); 6.62 (d, 1H); 7.27 (d, 1H); 7.44 (d, 1H); 7.55 (dd, 1H); 8.27 (s, 1H); 8.50 (d, 1H).

### Intermediate 17

### 4-{[(3R)-1,3-Dimethyl-2-oxo-4-(propan-2-yl)-1,2,3,4-tetrahydropyrido[2,3-b]pyrazin-6-yl]amino}-3-methoxybenzoic acid

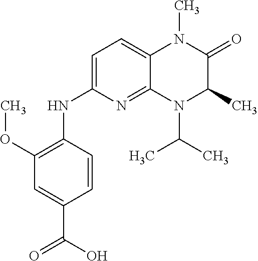 [SMILES: COc1cc(C(=O)O)ccc1Nc1ccc2c(n1)N(C(C)C)[C@H](C)C(=O)N2C]

Analogously to the preparation of Intermediate 7, 4-{[(3R)-1,3-dimethyl-2-oxo-4-(propan-2-yl)-1,2,3,4-tetrahydropyrido[2,3-b]pyrazin-6-yl]amino}-3-methoxybenzoic acid was prepared from 442 mg of Intermediate 16 and 5.5 ml of 2N lithium hydroxide solution in 5 ml of THF and 15 ml of methanol. This gave 407 mg of 4-{[(3R)-1,3-dimethyl-2-oxo-4-(propan-2-yl)-1,2,3,4-tetrahydropyrido[2,3-b]pyrazin-6-yl]amino}-3-methoxybenzoic acid, which was used in the next stage without further purification.

UPLC-MS: Rt=1.11 min (M⁺+1=385)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 18

### Methyl 4-{[(3R)-4-cyclopentyl-1,3-dimethyl-2-oxo-1,2,3,4-tetrahydropyrido[2,3-b]pyrazin-6-yl]amino}benzoate

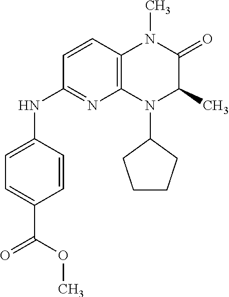 [SMILES: COC(=O)c1ccc(Nc2ccc3c(n2)N(C2CCCC2)[C@H](C)C(=O)N3C)cc1]

A suspension of 1.335 g of Intermediate 5, 1.443 g of methyl 4-aminobenzoate (CAS 619-45-4), 214 mg of palladium(II) acetate, 7.774 g of caesium carbonate and 594 mg of (+)-BINAP in 75 ml of toluene was stirred at 110° C. under an argon atmosphere for 3 hours. The reaction solution was filtered off, the residue was washed with ethyl acetate and the combined organic phases were evaporated to dryness. The residue was purified by RP-HPLC chromatography (column: X-Bridge C18 5 μm 100×30 mm, mobile phase: acetonitrile/water (0.1% by volume formic acid) gradient). This gave 938 mg of methyl 4-{[(3R)-4-cyclopentyl-1,3-dimethyl-2-oxo-1,2,3,4-tetrahydropyrido[2,3-b]pyrazin-6-yl]amino}benzoate.

UPLC-MS: Rt=1.34 min (M⁺+1=395)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 19

### 4-{[(3R)-4-Cyclopentyl-1,3-dimethyl-2-oxo-1,2,3,4-tetrahydropyrido[2,3-b]pyrazin-6-yl]amino}benzoic acid

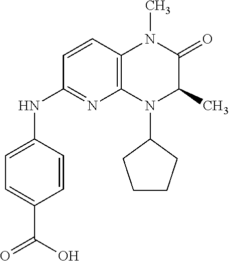 [SMILES: C[C@@H]1C(=O)N(C)c2ccc(Nc3ccc(C(=O)O)cc3)nc2N1C1CCCC1]

At RT, 11.9 ml of a 2N lithium hydroxide solution were added to a solution of 938 mg of Intermediate 18 in 10 ml of THF and 30 ml of methanol, and the mixture was stirred at 55° C. for 6 hours. The mixture was adjusted to pH=7 using 1N hydrochloric acid and extracted twice with ethyl acetate. The combined organic phases were dried over sodium sulphate and the solvent was removed completely under reduced pressure. This gave 1.09 g of 4-{[(3R)-4-cyclopentyl-1,3-dimethyl-2-oxo-1,2,3,4-tetrahydropyrido[2,3-b]pyrazin-6-yl]amino)}benzoic acid, which was used in the next stage without further purification.

UPLC-MS: Rt=1.10 min (M⁺+1=381)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 20

### Methyl 4-{[(3R)-4-isopropyl-1,3-dimethyl-2-oxo-1,2,3,4-tetrahydropyrido[2,3-b]pyrazin-6-yl]amino}benzoate

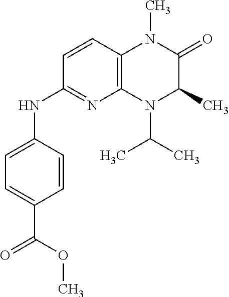 [SMILES: COC(=O)c1ccc(Nc2ccc3c(n2)N(C(C)C)[C@H](C)C(=O)N3C)cc1]

A suspension of 800 mg of Intermediate 15, 953 mg of methyl 4-aminobenzoate, 142 mg of palladium(II) acetate, 5.14 g of caesium carbonate and 393 mg of (+)-BINAP in 44 ml of toluene was stirred at 110° C. under an argon atmosphere for 3 hours. The reaction solution was filtered off, the residue was washed with ethyl acetate and the combined organic phases were evaporated to dryness. The residue was purified by RP-HPLC chromatography (column: X-Bridge C18 5 μm 100×30 mm, mobile phase: acetonitrile/water (0.1% by volume formic acid) gradient). This gave 404 mg of methyl 4-{[(3R)-4-isopropyl-1,3-dimethyl-2-oxo-1,2,3,4-tetrahydropyrido[2,3-b]pyrazin-6-yl]amino}benzoate.

UPLC-MS: Rt=1.26 min (M⁺+1=369)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 21

### 4-{[(3R)-4-Isopropyl-1,3-dimethyl-2-oxo-1,2,3,4-tetrahydropyrido[2,3-b]pyrazin-6-yl]amino}benzoic acid

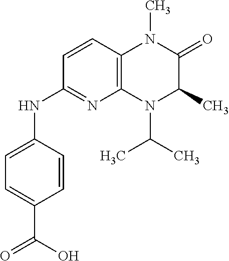 [SMILES: CC(C)N1c2nc(Nc3ccc(C(=O)O)cc3)ccc2N(C)C(=O)[C@H]1C]

At RT, 5.5 ml of a 2N lithium hydroxide solution were added to a solution of 404 mg of Intermediate 20 in 6 ml of THF and 18 ml of methanol, and the mixture was stirred at 55° C. for 6 hours. The mixture was adjusted to pH=7 using 1N hydrochloric acid and extracted twice with ethyl acetate. The combined organic phases were dried over sodium sulphate and the solvent was removed completely under reduced pressure. This gave 427 mg of 4-{[(3R)-4-isopropyl-1,3-dimethyl-2-oxo-1,2,3,4-tetrahydropyrido[2,3-b]pyrazin-6-yl]amino)}benzoic acid, which was used in the next stage without further purification.

UPLC-MS: Rt=1.04 min (M⁺+1=355)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 22

### N-(2,6-Dichloropyridin-3-yl)-N2-(tetrahydro-2H-pyran-4-yl)-D-alaninamide

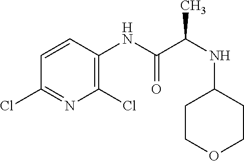 [SMILES: C[C@@H](NC1CCOCC1)C(=O)Nc1ccc(Cl)nc1Cl]

Analogously to the preparation of Intermediate 3, N-(2,6-dichloropyridin-3-yl)-N2-(tetrahydro-2H-pyran-4-yl)-D-alaninamide was prepared from 5 g of Intermediate 2, 2.4 g of tetrahydro-4H-pyran-4-one, 3 g of sodium acetate and 11.8 g of sodium triacetoxyborohydride in 267 ml of dichloromethane at 0° C. This gave 5 g of N-(2,6-dichloropyridin-3-yl)-N2-(tetrahydro-2H-pyran-4-yl)-D-alaninamide.

### Larger Reaction:

At 0° C., 12.1 g of sodium acetate and 47 g of sodium triacetoxyborohydride were added to a suspension of 20 g of Intermediate 2 and 9.6 g tetrahydro-4H-pyran-4-one in 1.07 l of dichloromethane. The mixture was stirred for 16 hours while warming to RT. The reaction was poured carefully into a saturated sodium bicarbonate solution and stirred. The phases were separated and the aqueous phase was extracted once with dichloromethane. The combined organic phases were dried over sodium sulphate and the solvent was removed completely under reduced pressure. The residue was purified by chromatography on silica gel (hexane/ethyl acetate gradient). This gave 15 g of N-(2,6-dichloropyridin-3-yl)-N2-(tetrahydro-2H-pyran-4-yl)-D-alaninamide.

¹H NMR (400 MHz, CDCl₃): δ=1.35-1.57 (m, 2H); 1.44 (d, 3H); 1.84 (dq, 1H); 1.95 (dq, 1H); 2.63-2.82 (m, 1H); 3.38 (td, 1H); 3.45 (q, 1H); 3.91-4.08 (m, 2H); 7.28 (d, 1H); 8.84 (d, 1H).

### Intermediate 23

### (3R)-6-Chloro-3-methyl-4-(tetrahydro-2H-pyran-4-yl)-3,4-dihydropyrido[2,3-b]pyrazin-2(1H)-one

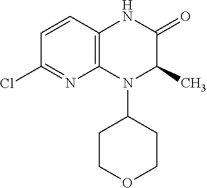 [SMILES: C[C@@H]1C(=O)Nc2ccc(Cl)nc2N1C1CCOCC1]

Analogously to the synthesis of Intermediate 4, (3R)-6-chloro-3-methyl-4-(tetrahydro-2H-pyran-4-yl)-3,4-dihydropyrido[2,3-b]pyrazin-2(1H)-one was prepared from 5 g of Intermediate 22 and 20.3 ml of N,N-diisopropylethylamine in 109 ml of DMF after 15 hours at a bath temperature of 175° C. This gave 1.9 g of (3R)-6-chloro-3-methyl-4-(tetrahydro-2H-pyran-4-yl)-3,4-dihydropyrido[2,3-b]pyrazin-2(1H)-one.

### Larger Reaction:

A solution of 7.8 g of Intermediate 22 and 31.7 ml of N,N-diisopropylethylamine in 170 ml of DMF was divided into 4 individual sealed pressure vessels and heated at a bath temperature of 175° C. for 10 hours. After cooling to RT, the solutions were re-combined, diluted with ethyl acetate and extracted three times with semisaturated sodium chloride solution. The organic phase was dried over sodium sulphate and the solvent was removed completely under reduced pressure. The residue was purified by chromatography on silica gel (dichloromethane/methanol gradient). This gave 4.1 g of (3R)-6-chloro-3-methyl-4-(tetrahydro-2H-pyran-4-yl)-3,4-dihydropyrido[2,3-b]pyrazin-2(1H)-one.

¹H NMR (300 MHz, CDCl₃): δ=1.32 (d, 3H); 1.65 (d, 1H); 1.82 (dq, 1H); 1.98 (dq, 1H); 2.07 (d, 1H); 3.57 (qd, 2H); 4.03-4.12 (m, 2H); 4.25 (q, 1H); 4.55 (tt, 1H); 6.65 (d, 1H); 6.92 (d, 1H); 8.92 (s, 1H).

### Intermediate 24

### (3R)-6-Chloro-1,3-dimethyl-4-(tetrahydro-2H-pyran-4-yl)-3,4-dihydropyrido[2,3-b]pyrazin-2(1H)-one

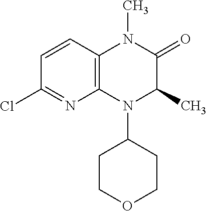 [SMILES: C[C@@H]1C(=O)N(C)c2ccc(Cl)nc2N1C1CCOCC1]

Analogously to the preparation of Intermediate 5, (3R)-6-chloro-1,3-dimethyl-4-(tetrahydro-2H-pyran-4-yl)-3,4-dihydropyrido[2,3-b]pyrazin-2(1H)-one was prepared from 4.65 g of Intermediate 23, 941 mg of sodium hydride (60% in white oil) and 1.46 ml of methyl iodide in 198 ml of DMF. After aqueous work-up, this gave 4.64 g of (3R)-6-chloro-1,3-dimethyl-4-(tetrahydro-2H-pyran-4-yl)-3,4-dihydropyrido[2,3-b]pyrazin-2(1H)-one.

### Synthesis of More Material:

A solution of 3.2 g of Intermediate 23, 647 mg of sodium hydride (60% in white oil) and 1.01 ml of methyl iodide in 137 ml of DMF was stirred at RT for 16 hours. The reaction was poured into water and extracted three times with ethyl acetate. The combined organic phases were washed with saturated ammonium chloride solution and semisaturated sodium chloride solution and dried over sodium sulphate, and the solvent was removed completely under reduced pressure. This gave 2.8 g of (3R)-6-chloro-1,3-dimethyl-4-(tetrahydro-2H-pyran-4-yl)-3,4-dihydropyrido[2,3-b]pyrazin-2(1H)-one.

¹H NMR (300 MHz, CDCl₃): δ=1.24 (d, 3H); 1.66 (dq, 1H); 1.82 (dq, 1H); 1.97 (qd, 1H); 2.06 (dq, 1H); 3.32 (s, 3H); 3.57 (tdd, 2H); 4.01-4.13 (m, 2H); 4.32 (q, 1H); 4.55 (tt, 1H); 6.70 (d, 1H); 7.01 (d, 1H).

### Intermediate 25

### Methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzoate

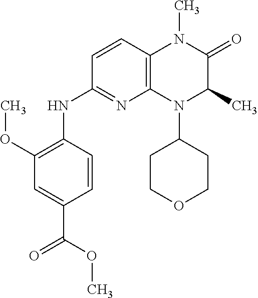 [SMILES: COC(=O)c1ccc(Nc2ccc3c(n2)N(C2CCOCC2)[C@H](C)C(=O)N3C)c(OC)c1]

A suspension of 2.2 g of Intermediate 24, 2.56 g of methyl 4-amino-3-methoxybenzoate (CAS 41608-64-4), 0.317 g of palladium(II) acetate, 11.5 g of caesium carbonate and 0.88 g of (+)-BINAP in 158 ml of toluene was stirred at 120° C. under an argon atmosphere for 5 hours. The reaction solution was added to water and extracted twice with ethyl acetate, and the combined organic phases were washed with saturated sodium chloride solution, dried over sodium sulphate and evaporated to dryness. The residue was purified by chromatography on silica gel (hexane/ethyl acetate gradient). This gave 2 g of methyl 4-{[(3R)-1,3-Dimethyl-2-oxo-4-(tetrahydro-2H-pyran-4-yl)-1,2,3,4-tetrahydropyrido[2,3-b]pyrazin-6-yl]amino}-3-methoxybenzoate.

¹H NMR (300 MHz, CDCl₃): δ=1.25 (d, 3H); 1.73 (d, 1H); 1.86 (dq, 1H); 2.02 (dq, 1H); 2.16 (d, 1H); 3.33 (s, 3H); 3.62 (qd, 2H); 3.92 (s, 3H); 3.99 (s, 3H); 4.08-4.17 (m, 2H); 4.33 (q, 1H); 4.59 (tt, 1H); 6.28 (d, 1H); 7.06 (d, 1H); 7.17 (s, 1H); 7.55 (d, 1H); 7.66 (dd, 1H); 8.37 (d, 1H).

### Intermediate 26

### 4-{[(3R)-1,3-Dimethyl-2-oxo-4-(tetrahydro-2H-pyran-4-yl)-1,2,3,4-tetrahydropyrido[2,3-b]pyrazin-6-yl]amino}-3-methoxybenzoic acid

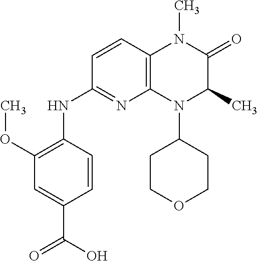 [SMILES: COc1cc(C(=O)O)ccc1Nc1ccc2c(n1)N(C1CCOCC1)[C@H](C)C(=O)N2C]

A solution of 1.35 g of Intermediate 25 and 30.6 ml of 1N of aqueous lithium hydroxide solution in 10 ml of THF and 72 ml of methanol was stirred at 60° C. for 5 hours. The reaction was diluted with water and extracted twice with ethyl acetate. The separated aqueous phase was adjusted to pH<4 by addition of dilute hydrochloric acid and extracted three times with ethyl acetate. The combined organic phases were washed with semisaturated sodium chloride solution and dried over sodium sulphate, and the solvent was removed under reduced pressure. This gave 1.18 g of 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzoic acid.

UPLC-MS: Rt=1.01 min (M⁺+1=427)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

¹H NMR (400 MHz, DMSO-d6): δ=1.10 (d, 3H); 1.62 (bd, 1H); 1.79 (qd, 1H); 1.90-2.03 (m, 2H); 3.21 (s, 3H); 3.39-3.53 (m, 2H); 3.92 (s, 3H); 3.94-4.06 (m, 2H); 4.25 (q, 1H); 4.38 (tt, 1H); 6.65 (d, 1H); 7.29 (d, 1H); 7.45 (d, 1H); 7.52 (dd, 1H); 8.25 (s, 1H); 8.48 (d, 1H); 12.21 (bs, 1H).

### Intermediate 27

### 4-Nitro-N-{trans-4-[4-(cyclopropylmethyl)piperazin-1-yl]cyclohexyl}benzenesulphonamide

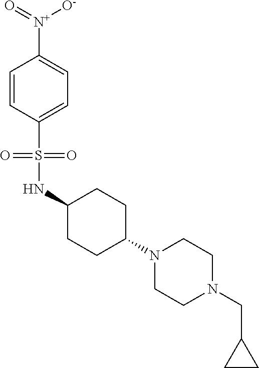 [SMILES: O=[N+]([O-])c1ccc(S(=O)(=O)N[C@H]2CC[C@H](N3CCN(CC4CC4)CC3)CC2)cc1]

At 0° C., 3.58 ml of triethylamine were added to a solution of 1.5 g of 4-nitrobenzenesulphonyl chloride (CAS 98-74-8) and 1.68 g of trans-4-[4-(cyclopropylmethyl)piperazin-1-yl]cyclohexanamine (CAS 876461-31-3, prepared analogously to WO2012049153) in 37.5 ml of dichloromethane, and the mixture was stirred for 16 hours, the temperature being slowly increased to RT. The reaction was diluted with dichloromethane and washed with water and saturated sodium chloride solution, dried over sodium sulphate and evaporated to dryness. The residue that remained was purified by chromatography on silica gel (dichloromethane/methanol gradient). This gave 575 mg of 4-nitro-N-{trans-4-[4-(cyclopropylmethyl)piperazin-1-yl]cyclohexyl}benzenesulphonamide.

¹H NMR (300 MHz, DMSO-d6): δ=−0.02-0.07 (m, 2H); 0.37-0.47 (m, 2H); 0.70-0.83 (m, 1H); 1.04-1.25 (m, 4H); 1.58-1.74 (m, 4H); 2.03-2.16 (m, 3H); 2.28-2.47 (m, 7H); 2.87-3.02 (m, 1H); 8.01-8.10 (m, 3H); 8.40 (d, 2H).

### Intermediate 28

### 4-Amino-N-{trans-4-[4-(cyclopropylmethyl)piperazin-1-yl]cyclohexyl}benzenesulphonamide

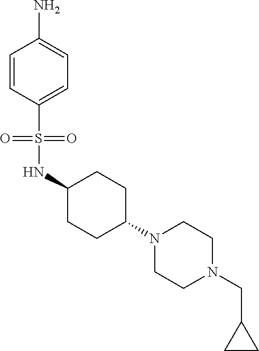 [SMILES: Nc1ccc(S(=O)(=O)N[C@H]2CC[C@H](N3CCN(CC4CC4)CC3)CC2)cc1]

A suspension of 560 mg of Intermediate 28 and 56 mg of palladium (10% on activated carbon) in 13 ml of methanol was shaken under a hydrogen atmosphere at RT for 10 hours. The mixture was filtered off through kieselguhr and the solution was evaporated to dryness. This gave 500 mg of 4-amino-N-{trans-4-[4-(cyclopropylmethyl)piperazin-1-yl]cyclohexyl}benzenesulphonamide.

¹H NMR (300 MHz, DMSO-d6): δ=−0.01-0.06 (m, 2H); 0.38-0.46 (m, 2H); 0.71-0.82 (m, 1H); 1.01-1.17 (m, 4H); 1.57-1.73 (m, 4H); 2.00-2.13 (m+d, 3H); 2.28-2.46 (m, 7H); 2.67-2.78 (m, 1H); 3.16 (d, 1H); 5.86 (s, 2H); 6.58 (d, 2H); 7.08 (d, 1H); 7.41 (d, 2H).

### Intermediate 29

### Methyl 4-{[(3R)-4-cyclohexyl-1,3-dimethyl-2-oxo-1,2,3,4-tetrahydropyrido[2,3-b]pyrazin-6-yl]amino}benzoate

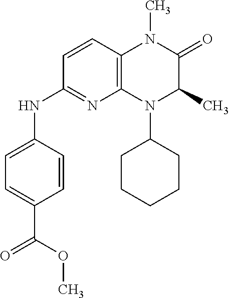 [SMILES: COC(=O)c1ccc(Nc2ccc3c(n2)N(C2CCCCC2)[C@H](C)C(=O)N3C)cc1]

Analogously to the preparation of Intermediate 6, methyl 4-{[(3R)-4-cyclohexyl-1,3-dimethyl-2-oxo-1,2,3,4-tetrahydropyrido[2,3-b]pyrazin-6-yl]amino}benzoate was prepared from 450 mg of Intermediate 10, 463 mg of methyl 4-aminobenzoate, 69 mg of palladium(II) acetate, 2.5 g of caesium carbonate and 191 mg of (+)-BINAP in 15 ml of toluene after 2.5 hours of stirring at 110° C. under an argon atmosphere. Purification by chromatography on silica gel (hexane/ethyl acetate gradient) gave 400 mg of methyl 4-{[(3R)-4-cyclohexyl-1,3-dimethyl-2-oxo-1,2,3,4-tetrahydropyrido[2,3-b]pyrazin-6-yl]amino}benzoate.

¹H NMR (400 MHz, DMSO-d6): δ=1.09 (d, 3H); 1.15-1.32 (m, 1H); 1.31-1.57 (m, 3H); 1.57-1.78 (m, 3H); 1.78-1.97 (m, 2H); 2.06-2.19 (m, 1H); 3.21 (s, 3H); 3.79 (s, 3H); 4.13-4.31 (m, 2H); 6.30 (d, 1H); 7.29 (d, 1H); 7.73-7.86 (m, 4H); 9.35 (s, 1H).

### Intermediate 30

### 4-{[(3R)-4-Cyclohexyl-1,3-dimethyl-2-oxo-1,2,3,4-tetrahydropyrido[2,3-b]pyrazin-6-yl]amino}benzoic acid

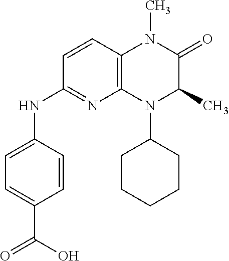 [SMILES: C[C@@H]1C(=O)N(C)c2ccc(Nc3ccc(C(=O)O)cc3)nc2N1C1CCCCC1]

Analogously to the preparation of Intermediate 7, 4-{[(3R)-4-cyclohexyl-1,3-dimethyl-2-oxo-1,2,3,4-tetrahydropyrido[2,3-b]pyrazin-6-yl]amino}benzoic acid was prepared from 400 mg of Intermediate 29 and 9.8 ml of aqueous 1N lithium hydroxide solution in 3.5 ml of THF and 35 ml of methanol. This gave 390 mg of 4-{[(3R)-4-cyclohexyl-1,3-dimethyl-2-oxo-1,2,3,4-tetrahydropyrido[2,3-b]pyrazin-6-yl]amino}benzoic acid.

UPLC-MS: Rt=1.14 min (M⁺+1=395)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 31

### Methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}benzoate

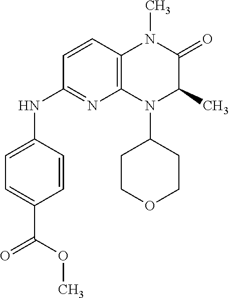 [SMILES: COC(=O)c1ccc(Nc2ccc3c(n2)N(C2CCOCC2)[C@H](C)C(=O)N3C)cc1]

Analogously to the preparation of Intermediate 25, methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}benzoate was prepared from 1.8 g of Intermediate 24, 1.75 g of methyl 4-aminobenzoate, 260 mg of palladium(II) acetate, 9.4 g of caesium carbonate and 720 mg of (+)-BINAP in 129 ml of toluene after 5 hours of stirring at 120° C. under an argon atmosphere. Purification by chromatography on silica gel (dichloromethane/methanol gradient) gave 1.2 g of methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}benzoate.

¹H NMR (300 MHz, CDCl₃): δ=1.25 (d, 3H); 1.70 (d, 1H); 1.86 (dq, 1H); 2.02 (dq, 1H); 2.13 (d, 1H); 3.33 (s, 3H); 3.54-3.69 (m, 2H); 3.91 (s, 3H); 4.11 (dt, 2H); 4.33 (q, 1H); 4.60 (bs, 1H); 6.34 (d, 1H); 7.10 (d, 1H); 7.45 (d, 2H); 7.98 (d, 2H).

### Intermediate 32

### 4-{[(3R)-1,3-Dimethyl-2-oxo-4-(tetrahydro-2H-pyran-4-yl)-1,2,3,4-tetrahydropyrido[2,3-b]pyrazin-6-yl]amino}benzoic acid

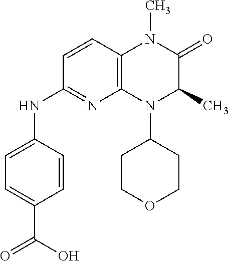 [SMILES: C[C@@H]1C(=O)N(C)c2ccc(Nc3ccc(C(=O)O)cc3)nc2N1C1CCOCC1]

Analogously to the preparation of Intermediate 26, 4-{[(3R)-1,3-dimethyl-2-oxo-4-(tetrahydro-2H-pyran-4-yl)-1,2,3,4-tetrahydropyrido[2,3-b]pyrazin-6-yl]amino}benzoic acid was prepared from 1.15 g of Intermediate 31 and 28 ml of 1N aqueous lithium hydroxide solution in 8.8 ml of THF and 66 ml of methanol. This gave 850 mg of 4-{[(3R)-1,3-dimethyl-2-oxo-4-(tetrahydro-2H-pyran-4-yl)-1,2,3,4-tetrahydropyrido[2,3-b]pyrazin-6-yl]amino)}benzoic acid.

¹H NMR (400 MHz, DMSO-d6): δ=1.10 (d, 3H); 1.63 (bd, 1H); 1.82 (dq, 1H); 1.92-2.05 (m, 2H); 3.21 (s, 3H); 3.48 (dq, 2H); 3.95-4.09 (m, 2H); 4.26 (q, 1H); 4.40 (tt, 1H); 6.33 (d, 1H); 7.30 (d, 1H); 7.72 (d, 2H); 7.80 (d, 2H); 9.28 (s, 1H); 12.31 (bs, 1H).

### Intermediate 33

### Methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzoate

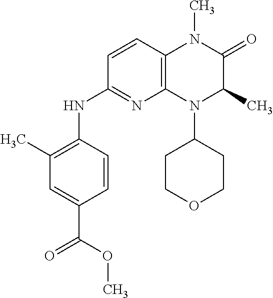 [SMILES: COC(=O)c1ccc(Nc2ccc3c(n2)N(C2CCOCC2)[C@H](C)C(=O)N3C)c(C)c1]

Analogously to the preparation of Intermediate 25, methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzoate was prepared from 848 mg of Intermediate 24, 900 mg of methyl 4-amino-3-methylbenzoate (CAS 18595-14-7), 122 mg of palladium(II) acetate, 4.4 g of caesium carbonate and 339 mg of (+)-BINAP in 61 ml of toluene after 4 hours of stirring at 120° C. under an argon atmosphere. Purification by chromatography on silica gel (hexane/ethyl acetate gradient) gave 575 mg of methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzoate.

¹H NMR (300 MHz, CDCl₃): δ=1.25 (d, 3H); 1.68 (d, 1H); 1.84 (dq, 1H); 2.00 (dq, 1H); 2.08 (d, 1H); 2.35 (s, 3H); 3.32 (s, 3H); 3.56 (t, 2H); 3.90 (s, 3H); 4.05-4.18 (m, 2H); 4.31 (q, 1H); 4.56 (t, 1H); 6.22 (s, 1H); 6.34 (d, 1H); 7.06 (d, 1H); 7.80-7.99 (m, 3H).

### Intermediate 34

### 4-{[(3R)-1,3-Dimethyl-2-oxo-4-(tetrahydro-2H-pyran-4-yl)-1,2,3,4-tetrahydropyrido[2,3-b]pyrazin-6-yl]amino}-3-methylbenzoic acid

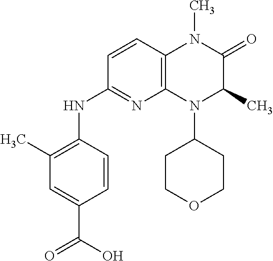 [SMILES: Cc1cc(C(=O)O)ccc1Nc1ccc2c(n1)N(C1CCOCC1)[C@H](C)C(=O)N2C]

Analogously to the preparation of Intermediate 26, 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzoic acid was prepared from 550 mg of Intermediate 33 and 12.3 ml of aqueous 1N lithium hydroxide solution in 3.9 ml of THF and 29 ml of methanol. This gave 440 mg of 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzoic acid.

UPLC-MS: Rt=0.97 min (M⁺+1=411)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 35

### N2-Cycloheptyl-N1-(2,6-dichloropyridin-3-yl)-D-alaninamide

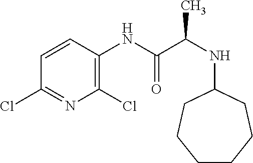 [SMILES: C[C@@H](NC1CCCCCC1)C(=O)Nc1ccc(Cl)nc1Cl]

Analogously to the preparation of Intermediate 3, N2-cycloheptyl-N1-(2,6-dichloropyridin-3-yl)-D-alaninamide was prepared from 1.5 g of Intermediate 2, 809 mg of cycloheptanone, 909 mg of sodium acetate and 3.5 g of sodium triacetoxyborohydride in 80 ml of dichloromethane at 0° C. This gave 1.4 g of N2-cycloheptyl-N1-(2,6-dichloropyridin-3-yl)-D-alaninamide.

¹H NMR (400 MHz, DMSO-d6): δ=1.26 (d, 3H); 1.29-1.42 (m, 4H); 1.42-1.55 (m, 4H); 1.55-1.69 (m, 3H); 1.75-1.88 (m, 2H); 2.56-2.67 (m, 1H); 3.30 (m, 1H); 7.58 (d, 1H); 8.68 (d, 1H).

### Intermediate 36

### (3R)-6-Chloro-4-cycloheptyl-3-methyl-3,4-dihydropyrido[2,3-b]pyrazin-2(1H)-one

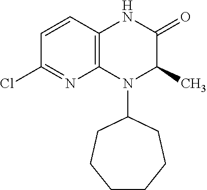 [SMILES: C[C@@H]1C(=O)Nc2ccc(Cl)nc2N1C1CCCCCC1]

Analogously to the synthesis of Intermediate 4, (3R)-6-chloro-4-cycloheptyl-3-methyl-3,4-dihydropyrido[2,3-b]pyrazin-2(1H)-one was prepared from 1.4 g of Intermediate 35 and 5.77 ml of N,N-diisopropylethylamine in 70 ml of DMF by heating for 72 hours at a bath temperature of 170° C. This gave 1.18 g of (3R)-6-chloro-4-cycloheptyl-3-methyl-3,4-dihydropyrido[2,3-b]pyrazin-2(1H)-one.

¹H NMR (300 MHz, DMSO-d6): δ=1.16 (d, 3H); 1.37-1.63 (m, 6H); 1.63-2.00 (m, 6H); 3.96-4.09 (m, 1H); 4.17 (q, 1H); 6.64 (d, 1H); 6.98 (d, 1H); 10.57 (s, 1H).

### Intermediate 37

### (3R)-6-Chloro-4-cycloheptyl-1,3-dimethyl-3,4-dihydropyrido[2,3-b]pyrazin-2(1H)-one

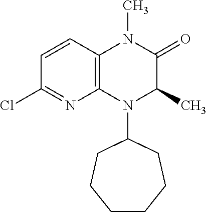 [SMILES: C[C@@H]1C(=O)N(C)c2ccc(Cl)nc2N1C1CCCCCC1]

In analogy to the preparation of Intermediate 5, (3R)-6-chloro-4-cycloheptyl-1,3-dimethyl-3,4-dihydropyrido[2,3-b]pyrazin-2(1H)-one was prepared from 1.18 g of Intermediate 36, 241 mg of sodium hydride (60% in white oil) and 0.38 ml of methyl iodide in 50 ml of DMF. Purification by chromatography on silica gel (hexane/ethyl acetate 3:1) gave 1.11 g of (3R)-6-chloro-4-cycloheptyl-1,3-dimethyl-3,4-dihydropyrido[2,3-b]pyrazin-2(1H)-one.

¹H NMR (300 MHz, DMSO-d6): δ=1.13 (d, 3H); 1.38-1.63 (m, 6H); 1.63-1.84 (m, 4H); 1.83-2.03 (m, 2H); 3.21 (s, 3H); 4.00-4.14 (m, 1H); 4.32 (q, 1H); 6.75 (d, 1H); 7.29 (d, 1H).

### Intermediate 38

### Methyl 4-{[(3R)-4-cycloheptyl-1,3-dimethyl-2-oxo-1,2,3,4-tetrahydropyrido[2,3-b]pyrazin-6-yl]amino}-3-methoxybenzoate

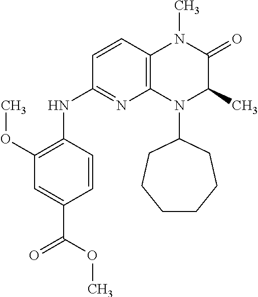 [SMILES: COC(=O)c1ccc(Nc2ccc3c(n2)N(C2CCCCCC2)[C@H](C)C(=O)N3C)c(OC)c1]

Analogously to the preparation of Intermediate 6, methyl 4-{[(3R)-4-cycloheptyl-1,3-dimethyl-2-oxo-1,2,3,4-tetrahydropyrido[2,3-b]pyrazin-6-yl]amino}-3-methoxybenzoate was prepared from 500 mg of Intermediate 37, 589 mg of methyl 4-amino-3-methoxybenzoate, 73 mg of palladium(II) acetate, 2.7 g of caesium carbonate and 202 mg of (+)-BINAP in 15 ml of toluene by 2.5 hours of stirring at 110° C. under an argon atmosphere. Purification by chromatography on silica gel (hexane/ethyl acetate gradient) gave 540 mg of methyl 4-{[(3R)-4-cycloheptyl-1,3-dimethyl-2-oxo-1,2,3,4-tetrahydropyrido[2,3-b]pyrazin-6-yl]amino}-3-methoxybenzoate.

¹H NMR (400 MHz, DMSO-d6): δ=1.09 (d, 3H); 1.39-1.81 (m, 10H); 1.81-2.11 (m, 2H); 3.20 (s, 3H); 3.81 (s, 3H); 3.93 (s, 3H); 4.19-4.35 (m, 2H); 6.63 (d, 1H); 7.28 (d, 1H); 7.45 (d, 1H); 7.49 (dd, 1H); 8.29 (s, 1H); 8.54 (d, 1H).

### Intermediate 39

### 4-{[(3R)-4-Cycloheptyl-1,3-dimethyl-2-oxo-1,2,3,4-tetrahydropyrido[2,3-b]pyrazin-6-yl]amino}-3-methoxybenzoic acid

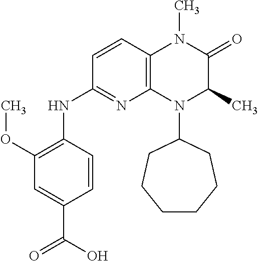 [SMILES: COc1cc(C(=O)O)ccc1Nc1ccc2c(n1)N(C1CCCCCC1)[C@H](C)C(=O)N2C]

Analogously to the preparation of Intermediate 7, 4-{[(3R)-4-cycloheptyl-1,3-dimethyl-2-oxo-1,2,3,4-tetrahydropyrido[2,3-b]pyrazin-6-yl]amino}-3-methoxybenzoic acid was prepared from 540 mg of Intermediate 38 and 11.9 ml of 1N lithium hydroxide solution in 5 ml of THF and 40 ml of methanol. This gave 523 mg of 4-{[(3R)-4-cycloheptyl-1,3-dimethyl-2-oxo-1,2,3,4-tetrahydropyrido[2,3-b]pyrazin-6-yl]amino}-3-methoxybenzoic acid.

UPLC-MS: Rt=1.27 min (M⁺+1=439)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 40

### 4-{[(3R)-4-Cycloheptyl-1,3-dimethyl-2-oxo-1,2,3,4-tetrahydropyrido[2,3-b]pyrazin-6-yl]amino}benzoic acid

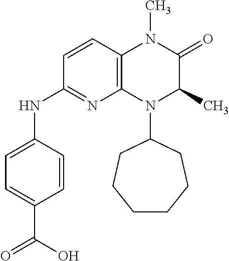 [SMILES: C[C@@H]1C(=O)N(C)c2ccc(Nc3ccc(C(=O)O)cc3)nc2N1C1CCCCCC1]

Analogously to the preparation of Intermediate 6, 4-{[(3R)-4-cycloheptyl-1,3-dimethyl-2-oxo-1,2,3,4-tetrahydropyrido[2,3-b]pyrazin-6-yl]amino}benzoic acid was prepared as follows: Starting with 500 mg of Intermediate 37, 491 mg of methyl 4-aminobenzoate, 73 mg of palladium(II) acetate, 2.7 g of caesium carbonate and 202 mg of (+)-BINAP in 15 ml of toluene, the title compound was prepared by stirring under an argon atmosphere at 110° C. for 2.5 hours. Purification by chromatography on silica gel (hexane/ethyl acetate gradient) gave 630 mg of methyl 4-{[(3R)-4-cycloheptyl-1,3-dimethyl-2-oxo-1,2,3,4-tetrahydropyrido[2,3-b]pyrazin-6-yl]amino}benzoate (¹H NMR (400 MHz, DMSO-d6): δ=1.10 (d, 3H); 1.39-1.82 (m, 10H); 1.81-2.14 (m, 2H); 3.20 (s, 3H); 3.79 (s, 3H); 4.18-4.39 (m, 2H); 6.30 (d, 1H); 7.28 (d, 1H); 7.71-7.86 (m, 4H); 9.33 (s, 1H)). This was reacted analogously to the preparation of Intermediate 7 with 14.9 ml of aqueous 1N lithium hydroxide solution in 5 ml of THF and 40 ml of methanol. This gave 609 mg of 4-{[(3R)-4-cycloheptyl-1,3-dimethyl-2-oxo-1,2,3,4-tetrahydropyrido[2,3-b]pyrazin-6-yl]amino}benzoic acid.

UPLC-MS: Rt=1.19 min (M⁺+1=409)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 41

### N2-Benzyl-N-(2,6-dichloropyridin-3-yl)-D-alaninamide

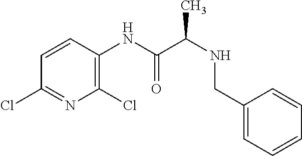 [SMILES: C[C@@H](NCc1ccccc1)C(=O)Nc1ccc(Cl)nc1Cl]

Analogously to the preparation of Intermediate 3, N2-benzyl-N-(2,6-dichloropyridin-3-yl)-D-alaninamide was prepared from 1.5 g of Intermediate 2, 765 mg of benzaldehyde, 909 mg of sodium acetate and 3.5 g of sodium triacetoxyborohydride in 80 ml of dichloromethane at 0° C. This gave 1.5 g of N2-benzyl-N-(2,6-dichloropyridin-3-yl)-D-alaninamide.

¹H NMR (400 MHz, DMSO-d6): δ=1.29 (d, 3H); 3.29 (q, 1H); 3.76 (s, 2H); 7.23 (t, 1H); 7.32 (t, 2H); 7.39 (d, 2H); 7.58 (d, 1H); 8.59 (d, 1H).

### Intermediate 42

### (3R)-4-Benzyl-6-chloro-3-methyl-3,4-dihydropyrido[2,3-b]pyrazin-2(1H)-one

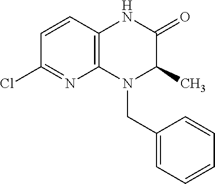 [SMILES: C[C@@H]1C(=O)Nc2ccc(Cl)nc2N1Cc1ccccc1]

Analogously to the synthesis of Intermediate 4, (3R)-4-benzyl-6-chloro-3-methyl-3,4-dihydropyrido[2,3-b]pyrazin-2(1H)-one was prepared from 1.4 g of Intermediate 41 and 5.88 ml of N,N-diisopropylethylamine in 100 ml of DMF by heating for 72 hours at a bath temperature of 170° C. This gave 1.14 g of (3R)-4-benzyl-6-chloro-3-methyl-3,4-dihydropyrido[2,3-b]pyrazin-2(1H)-one.

¹H NMR (300 MHz, DMSO-d6): δ=1.18 (d, 3H); 3.95 (q, 1H); 4.29 (d, 1H); 5.10 (d, 1H); 6.71 (d, 1H); 7.04 (d, 1H); 7.23-7.33 (m, 1H); 7.33-7.41 (m, 4H); 10.70 (s, 1H).

### Intermediate 43

### (3R)-4-Benzyl-6-chloro-1,3-dimethyl-3,4-dihydropyrido[2,3-b]pyrazin-2(1H)-one

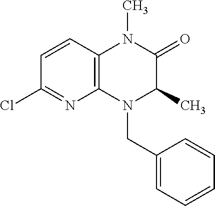 [SMILES: C[C@@H]1C(=O)N(C)c2ccc(Cl)nc2N1Cc1ccccc1]

In analogy to the preparation of Intermediate 5, (3R)-4-benzyl-6-chloro-1,3-dimethyl-3,4-dihydropyrido[2,3-b]pyrazin-2(1H)-one was prepared from 1.14 g of Intermediate 42, 238 mg of sodium hydride (60% in white oil) and 0.37 ml of methyl iodide in 50 ml of DMF. Purification by chromatography on silica gel (hexane/ethyl acetate 3:1) gave 1.15 g of (3R)-4-benzyl-6-chloro-1,3-dimethyl-3,4-dihydropyrido[2,3-b]pyrazin-2(1H)-one. 1H NMR (300 MHz, DMSO-d6): δ=1.15 (d, 3H); 3.24 (s, 3H); 4.08 (q, 1H); 4.28 (d, 1H); 5.11 (d, 1H); 6.82 (d, 1H); 7.22-7.42 (m, 6H).

### Intermediate 44

### Methyl 4-{[(3R)-4-benzyl-1,3-dimethyl-2-oxo-1,2,3,4-tetrahydropyrido[2,3-b]pyrazin-6-yl]amino}-3-methoxybenzoate

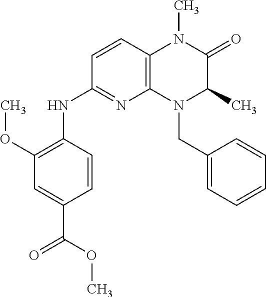 [SMILES: COC(=O)c1ccc(Nc2ccc3c(n2)N(Cc2ccccc2)[C@H](C)C(=O)N3C)c(OC)c1]

Analogously to the preparation of Intermediate 6, methyl 4-{[(3R)-4-benzyl-1,3-dimethyl-2-oxo-1,2,3,4-tetrahydropyrido[2,3-b]pyrazin-6-yl]amino}-3-methoxybenzoate was prepared from 500 mg of Intermediate 43, 600 mg of methyl 4-amino-3-methoxybenzoate, 74 mg of palladium(II) acetate, 2.7 g of caesium carbonate and 206 mg of (+)-BINAP in 15 ml of toluene after 2.5 hours of stirring at 110° C. under an argon atmosphere. Purification by chromatography on silica gel (hexane/ethyl acetate gradient) gave 500 mg of methyl 4-{[(3R)-4-benzyl-1,3-dimethyl-2-oxo-1,2,3,4-tetrahydropyrido[2,3-b]pyrazin-6-yl]amino}-3-methoxybenzoate.

¹H NMR (400 MHz, DMSO-d6): δ=1.14 (d, 3H); 3.25 (s, 3H); 3.79 (s, 3H); 3.90 (s, 3H); 4.08 (q, 1H); 4.34 (d, 1H); 5.13 (d, 1H); 6.65 (d, 1H); 7.21-7.43 (m, 8H); 8.11 (d, 1H); 8.26 (s, 1H).

### Intermediate 45

### 4-{[(3R)-4-Benzyl-1,3-dimethyl-2-oxo-1,2,3,4-tetrahydropyrido[2,3-b]pyrazin-6-yl]amino}-3-methoxybenzoic acid

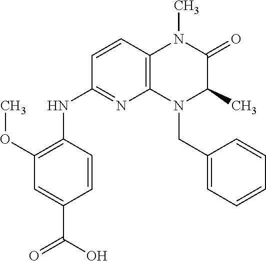 [SMILES: COc1cc(C(=O)O)ccc1Nc1ccc2c(n1)N(Cc1ccccc1)[C@H](C)C(=O)N2C]

In analogy to the preparation of Intermediate 7, 4-{[(3R)-4-benzyl-1,3-dimethyl-2-oxo-1,2,3,4-tetrahydropyrido[2,3-b]pyrazin-6-yl]amino}-3-methoxybenzoic acid was prepared proceeding from 500 mg of Intermediate 44 and 11.2 ml of aqueous 1N lithium hydroxide solution in 5 ml of THF and 50 ml of methanol. This gave 484 mg of 4-{[(3R)-4-benzyl-1,3-dimethyl-2-oxo-1,2,3,4-tetrahydropyrido[2,3-b]pyrazin-6-yl]amino}-3-methoxybenzoic acid.

UPLC-MS: Rt=1.16 min (M⁺+1=433)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 46

### Methyl 4-{[(3R)-4-benzyl-1,3-dimethyl-2-oxo-1,2,3,4-tetrahydropyrido[2,3-b]pyrazin-6-yl]amino}benzoate

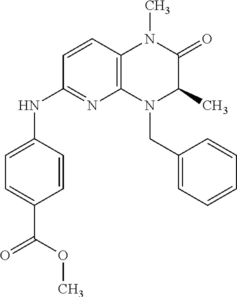 [SMILES: COC(=O)c1ccc(Nc2ccc3c(n2)N(Cc2ccccc2)[C@H](C)C(=O)N3C)cc1]

Analogously to the preparation of Intermediate 6, methyl 4-{[(3R)-4-benzyl-1,3-dimethyl-2-oxo-1,2,3,4-tetrahydropyrido[2,3-b]pyrazin-6-yl]amino}benzoate was prepared from 500 mg of Intermediate 43, 501 mg of methyl 4-aminobenzoate, 74 mg of palladium(II) acetate, 2.7 g of caesium carbonate and 206 mg of (+)-BINAP in 15 ml of toluene by 2.5 hours of stirring at 110° C. under an argon atmosphere. Purification by chromatography on silica gel (hexane/ethyl acetate gradient) gave 500 mg of methyl 4-{[(3R)-4-benzyl-1,3-dimethyl-2-oxo-1,2,3,4-tetrahydropyrido[2,3-b]pyrazin-6-yl]amino}benzoate.

¹H NMR (400 MHz, DMSO-d6): δ=1.15 (d, 3H); 3.25 (s, 3H); 3.77 (s, 3H); 4.09 (q, 1H); 4.37 (d, 1H); 5.16 (d, 1H); 6.33 (d, 1H); 7.22-7.40 (m, 6H); 7.52 (d, 2H); 7.69 (d, 2H); 9.26 (s, 1H).

### Intermediate 47

### 4-{[(3R)-4-Benzyl-1,3-dimethyl-2-oxo-1,2,3,4-tetrahydropyrido[2,3-b]pyrazin-6-yl]amino}benzoic acid

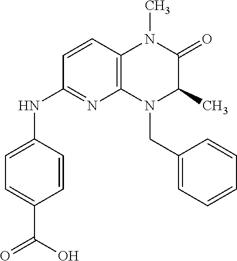 [SMILES: C[C@@H]1C(=O)N(C)c2ccc(Nc3ccc(C(=O)O)cc3)nc2N1Cc1ccccc1]

Analogously to the preparation of Intermediate 7, 4-{[(3R)-4-benzyl-1,3-dimethyl-2-oxo-1,2,3,4-tetrahydropyrido[2,3-b]pyrazin-6-yl]amino}benzoic acid was prepared from 500 mg of Intermediate 46 and 12 ml of aqueous 1N lithium hydroxide solution in 5 ml of THF and 50 ml of methanol. This gave 483 mg of 4-{[(3R)-4-benzyl-1,3-dimethyl-2-oxo-1,2,3,4-tetrahydropyrido[2,3-b]pyrazin-6-yl]amino}benzoic acid.

UPLC-MS: Rt=1.08 min (M⁺+1=403)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 48

### N-(2,6-Dichloropyridin-3-yl)-2-oxopropanamide

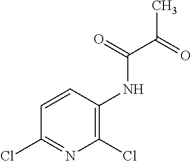

At 0° C., 14.6 ml of thionyl chloride were added slowly to a solution of 17.6 g of pyruvic acid in 150 ml of DMF. The mixture was stirred for 15 minutes, and 16.3 g of 2,6-dichloropyridine-3-amine (CAS 62476-56-6) were then added. The mixture was left stirring at RT for 16 hours and poured into 300 ml of ice-water. The precipitate was filtered off and washed with water. This gave 9.8 g of N-(2,6-dichloropyridin-3-yl)-2-oxopropanamide.

¹H NMR (300 MHz, DMSO-d6): δ=2.44 (s, 3H); 7.65 (d, 1H); 8.28 (d, 1H); 10.03 (bs, 1H).

### Intermediate 49

### N-(2,6-Dichloropyridin-3-yl)-N2-(2-methoxyethyl)alaninamide

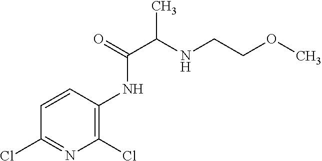 [SMILES: COCCNC(C)C(=O)Nc1ccc(Cl)nc1Cl]

At RT, 2.16 g of sodium triacetoxyborohydride were added to a solution of 1.7 g of Intermediate 48 and 603 mg of 2-methoxyethylamine in 52 ml of 1,2-dichloroethane and 0.42 ml of acetic acid. The mixture was stirred for 16 hours. The reaction was stirred into water and extracted with dichloromethane. The organic phase was washed with sodium bicarbonate solution and water and dried over sodium sulphate, and the solvent was removed under reduced pressure. This gave 2.13 g of N-(2,6-dichloropyridin-3-yl)-N2-(2-methoxyethyl)alaninamide.

UPLC-MS: Rt=0.62 min (M⁺+1=292/294/296)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 50

### 6-Chloro-4-(2-methoxyethyl)-3-methyl-3,4-dihydropyrido[2,3-b]pyrazin-2(1H)-one

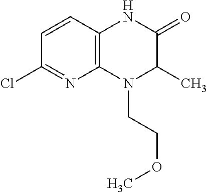 [SMILES: COCCN1c2nc(Cl)ccc2NC(=O)C1C]

Analogously to the synthesis of Intermediate 4, 6-chloro-4-(2-methoxyethyl)-3-methyl-3,4-dihydropyrido[2,3-b]pyrazin-2(1H)-one was prepared from 2.9 g of Intermediate 49 and 13.8 ml of N,N-diisopropylethylamine in 5 ml of DMF by heating for 72 hours at a bath temperature of 170° C. This gave 1.0 g of 6-chloro-4-(2-methoxyethyl)-3-methyl-3,4-dihydropyrido[2,3-b]pyrazin-2(1H)-one.

¹H NMR (300 MHz, DMSO-d6): δ=1.21 (d, 3H); 3.19-3.31 (m+s, 4H); 3.45-3.59 (m, 2H); 3.99 (dt, 1H); 4.14 (q, 1H); 6.65 (d, 1H); 6.97 (d, 1H); 10.62 (bs, 1H).

### Intermediate 51

### 6-Chloro-4-(2-methoxyethyl)-1,3-dimethyl-3,4-dihydropyrido[2,3-b]pyrazin-2(1H)-one

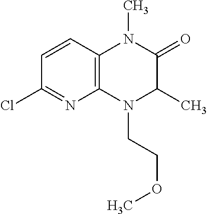 [SMILES: COCCN1c2nc(Cl)ccc2N(C)C(=O)C1C]

Analogously to the preparation of Intermediate 5, 6-chloro-4-(2-methoxyethyl)-1,3-dimethyl-3,4-dihydropyrido[2,3-b]pyrazin-2(1H)-one was prepared from 1.0 g of Intermediate 50, 256 mg of sodium hydride (60% in white oil) and 0.37 ml of methyl iodide in 9 ml of DMF. Purification by chromatography on silica gel (hexane/ethyl acetate gradient) gave 730 mg of 6-chloro-4-(2-methoxyethyl)-1,3-dimethyl-3,4-dihydropyrido[2,3-b]pyrazin-2(1H)-one.

¹H NMR (300 MHz, DMSO-d6): δ=1.17 (d, 3H); 3.19-3.31 (m+2s, 7H); 3.45-3.60 (m, 2H); 4.02 (dt, 1H); 4.28 (q, 1H); 6.77 (d, 1H); 7.29 (d, 1H).

### Intermediate 52

### Ethyl 4-{[4-(2-methoxyethyl)-1,3-dimethyl-2-oxo-1,2,3,4-tetrahydropyrido[2,3-b]pyrazin-6-yl]amino}benzoate

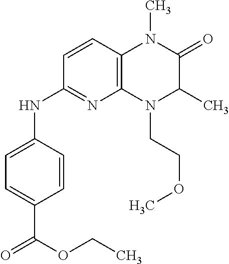 [SMILES: CCOC(=O)c1ccc(Nc2ccc3c(n2)N(CCOC)C(C)C(=O)N3C)cc1]

Analogously to the preparation of Intermediate 6, ethyl 4-{[4-(2-methoxyethyl)-1,3-dimethyl-2-oxo-1,2,3,4-tetrahydropyrido[2,3-b]pyrazin-6-yl]amino}benzoate was prepared from 2 g of Intermediate 51, 2.37 g of ethyl 4-aminobenzoate, 316 mg of palladium(II) acetate, 11.5 g of caesium carbonate and 877 mg of (+)-BINAP in 158 ml of toluene by 5 hours of stirring at 120° C. under an argon atmosphere. Purification by chromatography on silica gel (hexane/ethyl acetate gradient) gave 2.3 g of ethyl 4-{[4-(2-methoxyethyl)-1,3-dimethyl-2-oxo-1,2,3,4-tetrahydropyrido[2,3-b]pyrazin-6-yl]amino}benzoate.

UPLC-MS: Rt=1.21 min (M⁺+1=399)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 53

### 4-{[4-(2-Methoxyethyl)-1,3-dimethyl-2-oxo-1,2,3,4-tetrahydropyrido[2,3-b]pyrazin-6-yl]amino}benzoic acid

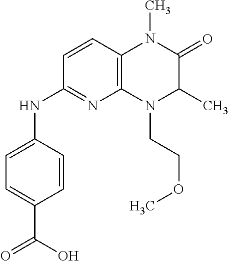 [SMILES: COCCN1c2nc(Nc3ccc(C(=O)O)cc3)ccc2N(C)C(=O)C1C]

Analogously to the preparation of Intermediate 7, 4-{[4-(2-methoxyethyl)-1,3-dimethyl-2-oxo-1,2,3,4-tetrahydropyrido[2,3-b]pyrazin-6-yl]amino}benzoic acid was prepared from 2.3 g of Intermediate 52 and 14.4 ml of aqueous 2N sodium hydroxide solution in 109 ml of ethanol. This gave 0.9 g of 4-{[4-(2-methoxyethyl)-1,3-dimethyl-2-oxo-1,2,3,4-tetrahydropyrido[2,3-b]pyrazin-6-yl]amino}benzoic acid.

¹H NMR (300 MHz, DMSO-d6, selected signals): δ=1.14 (d, 3H); 3.21 (s, 3H); 3.28 (s, 3H); 3.53-3.67 (m, 2H); 4.05 (dt, 1H); 4.20 (q, 1H); 6.29 (d, 1H); 7.25 (d, 1H); 7.66 (d, 2H); 7.79 (d, 2H); 9.25 (s, 1H); 12.34 (bs, 1H).

### Intermediate 54

### tert-Butyl 4-({(2R)-1-[(2,6-dichloropyridin-3-yl)amino]-1-oxopropan-2-yl}amino)piperidine-1-carbonate

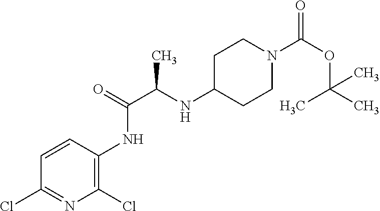 [SMILES: C[C@@H](NC1CCN(C(=O)OC(C)(C)C)CC1)C(=O)Nc1ccc(Cl)nc1Cl]

Analogously to the preparation of Intermediate 3, tert-butyl 4-({(2R)-1-[(2,6-dichloropyridin-3-yl)amino]-1-oxopropan-2-yl}amino)piperidine-1-carbonate was prepared from 2 g of Intermediate 2, 2.02 g of 1-Boc-4-piperidin-1-one (CAS 79099-07-3), 1.21 g of sodium acetate and 4.7 g of sodium triacetoxyborohydride in 60 ml of dichloromethane at 0° C. This gave 4.1 g of tert-butyl 4-({(2R)-1-[(2,6-dichloropyridin-3-yl)amino]-1-oxopropan-2-yl}amino)piperidine-1-carbonate as a crude product which was used without further purification for the next step.

¹H NMR (400 MHz, DMSO-d6): δ=1.10.1.25 (m, 2H); 1.27 (d, 3H); 1.38 (s, 9H); 1.74 (bd, 1H); 1.89 (bd, 1H); 2.67-2.83 (bs, 2H); 3.39 (q, 1H); 3.80-3.90 (m, 2H); 7.58 (d, 1H); 8.66 (d, 1H).

### Intermediate 55

### tert-Butyl 4-[(3R)-6-chloro-3-methyl-2-oxo-2,3-dihydropyrido[2,3-b]pyrazin-4(1H)-yl]piperidine-1-carbonate

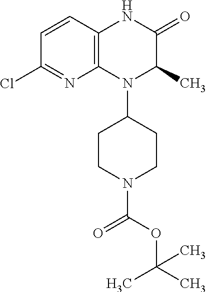 [SMILES: C[C@@H]1C(=O)Nc2ccc(Cl)nc2N1C1CCN(C(=O)OC(C)(C)C)CC1]

Analogously to the synthesis of Intermediate 4, tert-butyl 4-[(3R)-6-chloro-3-methyl-2-oxo-2,3-dihydropyrido[2,3-b]pyrazin-4(1H)-yl]piperidine-1-carbonate was prepared from 1.02 g of Intermediate 54 and 3.4 ml of N,N-diisopropylethylamine in 5 ml of DMF by heating for 18 hours at a bath temperature of 170° C. This gave 577 mg of tert-butyl 4-[(3R)-6-chloro-3-methyl-2-oxo-2,3-dihydropyrido[2,3-b]pyrazin-4(1H)-yl]piperidine-1-carbonate.

¹H NMR (300 MHz, DMSO-d6): δ=1.14 (d, 3H); 1.41 (s, 9H); 1.53-1.62 (m, 1H); 1.65-1.77 (m, 1H); 1.82-1.93 (m, 2H); 2.68-2.90 (bs, 2H); 3.98-4.10 (m, 2H); 4.10-4.20 (m, 2H); 6.69 (d, 1H); 7.02 (d, 1H); 10.58 (s, 1H).

### Intermediate 56

### tert-Butyl 4-[(3R)-6-chloro-1,3-dimethyl-2-oxo-2,3-dihydropyrido[2,3-b]pyrazin-4(1H)-yl]piperidine-1-carbonate

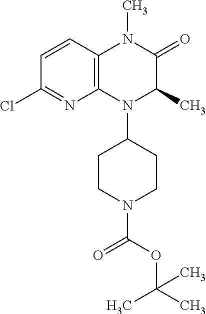 [SMILES: C[C@@H]1C(=O)N(C)c2ccc(Cl)nc2N1C1CCN(C(=O)OC(C)(C)C)CC1]

Analogously to the preparation of Intermediate 5, tert-butyl 4-[(3R)-6-chloro-1,3-dimethyl-2-oxo-2,3-dihydropyrido[2,3-b]pyrazin-4(1H)-yl]piperidine-1-carbonate was prepared from 573 mg of Intermediate 55, 98 mg of sodium hydride (60% in white oil) and 0.14 ml of methyl iodide in 6.6 ml of DMF. Purification by chromatography on silica gel (hexane/ethyl acetate gradient) gave 460 mg of tert-butyl 4-[(3R)-6-chloro-1,3-dimethyl-2-oxo-2,3-dihydropyrido[2,3-b]pyrazin-4(1H)-yl]piperidine-1-carbonate.

¹H NMR (300 MHz, DMSO-d6): δ=1.11 (d, 3H); 1.41 (s, 9H); 1.55-1.63 (m, 1H); 1.70 (qd, 1H); 1.81-1.93 (m, 2H); 2.71-2.91 (bs, 2H); 3.22 (s, 3H); 3.99-4.11 (m, 2H); 4.19 (tt, 1H); 4.30 (q, 1H); 6.80 (d, 1H); 7.33 (d, 1H).

### Intermediate 57

### 2-Bromo-N-(2,6-dichloropyridin-3-yl)propanamide

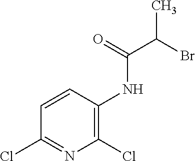

At RT, 20.3 g of 2-bromopropionyl bromide (CAS 563-76-8) were added slowly to a solution of 8.5 g of 3-amino-2,6-dichloropyridine (CAS 62476-59-9) in 200 ml of THF and 12.7 ml of pyridine. The mixture was left stirring at RT for 72 hours. Water was then added, and the mixture was extracted with ethyl acetate. The organic phase was dried over sodium sulphate and evaporated to dryness. The residue was purified by chromatography on silica gel (dichloromethane). This gave 8.2 g of 2-bromo-N-(2,6-dichloropyridin-3-yl)propanamide.

¹H NMR (300 MHz, DMSO-d6): δ=1.76 (d, 3H); 4.94 (q, 1H); 7.60 (d, 1H); 8.22 (d, 1H); 10.17 (s, 1H).

### Intermediate 58

### N-(2,6-Dichloropyridin-3-yl)-N2-phenylalaninamide

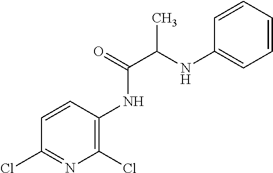 [SMILES: CC(Nc1ccccc1)C(=O)Nc1ccc(Cl)nc1Cl]

A solution of 2.7 g of Intermediate 57 and 759 mg of aniline in 27 ml of toluene and 2.7 ml of diisopropylethylamine was stirred at 140° C. for 3 hours. After cooling to RT, water was added and the mixture was extracted with ethyl acetate. The organic phase was dried over sodium sulphate and evaporated to dryness. The residue was purified by chromatography on silica gel (dichloromethane). This gave 3.1 g of N-(2,6-dichloropyridin-3-yl)-N2-phenylalaninamide which was sufficiently pure for further reactions.

¹H NMR (300 MHz, DMSO-d6): δ=1.44 (d, 3H); 4.12 (qi, 1H); 6.11 (d, 1H); 6.64 (d, 2H); 6.99 (t, 1H); 7.10 (t, 2H); 7.56 (d, 1H); 8.29 (d, 1H); 9.79 (s, 1H).

### Intermediate 59

### 6-Chloro-3-methyl-4-phenyl-3,4-dihydropyrido[2,3-b]pyrazin-2(1H)-one

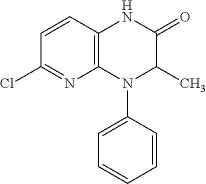 [SMILES: CC1C(=O)Nc2ccc(Cl)nc2N1c1ccccc1]

Analogously to the synthesis of Intermediate 4, 6-chloro-3-methyl-4-phenyl-3,4-dihydropyrido[2,3-b]pyrazin-2(1H)-one was prepared from 1.8 g of Intermediate 58 and 12.3 ml of N,N-dicyclohexylmethylamine in 10 ml of DMF by heating for 18 hours at a bath temperature of 170° C. This gave 350 mg of 6-chloro-3-methyl-4-phenyl-3,4-dihydropyrido[2,3-b]pyrazin-2(1H)-one.

¹H NMR (300 MHz, DMSO-d6): δ=1.29 (d, 3H); 4.48 (q, 1H); 6.84 (d, 1H); 7.17 (d, 1H); 7.22 (t, 1H); 7.33 (d, 2H); 7.41 (t, 2H); 10.82 (s, 1H).

### Intermediate 60

### 6-Chloro-1,3-dimethyl-4-phenyl-3,4-dihydropyrido[2,3-b]pyrazin-2(1H)-one

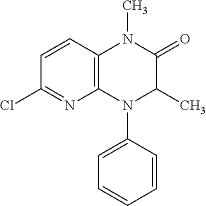 [SMILES: CC1C(=O)N(C)c2ccc(Cl)nc2N1c1ccccc1]

Analogously to the preparation of Intermediate 5, 6-chloro-1,3-dimethyl-4-phenyl-3,4-dihydropyrido[2,3-b]pyrazin-2(1H)-one was prepared from 500 mg of Intermediate 59 (obtained from 2 reactions), 120 mg of sodium hydride (60% in white oil) and 0.171 ml of methyl iodide in 9 ml of DMF. Chromatography on silica gel (hexane/ethyl acetate gradient) gave 380 mg of 6-chloro-1,3-dimethyl-4-phenyl-3,4-dihydropyrido[2,3-b]pyrazin-2(1H)-one.

¹H NMR (300 MHz, DMSO-d6): δ=1.29 (d, 3H); 3.32 (s, 3H); 4.60 (q, 1H); 6.96 (d, 1H); 7.21 (t, 1H); 7.33 (d, 2H); 7.41/t, 2H); 7.50 (d, 1H).

### Intermediate 61

### 4-Nitro-N-[2-(pyridin-3-yl)ethyl]benzenesulphonamide

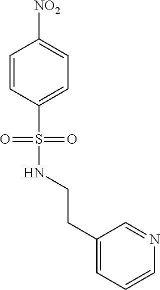 [SMILES: O=[N+]([O-])c1ccc(S(=O)(=O)NCCc2cccnc2)cc1]

Analogously to the preparation of Intermediate 27, 4-nitro-N-[2-(pyridin-3-yl)ethyl]benzenesulphonamide was prepared from 1.04 g of 4-nitrobenzenesulphonyl chloride and 600 mg of 2-(pyridin-3-yl)ethanamine (CAS 20173-24-4) using 2.5 ml of triethylamine in 26 ml of dichloromethane. This gave 730 mg of 4-nitro-N-[2-(pyridin-3-yl)ethyl]benzenesulphonamide which was used without further purification for the next step.

¹H NMR (400 MHz, DMSO-d6): δ=2.71 (t, 2H); 3.09 (t, 2H); 7.26 (dd, 1H); 7.58 (bd, 1H); 7.99 (d, 2H); 8.10 (bs, 1H); 8.34-8.41 (m, 4H).

### Intermediate 62

### 4-Amino-N-[2-(pyridin-3-yl)ethyl]benzenesulphonamide

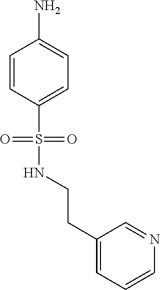 [SMILES: Nc1ccc(S(=O)(=O)NCCc2cccnc2)cc1]

Analogously to the preparation of Intermediate 28, 4-amino-N-[2-(pyridin-3-yl)ethyl]benzenesulphonamide was prepared by reduction of 730 mg of Intermediate 61 with hydrogen on 93 mg of palladium (10% on activated carbon) in 22 ml of methanol. This gave 600 mg of 4-amino-N-[2-(pyridin-3-yl)ethyl]benzenesulphonamide which was used without further purification for the next step.

¹H NMR (300 MHz, DMSO-d6): δ=2.67 (t, 2H); 2.89 (q, 2H); 5.93 (bs, 2H); 6.59 (d, 2H); 7.22 8t, 1H); 7.28 (dd, 1H); 7.39 (d, 2H); 0.58 (bd, 1H); 8.34-8.43 (m, 2H).

### Intermediate 63

### N-[2-(4-Methylpiperazin-1-yl)ethyl]-4-nitrobenzenesulphonamide

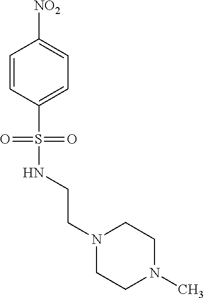 [SMILES: CN1CCN(CCNS(=O)(=O)c2ccc([N+](=O)[O-])cc2)CC1]

Analogously to the preparation of Intermediate 27, N-[2-(4-methylpiperazin-1-yl)ethyl]-4-nitrobenzenesulphonamide was prepared from 3.5 g of 4-nitrobenzenesulphonyl chloride and 2.36 g of 2-(4-methylpiperazin-1-yl)ethanamine (CAS 934-98-5) using 8.4 ml of triethylamine in 87.5 ml of dichloromethane. Purification by chromatography on silica gel (dichloromethane/methanol gradient) gave 4.79 g of N-[2-(4-methylpiperazin-1-yl)ethyl]-4-nitrobenzenesulphonamide.

¹H NMR (400 MHz, DMSO-d6): δ=2.09 (s, 3H); 2.15-2.31 (m+t, 8H); 2.92 (t, 2H); 8.05 (d, 2H); 8.41 (d, 2H).

### Intermediate 64

### 4-Amino-N-[2-(4-methylpiperazin-1-yl)ethyl]benzenesulphonamide

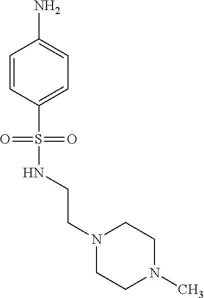 [SMILES: CN1CCN(CCNS(=O)(=O)c2ccc(N)cc2)CC1]

Analogously to the preparation of Intermediate 28, 4-amino-N-[2-(4-methylpiperazin-1-yl)ethyl]benzenesulphonamide was prepared by reduction of 4.79 g of Intermediate 63 with hydrogen on 474 mg of palladium (10% on activated carbon) in 143 ml of methanol. This gave 4.49 g of 4-amino-N-[2-(4-methylpiperazin-1-yl)ethyl]benzenesulphonamide which was used without further purification for the next step.

¹H NMR (300 MHz, DMSO-d6): δ=2.11 (s, 3H); 2.17-2.32 (m, 10H); 2.74 (q, 2H); 5.90 (s, 2H); 6.60 (d, 2H); 6.88 (t, 1H); 7.41 (d, 2H).

### Intermediate 65

### 4-Nitro-N-(pyridin-2-ylmethyl)benzenesulphonamide

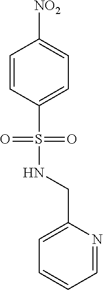 [SMILES: O=[N+]([O-])c1ccc(S(=O)(=O)NCc2ccccn2)cc1]

Analogously to the preparation of Intermediate 27, 4-nitro-N-(pyridin-2-ylmethyl)benzenesulphonamide was prepared from 3.9 g of 4-nitrobenzenesulphonyl chloride and 2 g of 2-(pyridin-3-yl)methanamine (CAS 3731-51-9) using 9.4 ml of triethylamine in 98 ml of dichloromethane. This gave 1.57 g of 4-nitro-N-(pyridin-2-ylmethyl)benzenesulphonamide which was used without further purification for the next step.

¹H NMR (400 MHz, DMSO-d6): δ=4.18 (s, 2H); 7.21 (dd, 1H); 7.31 (d, 1H); 7.70 (dt, 1H); 8.00 (d, 2H); 8.35 (d, 2H); 8.38 (bd, 1H); 8.68 (bs, 1H).

### Intermediate 66

### 4-Amino-N-(pyridin-2-ylmethyl)benzenesulphonamide

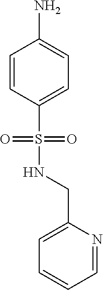 [SMILES: Nc1ccc(S(=O)(=O)NCc2ccccn2)cc1]

Analogously to the preparation of Intermediate 28, 4-amino-N-(pyridin-2-ylmethyl)benzenesulphonamide was prepared by reduction of 1.47 g of Intermediate 65 with hydrogen on 212 mg of palladium (10% on activated carbon) in 49 ml of methanol. This gave 1.3 g of 4-amino-N-(pyridin-2-ylmethyl)benzenesulphonamide which was used without further purification for the next step.

¹H NMR (300 MHz, DMSO-d6): δ=3.97 (s, 2H); 5.94 (s, 2H); 6.58 (d, 2H); 7.24 (dd, 1H); 7.37 (d, 1H); 7.42 (d, 2H); 7.68-7.79 (m, 2H); 8.43 (bd, 1H).

### Intermediate 67

### tert-Butyl [4-(4,4-difluoropiperidin-1-yl)cyclohexyl]carbamate, cis/trans isomer mixture

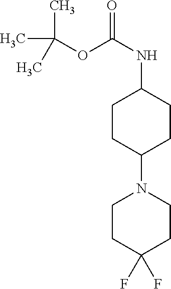 [SMILES: CC(C)(C)OC(=O)NC1CCC(N2CCC(F)(F)CC2)CC1]

At RT, 4.48 g of sodium triacetoxyborohydride and a little acetic acid were added a little at a time to a solution of 2.26 g of 4,4-difluoropiperidine hydrochloride (CAS 144230-52-4) and 2 g of tert-butyl (4-oxocyclohexyl)carbamate (CAS 179321-49-4) in 50 ml of dichloromethane and 1.77 ml of triethylamine. The mixture was stirred for 14 hours, and 50 ml of methanol were then added. The mixture was stirred for 1 hour and diluted with dichloromethane. The reaction was washed with 1 N aqueous sodium hydroxide solution, water and saturated sodium chloride solution and dried over sodium sulphate, and the solvent was removed completely under reduced pressure. This gave 3.1 g of tert-butyl [4-(4,4-difluoropiperidin-1-yl)cyclohexyl]carbamate as a cis/trans isomer mixture.

UPLC-MS: Rt=0.68 min (M⁺+1=319)

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 68

### 4-(4,4-Difluoropiperidin-1-yl)cyclohexanamine, cis/trans isomer mixture

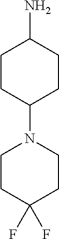 [SMILES: NC1CCC(N2CCC(F)(F)CC2)CC1]

11.3 ml of trifluoroacetic acid were added to 3.1 g of Intermediate 67 in 90 ml of dichloromethane, and the mixture was stirred at boiling point for 5 hours. The reaction was then evaporated to dryness and the residue was taken up in ethyl acetate. The mixture was extracted with saturated sodium bicarbonate solution. The aqueous phase was then extracted three times with dichloromethane. The combined dichloromethane phases were dried over sodium sulphate and the solvent was removed completely under reduced pressure. This gave 920 mg of 4-(4,4-difluoropiperidin-1-yl)cyclohexanamine as a cis/trans isomer mixture.

UPLC-MS: Rt=0.91+0.87 min (M⁺+1=219): cis and trans isomers

Instrument: Waters Acquity UPLC-MS SQD; column: Acquity UPLC BEH C18 1.7 50×2.1 mm; mobile phase A: water+0.1% by volume of ammonia, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 69

### 3-Methoxy-4-nitrobenzenethiol

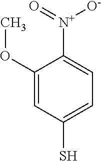

A mixture of 10 g of 5-fluoro-2-nitroanisole, 1.4 g of sulphur, 10.1 g of sodium sulphide nonahydrate and 2.34 g of sodium hydroxide in 200 ml of ethanol was stirred at boiling point for 2 hours. After cooling, 100 ml of hydrochloric acid (10% strength in water) were added and the mixture was extracted with ethyl acetate. The organic phase was washed with hydrochloric acid (10% strength in water) and dried over sodium sulphate, and the solvent was removed completely under reduced pressure. This gave 10.74 g of the title compound as a crude product which was reacted in the next step without further purification.

¹H NMR (300 MHz, CDCl₃): δ=3.92 (s, 3H); 7.25 (d, 1H); 7.51 (s, 1H); 7.92 (d, 1H).

### Intermediate 70

### 3-Methoxy-4-nitrobenzenesulphonic acid

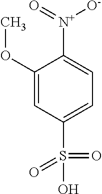

A solution of 10.74 g of Intermediate 69 and 45.6 ml of hydrogen peroxide solution (30% strength solution in water) in 91.3 ml of acetic acid was stirred at boiling point for 2 hours. After cooling, the solution was made alkaline with aqueous sodium hydroxide solution and extracted three times with ethyl acetate. The aqueous phase was stirred into ice-cold hydrochloric acid and the pH was adjusted to <7. The mixture was extracted with ethyl acetate, the organic phase was washed with saturated sodium chloride solution and dried over sodium sulphate and the solvent was removed completely under reduced pressure. The residue was precipitated from ethyl acetate/dichloromethane and filtered off. This gave 1.45 g of 3-methoxy-4-nitrobenzenesulphonic acid.

¹H NMR (300 MHz, DMSO-d6): δ=3.93 (s, 3H); 7.32 (dd, 1H); 7.45 (d, 1H); 7.85 (d, 1H).

### Intermediate 71

### 3-Methoxy-N-(1-methylpiperidin-4-yl)-4-nitrobenzenesulphonamide

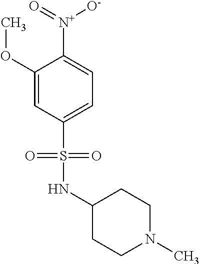 [SMILES: COc1cc(S(=O)(=O)NC2CCN(C)CC2)ccc1[N+](=O)[O-]]

A solution of 800 mg of Intermediate 70 in 1.5 ml of thionyl chloride was stirred at boiling point for 5 hours. The remaining thionyl chloride was then removed under reduced pressure. The solid that remained, which consisted of crude 3-methoxy-4-nitrobenzenesulphonyl chloride, was stirred with 454 mg of 4-amino-1-methylpiperidine and 1.45 ml of triethylamine in 20 ml of dichloromethane at RT for 1 hour. The reaction was diluted with water, the phases were separated and the aqueous phase was extracted with dichloromethane. The combined organic phases were dried over sodium sulphate and the solvent was removed under reduced pressure. Purification by chromatography on silica gel (ethyl acetate/ethanol gradient) gave 500 mg of 3-methoxy-N-(1-methylpiperidin-4-yl)-4-nitrobenzenesulphonamide.

UPLC-MS: Rt=0.65 min (M⁺+1=330)

Instrument: Waters Acquity UPLC-MS SQD; column: Acquity UPLC BEH C18 1.7 50×2.1 mm; mobile phase A: water+0.1% by volume of formic acid, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 72

### 4-Amino-3-methoxy-N-(1-methylpiperidin-4-yl)-benzenesulphonamide

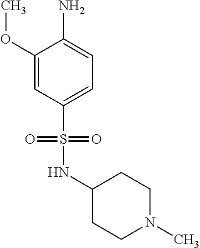 [SMILES: COc1cc(S(=O)(=O)NC2CCN(C)CC2)ccc1N]

A suspension of 500 mg of Intermediate 71 and 50 mg of palladium (10% on activated carbon) in 50 ml of ethanol was shaken under a hydrogen atmosphere at RT for 4 days. The mixture was filtered off through kieselguhr and the solution was evaporated to dryness. This gave 340 mg of 4-amino-3-methoxy-N-(1-methylpiperidin-4-yl)benzenesulphonamide.

UPLC-MS: Rt=0.48 min (M⁺+1=300)

Instrument: Waters Acquity UPLC-MS SQD; column: Acquity UPLC BEH C18 1.7 50×2.1 mm; mobile phase A: water+0.1% by volume of formic acid, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 73

### 1-[(3-Methoxy-4-nitrophenyl)sulphonyl]-4-methylpiperazine

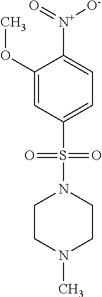 [SMILES: COc1cc(S(=O)(=O)N2CCN(C)CC2)ccc1[N+](=O)[O-]]

A solution of 800 mg of Intermediate 70 in 1.5 ml of thionyl chloride was stirred at boiling point for 5 hours. The remaining thionyl chloride was then removed under reduced pressure. The solid that remained, which consisted of crude 3-methoxy-4-nitrobenzenesulphonyl chloride, was stirred with 400 mg of 1-methylpiperazine and 1.45 ml of triethylamine in 20 ml of dichloromethane at RT for 1 hour. The reaction was diluted with water, the phases were separated and the aqueous phase was extracted with dichloromethane. The combined organic phases were dried over sodium sulphate and the solvent was removed under reduced pressure. This gave 1.1 g of 1-[(3-methoxy-4-nitrophenyl)sulphonyl]-4-methylpiperazine as a crude product which could be used without further purification for the next step.

¹H NMR (400 MHz, DMSO-d6): δ=2.15 (s, 3H); 2.32-2.42 (m, 4H); 2.95-3.05 (m, 4H); 4.03 (s, 3H); 7.46 (dd, 1H); 7.50 (d, 1H); 8.12 (d, 1H).

### Intermediate 74

### 1-[(4-Amino-3-methoxyphenyl)sulphonyl]-4-methylpiperazine

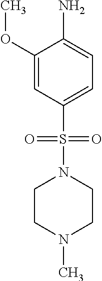 [SMILES: COc1cc(S(=O)(=O)N2CCN(C)CC2)ccc1N]

A suspension of 1.1 g of Intermediate 73 and 100 mg of palladium (10% on activated carbon) in 50 ml of ethanol was shaken under a hydrogen atmosphere at RT for 4 days. The mixture was filtered off through kieselguhr and the solution was evaporated to dryness. This gave 580 mg of 1-[(4-amino-3-methoxyphenyl)sulphonyl]-4-methylpiperazine.

¹H NMR (300 MHz, DMSO-d6): δ=2.14 (s, 3H); 2.30-2.41 (m, 4H); 2.76-2.89 (m, 4H); 3.82 (s, 3H); 5.72 (bs, 2H); 6.72 (d, 1H); 6.96 (d, 1H); 7.07 (dd, 1H).

### Intermediate 75

### N-{trans-4-[4-(Cyclopropylmethyl)piperazin-1-yl]cyclohexyl}-3-methoxy-4-nitrobenzenesulphonamide

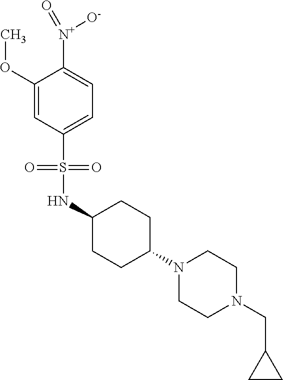 [SMILES: COc1cc(S(=O)(=O)N[C@H]2CC[C@H](N3CCN(CC4CC4)CC3)CC2)ccc1[N+](=O)[O-]]

A solution of 800 mg of Intermediate 70 in 1.5 ml of thionyl chloride was stirred at boiling point for 5 hours. The remaining thionyl chloride was then removed under reduced pressure. The solid that remained, which consisted of crude 3-methoxy-4-nitrobenzenesulphonyl chloride, was stirred with 943 mg of trans-4-[4-(cyclopropylmethyl)piperazin-1-yl]cyclohexanamine (CAS 876461-31-3, prepared analogously to WO2012049153) and 1.44 ml of triethylamine in 20 ml of dichloromethane at RT for 4 hours. The reaction was diluted with water, the phases were separated and the aqueous phase was extracted with dichloromethane. The combined organic phases were dried over sodium sulphate and the solvent was removed under reduced pressure. Chromatography on silica gel (ethyl acetate/ethanol gradient) gave 560 mg of N-{trans-4-[4-(cyclopropylmethyl)piperazin-1-yl]cyclohexyl}-3-methoxy-4-nitrobenzenesulphonamide.

UPLC-MS: Rt=0.74 min (M⁺+1=453)

Instrument: Waters Acquity UPLC-MS SQD; column: Acquity UPLC BEH C18 1.7 50×2.1 mm; mobile phase A: water+0.1% by volume of formic acid, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 76

### 4-Amino-N-{trans-4-[4-(cyclopropylmethyl)piperazin-1-yl]cyclohexyl}-3-methoxybenzenesulphonamide

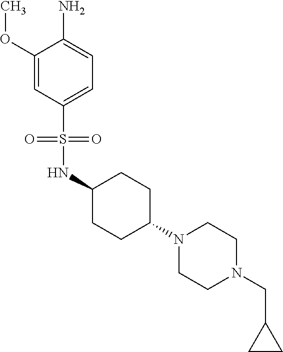 [SMILES: COc1cc(S(=O)(=O)N[C@H]2CC[C@H](N3CCN(CC4CC4)CC3)CC2)ccc1N]

A suspension of 560 mg of Intermediate 75 and 56 mg of palladium (10% on activated carbon) in 50 ml of ethanol was stirred under a hydrogen atmosphere at RT for 4 days. The mixture was filtered off through kieselguhr and the solution was evaporated to dryness. This gave 460 mg of 4-amino-N-{trans-4-[4-(cyclopropylmethyl)piperazin-1-yl]cyclohexyl}-3-methoxybenzenesulphonamide.

UPLC-MS: Rt=0.56 min (M⁺+1=423)

Instrument: Waters Acquity UPLC-MS SQD; column: Acquity UPLC BEH C18 1.7 50×2.1 mm; mobile phase A: water+0.1% by volume of formic acid, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

To prepare the working examples mentioned below, use was furthermore made of the amines shown in Table 1 below which are either commercially available or may be prepared by or analogously to the procedures quoted.

TABLE 1

| Amine | | | Preparation |
|---|---|---|---|
| No. | Structure | CAS Number | Procedure |
| 1 | 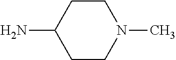 | 41838-46-4 | |
| 2 | 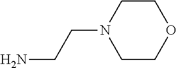 | 2038-03-1 | |
| 3 | 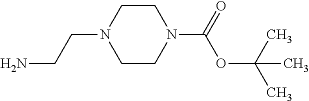 [SMILES: CC(C)(C)OC(=O)N1CCN(CCN)CC1] | 192130-34-1 | |
| 4 | 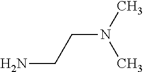 | 108-00-9 | |
| 5 | 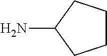 | 1003-03-8 | |
| 6 | 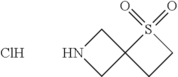 | 1352546-75-8 | |
| 7 | 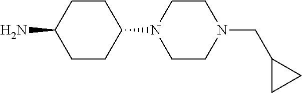 [SMILES: N[C@H]1CC[C@H](N2CCN(CC3CC3)CC2)CC1] | 876461-31-3 | analogously to WO2012049153 |
| 8 | 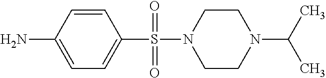 [SMILES: CC(C)N1CCN(S(=O)(=O)c2ccc(N)cc2)CC1] | 524719-43-3 | analogously to US20030225106 |
| 9 | 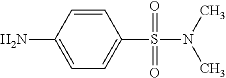 | 1709-59-7 | |
| 10 | 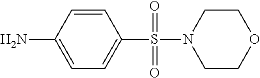 [SMILES: Nc1ccc(S(=O)(=O)N2CCOCC2)cc1] | 21626-70-0 | |
| 11 | 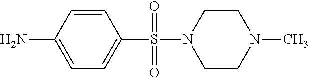 [SMILES: CN1CCN(S(=O)(=O)c2ccc(N)cc2)CC1] | 21623-68-7 | |
| 12 | 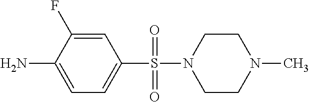 [SMILES: CN1CCN(S(=O)(=O)c2ccc(N)c(F)c2)CC1] | 486422-39-3 | J. Med. Chem. (2012), 55, p. 9107ff |
| 13 | 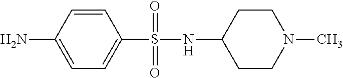 [SMILES: CN1CCC(NS(=O)(=O)c2ccc(N)cc2)CC1] | 1062245-55-9 | WO2008052847, Example 66, steps a + b |
| 14 | 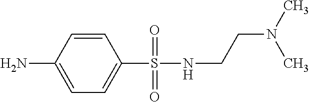 [SMILES: CN(C)CCNS(=O)(=O)c1ccc(N)cc1] | 77837-46-8 | J. Am. Chem. Soc. (2006), 128, p. 8320ff |
| 15 | 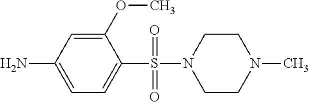 [SMILES: COc1cc(N)ccc1S(=O)(=O)N1CCN(C)CC1] | 97630-54-1 | Eur. Pat. Appl. 138720 (1985) |
| 16 | 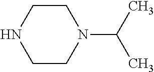 | 4318-42-7 | |
| 17 | 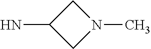 | 959957-92-7 | |
| 18 | 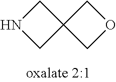 | 1045709-32-7 | |
| 19 | 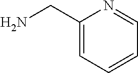 | 3731-51-9 | |
| 20 | 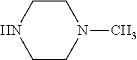 | 109-01-3 | |
| 21 | 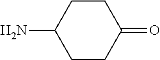 | 87976-86-1 | |
| 22 | 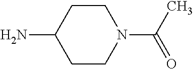 | 160357-94-8 | |
| 23 | 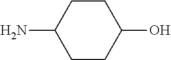 | 6850-65-3 | |
| 24 | 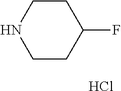 | 57395-89-8 | |
| 25 | 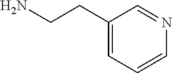 | 20173-24-4 | |
| 26 | 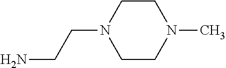 | 934-98-5 | |
| 27 | 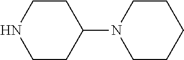 [SMILES: C1CCN(C2CCNCC2)CC1] | 4897-50-1 | |
| 28 | 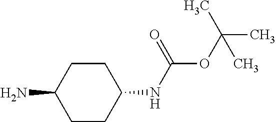 | 177906-48-8 | |
| 29 | 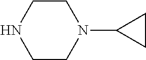 | 20327-23-5 | |
| 30 | 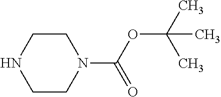 | 57260-71-6 | |

### Preparation of the Compounds According to the Invention:

### Example 1

### 4-{[(3R)-4-Cyclopentyl-1,3-dimethyl-2-oxo-1,2,3,4-tetrahydropyrido[2,3-b]pyrazin-6-yl]amino}-3-methoxy-N-(1-methylpiperidin-4-yl)benzamide

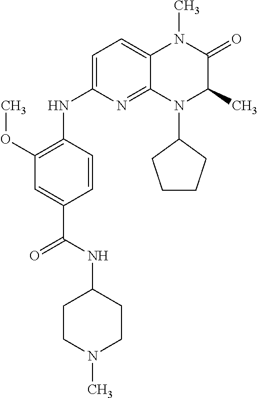 [SMILES: COc1cc(C(=O)NC2CCN(C)CC2)ccc1Nc1ccc2c(n1)N(C1CCCC1)[C@H](C)C(=O)N2C]

A solution of 1.0 g of Intermediate 7, 695 mg of 4-amino-1-methylpiperidine (Amine No. 1), 1.68 g of potassium carbonate and 1.96 g of TBTU in 100 ml of DMF was stirred at RT for 2 hours. The reaction was diluted with dichloromethane and washed with water and saturated sodium bicarbonate solution. The organic phase was evaporated to dryness and the residue was purified by RP-HPLC chromatography (column: X-Bridge C18 5 μm 100×30 mm, mobile phase: acetonitrile/water (0.2% by volume ammonia) gradient). This gave 400 mg of 4-{[(3R)-4-cyclopentyl-1,3-dimethyl-2-oxo-1,2,3,4-tetrahydropyrido[2,3-b]pyrazin-6-yl]amino}-3-methoxy-N-(1-methylpiperidin-4-yl)benzamide.

¹H NMR (300 MHz, DMSO-d6): δ=1.07 (d, 3H); 1.50-1.80 (m, 10H); 1.85-2.05 (m, 4H); 2.71-2.83 (m, 2H); 3.21 (s, 1H); 3.65-3.79 (m, 1H); 4.20 (q, 1H); 4.38 (qi, 1H); 6.59 (d, 1H); 7.27 (d, 1H); 7.40 (dd, 1H); 7.45 (s, 1H); 8.02 (d, 1H); 8.04 (s, 1H); 8.35 (d, 1H);

### Example 2

### 4-{[(3R)-1,3-Dimethyl-2-oxo-4-(propan-2-yl)-1,2,3,4-tetrahydropyrido[2,3-b]pyrazin-6-yl]amino}-3-methoxy-N-(1-methylpiperidin-4-yl)benzamide

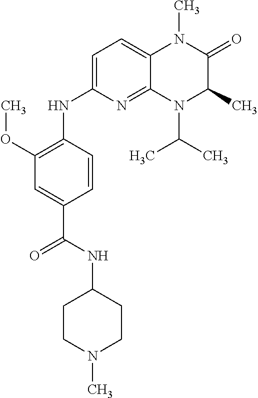 [SMILES: COc1cc(C(=O)NC2CCN(C)CC2)ccc1Nc1ccc2c(n1)N(C(C)C)[C@H](C)C(=O)N2C]

Analogously to the preparation of Example 1, 4-{[(3R)-1,3-dimethyl-2-oxo-4-(propan-2-yl)-1,2,3,4-tetrahydropyrido[2,3-b]pyrazin-6-yl]amino}-3-methoxy-N-(1-methylpiperidin-4-yl)benzamide was prepared from 100 mg of Intermediate 17, 74 mg of 4-amino-1-methylpiperidine (Amine No. 1), 209 mg of TBTU and 180 mg of potassium carbonate in 3 ml of DMF. Purification by RP-HPLC (column: X-Bridge C18 5 μm 100×30 mm, mobile phase: acetonitrile/water (0.2% by volume of formic acid) gradient) gave 35 mg of 4-{[(3R)-1,3-dimethyl-2-oxo-4-(propan-2-yl)-1,2,3,4-tetrahydropyrido[2,3-b]pyrazin-6-yl]amino}-3-methoxy-N-(1-methylpiperidin-4-yl)benzamide.

¹H NMR (300 MHz, DMSO-d6): δ=1.08 (d, 3H); 1.25 (d, 3H); 1.32 (d, 3H); 1.59-1.73 (m, 2H); 1.81-1.90 (m, 2H); 2.45 (s, 3H); 3.03-3.14 (m, 2H); 3.22 (s, 3H); 3.70 (s, 3H); 3.79-3.91 (m, 1H); 4.29 (q, 1H); 4.52-4.64 (m, 1H); 7.15 (s, 1H); 7.39 (d, 1H); 7.42-7.47 (m, 1H); 8.06 (d, 1H); 8.28 (d, 1H);

### Example 3

### 4-{[(3R)-1,3-Dimethyl-2-oxo-4-(propan-2-yl)-1,2,3,4-tetrahydropyrido[2,3-b]pyrazin-6-yl]amino}-3-methoxy-N-[2-(morpholin-4-yl)ethyl]benzamide

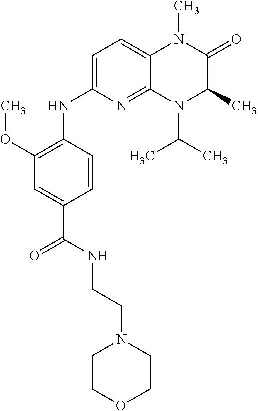 [SMILES: COc1cc(C(=O)NCCN2CCOCC2)ccc1Nc1ccc2c(n1)N(C(C)C)[C@H](C)C(=O)N2C]

Analogously to the preparation of Example 1, 4-{[(3R)-1,3-dimethyl-2-oxo-4-(propan-2-yl)-1,2,3,4-tetrahydropyrido[2,3-b]pyrazin-6-yl]amino}-3-methoxy-N-[2-(morpholin-4-yl)ethyl]benzamide was prepared from 100 mg of Intermediate 17, 84 mg of 2-(morpholin-4-yl)-ethanamine (Amine No. 2), 209 mg of TBTU and 180 mg of potassium carbonate in 3 ml of DMF. Purification by RP-HPLC (column: X-Bridge C18 5 μm 100×30 mm, mobile phase: acetonitrile/water (0.2% by volume of formic acid) gradient) gave 5 mg of 4-{[(3R)-1,3-dimethyl-2-oxo-4-(propan-2-yl)-1,2,3,4-tetrahydropyrido[2,3-b]pyrazin-6-yl]amino}-3-methoxy-N-[2-(morpholin-4-yl)ethyl]benzamide.

¹H NMR (300 MHz, DMSO-d6, selected signals): δ=1.10 (d, 3H); 1.26 (d, 3H); 1.34 (d, 3H); 2.37-2.48 (m, 6H); 3.21 (s, 3H); 3.53-3.62 (m, 4H); 4.26 (q, 1H); 4.59 (sp, 1H); 6.57 (d, 1H); 7.26 (d, 1H); 7.40-7.46 (m, 2H); 8.06 (s, 1H); 8.23 (t, 1H); 8.41 (d, 1H);

### Example 4

### 1-tert-Butyl 4-{2-[(4-{[(3R)-1,3-dimethyl-2-oxo-4-(propan-2-yl)-1,2,3,4-tetrahydropyrido[2,3-b]pyrazin-6-yl]amino}-3-methoxybenzoyl)amino]ethyl}piperazinecarboxylate

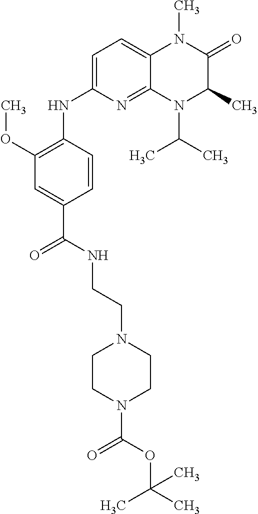 [SMILES: COc1cc(C(=O)NCCN2CCN(C(=O)OC(C)(C)C)CC2)ccc1Nc1ccc2c(n1)N(C(C)C)[C@H](C)C(=O)N2C]

Analogously to the preparation of Example 1, 1-tert-butyl 4-{2-[(4-{[(3R)-1,3-dimethyl-2-oxo-4-(propan-2-yl)-1,2,3,4-tetrahydropyrido[2,3-b]pyrazin-6-yl]amino}-3-methoxybenzoyl)amino]ethyl}piperazinecarboxylate was prepared from 100 mg of Intermediate 17, 149 mg of tert-butyl 4-(2-aminoethyl)piperazine-1-carboxylate (Amine No. 3), 209 mg of TBTU and 180 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 15 mg of 1-tert-butyl 4-{2-[(4-{[(3R)-1,3-dimethyl-2-oxo-4-(propan-2-yl)-1,2,3,4-tetrahydropyrido[2,3-b]pyrazin-6-yl]amino}-3-methoxybenzoyl)amino]ethyl}piperazinecarboxylate.

¹H NMR (300 MHz, DMSO-d6, selected signals): δ=1.09 (d, 3H); 1.25 (d, 3H); 1.33 (d, 3H); 2.29-2.42 (m, 5H); 3.20 (s, 3H); 3.24-3.42 (m, 4H); 3.91 (s, 3H); 4.25 (q, 1H); 4.58 (sp, 1H); 6.57 (d, 1H); 7.25 (d, 1H); 7.39-7.47 (m, 2H); 8.06 (s, 1H); 8.23 (t, 1H); 8.40 (d, 1H);

### Example 5

### N-[2-(Dimethylamino)ethyl]-4-{[(3R)-1,3-dimethyl-2-oxo-4-(propan-2-yl)-1,2,3,4-tetrahydropyrido[2,3-b]pyrazin-6-yl]amino}-3-methoxybenzamide

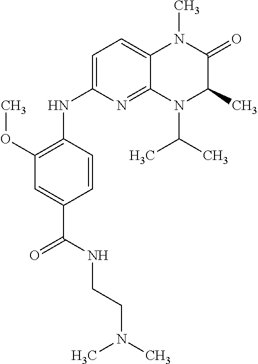 [SMILES: COc1cc(C(=O)NCCN(C)C)ccc1Nc1ccc2c(n1)N(C(C)C)[C@H](C)C(=O)N2C]

Analogously to the preparation of Example 1, N-[2-(dimethylamino)ethyl]-4-{[(3R)-1,3-dimethyl-2-oxo-4-(propan-2-yl)-1,2,3,4-tetrahydropyrido[2,3-b]pyrazin-6-yl]amino}-3-methoxybenzamide was prepared from 100 mg of Intermediate 17, 60 mg of N,N-dimethylethane-1,2-diamine (Amine No. 4), 209 mg of TBTU and 180 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 10 mg of N-[2-(dimethylamino)ethyl]-4-{[(3R)-1,3-dimethyl-2-oxo-4-(propan-2-yl)-1,2,3,4-tetrahydropyrido[2,3-b]pyrazin-6-yl]amino}-3-methoxybenzamide.

¹H NMR (300 MHz, DMSO-d6): δ=1.10 (d, 3H); 1.26 (d, 3H); 1.34 (d, 3H); 2.18 (s, 6H); 2.39 (t, 2H); 3.21 (s, 3H); 3.30-3.39 (m, 2H); 3.92 (s, 3H); 4.26 (q, 1H); 4.59 (sp, 1H); 6.56 (d, 1H); 7.26 (d, 1H); 7.41-7.46 (m, 2H); 8.03 (s, 1H); 8.18 (t, 1H); 8.40 (d, 1H);

### Example 6

### N-Cyclopentyl-4-{[(3R)-1,3-dimethyl-2-oxo-4-(propan-2-yl)-1,2,3,4-tetrahydropyrido[2,3-b]pyrazin-6-yl]amino}-3-methoxybenzamide

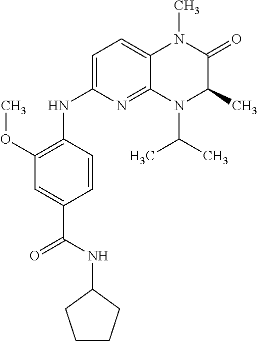 [SMILES: COc1cc(C(=O)NC2CCCC2)ccc1Nc1ccc2c(n1)N(C(C)C)[C@H](C)C(=O)N2C]

Analogously to the preparation of Example 1, N-cyclopentyl-4-{[(3R)-1,3-dimethyl-2-oxo-4-(propan-2-yl)-1,2,3,4-tetrahydropyrido[2,3-b]pyrazin-6-yl]amino}-3-methoxybenzamide was prepared from 100 mg of Intermediate 17, 55 mg of cyclopentylamine (Amine No. 5), 209 mg of TBTU and 180 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 10 mg of N-cyclopentyl-4-{[(3R)-1,3-dimethyl-2-oxo-4-(propan-2-yl)-1,2,3,4-tetrahydropyrido[2,3-b]pyrazin-6-yl]amino}-3-methoxybenzamide.

¹H NMR (300 MHz, DMSO-d6): δ=1.10 (d, 3H); 1.26 (d, 3H); 1.35 8d, 3H); 1.47-1.60 (m, 4H); 1.64-1.75 (m, 2H); 1.84-1.95 (m, 2H); 3.21 (s, 3H); 3.92 (s, 3H); 4.18-4.30 (m, 2H); 4.60 (sp, 1H); 6.56 (d, 1H); 7.26 (d, 1H); 7.43-7.48 (m, 2H); 8.01 (s, 1H); 8.03 (d, 1H); 8.40 (d, 1H);

### Example 7

### (3R)-4-Cyclopentyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one

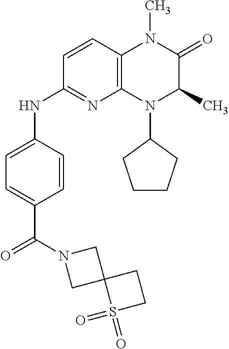 [SMILES: C[C@@H]1C(=O)N(C)c2ccc(Nc3ccc(C(=O)N4CC5(CCS5(=O)=O)C4)cc3)nc2N1C1CCCC1]

371 mg of TBTU were added to 200 mg of Intermediate 19 in 5 ml of DMF, and the solution was shaken at room temperature for 15 min. 275 mg of 1-thia-6-azaspiro[3.3]heptane 1,1-dioxide hydrochloride (Amine No. 6) and 458 μl of N,N-diisopropylethylamine were added, and the reaction mixture was stirred at room temperature for 3 h. The mixture was concentrated and the residue was purified chromatographically in two steps (1. Column: Biotage KP-Sil 10 g. Mobile phase: dichloromethane/methanol gradient. 2. Column: Interchim PF-15 SIHP/12 g. Mobile phase: acetonitrile/water (0.1% of formic acid) gradient). This gave 30 mg of (3R)-4-cyclopentyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one.

¹H NMR (300 MHz, DMSO-d6): δ=1.08 (d, 3H), 1.51-1.82 (m, 6H), 1.91-2.10 (m, 2H), 2.35-2.46 (m, 2H), 3.21 (s, 3H), 4.05-4.16 (m, 2H), 4.21 (q, 1H), 4.27-4.79 (m, 5H), 6.32 (d, 1H), 7.28 (d, 1H), 7.47-7.60 (m, 2H), 7.63-7.75 (m, 2H), 9.19 (s, 1H).

### Example 8

### (3R)-6-({4-[(1,1-Dioxido-1-thia-6-azaspiro[3.3]hept-6-yl)carbonyl]-2-methoxyphenyl}amino)-4-isopropyl-1,3-dimethyl-3,4-dihydropyrido[2,3-b]pyrazin-2(1H)-one

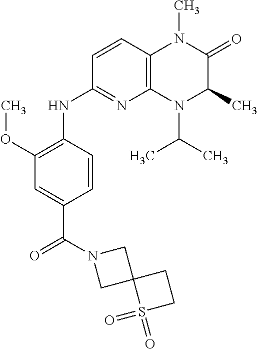 [SMILES: COc1cc(C(=O)N2CC3(CCS3(=O)=O)C2)ccc1Nc1ccc2c(n1)N(C(C)C)[C@H](C)C(=O)N2C]

Analogously to Example 7, (3R)-6-({4-[(1,1-dioxido-1-thia-6-azaspiro[3.3]hept-6-yl)carbonyl]-2-methoxyphenyl}amino)-4-isopropyl-1,3-dimethyl-3,4-dihydropyrido[2,3-b]pyrazin-2(1H)-one was prepared from 200 mg of Intermediate 17, 272 mg of 1-thia-6-azaspiro[3.3]heptane 1,1-dioxide hydrochloride (Amine No. 6), 367 mg of TBTU and 453 μl of N,N-diisopropylethylamine in 5 ml of tetrahydrofuran. Purification by RP chromatography (column: Interchim PF-15 SIHP/12 g. Mobile phase: acetonitrile/water (1% of formic acid) gradient) gave 49 mg of (3R)-6-({4-[(1,1-dioxido-1-thia-6-azaspiro[3.3]hept-6-yl)carbonyl]-2-methoxyphenyl}amino)-4-isopropyl-1,3-dimethyl-3,4-dihydropyrido[2,3-b]pyrazin-2(1H)-one.

¹H NMR (300 MHz, DMSO-d6): δ=1.10 (d, 3H), 1.26 (d, 3H), 1.33 (d, 3H), 2.36-2.48 (m, 2H), 3.21 (s, 3H), 3.92 (s, 3H), 4.11 (dd, 2H), 4.26 (q, 1H), 4.31-4.91 (m, 5H), 6.59 (d, 1H), 7.18-7.30 (m, 3H), 8.11-8.17 (m, 1H), 8.40-8.46 (m, 1H).

### Example 9

### (3R)-6-({4-[(1,1-Dioxido-1-thia-6-azaspiro[3.3]hept-6-yl)carbonyl]phenyl}amino)-4-isopropyl-1,3-dimethyl-3,4-dihydropyrido[2,3-b]pyrazin-2(1H)-one

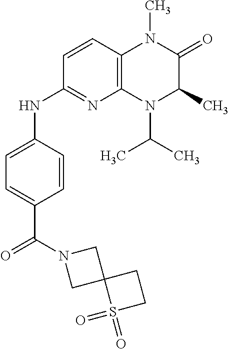 [SMILES: CC(C)N1c2nc(Nc3ccc(C(=O)N4CC5(CCS5(=O)=O)C4)cc3)ccc2N(C)C(=O)[C@H]1C]

Analogously to Example 7, (3R)-6-({4-[(1,1-dioxido-1-thia-6-azaspiro[3.3]hept-6-yl)carbonyl]phenyl}amino)-4-isopropyl-1,3-dimethyl-3,4-dihydropyrido[2,3-b]pyrazin-2(1H)-one was prepared from 142 mg of Intermediate 21, 209 mg of 1-thia-6-azaspiro[3.3]heptane 1,1-dioxide hydrochloride (Amine No. 6), 283 mg of TBTU and 349 μl of N,N-diisopropylethylamine in 4 ml of tetrahydrofuran. Purification by RP chromatography (column: Interchim PF-15 SIHP/12 g. Mobile phase: acetonitrile/water (1% of formic acid) gradient) gave 52 mg of (3R)-6-({4-[(1,1-dioxido-1-thia-6-azaspiro[3.3]hept-6-yl)carbonyl]phenyl}amino)-4-isopropyl-1,3-dimethyl-3,4-dihydropyrido[2,3-b]pyrazin-2(1H)-one.

¹H NMR (300 MHz, DMSO-d6): δ=1.10 (d, 3H), 1.27 (d, 3H), 1.35 (d, 3H), 2.37-2.46 (m, 2H), 3.21 (s, 3H), 4.05-4.16 (m, 2H), 4.26 (q, 1H), 4.31-4.71 (m, 5H), 6.29 (d, 1H), 7.27 (d, 1H), 7.53-7.59 (m, 2H), 7.67-7.74 (m, 2H), 9.19 (s, 1H).

### Example 10

### (3R)-4-Cyclopentyl-6-({4-[(1,1-dioxido-1-thia-6-azaspiro[3.3]hept-6-yl)carbonyl]-2-methoxyphenyl}amino)-1,3-dimethyl-3,4-dihydropyrido[2,3-b]pyrazin-2(1H)-one

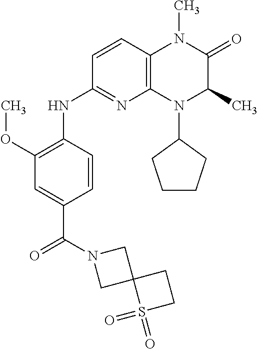 [SMILES: COc1cc(C(=O)N2CC3(CCS3(=O)=O)C2)ccc1Nc1ccc2c(n1)N(C1CCCC1)[C@H](C)C(=O)N2C]

Analogously to Example 7, (3R)-4-cyclopentyl-6-({4-[(1,1-dioxido-1-thia-6-azaspiro[3.3]hept-6-yl)carbonyl]-2-methoxyphenyl}amino)-1,3-dimethyl-3,4-dihydropyrido[2,3-b]pyrazin-2(1H)-one was prepared from 200 mg of Intermediate 7, 255 mg of 1-thia-6-azaspiro[3.3]heptane 1,1-dioxide hydrochloride (Amine No. 6), 344 mg of TBTU and 424 μl of N,N-diisopropylethylamine in 5 ml of DMF. Purification by RP chromatography (column: Interchim PF-15 SIHP/12 g. Mobile phase: acetonitrile/water (1% of formic acid) gradient) gave the product in contaminated form. Subsequent RP-HPLC (column: X-Bridge C18 5 μm 100×30 mm, mobile phase: acetonitrile/water (0.2% by volume of formic acid) gradient) gave 3.7 mg of (3R)-4-cyclopentyl-6-({4-[(1,1-dioxido-1-thia-6-azaspiro[3.3]hept-6-yl)carbonyl]-2-methoxyphenyl}amino)-1,3-dimethyl-3,4-dihydropyrido[2,3-b]pyrazin-2(1H)-one.

¹H NMR (300 MHz, DMSO-d6): δ=1.08 (d, 3H), 1.53-1.77 (m, 6H), 1.89-2.06 (m, 2H), 2.38-2.46 (m, 2H, partially superimposed by DMSO peak) 3.21 (s, 3H), 3.91 (s, 3H), 4.06-4.15 (m, 2H), 4.20 (q, 1H), 4.28-4.73 (m, 5H), 6.62 (s, 1H), 7.21 (d, 2H), 7.27 (d, 1H), 8.12 (s, 1H), 8.34 (s, 1H).

### Example 11

### 4-{[(3R)-4-Cyclopentyl-1,3-dimethyl-2-oxo-1,2,3,4-tetrahydropyrido[2,3-b]pyrazin-6-yl]amino}-N-(1-methylpiperidin-4-yl)benzamide

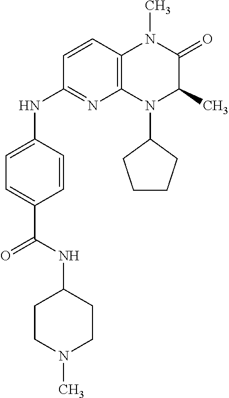 [SMILES: C[C@@H]1C(=O)N(C)c2ccc(Nc3ccc(C(=O)NC4CCN(C)CC4)cc3)nc2N1C1CCCC1]

Analogously to Example 7, 4-{[(3R)-4-cyclopentyl-1,3-dimethyl-2-oxo-1,2,3,4-tetrahydropyrido[2,3-b]pyrazin-6-yl]amino}-N-(1-methylpiperidin-4-yl)benzamide was prepared from 200 mg of Intermediate 19, 120 mg of 4-amino-1-methylpiperidine (Amine No. 1), 371 mg of TBTU and 275 μl of N,N-diisopropylethylamine in 5 ml of DMF. RP chromatography (column: Interchim PF-15 SIHP/12 g. Mobile phase: acetonitrile/water (1% of formic acid) gradient) gave the product in contaminated form. Subsequent purification by RP-HPLC (column: X-Bridge C18 5 μm 100×30 mm, mobile phase: acetonitrile/water (0.2% by volume of formic acid) gradient) gave 8 mg of 4-{[(3R)-4-cyclopentyl-1,3-dimethyl-2-oxo-1,2,3,4-tetrahydropyrido[2,3-b]pyrazin-6-yl]amino}-N-(1-methylpiperidin-4-yl)benzamide. Some of the product (about 50%) is present as hydroformate.

¹H NMR (300 MHz, DMSO-d6): δ=1.08 (d, 3H), 1.55-1.88 (m, 10H), 2.01 (m, 2H), 2.24-2.39 (m, 5H), 2.97 (m, 2H), 3.21 (s, 3H, superimposed by water peak), 3.81 (d, 1H), 4.21 (q, 1H), 4.35-4.49 (m, 1H), 6.31 (d, 1H), 7.27 (d, 1H), 7.62-7.69 (m, 2H), 7.71-7.78 (m, 2H), 7.99 (d, 1H), 9.07 (s, 1H).

### Example 12

### 4-{[(3R)-4-Cyclohexyl-1,3-dimethyl-2-oxo-1,2,3,4-tetrahydropyrido[2,3-b]pyrazin-6-yl]amino}-3-methoxy-N-(1-methylpiperidin-4-yl)benzamide

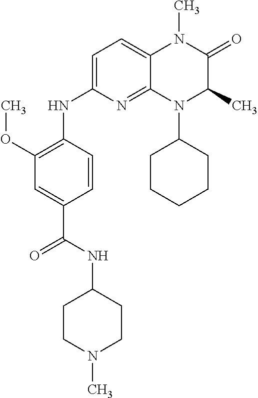 [SMILES: COc1cc(C(=O)NC2CCN(C)CC2)ccc1Nc1ccc2c(n1)N(C1CCCCC1)[C@H](C)C(=O)N2C]

Analogously to the preparation of Example 1, 4-{[(3R)-4-cyclohexyl-1,3-dimethyl-2-oxo-1,2,3,4-tetrahydropyrido[2,3-b]pyrazin-6-yl]amino}-3-methoxy-N-(1-methylpiperidin-4-yl)benzamide was prepared from 60 mg of Intermediate 12, 40 mg of 4-amino-1-methylpiperidine (Amine No. 1), 113 mg of TBTU and 98 mg of potassium carbonate in 3 ml of DMF. The reaction solution was added to water and the product as precipitate was filtered off with suction. This gave 42 mg of 4-{[(3R)-4-cyclohexyl-1,3-dimethyl-2-oxo-1,2,3,4-tetrahydropyrido[2,3-b]pyrazin-6-yl]amino}-3-methoxy-N-(1-methylpiperidin-4-yl)benzamide.

¹H NMR (300 MHz, DMSO-d6): δ=1.08 (d, 3H); 1.13-1.29 (m, 1H); 1.31-1.49 (m, 3H); 1.49-2.00 (m, 12H); 2.05-2.14 (m, 1H); 2.16 (s, 3H); 2.73-2.83 (m, 2H); 3.20 (s, 3H); 3.66-3.77 (m, 1H); 4.14-4.29 (m, 2H); 6.57 (d, 1H); 7.25 (d, 1H); 7.41 (dd, 1H); 7.45 (d, 1H); 8.04 (d, 1H); 8.07 (s, 1H); 8.48 (d, 1H).

### Example 13

### 4-{[(3R)-1,3-Dimethyl-2-oxo-4-(tetrahydro-2H-pyran-4-yl)-1,2,3,4-tetrahydropyrido[2,3-b]pyrazin-6-yl]amino}-3-methoxy-N-(1-methylpiperidin-4-yl)benzamide

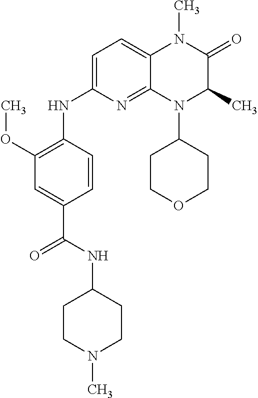 [SMILES: COc1cc(C(=O)NC2CCN(C)CC2)ccc1Nc1ccc2c(n1)N(C1CCOCC1)[C@H](C)C(=O)N2C]

A solution of 590 mg of Intermediate 26, 316 mg of 4-amino-1-methylpiperidine (Amine No. 1), 0.56 ml of triethylamine and 789 mg of HATU in 57 ml of DMF was stirred at RT for 72 hours. The mixture was added to semisaturated sodium chloride solution and extracted three times with ethyl acetate, the extract was washed with brine and dried over sodium sulphate and the solvent was removed completely under reduced pressure. The residue was purified by chromatography on silica gel (Biotage KP-NH column, mobile phase dichloromethane/methanol gradient). The resulting product was taken up in ethyl acetate and washed three more times with semisaturated sodium chloride solution. The organic phase was dried over sodium sulphate and the solvent was removed completely under reduced pressure. This gave 476 mg of 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(1-methylpiperidin-4-yl)benzamide.

¹H NMR (400 MHz, DMSO-d6): δ=1.09 (d, 3H); 1.50-1.69 (m, 3H); 1.70-1.84 (m, 3H); 1.86-2.06 (m, 4H); 2.17 (s, 3H); 2.73-2.85 (m, 2H); 3.21 (s, 3H); 3.66-3.82 (m, 1H); 3.92 (s, 3H); 3.94-4.08 (m, 2H); 4.25 (q, 1H); 4.40 (tt, 1H); 6.60 (d, 1H); 7.28 (d, 1H); 7.40-7.49 (m, 2H); 8.03 (d, 1H); 8.10 (s, 1H); 8.41 (d, 1H).

### Example 14

### N-{trans-4-[4-(Cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzamide

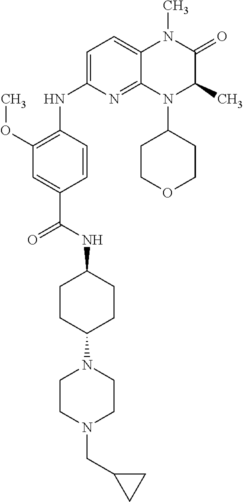 [SMILES: COc1cc(C(=O)N[C@H]2CC[C@H](N3CCN(CC4CC4)CC3)CC2)ccc1Nc1ccc2c(n1)N(C1CCOCC1)[C@H](C)C(=O)N2C]

A solution of 100 mg of Intermediate 26, 111 mg of trans-4-[4-(cyclopropylmethyl)piperazin-1-yl]cyclohexanamine (Amine No. 7), 0.13 ml of triethylamine and 134 mg of HATU in 9.6 ml of DMF was stirred at RT for 72 hours. The mixture was added to semisaturated sodium chloride solution and extracted three times with ethyl acetate, the extract was washed with brine and dried over sodium sulphate and the solvent was removed completely under reduced pressure. The residue was purified by RP-HPLC (column: X-Bridge C18 5 μm 100×30 mm, mobile phase: acetonitrile/water (0.1% by volume formic acid) gradient). This gave 36 mg of N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzamide.

¹H NMR (400 MHz, DMSO-d6): δ=0.02-0.11, 0.40-0.50 (m, 2H); 0.73-0.87 (m, 1H); 1.09 (d, 3H); 1.21-1.47 (m, 4H); 1.57-1.68 (m, 1H); 1.70-2.06 (m, 7H); 2.14-2.32 (m+d, 3H); 2.54 (s, 3H); 3.21 (s, 3H); 3.39-3.54 (m, 2H); 3.92 (s, 3H); 3.95-4.08 (m, 2H); 4.25 (q, 1H); 4.40 (tt, 1H); 6.60 (d, 1H); 7.27 (d, 1H); 7.39-7.48 (m, 2H); 7.99 (d, 1H); 8.10 (s, 1H); 8.41 (d, 1H).

### Example 15

### N-{trans-4-[4-(Cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide

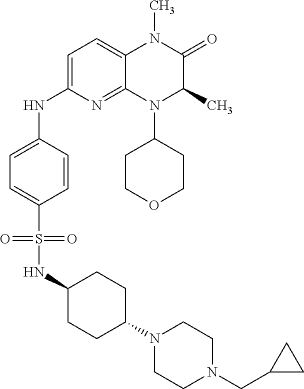 [SMILES: C[C@@H]1C(=O)N(C)c2ccc(Nc3ccc(S(=O)(=O)N[C@H]4CC[C@H](N5CCN(CC6CC6)CC5)CC4)cc3)nc2N1C1CCOCC1]

A suspension of 150 mg of Intermediate 24, 378 mg of 4-amino-N-{trans-4-[4-(cyclopropylmethyl)piperazin-1-yl]cyclohexyl}benzenesulphonamide (Intermediate 28), 22 mg of palladium(II) acetate, 785 mg of caesium carbonate and 60 mg of (+)-BINAP in 10.7 ml of toluene was stirred at 110° C. under an argon atmosphere for 11 hours. The reaction solution was filtered off, the residue was washed with ethyl acetate and the combined organic phases were evaporated to dryness. The residue was purified by RP-HPLC chromatography (column: X-Bridge C18 5 μm 100×30 mm, mobile phase: acetonitrile/water (0.1% by volume formic acid) gradient). This gave 95 mg of N-{4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide.

¹H NMR (400 MHz, DMSO-d6, selected signals): δ=0.01-0.08 (m, 2H); 0.38-0.48 (m, 2H); 0.71-0.85 (m, 1H); 1.04-1.2 (m, 7H); 1.59-1.74 (m, 5H); 1.80 (dq, 1H); 1.89-2.05 (m, 2H); 2.08-2.22 (m+d, 3H); 2.38-2.50 (m, 4H); 3.21 (s, 3H); 3.47 (q, 2H); 4.01 (bt, 2H); 4.26 (q, 1H); 4.38 (tt, 1H); 6.31 (d, 1H); 7.30 (d, 1H); 7.39 (d, 1H); 7.63 (d, 2H); 7.76 (d, 2H); 9.35 (s, 1H).

### Example 16

### (3R)-1,3-Dimethyl-6-[(4-{[4-(propan-2-yl)piperazin-1-yl]sulphonyl}phenyl)amino]-4-(tetrahydro-2H-pyran-4-yl)-3,4-dihydropyrido[2,3-b]pyrazin-2(1H)-one

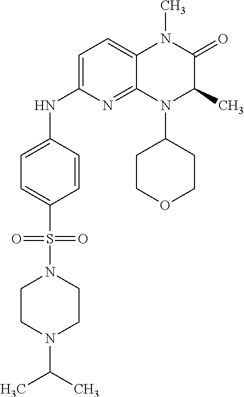 [SMILES: CC(C)N1CCN(S(=O)(=O)c2ccc(Nc3ccc4c(n3)N(C3CCOCC3)[C@H](C)C(=O)N4C)cc2)CC1]

A suspension of 150 mg of Intermediate 24, 273 mg of 4-{[4-(propan-2-yl)piperazin-1-yl]sulphonyl}aniline (Amine No. 8, preparation analogous to US20030225106), 21.6 mg of palladium(II) acetate, 785 mg of caesium carbonate and 60 mg of (+)-BINAP in 10.8 ml of toluene was stirred at 120° C. under an argon atmosphere for 3 hours. After cooling to RT, the mixture was added to water and extracted twice with ethyl acetate. The combined organic phases were washed with saturated aqueous sodium chloride solution and dried over sodium sulphate, and the solvent was removed completely under reduced pressure. The residue was purified by RP-HPLC chromatography (column: X-Bridge C18 5 μm 100×30 mm, mobile phase: acetonitrile/water (0.1% by volume formic acid) gradient). This gave 65 mg of (3R)-1,3-dimethyl-6-[(4-{[4-(propan-2-yl)piperazin-1-yl]sulphonyl}phenyl)amino]-4-(tetrahydro-2H-pyran-4-yl)-3,4-dihydropyrido[2,3-b]pyrazin-2(1H)-one.

¹H NMR (400 MHz, CDCl₃): δ=1.03 (d, 6H); 1.24 (d, 3H); 1.65-1.94 (m, 8H); 1.94-2.14 (m, 2H); 3.00-3.14 (m, 4H); 3.32 (s, 3H); 3.48-3.61 (m, 2H); 4.05-4.16 (m, 2H); 4.31 (q, 1H); 4.50 (tt, 1H); 6.29 (d, 1H); 6.82 (s, 1H); 7.06 (d, 1H); 7.54 (d, 2H); 7.62 (d, 2H).

### Example 17

### 4-{[(3R)-4-Cyclohexyl-1,3-dimethyl-2-oxo-1,2,3,4-tetrahydropyrido[2,3-b]pyrazin-6-yl]amino}-N,N-dimethylbenzenesulphonamide

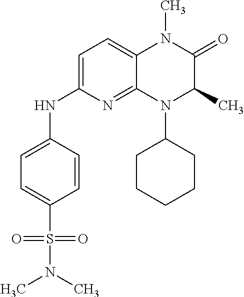 [SMILES: C[C@@H]1C(=O)N(C)c2ccc(Nc3ccc(S(=O)(=O)N(C)C)cc3)nc2N1C1CCCCC1]

Analogously to the preparation of Example 20, 4-{[(3R)-4-cyclohexyl-1,3-dimethyl-2-oxo-1,2,3,4-tetrahydropyrido[2,3-b]pyrazin-6-yl]amino}-N,N-dimethylbenzenesulphonamide was prepared from 160 mg of Intermediate 10, 218 mg of 4-amino-N,N-dimethylbenzenesulphonamide (Amine No. 9), 24.5 mg of palladium(II) acetate, 887 mg of caesium carbonate and 68 mg of (+)-BINAP in 3 ml of toluene under an argon atmosphere. Purification by RP-HPLC (column: X-Bridge C18 5 μm 100×30 mm, mobile phase: acetonitrile/water (0.2% by volume of formic acid) gradient) gave 105 mg of 4-{[(3R)-4-cyclohexyl-1,3-dimethyl-2-oxo-1,2,3,4-tetrahydropyrido[2,3-b]pyrazin-6-yl]amino}-N,N-dimethylbenzenesulphonamide.

¹H NMR (400 MHz, DMSO-d6): δ=1.09 (d, 3H); 1.16-1.28 (m, 1H); 1.32-1.55 (m, 3H); 1.60-1.77 (m, 3H); 1.78-1.92 (m, 2H); 2.07-2.15 (m, 1H); 2.57 (s, 6H); 3.21 (s, 3H); 4.18 (tt, 1H); 4.25 (q, 1H); 6.31 (d, 1H); 7.29 (d, 1H); 7.54 (d, 2H); 7.85 (d, 2H); 9.40 (s, 1H).

TABLE 2
The following examples were prepared analogously to Example 15 from the respective intermediates:

| | | | Intermediate/ | |
|---|---|---|---|---|
| Ex. | Structure | Name | Amine | Analytical data |
| 18 | 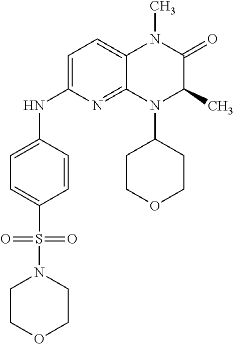 [SMILES: C[C@@H]1C(=O)N(C)c2ccc(Nc3ccc(S(=O)(=O)N4CCOCC4)cc3)nc2N1C1CCOCC1] | (3R)-1,3-dimethyl-6-{[4- (morpholin-4- ylsulphonyl)phenyl] amino}-4-(tetrahydro- 2H-pyran- 4-yl)-3,4- dihydropyrido[2,3- b]pyrazin-2(1H)-one | Intermediate 24; Amine No. 10 | ¹H NMR (400 MHz, DMSO-d6): δ = 1.10 (d, 3H); 1.64 (bd, 1H); 1.81 (dq, 1H); 1.92-2.04 (m, 2H); 2.80-2.89 (m, 4H); 3.22 (s, 3H); 3.41-3.56 (m, 2H); 3.59-3.67 (m, 4H); 3.93-4.06 (m, 2H); 4.27 (q, 1H); 4.31-4.42 (m, 1H); 6.35 (d, 1H); 7.32 (d, 1H); 7.56 (d, 2H); 7.83 (d, 2H); 9.45 (s, 1H). |
| 19 | 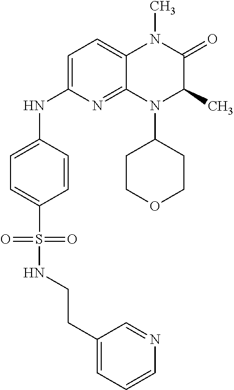 [SMILES: C[C@@H]1C(=O)N(C)c2ccc(Nc3ccc(S(=O)(=O)NCCc4cccnc4)cc3)nc2N1C1CCOCC1] | 4-{[(3R)-1,3-dimethyl-2- oxo-4-(tetrahydro-2H- pyran-4-yl)-1,2,3,4- tetrahydropyrido[2,3- b]pyrazin-6-yl]amino}-N- [2-(pyridin-3- yl)ethyl]benzene- sulphonamide | Intermediate 24; Intermediate 62 | ¹H NMR (400 MHz, DMSO-d6): δ = 1.10 (d, 3H); 1.63 (bd, 1H); 1.81 (dq, 1H); 1.90-2.05 (m, 2H); 2.69 (t, 2H); 2.97 (q, 2H); 3.21 (s, 3H); 3.47 (q, 2H); 4.00 (dt, 2H); 4.46 (q, 1H); 4.38 (tt, 1H); 6.32 (d, 1H); 7.27 (dd, 1H); 7.30 (d, 1H); 7.46 (t, 1H); 7.54-7.65 (m, 3H); 7.77 (d, 2H); 8.33-8.42 (m, 2H); 9.34 (s, 1H). |
| 20 | 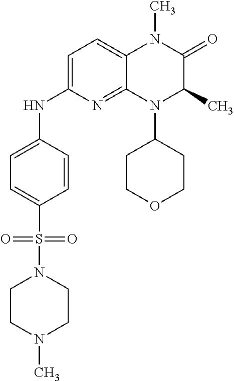 [SMILES: C[C@@H]1C(=O)N(C)c2ccc(Nc3ccc(S(=O)(=O)N4CCN(C)CC4)cc3)nc2N1C1CCOCC1] | (3R)-1,3-dimethyl-6-({4- [(4-methylpiperazin-1- yl)sulphonyl]phenyl} amino)-4-(tetrahydro-2H- pyran-4-yl)-3,4- dihydropyrido[2,3- b]pyrazin-2(1H)-one | Intermediate 24; Amine No. 11 | ¹H-NMR (400 MHz, DMSO-d6, sel. signals): δ = 1.10 (d, 3H); 1.64 (bd, 1H); 1.81 (dq, 1H); 1.90- 2.05 (m, 2H); 2.14 (s, 3H); 2.28-2.41 (m, 4H); 2.78- 2.92 (m, 4H); 3.22 (s, 3H); 4.00 (dt, 2H); 4.27 (q, 1H); 4.37 (tt, 1H); 6.34 (d, 1H); 7.31 (d, 1H); 7.55 (d, 2H); 7.82 (d, 2H); 9.42 (s, 1H). |
| 21 | 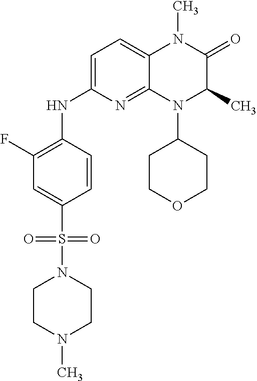 [SMILES: C[C@@H]1C(=O)N(C)c2ccc(Nc3ccc(S(=O)(=O)N4CCN(C)CC4)cc3F)nc2N1C1CCOCC1] | (3R)-6-({2-fluoro-4-[(4- methylpiperazin-1- yl)sulphonyl]phenyl} amino)-1,3-dimethyl-4- (tetrahydro-2H-pyran-4- yl)-3,4- dihydropyrido[2,3- b]pyrazin-2(1H)-one | Intermediate 24; Amine No. 12 | ¹H-NMR (400 MHz, DMSO-d6, sel. signals): δ = 1.09 (d, 3H); 1.61 (bd, 1H); 1.78 (dq, 1H); 1.88- 2.03 (m, 2H); 2.14 (s, 3H); 2.30-2.41 (m, 4H); 2.84- 2.95 (m, 4H); 3.22 (s, 3H); 3.90-4.03 (m, 2H); 4.22- 4.40 (m, 2H); 6.62 (d, 1H); 7.34 (d, 1H); 7.44 (dd, 1H); 7.51 (dd, 1H); 8.58 (t, 1H); 9.07 (d, 1H). |
| 22 | 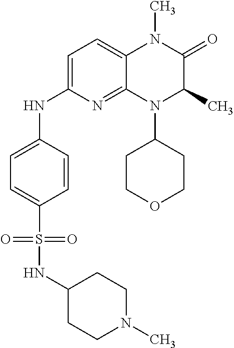 [SMILES: C[C@@H]1C(=O)N(C)c2ccc(Nc3ccc(S(=O)(=O)NC4CCN(C)CC4)cc3)nc2N1C1CCOCC1] | 4-{[(3R)-1,3-dimethyl-2- oxo-4-(tetrahydro-2H- pyran-4-yl)-1,2,3,4- tetrahydropyrido[2,3- b]pyrazin-6-yl]amino}-N- (1-methylpiperidin-4- yl)benzenesulphonamide | Intermediate 24; Amine No. 13 | ¹H NMR (400 MHz, CD₃OD): δ = 1.19 (d, 3H); 1.59-1.76 (m, 3H); 1.82- 2.00 (m, 3H); 2.01-2.14 (m, 2H); 2.54 (s, 3H); 2.55-2.66 (m, 1H); 3.53- 3.63 (m, 2H); 4.04-4.15 (m, 2H); 4.32 (q, 1H); 4.51 (tt, 1H); 6.33 (d, 1H); 7.29 (d, 1H); 7.71 (d, 2H); 7.79 (d, 2H). |
| 23 | 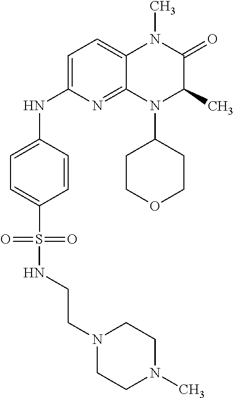 [SMILES: C[C@@H]1C(=O)N(C)c2ccc(Nc3ccc(S(=O)(=O)NCCN4CCN(C)CC4)cc3)nc2N1C1CCOCC1] | 4-{[(3R)-1,3-dimethyl-2- oxo-4-(tetrahydro-2H- pyran-4-yl)-1,2,3,4- tetrahydropyrido[2,3- b]pyrazin-6-yl]amino}-N- [2-(4-methylpiperazin-1- yl)ethyl]benzene- sulponamide | Intermediate 24; Intermediate 64 | ¹H NMR (400 MHz, DMSO-d6): δ = 1.10 (d, 3H); 1.63 (bd, 1H); 1.81 (dq, 1H); 1.90-2.05 (m, 2H); 2.16 (s, 3H); 2.23- 2.41 (m, 9H); 2.80 (q, 2H); 3.21 (s, 3H); 3.48 (q, 2H); 4.01 (dt, 2H); 4.26 (q, 1H); 4.38 (dt, 1H); 6.32 (d, 1H); 7.23 (t, 1H); 7.30 (d, 1H); 7.63 (d, 2H); 7.79 (d, 2H); 9.36 (s, 1H). |
| 24 | 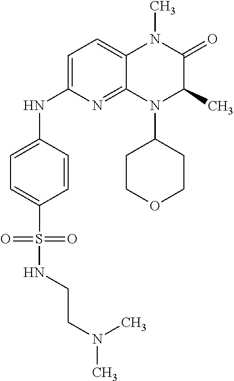 [SMILES: C[C@@H]1C(=O)N(C)c2ccc(Nc3ccc(S(=O)(=O)NCCN(C)C)cc3)nc2N1C1CCOCC1] | N-[2- (dimethylamino)ethyl]-4- {[(3R)-1,3-dimethyl-2- oxo-4-(tetrahydro-2H- pyran-4-yl)-1,2,3,4- tetrahydropyrido[2,3- b]pyrazin-6- yl]amino}benzene- sulphonamide | Intermediate 24; Amine No. 14 | ¹H NMR (400 MHz, DMSO-d6): δ = 1.10 (d, 3H); 1.63 (bd, 1H); 1.81 (dq, 1H); 1.89-2.05 (m, 2H); 2.10 (s, 6H); 2.29 (t, 2H); 2.80 (t, 2H); 3.21 (s, 3H); 3.48 (q, 2H); 4.01 (t, 2H); 4.26 (q, 1H); 4.38 (tt, 1H); 6.32 (d, 1H); 7.28 (bs, 1H); 7.30 (d, 1H); 7.63 (d, 2H); 7.79 (d, 2H); 9.36 (s, 1H). |
| 25 | 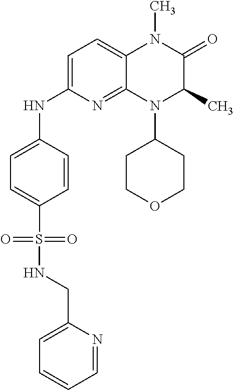 [SMILES: C[C@@H]1C(=O)N(C)c2ccc(Nc3ccc(S(=O)(=O)NCc4ccccn4)cc3)nc2N1C1CCOCC1] | 4-{[(3R)-1,3-dimethyl-2- oxo-4-(tetrahydro-2H- pyran-4-yl)-1,2,3,4- tetrahydropyrido[2,3- b]pyrazin-6-yl]amino}-N- (pyridin-2- ylmethyl)benzene- sulphonamide | Intermediate 24; Intermediate 66 | ¹H NMR (400 MHz, DMSO-d6): δ = 1.11 (d, 3H); 1.64 (bd, 1H); 1.81 (dq, 1H); 1.89-2.05 (m, 2H); 3.22 (s, 3H); 3.48 (q, 2H); 3.95-4.10 (m+d, 3H); 4.27 (q, 1H); 4.38 (tt, 1H); 6.31 (d, 1H); 7.23 (dd, 1H); 7.31 (d, 1H); 7.37 (d, 1H); 7.61-7.81 (m+2d, 5H); 7.97/t, 1H); 8.43 (bd, 1H); 9.34 (s, 1H). |
| 26 | 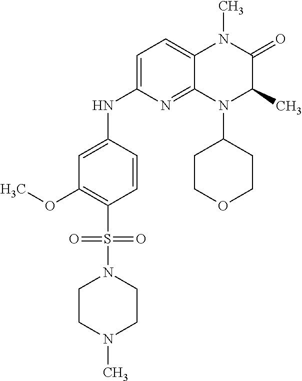 | (3R)-6-({3-methoxy-4- [(4-methylpiperazin-1- yl)sulphonyl]phenyl} amino)-1,3-dimethyl-4- (tetrahydro-2H-pyran-4- yl)-3,4- dihydropyrido[2,3- b]pyrazin-2(1H)-one | Intermediate 24; Amine No. 15 | ¹H-NMR (400 MHz, DMSO-d6, sel. signals): δ = 1.10 (d, 3H); 1.62 (bd, 1H); 1.82 (dq, 1H); 1.86- 2.02 (m, 2H); 2.16 (s, 3H); 2.26-2.39 (m, 4H); 2.96- 3.10 (m, 4H); 3.22 (s, 3H); 3.84 (s, 3H); 3.98 (dt, 2H); 4.25 (q, 1H); 4.40 (tt, 1H); 6.34 (d, 1H); 7.01 (d, 1H); 7.31 (d, 1H); 7.53 (d, 1H); 7.63 (dd, 1H); 9.33 (s, 1H). |
| 27 | 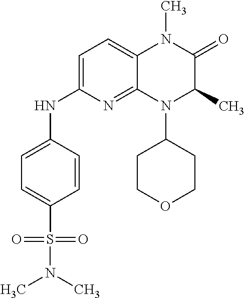 [SMILES: C[C@@H]1C(=O)N(C)c2ccc(Nc3ccc(S(=O)(=O)N(C)C)cc3)nc2N1C1CCOCC1] | 4-{[(3R)-1,3-dimethyl-2- oxo-4-(tetrahydro-2H- pyran-4-yl)-1,2,3,4- tetrahydropyrido[2,3- b]pyrazin-6-yl]amino}- N,N- dimethylbenzene- sulphonamide | Intermediate 24; Amine No. 9 | ¹H NMR (400 MHz, DMSO-d6): δ = 1.10 (d, 3H); 1.64 (bd, 1H); 1.81 (dq, 1H); 1.91-2.04 (m, 2H); 2.57 (s, 6H); 3.22 (s, 3H); 3.45 (dt, 1H); 3.50 (dt, 1H); 3.93-3.45 (m, 2H); 4.26 (q, 1H); 4.37 (tt, 1H); 6.34 (d, 1H); 7.31 (d, 1H); 7.57 (d, 2H); 7.82 (d, 2H); 9.41 (s, 1H). |

### Example 28

### (3R)-4-Cyclohexyl-6-[(2-methoxy-4-{[4-(propan-2-yl)piperazin-1-yl]carbonyl}phenyl)amino]-1,3-dimethyl-3,4-dihydropyrido[2,3-b]pyrazin-2(1H)-one

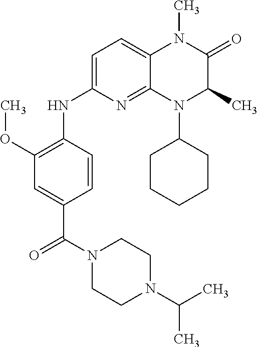 [SMILES: COc1cc(C(=O)N2CCN(C(C)C)CC2)ccc1Nc1ccc2c(n1)N(C1CCCCC1)[C@H](C)C(=O)N2C]

Analogously to the preparation of Example 1, (3R)-4-cyclohexyl-6-({4-[(4-isopropylpiperazin-1-yl)carbonyl]-2-methoxyphenyl}amino)-1,3-dimethyl-3,4-dihydropyrido[2,3-b]pyrazin-2(1H)-one was prepared from 50 mg of Intermediate 12, 39 mg of 1-isopropylpiperazine (Amine No. 16), 95 mg of TBTU and 81 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 30 mg of (3R)-4-cyclohexyl-6-({4-[(4-isopropylpiperazin-1-yl)carbonyl]-2-methoxyphenyl}amino)-1,3-dimethyl-3,4-dihydropyrido[2,3-b]pyrazin-2(1H)-one.

¹H NMR (400 MHz, DMSO-d6): δ=0.97 (d, 6H); 1.08 (d, 3H); 1.13-1.55 (m, 4H); 1.55-1.76 (m, 3H); 1.76-1.91 (m, 2H); 2.02-2.14 (m, 1H); 2.35-2.47 (m, 4H); 2.59-2.77 (m, 1H); 3.20 (s, 3H); 3.38-3.64 (m, 4H); 3.89 (s, 3H); 4.09-4.28 (m, 2H); 6.54 (d, 1H); 6.89 (d, 1H); 6.99 (d, 1H); 7.24 (d, 1H); 8.06 (s, 1H); 8.42 (d, 1H);

### Example 29

### (3R)-4-Cyclohexyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one

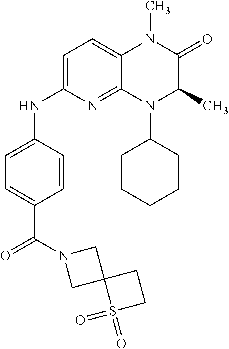 [SMILES: C[C@@H]1C(=O)N(C)c2ccc(Nc3ccc(C(=O)N4CC5(CCS5(=O)=O)C4)cc3)nc2N1C1CCCCC1]

Analogously to the preparation of Example 1, (3R)-4-cyclohexyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one was prepared from 55 mg of Intermediate 30, 96 mg of 1-thia-6-azaspiro[3.3]heptane 1,1-dioxide hydrochloride (Amine No. 6), 112 mg of TBTU and 96 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 37 mg of (3R)-4-cyclohexyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one.

¹H NMR (400 MHz, DMSO-d₆): δ=1.09 (d, 3H); 1.15-1.31 (m, 1H); 1.32-1.58 (m, 3H); 1.59-1.78 (m, 3H); 1.78-1.97 (m, 2H); 2.06-2.18 (m, 1H); 2.42 (t, 2H); 3.20 (s, 3H); 4.11 (t, 2H); 4.17-4.30 (m, 2H); 4.29-4.86 (m, 4H); 6.27 (d, 1H); 7.27 (d, 1H); 7.53 (d, 2H); 7.75 (d, 2H); 9.23 (bs, 1H);

### Example 30

### 4-{[(3R)-4-Cyclohexyl-1,3-dimethyl-2-oxo-1,2,3,4-tetrahydropyrido[2,3-b]pyrazin-6-yl]amino}-N-(1-methylazetidin-3-yl)benzamide

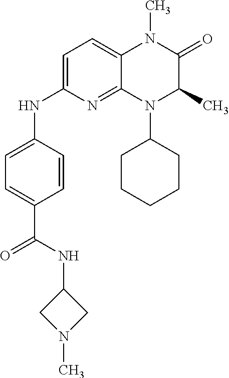 [SMILES: C[C@@H]1C(=O)N(C)c2ccc(Nc3ccc(C(=O)NC4CN(C)C4)cc3)nc2N1C1CCCCC1]

Analogously to the preparation of Example 1, 4-{[(3R)-4-cyclohexyl-1,3-dimethyl-2-oxo-1,2,3,4-tetrahydropyrido[2,3-b]pyrazin-6-yl]amino}-N-(1-methylazetidin-3-yl)benzamide was prepared from 55 mg of Intermediate 30, 57 mg of 1-methylazetidine-3-amine (Amine No. 17), 112 mg of TBTU and 96 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 22 mg of 4-{[(3R)-4-cyclohexyl-1,3-dimethyl-2-oxo-1,2,3,4-tetrahydropyrido[2,3-b]pyrazin-6-yl]amino}-N-(1-methylazetidin-3-yl)benzamide.

¹H NMR (400 MHz, DMSO-d₆): δ=1.08 (d, 3H); 1.15-1.31 (m, 1H); 1.31-1.54 (m, 3H); 1.54-1.76 (m, 3H); 1.77-1.95 (m, 2H); 2.06-2.15 (m, 1H); 2.24 (s, 3H); 2.94 (t, 2H); 3.19 (s, 3H); 3.53 (t, 2H); 4.15-4.28 (m, 2H); 4.39 (q, 1H); 6.26 (d, 1H); 7.25 (d, 1H); 7.67-7.77 (m, 4H); 8.48 (d, 1H); 9.13 (bs, 1H);

### Example 31

### (3R)-4-Cyclohexyl-1,3-dimethyl-6-[(4-{[4-(propan-2-yl)piperazin-1-yl]carbonyl}phenyl)amino]-3,4-dihydropyrido[2,3-b]pyrazin-2(1H)-one

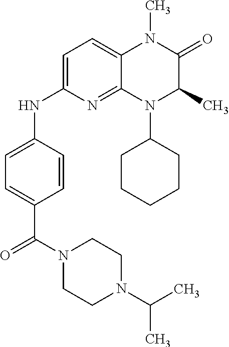 [SMILES: CC(C)N1CCN(C(=O)c2ccc(Nc3ccc4c(n3)N(C3CCCCC3)[C@H](C)C(=O)N4C)cc2)CC1]

Analogously to the preparation of Example 1, (3R)-4-cyclohexyl-6-({4-[(4-isopropylpiperazin-1-yl)carbonyl]phenyl}amino)-1,3-dimethyl-3,4-dihydropyrido[2,3-b]pyrazin-2(1H)-one was prepared from 50 mg of Intermediate 30, 85 mg of 1-isopropylpiperazine (Amine No. 16), 102 mg of TBTU and 88 mg of potassium carbonate in 3 ml of DMF. Purification by RP-HPLC (column: X-Bridge C18 5 μm 100×30 mm, mobile phase: acetonitrile/water (0.2% by volume of formic acid) gradient) gave 28 mg of (3R)-4-cyclohexyl-6-({4-[(4-isopropylpiperazin-1-yl)carbonyl]phenyl}amino)-1,3-dimethyl-3,4-dihydropyrido[2,3-b]pyrazin-2(1H)-one.

¹H NMR (400 MHz, DMSO-d6): δ=0.95-1.14 (m, 9H); 1.17-1.29 (m, 1H); 1.32-1.56 (m, 3H); 1.57-1.75 (m, 3H); 1.78-1.94 (m, 2H); 2.06-2.17 (m, 1H); 2.55-2.81 (m, 5H); 3.20 (s, 3H); 3.39-3.74 (m, 4H); 4.13-4.30 (m, 2H); 6.25 (d, 1H); 7.26 (d, 1H); 7.30 (d, 2H); 7.72 (d, 2H); 9.08 (s, 1H);

### Example 32

### (3R)-4-Cycloheptyl-6-{[2-methoxy-4-(2-oxa-6-azaspiro[3.3]hept-6-ylcarbonyl)phenyl]amino}-1,3-dimethyl-3,4-dihydropyrido[2,3-b]pyrazin-2(1H)-one

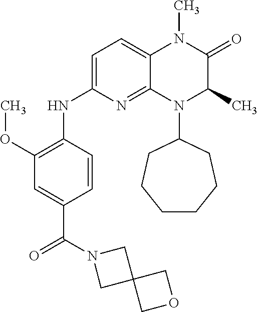 [SMILES: COc1cc(C(=O)N2CC3(COC3)C2)ccc1Nc1ccc2c(n1)N(C1CCCCCC1)[C@H](C)C(=O)N2C]

Analogously to the preparation of Example 1, (3R)-4-cycloheptyl-6-{[2-methoxy-4-(2-oxa-6-azaspiro[3.3]hept-6-ylcarbonyl)phenyl]amino}-1,3-dimethyl-3,4-dihydropyrido[2,3-b]pyrazin-2(1H)-one was prepared from 50 mg of Intermediate 39, 36 mg of 2-oxa-6-azaspiro[3.3]heptane oxalate (2:1) (Amine No. 18), 101 mg of TBTU and 87 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 29 mg of (3R)-4-cycloheptyl-6-{[2-methoxy-4-(2-oxa-6-azaspiro[3.3]hept-6-ylcarbonyl)phenyl]amino}-1,3-dimethyl-3,4-dihydropyrido[2,3-b]pyrazin-2(1H)-one.

¹H NMR (400 MHz, DMSO-d6): δ=1.09 (d, 3H); 1.39-1.82 (m, 10H); 1.82-1.97 (m, 1H); 1.98-2.13 (m, 1H); 3.20 (s, 3H); 3.90 (s, 3H); 4.10-4.37 (m, 4H); 4.50 (bs, 2H); 4.68 (s, 4H); 6.57 (d, 1H); 7.12 (dd, 1H); 7.18 (d, 1H); 7.26 (d, 1H); 8.13 (s, 1H); 8.44 (d, 1H).

### Example 33

### (3R)-4-Cycloheptyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one

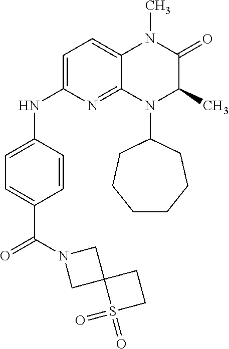 [SMILES: C[C@@H]1C(=O)N(C)c2ccc(Nc3ccc(C(=O)N4CC5(CCS5(=O)=O)C4)cc3)nc2N1C1CCCCCC1]

Analogously to the preparation of Example 1, (3R)-4-cycloheptyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one was prepared from 60 mg of Intermediate 40, 101 mg of 1-thia-6-azaspiro[3.3]heptane 1,1-dioxide hydrochloride (Amine No. 6), 118 mg of TBTU and 102 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 32 mg of (3R)-4-cycloheptyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one.

¹H NMR (400 MHz, DMSO-d6): δ=1.10 (d, 3H); 1.36-1.83 (m, 10H); 1.83-1.99 (m, 1H); 2.00-2.15 (m, 1H); 2.41 (t, 2H); 3.20 (s, 3H); 4.11 (t, 2H); 4.24 (q, 1H); 4.26-4.86 (m, 5H); 6.27 (d, 1H); 7.27 (d, 1H); 7.52 (d, 2H); 7.73 (d, 2H); 9.22 (s, 1H).

### Example 34

### 4-{[(3R)-4-Benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}-3-methoxybenzamide

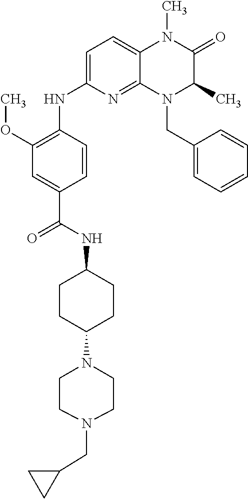 [SMILES: COc1cc(C(=O)N[C@H]2CC[C@H](N3CCN(CC4CC4)CC3)CC2)ccc1Nc1ccc2c(n1)N(Cc1ccccc1)[C@H](C)C(=O)N2C]

Analogously to the preparation of Example 1, 4-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}-3-methoxybenzamide was prepared from 48 mg of Intermediate 45, 66 mg of 4-[4-(cyclopropylmethyl)piperazin-1-yl]cyclohexanamine (Amine No. 7), 89 mg of TBTU and 77 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 33 mg of 4-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}-3-methoxybenzamide.

¹H NMR (400 MHz, CDCl₃): δ=0.12-0.23 (m, 2H); 0.52-0.61 (m, 2H); 0.87-1.02 (m, 1H); 1.20-1.34 (m, 2H); 1.24 (d, 3H); 1.47 (q, 2H); 2.02 (d, 2H); 2.18 (d, 2H); 2.32-2.45 (m, 3H); 2.58-2.89 (m, 8H); 3.34 (s, 3H); 3.84-3.95 (m, 1H); 3.97 (s, 3H); 4.09 (q, 1H); 4.21 (d, 1H); 5.42 (d, 1H); 5.82 (d, 1H); 6.30 (d, 1H); 7.04-7.11 (m, 2H); 7.28-7.54 (m, 7H); 8.11 (d, 1H).

### Example 35

### 4-{[(3R)-1,3-Dimethyl-2-oxo-4-(tetrahydro-2H-pyran-4-yl)-1,2,3,4-tetrahydropyrido[2,3-b]pyrazin-6-yl]amino}-N-(pyridin-2-ylmethyl)benzamide

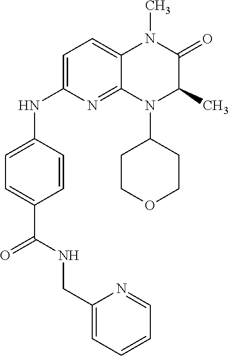 [SMILES: C[C@@H]1C(=O)N(C)c2ccc(Nc3ccc(C(=O)NCc4ccccn4)cc3)nc2N1C1CCOCC1]

Analogously to the preparation of Example 1, 4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(pyridin-2-ylmethyl)benzamide was prepared from 100 mg of Intermediate 32, 55 mg of 1-(pyridin-2-yl)methanamine (Amine No. 19), 143 mg of HATU and 102 mg of triethylamine in 10 ml of DMF. Purification by RP-HPLC (column: X-Bridge C18 5 μm 100×30 mm, mobile phase: acetonitrile/water (0.2% by volume of formic acid) gradient) gave 74 mg of 4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(pyridin-2-ylmethyl)benzamide.

¹H NMR (400 MHz, DMSO-d6): δ=1.10 (d, 3H); 1.65 (bd, 1H); 1.80 (dq, 1H); 1.96 (dq, 1H); 1.04 (bd, 1H); 3.21 (s, 3H); 3.45-3.58 (m, 2H); 3.96-4.10 (m, 2H); 4.26 (q, 1H); 4.43 (tt, 1H); 4.59 (d, 2H); 6.31 (dm 1H); 7.29 (d, 1H); 7.36 (dd, 1H); 7.40 (d, 1H); 7.73 (d, 2H); 7.81-7.90 (m, 3H); 8.55 (dd, 1H); 8.92 (t, 1H); 9.18 (s, 1H).

### Example 36

### (3R)-1,3-Dimethyl-6-({2-methyl-4-[(4-methylpiperazin-1-yl)carbonyl]phenyl}amino)-4-(tetrahydro-2H-pyran-4-yl)-3,4-dihydropyrido[2,3-b]pyrazin-2(1H)-one

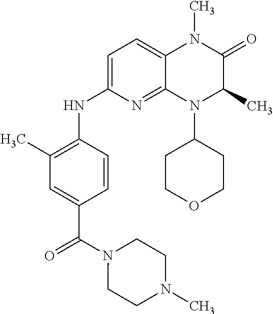 [SMILES: Cc1cc(C(=O)N2CCN(C)CC2)ccc1Nc1ccc2c(n1)N(C1CCOCC1)[C@H](C)C(=O)N2C]

Analogously to the preparation of Example 1, (3R)-1,3-dimethyl-6-({2-methyl-4-[(4-methylpiperazin-1-yl)carbonyl]phenyl}amino)-4-(tetrahydro-2H-pyran-4-yl)-3,4-dihydropyrido[2,3-b]pyrazin-2(1H)-one was prepared from 100 mg of Intermediate 34, 49 mg of 1-methylpiperazine (Amine No. 20), 139 mg of HATU and 98 mg of triethylamine in 7.5 ml of DMF. Purification by RP-HPLC (column: X-Bridge C18 5 μm 100×30 mm, mobile phase: acetonitrile/water (0.2% by volume of formic acid) gradient) gave 58 mg of (3R)-1,3-dimethyl-6-({2-methyl-4-[(4-methylpiperazin-1-yl)carbonyl]phenyl}amino)-4-(tetrahydro-2H-pyran-4-yl)-3,4-dihydropyrido[2,3-b]pyrazin-2(1H)-one.

¹H NMR (400 MHz, DMSO-d6, selected signals): δ=1.08 (d, 3H); 2.56 (bd, 1H); 1.73 (dq, 1H); 1.83-1.97 (m, 2H); 2.28 (s, 3H); 2.34-2.46 (m, 3H); 3.27 (t, 2H); 3.38 (t, 2H); 3.93 (dd, 2H); 4.32 (q, 1H); 4.29 (tt, 1H); 6.42 (dd, 1H); 7.26 (d, 1H); 7.22 (s, 1H); 7.26 (d, 1H); 7.91 (s, 1H); 7.98 (dd, 1H).

### Example 37

### N-{trans-4-[4-(Cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzamide

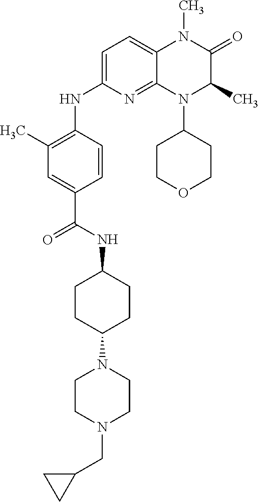 [SMILES: Cc1cc(C(=O)N[C@H]2CC[C@H](N3CCN(CC4CC4)CC3)CC2)ccc1Nc1ccc2c(n1)N(C1CCOCC1)[C@H](C)C(=O)N2C]

Analogously to the preparation of Example 1, N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzamide was prepared from 100 mg of Intermediate 34, 116 mg of trans-4-[4-(cyclopropylmethyl)piperazin-1-yl]cyclohexanamine (Amine No. 7), 139 mg of HATU and 98 mg of triethylamine in 7.5 ml of DMF. Purification by RP-HPLC (column: X-Bridge C18 5 μm 100×30 mm, mobile phase: acetonitrile/water (0.2% by volume of formic acid) gradient) gave 58 mg of N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzamide.

¹H NMR (400 MHz, DMSO-d6, selected signals): δ=0.03-0.09 (m, 2H); 0.42-0.48 (m, 2H); 0.75-0.86 (m, 1H); 1.08 (d, 3H); 1.23-1.42 (m, 4H); 1.57 (bd, 1H); 1.73 (dq, 1H); 1.79-1.97 (m, 6H); 2.17 (d, 2H); 2.19-2.28 (m, 1H); 2.30 (s, 3H); 3.32 (dt, 2H); 3.40 (dt, 2H); 3.63-3.76 (m, 2H); 3.91-3.99 (m, 2H); 4.22 (q, 1H); 4.33 (tt, 1H); 6.44 (d, 1H); 7.27 (d, 1H); 7.60 (dd, 1H); 7.67 (s, 1H); 7.87 (s, 1H); 7.92 (d, 1H); 8.03 (d, 1H).

### Example 38

### 4-{[(3R)-1,3-Dimethyl-2-oxo-4-(tetrahydro-2H-pyran-4-yl)-1,2,3,4-tetrahydropyrido[2,3-b]pyrazin-6-yl]amino}-3-methoxy-N-(4-oxocyclohexyl)benzamide

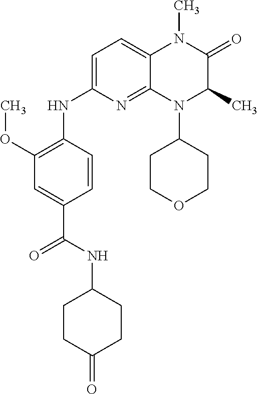 [SMILES: COc1cc(C(=O)NC2CCC(=O)CC2)ccc1Nc1ccc2c(n1)N(C1CCOCC1)[C@H](C)C(=O)N2C]

Analogously to the preparation of Example 1, 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(4-oxocyclohexyl)benzamide was prepared from 200 mg of Intermediate 26, 80 mg of 4-aminocyclohexanone (Amine No. 21), 267 mg of HATU and 190 mg of triethylamine in 19 ml of DMF. Purification by RP-HPLC (column: X-Bridge C18 5 μm 100×30 mm, mobile phase: acetonitrile/water (0.2% by volume of formic acid) gradient) gave 36 mg of 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(4-oxocyclohexyl)benzamide.

¹H NMR (400 MHz, DMSO-d6, selected signals): δ=1.09 (d, 3H); 1.63 (bd, 1H); 1.71-1.90 (m, 4H); 1.90-2.05 (m, 2H); 2.05-2.17 (m, 2H); 2.22-2.33 (m, 2H); 3.21 (s, 3H); 3.41-3.54 (m, 2H); 3.93 (s, 3H); 3.94-4.08 (m, 2H); 4.25 (q, 1H); 4.40 (tt, 1H); 6.61 (m, 1H); 7.28 (m, 1H); 7.42-7.50 (m, 2H); 8.08-8.16 (m, 2H); 8.42 (d, 1H).

### Example 39

### N-(1-Acetylpiperidin-4-yl)-4-{[(3R)-4-cyclohexyl-1,3-dimethyl-2-oxo-1,2,3,4-tetrahydropyrido[2,3-b]pyrazin-6-yl]amino}-3-methoxybenzamide

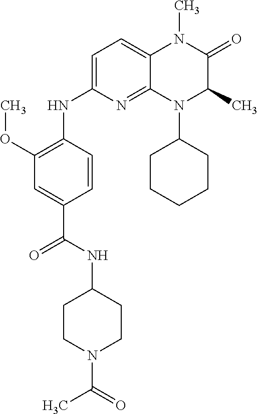 [SMILES: COc1cc(C(=O)NC2CCN(C(C)=O)CC2)ccc1Nc1ccc2c(n1)N(C1CCCCC1)[C@H](C)C(=O)N2C]

Analogously to the preparation of Example 1, N-(1-acetylpiperidin-4-yl)-4-{[(3R)-4-cyclohexyl-1,3-dimethyl-2-oxo-1,2,3,4-tetrahydropyrido[2,3-b]pyrazin-6-yl]amino}-3-methoxybenzamide was prepared from 50 mg of Intermediate 12, 42 mg of 1-(4-aminopiperidin-1-yl)ethanone (Amine No. 22), 95 mg of TBTU and 81 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 31 mg of N-(1-acetylpiperidin-4-yl)-4-{[(3R)-4-cyclohexyl-1,3-dimethyl-2-oxo-1,2,3,4-tetrahydropyrido[2,3-b]pyrazin-6-yl]amino}-3-methoxybenzamide.

¹H NMR (400 MHz, DMSO-d6): δ=1.08 (d, 3H); 1.23 (t, 1H); 1.30-1.58 (m, 5H); 1.58-1.74 (m, 3H); 1.74-1.94 (m, 4H); 2.02 (s, 3H); 2.09 (d, 1H); 2.64 (t, 1H); 3.13 (t, 1H); 3.20 (s, 3H); 3.84 (d, 1H); 3.93 (s, 3H); 3.96-4.12 (m, 1H); 4.12-4.30 (m, 2H); 4.38 (d, 1H); 6.58 (d, 1H); 7.26 (d, 1H); 7.41 (dd, 1H); 7.45 (d, 1H); 8.03-8.13 (m, 2H); 8.50 (d, 1H).

### Example 40

### (3R)-4-Cycloheptyl-6-[(2-methoxy-4-{[4-(propan-2-yl)piperazin-1-yl]carbonyl}phenyl)amino]-1,3-dimethyl-3,4-dihydropyrido[2,3-b]pyrazin-2(1H)-one

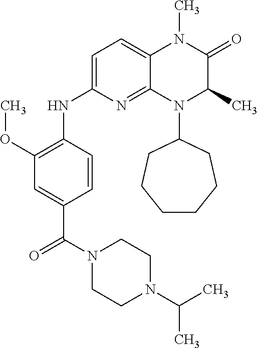 [SMILES: COc1cc(C(=O)N2CCN(C(C)C)CC2)ccc1Nc1ccc2c(n1)N(C1CCCCCC1)[C@H](C)C(=O)N2C]

Analogously to the preparation of Example 1, (3R)-4-cycloheptyl-6-[(2-methoxy-4-{[4-(propan-2-yl)piperazin-1-yl]carbonyl}phenyl)amino]-1,3-dimethyl-3,4-dihydropyrido[2,3-b]pyrazin-2(1H)-one was prepared from 55 mg of Intermediate 39, 41 mg of 1-isopropylpiperazine (Amine No. 16), 101 mg of TBTU and 87 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 42 mg of (3R)-4-cycloheptyl-6-[(2-methoxy-4-{[4-(propan-2-yl)piperazin-1-yl]carbonyl}phenyl)amino]-1,3-dimethyl-3,4-dihydropyrido[2,3-b]pyrazin-2(1H)-one.

1H NMR (400 MHz, DMSO-d6): δ=0.97 (d, 6H); 1.09 (d, 3H); 1.41-1.80 (m, 10H); 1.89 (q, 1H); 1.97-2.09 (m, 1H); 2.38-2.48 (m, 4H); 2.68 (qi, 1H); 3.20 (s, 3H); 3.40-3.61 (m, 4H); 3.89 (s, 3H); 4.22 (q, 1H); 4.22-4.32 (m, 1H); 6.52 (d, 1H); 6.88 (dd, 1H); 6.99 (d, 1H); 7.24 (d, 1H); 8.02 (s, 1H); 8.37 (d, 1H).

### Example 41

### (3R)-4-Benzyl-1,3-dimethyl-6-{[4-(2-oxa-6-azaspiro[3.3]hept-6-ylcarbonyl)phenyl]amino}-3,4-dihydropyrido[2,3-b]pyrazin-2(1H)-one

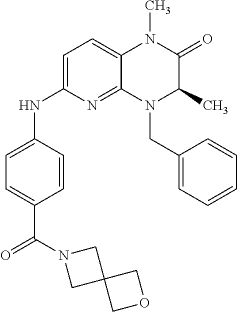 [SMILES: C[C@@H]1C(=O)N(C)c2ccc(Nc3ccc(C(=O)N4CC5(COC5)C4)cc3)nc2N1Cc1ccccc1]

Analogously to the preparation of Example 1, (3R)-4-benzyl-1,3-dimethyl-6-{[4-(2-oxa-6-azaspiro[3.3]hept-6-ylcarbonyl)phenyl]amino}-3,4-dihydropyrido[2,3-b]pyrazin-2(1H)-one was prepared from 55 mg of Intermediate 47, 36 mg of 2-oxa-6-azaspiro[3.3]heptane oxalate (2:1) (Amine No. 18), 100 mg of TBTU and 86 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 17 mg of (3R)-4-benzyl-1,3-dimethyl-6-{[4-(2-oxa-6-azaspiro[3.3]hept-6-ylcarbonyl)phenyl]amino}-3,4-dihydropyrido[2,3-b]pyrazin-2(1H)-one.

¹H NMR (400 MHz, DMSO-d6): δ=1.15 (d, 3H); 3.25 (s, 3H); 4.08 (q, 1H); 4.13-4.31 (m, 2H); 4.35 (d, 1H); 4.36-4.54 (m, 2H); 4.68 (s, 4H); 5.15 (d, 1H); 6.30 (d, 1H); 7.22-7.42 (m, 8H); 7.46 (m, 2H); 9.08 (s, 1H).

### Example 42

### 4-{[(3R)-4-Benzyl-1,3-dimethyl-2-oxo-1,2,3,4-tetrahydropyrido[2,3-b]pyrazin-6-yl]amino}-N-(4-hydroxycyclohexyl)benzamide

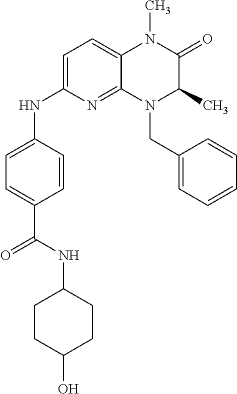 [SMILES: C[C@@H]1C(=O)N(C)c2ccc(Nc3ccc(C(=O)NC4CCC(O)CC4)cc3)nc2N1Cc1ccccc1]

Analogously to the preparation of Example 1, 4-{[(3R)-4-benzyl-1,3-dimethyl-2-oxo-1,2,3,4-tetrahydropyrido[2,3-b]pyrazin-6-yl]amino}-N-(4-hydroxycyclohexyl)benzamide was prepared from 50 mg of Intermediate 47, 36 mg of 4-aminocyclohexanol (Amine No. 23), 100 mg of TBTU and 86 mg of potassium carbonate in 3 ml of DMF. Purification by RP-HPLC (column: X-Bridge C18 5 μm 100×30 mm, mobile phase: acetonitrile/water (0.2% by volume of ammonia) gradient) gave 7 mg of 4-{[(3R)-4-benzyl-1,3-dimethyl-2-oxo-1,2,3,4-tetrahydropyrido[2,3-b]pyrazin-6-yl]amino}-N-(4-hydroxycyclohexyl)benzamide.

¹H NMR (400 MHz, DMSO-d6): δ=1.13 (d, 3H); 1.15-1.42 (m, 4H); 1.71-1.88 (m, 4H); 3.23 (s, 3H); 3.56-3.76 (m, 1H); 3.99 (q, 1H); 4.30 (d, 1H); 4.55 (d, 1H); 5.22 (d, 1H); 6.30 (d, 1H); 7.23-7.44 (m, 6H); 7.53 (d, 2H); 7.65 (d, 2H); 7.84 (d, 1H); 9.10 (s, 1H).

### Example 43

### (3R)-4-Benzyl-6-({4-[(4-fluoropiperidin-1-yl)carbonyl]-2-methoxyphenyl}amino)-1,3-dimethyl-3,4-dihydropyrido[2,3-b]pyrazin-2(1H)-one

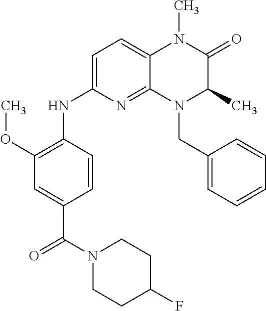 [SMILES: COc1cc(C(=O)N2CCC(F)CC2)ccc1Nc1ccc2c(n1)N(Cc1ccccc1)[C@H](C)C(=O)N2C]

Analogously to the preparation of Example 1, (3R)-4-benzyl-6-({4-[(4-fluoropiperidin-1-yl)carbonyl]-2-methoxyphenyl}amino)-1,3-dimethyl-3,4-dihydropyrido[2,3-b]pyrazin-2(1H)-one was prepared from 48 mg of Intermediate 45, 40 mg of 4-fluoropiperidine (Amine No. 24), 89 mg of TBTU and 77 mg of potassium carbonate in 3 ml of DMF. Purification by RP-HPLC (column: X-Bridge C18 5 μm 100×30 mm, mobile phase: acetonitrile/water (0.2% by volume of formic acid) gradient) gave 29 mg of (3R)-4-benzyl-6-({4-[(4-fluoropiperidin-1-yl)carbonyl]-2-methoxyphenyl}amino)-1,3-dimethyl-3,4-dihydropyrido[2,3-b]pyrazin-2(1H)-one.

¹H NMR (400 MHz, DMSO-d6): δ=1.13 (d, 3H); 1.60-1.79 (m, 2H); 1.79-2.02 (m, 2H); 3.24 (s, 3H); 3.39-3.67 (m, 4H); 3.86 (s, 3H); 4.07 (q, 1H); 4.32 (d, 1H); 4.76-4.89 (m, 0.5H); 4.93-5.04 (m, 0.5H); 5.12 (d, 1H); 6.56 (d, 1H); 6.72 (dd, 1H); 6.97 (d, 1H); 7.19-7.40 (m, 6H); 7.94-8.04 (m, 2H).

### Example 44

### 4-{[(3R)-4-Cycloheptyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide

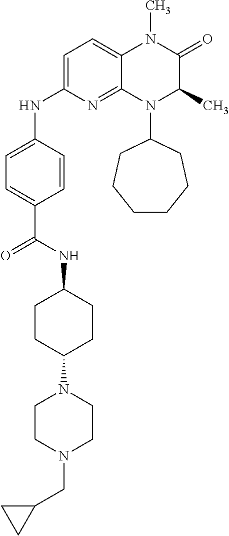 [SMILES: C[C@@H]1C(=O)N(C)c2ccc(Nc3ccc(C(=O)N[C@H]4CC[C@H](N5CCN(CC6CC6)CC5)CC4)cc3)nc2N1C1CCCCCC1]

Analogously to the preparation of Example 1, 4-{[(3R)-4-cycloheptyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide was prepared from 60 mg of Intermediate 40, 87 mg of trans-4-[4-(cyclopropylmethyl)piperazin-1-yl]cyclohexanamine (Amine No. 7), 118 mg of TBTU and 102 mg of potassium carbonate in 3 ml of DMF. Purification by RP-HPLC (column: Acquity BEH C18 1.7 50×2.1 mm, mobile phase: acetonitrile/water (0.2% by volume of ammonia) gradient) gave 14 mg of 4-{[(3R)-4-cycloheptyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide.

¹H NMR (400 MHz, CDCl₃): δ=0.13-0.22 (m, 2H), 0.54-0.62 (m, 2H), 0.91-1.02 (m, 1H), 1.23 (d, 3H), 1.24-1.34 (m, 2H), 1.38-1.90 (m, 14H), 1.97-2.09 (m, 2H), 2.13-2.25 (m, 2H), 2.33-2.48 (m, 3H), 2.66-2.91 (m, 8H), 3.30 (s, 3H), 3.85-4.01 (m, 1H), 4.32 (q, 1H), 4.36-4.45 (m, 1H), 5.85 (d, 1H), 6.24 (d, 1H), 6.52 (s, 1H), 7.02 (d, 1H), 7.48 (d, 2H), 7.68 (d, 2H).

### Example 45

### (3R)-6-({2-Methoxy-4-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one

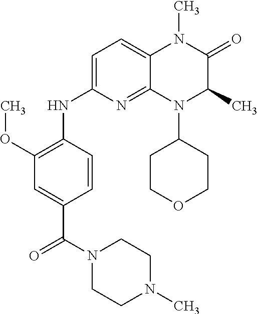 [SMILES: COc1cc(C(=O)N2CCN(C)CC2)ccc1Nc1ccc2c(n1)N(C1CCOCC1)[C@H](C)C(=O)N2C]

A solution of 590 mg of Intermediate 26, 277 mg of 1-methylpiperidine (Amine No. 20), 789 mg of HATU and 560 mg of triethylamine in 57 ml of DMF was stirred at RT for 72 hours. The mixture was added to semisaturated sodium chloride solution and extracted three times with ethyl acetate, the extract was washed with brine and dried over sodium sulphate and the solvent was removed completely under reduced pressure. The residue was purified by chromatography on silica gel (Biotage KP-NH column, mobile phase dichloromethane/methanol gradient). The resulting product was taken up in ethyl acetate and washed three more times with semisaturated sodium chloride solution. The organic phase was dried over sodium sulphate and the solvent was removed completely under reduced pressure. This gave 503 mg of (3R)-6-({2-methoxy-4-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one.

¹H NMR (400 MHz, DMSO-d6): δ=1.08 (d, 3H); 1.61 (bd, 1H); 1.77 (dq, 1H); 1.86-2.01 (m, 2H); 2.20 (s, 3H); 2.27-2.37 (m, 4H); 3.20 (s, 3H); 3.34-3-47 (m, 2H); 3.47-3.58 (m, 4H); 3.88 (s, 3H); 3.91-4.04 (m, 2H); 4.23 (q, 1H); 4.35 (tt, 1H); 6.56 (d, 1H); 6.92 (dd, 1H); 6.99 (d, 1H); 7.26 (d, 1H); 8.07 (s, 1H); 8.34 (d, 1H).

TABLE 3
The following examples were prepared analogously to Example 1 from the respective
intermediates:

| | | | Intermediate/ | |
|---|---|---|---|---|
| Ex. | Structure | Name | Amine | Analytical data |
| 46 | 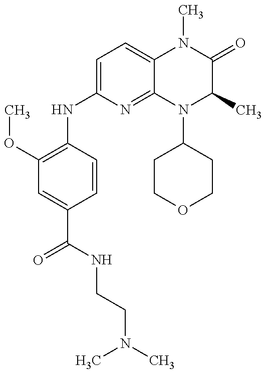 [SMILES: COc1cc(C(=O)NCCN(C)C)ccc1Nc1ccc2c(n1)N(C1CCOCC1)[C@H](C)C(=O)N2C] | N-[2- (Dimethyalmino)ethyl]-4- {[(3R)-1,3-dimethyl-2-oxo- 4-(tetrahydro-2H-pyran-4- yl)-1,2,3,4- tetrahydropyrido[2,3- b]pyrazin-6-yl]amino}-3- methoxybenzamide | Intermediate 26; Amine No. 4 | ¹H-NMR (400 MHz, DMSO- d6, sel. signals); δ = 1.09 (d, 3H); 1.63 (bd, 1H); 1.77 (dq, 1H); 1.86-2.05 (m, 2H); 2.33 (s, 6H); 2.59 (t, 2H); 3.21 (s, 3H); 3.36-3.54 (m, 4H); 3.92 (s, 3H); 4.25 (q, 1H); 4.40 (tt, 1H); 6.61 (d, 1H); 7.28 (d, 1H); 7.41-7.51 (m, 2H); 8.13 (s, 1H); 8.36 (t, 1H); 8.45 (d, 1H). |
| 47 | 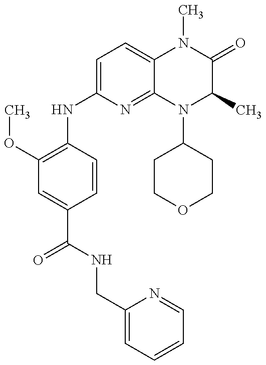 [SMILES: COc1cc(C(=O)NCc2ccccn2)ccc1Nc1ccc2c(n1)N(C1CCOCC1)[C@H](C)C(=O)N2C] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-3- methoxy-N-(pyridin-2- ylmethyl)benzamide | Intermediate 26; Amine No. 19, | ¹H NMR (400 MHz, DMSO- d6): δ = 1.09 (d, 1H); 1.64 (bd, 1H); 1.78 (dq, 1H); 1.94 (dq, 1H); 2.01 (bd, 1H); 3.21 (s, 3H); 3.49 (t, 2H); 4.02 (dt, 2H); 4.26 (q, 1H); 4.41 (tt, 1H); 4.60 (d, 2H); 6.63 (d, 1H); 7.29 /d, 1H); 7.32 (dd, 1H); 7.27 (d, 1H); 7.52-7.60 (m, 2H); .83 (dt, 1H); 8.17 (s, 1H); 8.50 (d, 1H); 8.54 (d, 1H); 9.01 (t, 1H). |
| 48 | 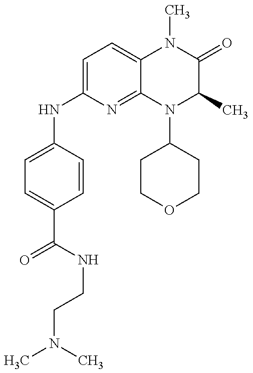 [SMILES: C[C@@H]1C(=O)N(C)c2ccc(Nc3ccc(C(=O)NCCN(C)C)cc3)nc2N1C1CCOCC1] | N-[2- (Dimethylamino)ethyl]-4- {[(3R)-1,3-dimethyl-2-oxo- 4-(tetrahydro-2H-pyran-4- yl)-1,2,3,4- tetrahydropyrido[2,3- b]pyrazin-6- yl]amino}benzamide | Intermediate 32; Amine No. 4 | UPLC-MS: Rt = 0.72 min (M⁺ + 1 = 467) |
| 49 | 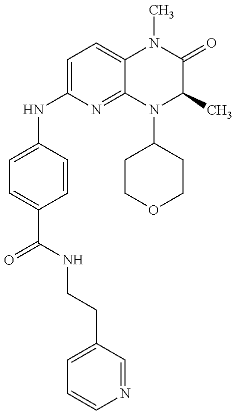 [SMILES: C[C@@H]1C(=O)N(C)c2ccc(Nc3ccc(C(=O)NCCc4cccnc4)cc3)nc2N1C1CCOCC1] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-N-[2- (pyridin-3- yl)ethyl]benzamide | Intermediate 32; Amine No. 25 | UPLC-MS: Rt = 0.74 min (M⁺ + 1 = 501) |
| 50 | 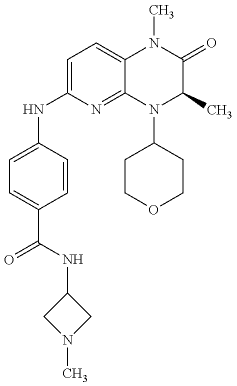 [SMILES: C[C@@H]1C(=O)N(C)c2ccc(Nc3ccc(C(=O)NC4CN(C)C4)cc3)nc2N1C1CCOCC1] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-N-(1- methylazetidin-3- yl)benzamide | Intermediate 32; Amine No. 17 | UPLC-MS: Rt = 0.72 min (M⁺ + 1 = 465) |
| 51 | 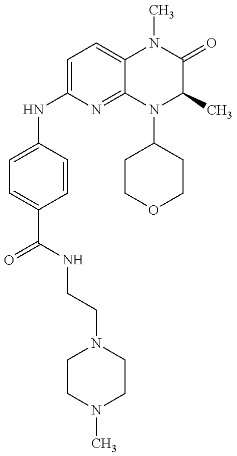 [SMILES: C[C@@H]1C(=O)N(C)c2ccc(Nc3ccc(C(=O)NCCN4CCN(C)CC4)cc3)nc2N1C1CCOCC1] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-N-[2- (4-methylpiperazin-1- yl)ethyl]benzamide | Intermediate 32; Amine No. 26 | UPLC-MS: Rt = 0.69 min (M⁺ + 1 = 522) |
| 52 | 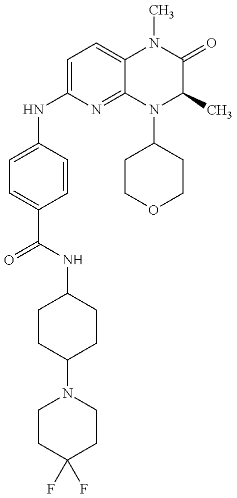 [SMILES: C[C@@H]1C(=O)N(C)c2ccc(Nc3ccc(C(=O)NC4CCC(N5CCC(F)(F)CC5)CC4)cc3)nc2N1C1CCOCC1] | N-[4-(4,4-Difluoropiperidin- 1-yl)cyclohexyl]-4-{[3R)- 1,3-dimethyl-2-oxo-4- (tetrahydro-2H-pyran-4-yl)- 1,2,3,4- tetrahydropyrido[2,3- b]pyrazin-6- yl]amino}benzamide | Intermediate 32; Intermediate 68 | UPLC-MS: Rt = 0.80 min (M⁺ + 1 = 597) |
| 53 | 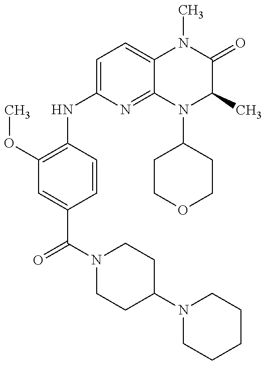 [SMILES: COc1cc(C(=O)N2CCC(N3CCCCC3)CC2)ccc1Nc1ccc2c(n1)N(C1CCOCC1)[C@H](C)C(=O)N2C] | (3R)-6-{[4-(1,4′-Bipiperidin- 1′-ylcarbonyl)-2- methoxyphenyl]amino}-1,3- dimethyl-4-(tetrahydro-2H- pyran-4-yl)-3,4- dihydropyrido[2,3- b]pyrazin-2(1H)-one | Intermediate 26; Amine No. 27 | UPLC-MS: Rt = 0.82 min (M⁺ = 577) |
| 54 | 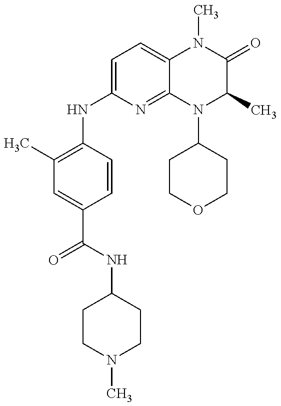 [SMILES: Cc1cc(C(=O)NC2CCN(C)CC2)ccc1Nc1ccc2c(n1)N(C1CCOCC1)[C@H](C)C(=O)N2C] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-3- methyl-N-(1- methylpiperidin-4- yl)benzamide | Intermediate 34; Amine No. 1 | UPLC-MS: Rt = 0.78 min (M⁺ + 1 = 507) |
| 55 | 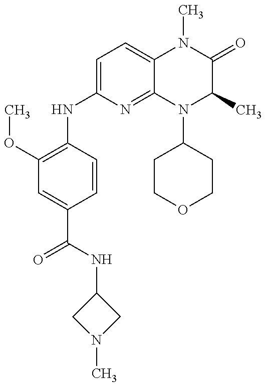 [SMILES: COc1cc(C(=O)NC2CN(C)C2)ccc1Nc1ccc2c(n1)N(C1CCOCC1)[C@H](C)C(=O)N2C] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-3- methoxy-N-(1- methylazetidin-3- yl)benzamide | Intermediate 26; Amine No. 17 | ¹H-NMR (400 MHz, DMSO- d6, sel. signals): δ = 1.09 (d, 3H); 1.63 (bd, 1H); 1.79 (dq, 1H); 1.87-2.05 (m, 2H); 2.67 (s, 3H); 3.21 (s, 3H); 3.77 (t, 2H); 3.39 (s, 3H); 3.94-4.11 (m, 4H); 4.26 (q, 1H); 4.40 (tt, 1H); 4.53-4.67 (m, 1H); 6.63 (d, 1H); 7.29 (d, 1H); 7.41- 7.50 (m, 2H); 8.19 (s, 1H); 8.46 (d, 1H); 8.74 (d, 1H). |
| 56 | 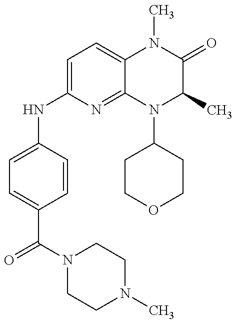 [SMILES: C[C@@H]1C(=O)N(C)c2ccc(Nc3ccc(C(=O)N4CCN(C)CC4)cc3)nc2N1C1CCOCC1] | (3R)-1,3-Dimethyl-6-({4- [(4-methylpiperazin-1- yl)carbonyl]phenyl}amino)- 4-(tetrahydro-2H-pyran-4- yl)-3,4-dihydropyrido[2,3- b]pyrazin-2(1H)-one | Intermediate 32; Amine No. 20 | UPLC-MS: Rt = 0.69 min (M⁺ + 1 = 479) |
| 57 | 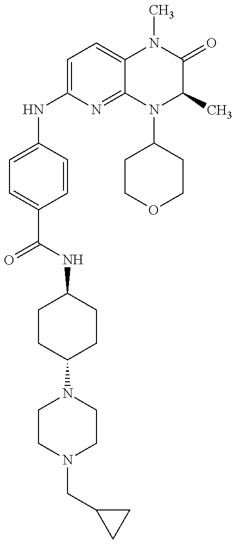 [SMILES: C[C@@H]1C(=O)N(C)c2ccc(Nc3ccc(C(=O)N[C@H]4CC[C@H](N5CCN(CC6CC6)CC5)CC4)cc3)nc2N1C1CCOCC1] | N-{trans-4-[4- (Cyclopropylmethyl)piper- azin-1-yl]cyclohexyl}-4- {[(3R)-1,3-dimethyl-2-oxo- 4-(tetrahydro-2H-pyran-4- yl)-1,2,3,4- tetrahydropyrido[2,3- b]pyrazin-6- yl]amino}benzamide | Intermediate 32; Amine No. 7 | UPLC-MS: Rt = 0.71 min (M⁺ + 1 = 616) |
| 58 | 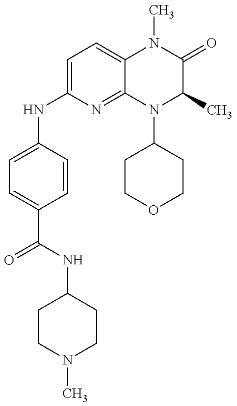 [SMILES: C[C@@H]1C(=O)N(C)c2ccc(Nc3ccc(C(=O)NC4CCN(C)CC4)cc3)nc2N1C1CCOCC1] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-N-(1- methylpiperidin-4- yl)benzamide | Intermediate 32; Amine No. 1 | UPLC-MS: Rt = 0.73 min (M⁺ + 1 = 493) |
| 59 | 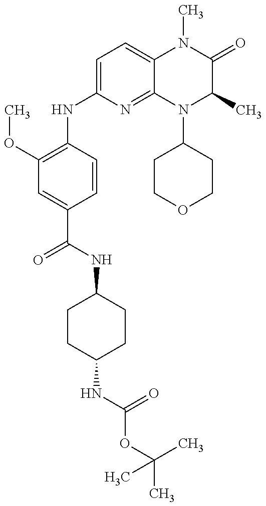 [SMILES: COc1cc(C(=O)N[C@H]2CC[C@H](NC(=O)OC(C)(C)C)CC2)ccc1Nc1ccc2c(n1)N(C1CCOCC1)[C@H](C)C(=O)N2C] | tert-Butyl {trans-4-[(4- {[(3R)-1,3-dimethyl-2-oxo- 4-(tetrahydro-2H-pyran-4- yl)-1,2,3,4- tetrahydropyrido[2,3- b]pyrazin-6-yl]amino}-3- methoxybenzoyl)amino] cyclohexyl}carbamate | Intermediate 26; Amine No. 28 | UPLC-MS: Rt = 1.23 min (M⁺ + 1 = 623) |
| 60 | 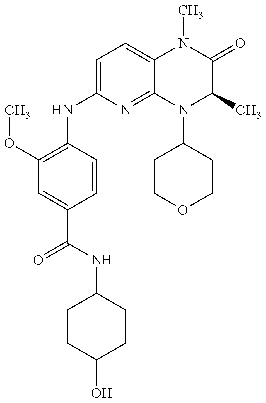 [SMILES: COc1cc(C(=O)NC2CCC(O)CC2)ccc1Nc1ccc2c(n1)N(C1CCOCC1)[C@H](C)C(=O)N2C] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-N-(4- hydroxycyclohexyl)-3- methoxybenzamide | Intermediate 26; Amine No. 23 | UPLC-MS: Rt = 0.98 min (M⁺ + 1 = 524) |
| 61 | 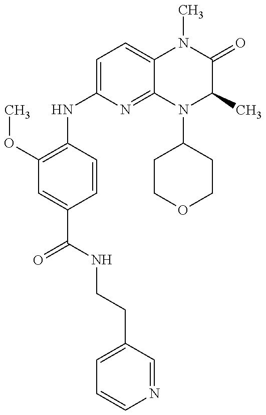 [SMILES: COc1cc(C(=O)NCCc2cccnc2)ccc1Nc1ccc2c(n1)N(C1CCOCC1)[C@H](C)C(=O)N2C] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-3- methoxy-N-[2-(pyridin-3- yl)ethyl]benzamide | Intermediate 26; Amine No. 25 | ¹H-NMR (400 MHz, DMSO- d6, sel. signals): δ = 1.09 (d, 3H); 1.63 (bd, 1H); 1.78 (dq, 1H); 1.86-2.05 (m, 2H); 2.91 (t, 2H); 3.21 (s, 3H); 3.91 (s, 3H); 4.01 (dt, 2H); 4.25 (q, 1H); 4.40 (tt, 1H); 6.61 (d, 1H); 7.28 (d, 1H); 7.37-7.48 (m, 3H); 7.70 (dt, 1H); 8.13 (s, 1H); 8.40-8.46 (m, 2H); 8.47 (dd, 1H); 8.52 (d, 1H). |
| 62 | 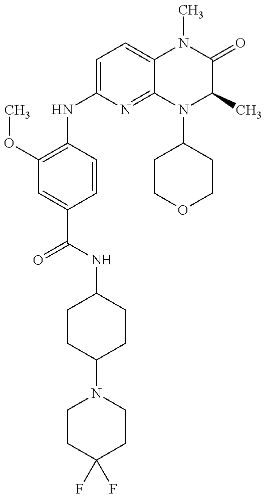 [SMILES: COc1cc(C(=O)NC2CCC(N3CCC(F)(F)CC3)CC2)ccc1Nc1ccc2c(n1)N(C1CCOCC1)[C@H](C)C(=O)N2C] | N-[4-(4,4-Difluoropiperidin- 1-yl)cyclohexyl]-4-{[(3R)- 1,3-dimethyl-2-oxo-4- (tetrahydro-2H-pyran-4-yl)- 1,2,3,4- tetrahydropyrido[2,3- b]pyrazin-6-yl]amino}-3- methoxybenzamide | Intermediate 26, Intermediate 68 | UPLC-MS: Rt = 0.86 min (M⁺ + 1 = 627) |
| 63 | 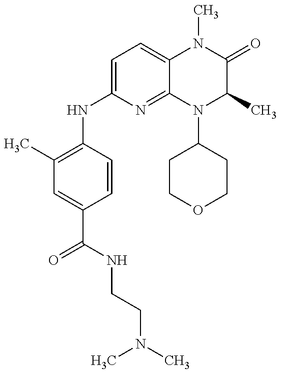 [SMILES: Cc1cc(C(=O)NCCN(C)C)ccc1Nc1ccc2c(n1)N(C1CCOCC1)[C@H](C)C(=O)N2C] | N-[2- (Dimethylamino)ethyl]-4- {[(3R)-1,3-dimethyl-2-oxo- 4-(tetrahydro-2H-pyran-4- yl)-1,2,3,4- tetrahydropyrido[2,3- b]pyrazin-6-yl]amino}-3- methylbenzamide | Intermediate 34; Amine No. 4 | ¹H-NMR (400 MHz, DMSO- d6, sel. signals): δ = 1.09 (d, 3H); 1.58 (bd, 1H); 1.74 (dq, 1H); 1.84-1.07 (m, 2H); 2.31 (s, 3H); 2.80 (t, 2H); 3.21 (s, 3H); 3.33 (dt, 2H); 3.37-3.50 (m, 4H); 3.92-4.00 (m, 2H); 4.23 (q, 1H); 4.34 (tt, 1H); 6.48 (d, 1H); 7.28 (d, 1H); 7.62 (dd, 1H); 7.67 (d, 1H); 7.91 (s, 1H); 8.11 (d, 1H); 8.29 (t, 1H). |
| 64 | 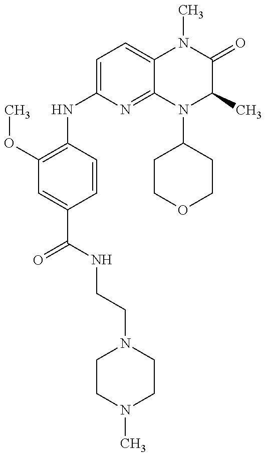 [SMILES: COc1cc(C(=O)NCCN2CCN(C)CC2)ccc1Nc1ccc2c(n1)N(C1CCOCC1)[C@H](C)C(=O)N2C] | 4-{[(3R)-1,3-Dimethyl-2- oxo-4-(tetrahydro-2H-pyran- 4-yl)-1,2,3,4- tetrahydropyrido[2,3- b]pyrazin-6-yl]amino}-3- methoxy-N-[2-(4- methylpiperazin-1- yl)ethyl]benzamide | Intermediate 26; Amine No. 26 | ¹H NMR (400 MHz, DMSO- d6): δ = 1.09 (d, 1H); 1.63 (bd, 1H); 1.78 (dq, 1H); 1.94 (dq, 1H); 2.00 (bd, 1H); 2.17 (s, 3H); 2.28-2.42 (m, 4H); 2.46 (t, 2H); 3.21 (s, 3H); 3.37 (q, 2H); 3.48 (dt, 2H); 3.92 (s, 3H); 3.96-4.07 (m, 2H); 4.25 (q, 1H); 4.40 (tt, 1H); 6.61 (d, 1H); 7.28 (d, 1H); 7.43 (dd, 1H); 7.45 (d, 1H); 8.10 (s, 1H); 8.22 (t, 1H); 8.44 (d, 1H). |
| 65 | 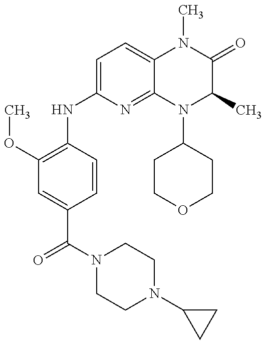 [SMILES: COc1cc(C(=O)N2CCN(C3CC3)CC2)ccc1Nc1ccc2c(n1)N(C1CCOCC1)[C@H](C)C(=O)N2C] | (3R)-6-({4-[(4- Cyclopropylpiperazin-1- yl)carbonyl]-2- methoxyphenyl}amino)-1,3- dimethyl-4-(tetrahydro-2H- pyran-4-yl)-3,4- dihydropyrido[2,3- b]pyrazin-2(1H)-one | Intermediate 26; Amine No. 29 | UPLC-MS: Rt = 0.82 min (M⁺ + 1 = 535) |
| 66 | 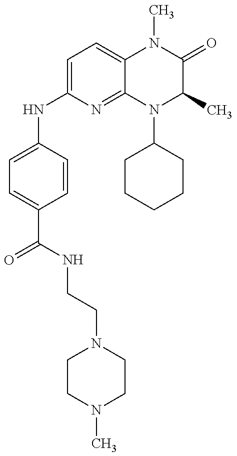 [SMILES: C[C@@H]1C(=O)N(C)c2ccc(Nc3ccc(C(=O)NCCN4CCN(C)CC4)cc3)nc2N1C1CCCCC1] | 4-{[(3R)-4-Cyclohexyl-1,3- dimethyl-2-oxo-1,2,3,4- tetrahydropyrido[2,3- b]pyrazin-6-yl]amino}-N-[2- (4-methylpiperazin-1- yl)ethyl]benzamide | Intermediate 30; Amine No. 26 | ¹H NMR (400 MHz, DMSO- d6): δ = 1.09 (d, 3H); 1.23 (t, 1H); 1.34-1.58 (m, 3H); 1.58- 1.79 (m, 3H); 1.79-1.97 (m, 2H); 2.05-2.17 (m, 1H); 2.13 (s, 3H); 2.19-2.47 (m, 9H); 3.20 (s, 3H); 4.13-4.31 (m, 2H); 6.26 (d, 1H); 7.26 (d, 1H); 7.65-7.79 (m, 4H); 8.13 (t, 1H); 9.14 (s, 1H). |
| 67 | 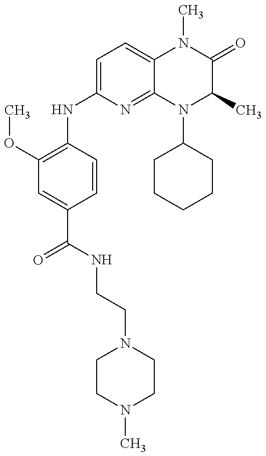 [SMILES: COc1cc(C(=O)NCCN2CCN(C)CC2)ccc1Nc1ccc2c(n1)N(C1CCCCC1)[C@H](C)C(=O)N2C] | 4-{[(3R)-4-Cyclohexyl-1,3- dimethyl-2-oxo-1,2,3,4- tetrahydropyrido[2,3- b]pyrazin-6-yl]amino}-3- methoxy-N-[2-(4- methylpiperazin-1- yl)ethyl]benzamide | Intermediate 12; Amine No. 26 | ¹H NMR (400 MHz, DMSO- d6): δ = 1.08 (d, 3H); 1.23 (t, 1H); 1.31-1.56 (m, 3H); 1.57- 1.77 (m, 3H); 1.77-1.96 (m, 2H); 2.13 (d, 1H); 2.14 (s, 3H); 2.22-2.48 (m, 9H); 3.20 (s, 3H); 3.92 (s, 3H); 4.12- 4.31 (m, 2H); 6.58 (d, 1H); 7.26 (d, 1H); 7.39 (d, 1H); 7.45 (d, 1H); 8.10 (s, 1H); 8.22 (t, 1H); 8.52 (d, 1H). |
| 68 | 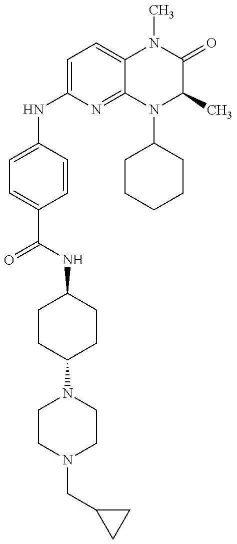 [SMILES: C[C@@H]1C(=O)N(C)c2ccc(Nc3ccc(C(=O)N[C@H]4CC[C@H](N5CCN(CC6CC6)CC5)CC4)cc3)nc2N1C1CCCCC1] | 4-{[(3R)-4-Cyclohexyl-1,3- dimethyl-2-oxo-1,2,3,4- tetrahydropyrido[2,3- b]pyrazin-6-yl]amino}-N- {trans-4-[4- (cyclopropylmethyl) piperazin-1- yl]cyclohexyl}benzamide | Intermediate 30; Amine No. 7 | ¹H NMR (400 MHz, CDCl₃): δ = 0.06-0.21 (m, 2H); 0.48- 0.61 (m, 2H); 0.83-0.98 (m, 1H); 1.11-1.35 (m, 5H); 1.23 (d, 3H); 1.35-1.69 (m, 7H); 1.69-1.82 (m, 2H); 1.82- 2.08 (m, 5H); 2.08-2.43 (m, 9H); 3.30 (s, 3H); 3.84-4.03 (m, 1H); 4.24-4.42 (m, 2H); 5.85 (d, 1H); 6.23 (d, 1H); 6.55 (s, 1H); 7.02 (d, 1H); 7.50 (d, 2H); 7.69 (d, 2H). |
| 69 | 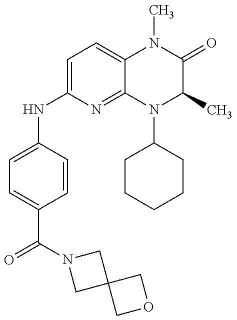 [SMILES: C[C@@H]1C(=O)N(C)c2ccc(Nc3ccc(C(=O)N4CC5(COC5)C4)cc3)nc2N1C1CCCCC1] | (3R)-4-Cyclohexyl-1,3- dimethyl-6-{[4-(2-oxa-6- azaspiro[3.3]hept-6- ylcarbonyl)phenyl]amino}- 3,4-dihydropyrido[2,3- b]pyrazin-2(1H)-one | Intermediate 30; Amine No. 18 | ¹H NMR (400 MHz, DMSO- d6): δ = 1.09 (d, 3H); 1.23 (t, 1H); 1.30-1.57 (m, 3H); 1.58- 1.76 (m, 3H); 1.77-1.97 (m, 2H); 2.12 (d, 1H); 3.20 (s, 3H); 4.09-4.33 (m, 4H); 4.38- 4.59 (m, 2H); 4.68 (s, 4H); 6.26 (d, 1H); 7.26 (d, 1H); 7.50 (d, 2H); 7.72 (d, 2H); 9.18 (s, 1H). |
| 70 | 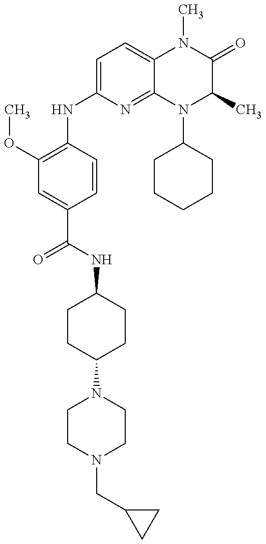 [SMILES: COc1cc(C(=O)N[C@H]2CC[C@H](N3CCN(CC4CC4)CC3)CC2)ccc1Nc1ccc2c(n1)N(C1CCCCC1)[C@H](C)C(=O)N2C] | 4-{[(3R)-4-Cyclohexyl-1,3- dimethyl-2-oxo-1,2,3,4- tetrahydropyrido[2,3- b]pyrazin-6-yl]amino}-N- {trans-4-[4- (cyclopropylmethyl) piperazin-1-yl]cyclohexyl}- 3-methoxybenzamide | Intermediate 12; Amine No. 7 | ¹H NMR (400 MHz, CDCl₃): δ = 0.12-0.24 (m, 2H); 0.51- 0.61 (m, 2H); 0.87-1.02 (m, 1H); 1.14-1.40 (m, 5H); 1.23 (d, 3H); 1.40-1.71 (m, 7H); 1.71-1.84 (m, 2H); 1.84- 2.11 (m, 5H); 2.15-2.30 (m, 3H); 2.30-2.49 (m, 3H); 3.31 (s, 3H); 3.86-4.04 (m, 1H); 3.99 (s, 3H); 4.25-4.44 (m, 2H); 5.85 (d, 1H); 6.22 (d, 1H); 7.03 (d, 1H); 7.10 (s, 1H); 7.20 (dd, 1H); 7.41 (d, 1H); 8.40 (d, 1H). |
| 71 | 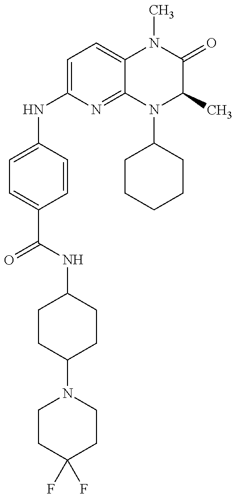 [SMILES: C[C@@H]1C(=O)N(C)c2ccc(Nc3ccc(C(=O)NC4CCC(N5CCC(F)(F)CC5)CC4)cc3)nc2N1C1CCCCC1] | 4-{[(3R)-4-Cyclohexyl-1,3- dimethyl-2-oxo-1,2,3,4- tetrahydropyrido[2,3- b]pyrazin-6-l]amino}-N-[4- (4,4-difluoropiperidin-1- yl)cyclohexyl]benzamide | Intermediate 30; Intermedate 68 | ¹H NMR (400 MHz, DMSO- d6): δ = 1.09 (d, 3H); 1.18- 1.30 (m, 1H); 1.31-1.75 (m, 12H); 1.75-2.03 (m, 10H); 3.20 (s, 4H); 3.92-4.08 (m, 1H); 4.14-4.31 (m, 2H); 6.27 (d, 1H); 7.26 (d, 1H); 7.66- 7.86 (m, 5H); 9.13 (s, 1H). |
| 72 | 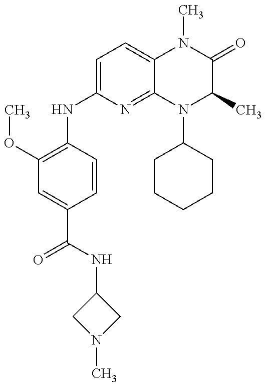 [SMILES: COc1cc(C(=O)NC2CN(C)C2)ccc1Nc1ccc2c(n1)N(C1CCCCC1)[C@H](C)C(=O)N2C] | 4-{[(3R)-4-Cyclohexyl-1,3- dimethyl-2-oxo-1,2,3,4- tetrahydropyrido[2,3- b]pyrazin-6-yl]amino}-3- methoxy-N-(1- methylazetidin-3- yl)benzamide | Intermediate 12; Amine No. 17 | ¹H NMR (400 MHz, DMSO- d6): δ = 1.08 (d, 3H); 1.23 (t, 1H); 1.31-1.57 (m, 3H); 1.58- 1.76 (m, 3H); 1.87 (t, 2H); 2.09 (d, 1H); 2.26 (s, 3H); 2.98 (t, 2H); 3.20 (s, 3H); 3.55 (t, 2H); 3.94 (s, 3H); 4.13- 4.30 (m, 2H); 4.41 (q, 1H); 6.59 (d, 1H); 7.26 (d, 1H); 7.43 (d, 1H); 7.48 (s, 1H); 8.11 (s, 1H); 8.50 (d, 1H); 8.56 (d, 1H). |
| 73 | 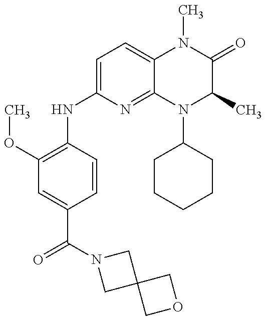 [SMILES: COc1cc(C(=O)N2CC3(COC3)C2)ccc1Nc1ccc2c(n1)N(C1CCCCC1)[C@H](C)C(=O)N2C] | (3R)-4-Cyclohexyl-6-{[2- methoxy-4-(2-oxa-6- azaspiro[3.3]hept-6- ylcarbonyl)phenyl]amino}- 1,3-dimethyl-3,4- dihydropyrido[2,3- b]pyrazin-2(1H)-one | Intermediate 12; Amine No. 18 | ¹H NMR (400 MHz, DMSO- d6): δ = 1.09 (d, 3H); 1.22 (t, 1H); 1.30-1.55 (m, 3H); 1.56- 1.76 (m, 3H); 1.86 (t, 2H); 2.09 (d, 1H); 3.20 (s, 3H); 3.91 (s, 3H); 4.09-4.29 (m, 4H); 4.46-4.57 (m, 2H); 4.69 (s, 4H); 6.58 (d, 1H); 7.13 (dd, 1H); 7.17 (d, 1H); 7.26 (d, 1H); 8.15 (s, 1H); 8.49 (d, 1H). |
| 74 | 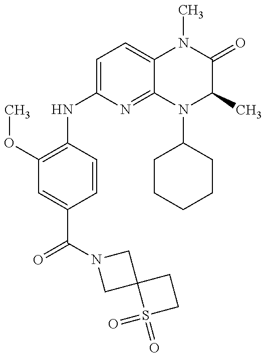 [SMILES: COc1cc(C(=O)N2CC3(CCS3(=O)=O)C2)ccc1Nc1ccc2c(n1)N(C1CCCCC1)[C@H](C)C(=O)N2C] | (3R)-4-Cyclohexyl-6-({4- [(1,1-dioxido-1-thia-6- azaspiro[3.3]hept-6- yl)carbonyl]-2- methoxyphenyl}amino)-1,3- dimethyl-3,4- dihydropyrido[2,3- b]pyrazin-2(1H)-one | Intermediate 12; Amine No. 6 | ¹H NMR (400 MHz, DMSO- d6): δ = 1.08 (d, 3H); 1.21 (t, 1H); 1.29-1.54 (m, 3H); 1.57- 1.76 (m, 3H); 1.85 (t, 2H); 2.09 (d, 1H); 2.37-2.48 (m, 2H); 3.20 (s, 3H); 3.92 (s, 3H); 4.05-4.28 (m, 4H); 4.28- 4.80 (m, 4H); 6.60 (d, 1H); 7.16 (d, 1H); 7.19 (s, 1H); 7.27 (d, 1H); 8.20 (s, 1H); 8.51 (d, 1H). |
| 75 | 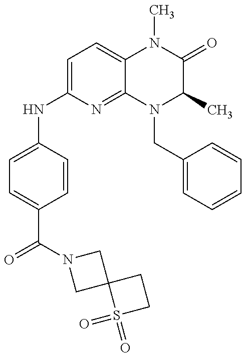 [SMILES: C[C@@H]1C(=O)N(C)c2ccc(Nc3ccc(C(=O)N4CC5(CCS5(=O)=O)C4)cc3)nc2N1Cc1ccccc1] | (3R)-4-Benzyl-6-({4-[(1,1- dioxido-1-thia-6- azaspiro[3.3]hept-6- yl)carbonyl]phenyl}amino)- 1,3-dimethyl-3,4- dihydropyrido[2,3- b]pyrazin-2(1H)-one | Intermediate 47; Amine No. 6 | ¹H NMR (400 MHz, DMSO- d6): δ = 1.16 (d, 3H); 2.41 (t, 2H); 3.25 (s, 3H); 4.05-4.17 (m, 3H); 4.26-4.69 (m, 4H); 4.37 (d, 1H); 5.14 (d, 1H); 6.30 (d, 1H); 7.20-7.43 (m, 8H); 7.47 (d, 2H); 9.14 (s, 1H). |
| 76 | 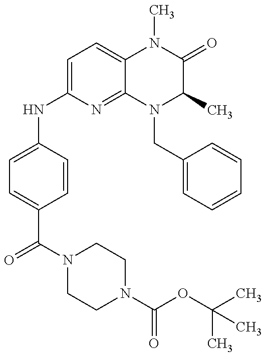 [SMILES: C[C@@H]1C(=O)N(C)c2ccc(Nc3ccc(C(=O)N4CCN(C(=O)OC(C)(C)C)CC4)cc3)nc2N1Cc1ccccc1] | tert-Butyl 4-(4-{[(3R)-4- benzyl-1,3-dimethyl-2-oxo- 1,2,3,4- tetrahydropyrido[2,3- b]pyrazin-6-yl]amino} benzoyl)piperazine-1- carboxylate | Intermediate 47; Amine No. 30 | ¹H NMR (400 MHz, DMSO- d6): δ = 1.15 (d, 3H); 1.41 (s, 9H); 2.41 (t, 2H); 3.25 (s, 3H); 3.32-3.40 (m, 4H); 3.40- 3.51 (m, 4H); 4.08 (q, 1H); 4.35 (d, 1H); 5.15 (d, 1H); 6.29 (d, 1H); 7.18 (d, 2H); 7.23-7.40 (m, 6H); 7.46 (d, 2H); 9.01 (s, 1H). |
| 77 | 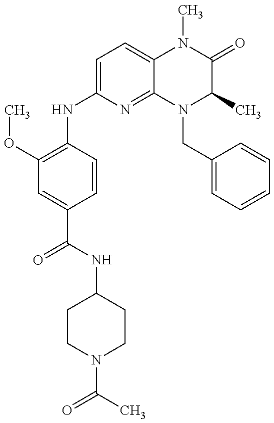 [SMILES: COc1cc(C(=O)NC2CCN(C(C)=O)CC2)ccc1Nc1ccc2c(n1)N(Cc1ccccc1)[C@H](C)C(=O)N2C] | N-(1-Acetylpiperidin-4-yl)- 4-{[(3R)-4-benzyl-1,3- dimethyl-2-oxo-1,2,3,4- tetrahydropyrido[2,3- b]pyrazin-6-yl]amino}-3- methoxybenzamide | Intermediate 45; Amine No. 22 | ¹H NMR (400 MHz, DMSO- d6): δ = 1.12 (d, 3H); 1.26- 1.54 (m, 2H); 1.80 (t, 2H); 2.00 (s, 3H); 2.64 (t, 1H); 3.11 (t, 1H); 3.24 (s, 3H); 3.82 (d, 1H); 3.90 (s, 3H); 3.93-4.08 (m, 2H); 4.28 (d, 1H); 4.34 (d, 1H); 5.21 (d, 1H); 6.61 (d, 1H); 7.24-7.43 (m, 6H); 8.02 (d, 1H); 8.09 (s, 1H); 8.18 (d, 1H). |
| 78 | 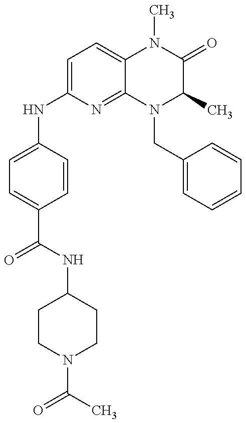 [SMILES: CC(=O)N1CCC(NC(=O)c2ccc(Nc3ccc4c(n3)N(Cc3ccccc3)[C@H](C)C(=O)N4C)cc2)CC1] | N-(1-Acetylpiperidin-4-yl)- 4-{[(3R)-4-benzyl-1,3- dimethyl-2-oxo-1,2,3,4- tetrahydropyrido[2,3- b]pyrazin-6- yl]amino}benzamide | Intermediate 47; Amine No. 22 | ¹H NMR (400 MHz, DMSO- d6): δ = 1.13 (d, 3H); 1.24- 1.52 (m, 2H); 1.79 (t, 2H); 2.00 (s, 3H); 2.65 (t, 1H); 3.11 (t, 1H); 3.24 (s, 3H); 3.81 (d, 1H); 3.91-4.06 (m, 2H); 4.24- 4.39 (m, 2H); 5.23 (d, 1H); 6.31 (d, 1H); 7.23-7.43 (m, 6H); 7.54 (d, 2H); 7.67 (d, 2H); 7.95 (d, 1H); 9.09 (s, 1H). |
| 79 | 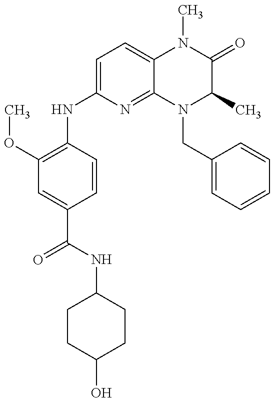 [SMILES: COc1cc(C(=O)NC2CCC(O)CC2)ccc1Nc1ccc2c(n1)N(Cc1ccccc1)[C@H](C)C(=O)N2C] | 4-{[(3R)-4-Benzyl-1,3- dimethyl-2-oxo-1,2,3,4- tetrahydropyrido[2,3- b]pyrazin-6-yl]amino}-N-(4- hydroxycyclohexyl)-3- methoxybenzamide | Intermediate 45; Amine No. 23 | ¹H NMR (400 MHz, DMSO- d6): δ = 1.12 (d, 3H); 1.16- 1.44 (m, 4H); 1.81 (t, 4H); 3.23 (s, 3H); 3.62-3.78 (m, 1H); 3.90 (s, 3H); 3.97 (q, 1H); 4.27 (d, 1H); 4.55 (d, 1H); 5.21 (d, 1H); 6.60 (d, 1H); 7.24-7.42 (m, 8H); 7.90 (d, 1H); 8.07 (s, 1H); 8.16 (d, 1H). |
| 80 | 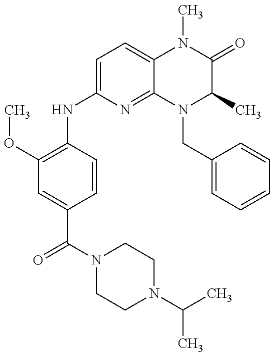 [SMILES: COc1cc(C(=O)N2CCN(C(C)C)CC2)ccc1Nc1ccc2c(n1)N(Cc1ccccc1)[C@H](C)C(=O)N2C] | (3R)-4-Benzyl-6-[(2- methoxy-4-{[4-(propan-2- yl)piperazin-1- yl]carbonyl}phenyl)amino]- 1,3-dimethyl-3,4-dihydro pyrido[2,3-b]pyrazin-2(1H)- one | Intermediate 45; Amine No. 16 | ¹H NMR( 400 MHz, CDCl₃): δ = 1.25 (d, 3H); 1.31 (d, 6H); 2.79-3.04 (m, 4H); 3.18- 3.31 (m, 1H); 3.34 (s, 3H); 3.93 (s, 3H); 3.95-4.05 (m, 4H); 4.11 (q, 1H); 4.22 (d, 1H); 5.40 (d, 1H); 6.29 (d, 1H); 6.88 (d, 1H); 7.02 (d, 1H); 7.08 (d, 1H); 7.29-7.44 (m, 6H); 8.08 (d, 1H). |
| 81 | 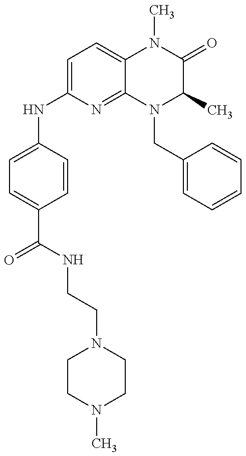 [SMILES: C[C@@H]1C(=O)N(C)c2ccc(Nc3ccc(C(=O)NCCN4CCN(C)CC4)cc3)nc2N1Cc1ccccc1] | 4-{[(3R)-4-Benzyl-1,3- dimethyl-2-oxo-1,2,3,4- tetrahydropyrido[2,3- b]pyrazin-6-yl]amino}-N-[2- (4-methylpiperazin-1- yl)ethyl]benzamide | Intermediate 47; Amine No. 26 | ¹H NMR (400 MHz, DMSO- d6): δ = 1.14 (d, 3H); 2.13 (s, 3H); 2.20-2.46 (m, 10H); 3.24 (s, 3H); 4.03 (q, 1H); 4.33 (d, 1H); 5.21 (d, 1H); 6.30 (d, 1H); 7.23-7.41 (m, 6H); 7.51 (d, 2H); 7.62 (d, 2H); 8.03 (t, 1H); 9.07 (s, 1H). |
| 82 | 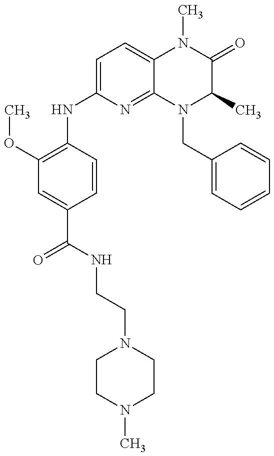 [SMILES: COc1cc(C(=O)NCCN2CCN(C)CC2)ccc1Nc1ccc2c(n1)N(Cc1ccccc1)[C@H](C)C(=O)N2C] | 4-{[(3R)-4-Benzyl-1,3- dimethyl-2-oxo-1,2,3,4- tetrahydropyrido[2,3- b]pyrazin-6-yl]amino}-3- methoxy-N-[2-(4- methylpiperazin-1- yl)ethyl]benzamide | Intermediate 45; Amine No. 26 | ¹H NMR (400 MHz, DMSO- d6): δ = 1.13 (d, 3H); 2.13 (s, 3H); 2.20-2.37 (m, 4H); 2.37- 2.47 (m, 5H); 3.24 (s, 3H); 3.89 (s, 3H); 4.02 (q, 1H); 4.30 (d, 1H); 5.19 (d, 1H); 6.59 (d, 1H); 7.24-7.31 (m, 3H); 7.31-7.39 (m, 4H); 7.40 (d, 1H); 8.05 (s, 1H); 8.09- 8.15 (m, 2H). |
| 83 | 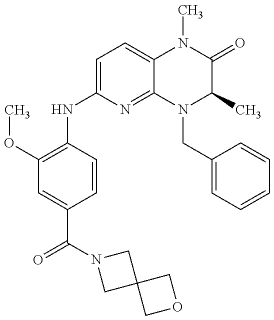 [SMILES: COc1cc(C(=O)N2CC3(COC3)C2)ccc1Nc1ccc2c(n1)N(Cc1ccccc1)[C@H](C)C(=O)N2C] | (3R)-4-Benzyl-6-{[2- methoxy-4-(2-oxa-6- azaspiro[3.3]hept-6- ylcarbonyl)phenyl]amino}- 1,3-dimethyl-3,4- dihydropyrido[2,3- b]pyrazin-2(1H)-one | Intermediate 45; Amine No. 18 | ¹H NMR (400 MHz, DMSO- d6): δ = 1.14 (d, 3H); 3.25 (s, 3H); 3.87 (s, 3H); 4.08 (q, 1H); 4.13-4.26 (m, 2H); 4.33 (d, 1H); 4.40-4.56 (m, 2H); 4.69 (s, 4H); 5.12 (d, 1H); 6.60 (d, 1H); 6.93 (dd, 1H); 7.14 (d, 1H); 7.23-7.32 (m, 2H); 7.32-7.38 (m, 4H); 8.01 (d, 1H); 8.09 (s, 1H). |
| 84 | 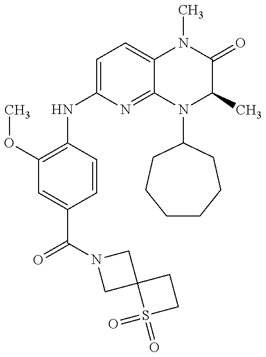 [SMILES: COc1cc(C(=O)N2CC3(CCS3(=O)=O)C2)ccc1Nc1ccc2c(n1)N(C1CCCCCC1)[C@H](C)C(=O)N2C] | (3R)-4-Cycloheptyl-6-({4- [(1,1-dioxido-1-thia-6- azaspiro[3.3]hept-6- yl)carbonyl]-2- methoxyphenyl}amino)-1,3- dimethyl-3,4- dihydropyrido[2,3- b]pyrazin-2(1H)-one | Intermediate 39; Amine No. 6 | ¹H NMR (400 MHz, DMSO- d6): δ = 1.09 (d, 3H); 1.33- 1.80 (m, 10H); 1.80-1.97 (m, 1H); 1.98-2.10 (m, 1H); 2.43 (t, 2H); 3.20 (s, 3H); 3.92 (s, 3H); 4.11 (t, 2H); 4.17-4.34 (m, 2H); 4.34-4.80 (m, 4H); 6.58 (d, 1H); 7.14 (dd, 1H); 7.20 (d, 1H); 7.26 (d, 1H); 8.19 (s, 1H); 8.46 (d, 1H). |
| 85 | 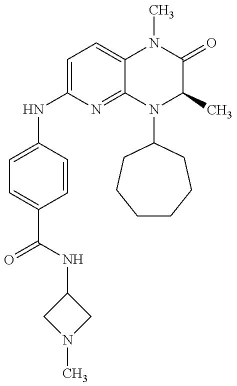 [SMILES: C[C@@H]1C(=O)N(C)c2ccc(Nc3ccc(C(=O)NC4CN(C)C4)cc3)nc2N1C1CCCCCC1] | 4-{[(3R)-4-Cycloheptyl-1,3- dimethyl-2-oxo-1,2,3,4- tetrahydropyrido[2,3- b]pyrazin-6-yl]amino}-N-(1- methylazetidin-3- yl)benzamide | Intermediate 40; Amine No. 17 | ¹H NMR (400 MHz, DMSO- d6): δ = 1.09 (d, 3H); 1.38- 1.83 (m, 10H); 1.83-1.98 (m, 1H); 2.01-2.15 (m, 1H); 2.25 (s, 3H); 2.95 (t, 2H); 3.20 (s, 3H); 3.54 (t, 2H); 4.24 (q, 1H); 4.28-4.46 (m, 2H); 6.26 (d, 1H); 7.26 (d, 1H); 7.69 (d, 2H); 7.74 (d, 2H); 8.49 (d, 1H); 9.13 (s, 1H). |
| 86 | 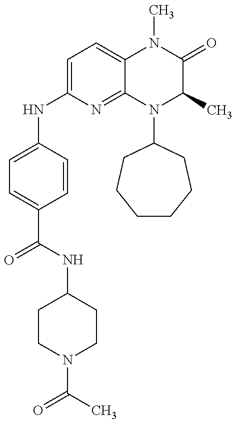 [SMILES: CC(=O)N1CCC(NC(=O)c2ccc(Nc3ccc4c(n3)N(C3CCCCCC3)[C@H](C)C(=O)N4C)cc2)CC1] | N-(1-Acetylpiperidin-4-yl)- 4-{[(3R)-4-cycloheptyl-1,3- dimethyl-2-oxo-1,2,3,4- tetrahydropyrido[2,3- b]pyrazin-6- yl]amino}benzamide | Intermediate 40; Amine No. 22 | ¹H NMR (400 MHz, DMSO- d6): δ = 1.09 (d, 3H); 1.27- 1.96 (m, 15H); 2.01 (s, 3H); 2.03-2.14 (m, 1H); 2.65 (t, 1H); 3.12 (t, 1H); 3.20 (s, 3H); 3.82 (d, 1H); 3.90-4.08 (m, 1H); 4.24 (q, 1H); 4.34 (d, 2H); 6.26 (d, 1H); 7.26 (d, 1H); 7.68 (d, 2H); 7.73 (d, 2H); 8.02 (d, 1H); 9.11 (s, 1H). |
| 87 | 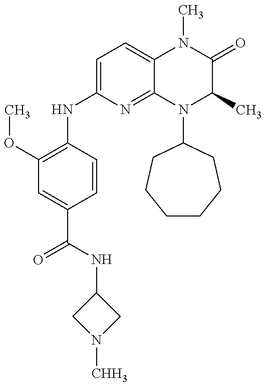 | 4-{[(3R)-4-Cycloheptyl-1,3- dimethyl-2-oxo-1,2,3,4- tetrahydropyrido[2,3- b]pyrazin-6-yl]amino}-3- methoxy-N-(1- methylazetidin-3- yl)benzamide | Intermediate 39; Amine No. 17 | ¹H NMR (400 MHz, DMSO- d6): δ = 1.09 (d, 3H); 1.39- 1.82 (m, 10H); 1.82-1.97 (m, 1H); 1.98-2.12 (m, 1H); 2.26 (s, 3H); 2.97 (t, 2H); 3.20 (s, 3H); 3.55 (t, 2H); 3.93 (s, 3H); 4.17-4.35 (m, 2H); 4.41 (q, 1H); 6.57 (d, 1H); 7.26 (d, 1H); 7.42 (dd, 1H); 7.47 (d, 1H); 8.08 (s, 1H); 8.44 (d, 1H); 8.55 (d, 1H). |
| 88 | 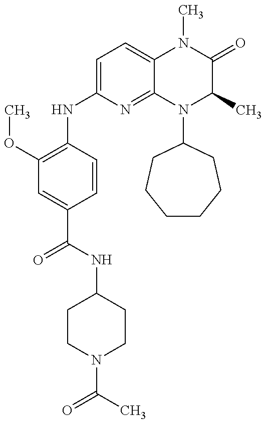 [SMILES: COc1cc(C(=O)NC2CCN(C(C)=O)CC2)ccc1Nc1ccc2c(n1)N(C1CCCCCC1)[C@H](C)C(=O)N2C] | N-(1-Acetylpiperidin-4-yl)- 4-{[(3R)-4-cycloheptyl-1,3- dimethyl-2-oxo-1,2,3,4- tetrahydropyrido[2,3- b]pyrazin-6-yl]amino}-3- methoxybenzamide | Intermediate 39; Amine No. 22 | ¹H NMR (400 MHz, DMSO- d6): δ = 1.09 (d, 3H); 1.29- 1.93 (m, 15H); 2.02 (s, 3H); 2.03-2.12 (m, 1H); 2.64 (t, 1H); 3.12 (t, 1H); 3.20 (s, 3H); 3.84 (d, 1H); 3.93 (s, 3H); 3.96-4.11 (m, 1H); 4.24 (q, 1H); 4.27-4.44 (m, 2H); 6.56 (d, 1H); 7.25 (d, 1H); 7.41 (d, 1H); 7.45 (s, 1H); 8.03-8.10 (m, 2H); 8.43 (d, 1H). |
| 89 | 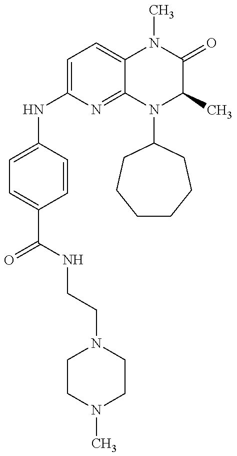 [SMILES: C[C@@H]1C(=O)N(C)c2ccc(Nc3ccc(C(=O)NCCN4CCN(C)CC4)cc3)nc2N1C1CCCCCC1] | 4-{[(3R)-4-Cycloheptyl-1,3- dimethyl-2-oxo-1,2,3,4- tetrahydropyrido[2,3- b]pyrazin-6-yl]amino}-N-{2- (4-methylpiperazin-1- yl)ethyl]benzamide | Intermediate 40; Amine No. 26 | ¹H NMR (400 MHz, DMSO- d6): δ = 1.09 (d, 3H); 1.39- 1.99 (m, 12H); 2.00-2.11 (m, 1H); 2.14 (s, 3H); 2.19-2.37 (m, 4H); 2.37-2.46 (m, 5H); 3.20 (s, 3H); 4.24 (q, 1H); 4.32 (t, 1H); 6.26 (d, 1H); 7.26 (d, 1H); 7.70 (s, 4H); 8.11 (t, 1H); 9.12 (s, 1H). |
| 90 | 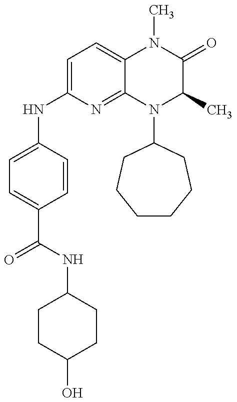 [SMILES: C[C@@H]1C(=O)N(C)c2ccc(Nc3ccc(C(=O)NC4CCC(O)CC4)cc3)nc2N1C1CCCCCC1] | 4-{[(3R)-4-Cycloheptyl-1,3- dimethyl-2-oxo-1,2,3,4- tetrahydropyrido[2,3- b]pyrazin-6-yl]amino}-N-(4- hydroxycyclohexyl)benz- amide | Intermediate 40; Amine No. 23 | ¹H NMR (400 MHz, DMSO- d6): δ = 1.09 (d, 3H); 1.14- 1.45 (m, 4H); 1.45-1.95 (m, 14H); 2.01-2.13 (m, 1H); 3.19 (s, 3H); 3.36-3.45 (m, 1H); 3.60-3.78 (m, 1H); 4.42 (q, 1H); 4.32 (t, 2H); 4.55 (d, 1H); 6.26 (d, 1H); 7.25 (d, 1H); 7.67 (d, 2H); 7.72 (d, 2H); 7.90 (d, 1H); 9.09 (s, 1H). |
| 91 | 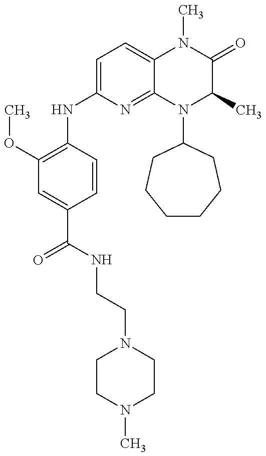 [SMILES: COc1cc(C(=O)NCCN2CCN(C)CC2)ccc1Nc1ccc2c(n1)N(C1CCCCCC1)[C@H](C)C(=O)N2C] | 4-{[(3R)-4-Cycloheptyl-1,3- dimethyl-2-oxo-1,2,3,4- tetrahydropyrido[2,3- b]pyrazin-6-yl]amino}-3- methoxy-N-[2-(4- methylpiperazin-1- yl)ethyl]benzamide | Intermediate 39; Amine No. 26 | ¹H NMR (400 MHz, DMSO- d6): δ = 1.09 (d, 3H); 1.40- 1.82 (m, 10H); 1.89 (q, 1H); 2.00-2.11 (m, 1H); 2.14 (s, 3H); 2.24-2.37 (m, 4H); 2.38- 2.47 (m, 5H); 3.20 (s, 3H); 3.32-3.41 (m, 3H); 3.92 (s, 3H); 4.23 (q, 1H); 4.30 (tt, 1H); 6.56 (d, 1H); 7.25 (d, 1H); 7.38 (dd, 1H); 7.44 (d, 1H); 8.04 (s, 1H); 8.16 (t, 1H); 8.44 (d, 1H). |
| 92 | 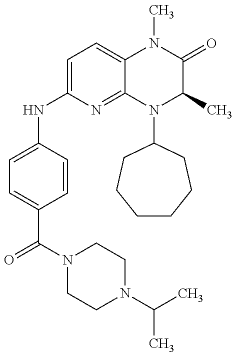 [SMILES: CC(C)N1CCN(C(=O)c2ccc(Nc3ccc4c(n3)N(C3CCCCCC3)[C@H](C)C(=O)N4C)cc2)CC1] | (3R)-4-Cylcoheptyl-1,3- dimethyl-6-[(4-{[4-(propan- 2-yl)piperazin-1- yl]carbonyl}phenyl)amino]- 3,4-dihydropyrido[2,3- b]pyrazin-2(1H)-one | Intermediate 40; Amine No. 16 | ¹H NMR (400 MHz, DMSO- d6): δ = 0.97 (d, 6H); 1.09 (d, 3H); 1.38-1.82 (m, 10H); 1.82-1.97 (m, 1H); 2.00- 2.13 (m, 1H); 2.35-2.46 (m, 4H); 2.68 (qi, 1H); 3.19 (s, 3H); 3.40-3.57 (m, 4H); 4.23 (q, 1H); 4.30 (t, 1H); 6.24 (d, 1H); 7.20-7.31 (m, 3H); 7.69 (d, 2H); 9.08 (s, 1H). |
| 93 | 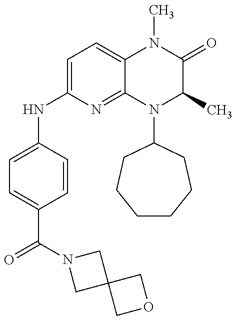 [SMILES: C[C@@H]1C(=O)N(C)c2ccc(Nc3ccc(C(=O)N4CC5(COC5)C4)cc3)nc2N1C1CCCCCC1] | (3R)-4-Cycloheptyl-1,3- dimethyl-6-{[4-(2-oxa-6- azaspiro[3.3]hept-6- ylcarbonyl)phenyl]amino}- 3,4-dihydropyrido[2,3- b]pyrazin-2(1H)-one | Intermediate 40; Amine No. 18 | ¹H NMR (400 MHz, DMSO- d6): δ = 1.10 (d, 3H); 1.38- 1.84 (m, 10H); 1.84-1.98 (m, 1H); 2.00-2.15 (m, 1H); 3.20 (s, 3H); 4.08-4.38 (m, 4H); 4.39-4.57 (m, 2H); 4.67 (s, 4H); 6.26 (d, 1H); 7.26 (d, 1H); 7.49 (d, 2H); 7.71 (d, 2H); 9.16 (s, 1H). |

### RP-HPLC Method:

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Example 94

### 4-{[(3R)-4-Benzyl-1,3-dimethyl-2-oxo-1,2,3,4-tetrahydropyrido[2,3-b]pyrazin-6-yl]amino}-N,N-dimethylbenzenesulphonamide

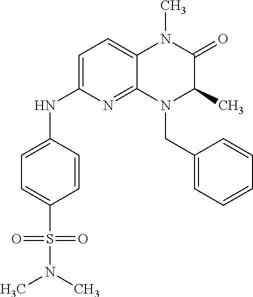 [SMILES: C[C@@H]1C(=O)N(C)c2ccc(Nc3ccc(S(=O)(=O)N(C)C)cc3)nc2N1Cc1ccccc1]

Analogously to the preparation of Example 15, 4-{[(3R)-4-benzyl-1,3-dimethyl-2-oxo-1,2,3,4-tetrahydropyrido[2,3-b]pyrazin-6-yl]amino}-N,N-dimethylbenzenesulphonamide was prepared from 150 mg of Intermediate 43, 199 mg of 4-amino-N,N-dimethylbenzenesulphonamide (Amine No. 9), 22 mg of palladium(II) acetate, 62 mg of (+)-BINAP and 810 mg of caesium carbonate in 3 ml of toluene. Purification by RP-HPLC (column: Acquity BEH C18 1.7 50×2.1 mm; mobile phase: acetonitrile/water (0.2% by volume ammonia) gradient) gave 56 mg of 4-{[(3R)-4-benzyl-1,3-dimethyl-2-oxo-1,2,3,4-tetrahydropyrido[2,3-b]pyrazin-6-yl]amino}-N,N-dimethylbenzenesulphonamide.

¹H NMR (300 MHz, DMSO-d6): δ=1.16 (d, 3H); 2.53 (s, 6H); 3.26 (s, 3H); 4.11 (q, 1H); 4.37 (d, 1H); 5.12 (d, 1H); 6.33 (d, 1H); 7.20-7.28 (m, 1H); 7.28-7.39 (m, 5H); 7.41 (d, 2H); 7.60 (d, 2H); 9.36 (s, 1H).

### Example 95

### 4-{[(3R)-4-Cycloheptyl-1,3-dimethyl-2-oxo-1,2,3,4-tetrahydropyrido[2,3-b]pyrazin-6-yl]amino}-N,N-dimethylbenzenesulphonamide

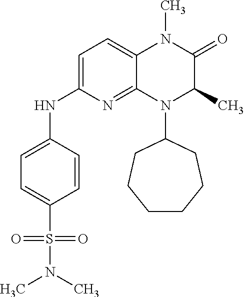 [SMILES: C[C@@H]1C(=O)N(C)c2ccc(Nc3ccc(S(=O)(=O)N(C)C)cc3)nc2N1C1CCCCCC1]

Analogously to the preparation of Example 20, 4-{[(3R)-4-cycloheptyl-1,3-dimethyl-2-oxo-1,2,3,4-tetrahydropyrido[2,3-b]pyrazin-6-yl]amino}-N,N-dimethylbenzenesulphonamide was prepared from 110 mg of Intermediate 37, 143 mg of 4-amino-N,N-dimethylbenzenesulphonamide (Amine No. 9), 16 mg of palladium(II) acetate, 45 mg of (+)-BINAP and 580 mg of caesium carbonate in 3 ml of toluene. Purification by RP-HPLC (column: Acquity BEH C18 1.7 50×2.1 mm; mobile phase: acetonitrile/water (0.2% by volume ammonia) gradient) gave 66 mg of 4-{[(3R)-4-cycloheptyl-1,3-dimethyl-2-oxo-1,2,3,4-tetrahydropyrido[2,3-b]pyrazin-6-yl]amino}-N,N-dimethylbenzenesulphonamide.

¹H NMR (300 MHz, DMSO-d6): δ=1.09 (d, 3H); 1.41-1.81 (m, 11H); 1.82-1.97 (m, 1H); 2.00-2.12 (m, 1H); 2.55 (s, 6H); 3.21 (s, 3H); 4.20-4.36 (m, 2H); 6.29 (d, 1H); 7.29 (d, 1H); 7.53 (d, 2H); 7.81 (d, 2H); 9.41 (s, 1H).

TABLE 4
The following examples were prepared analogously to Example 1 or analogously to Example
15 from the respective intermediates:

| | | | Analogously | |
|---|---|---|---|---|
| | | | to/ | |
| | | | Intermediate/ | |
| Ex. | Structure | Name | Amine | Analytical data |
| 96 | 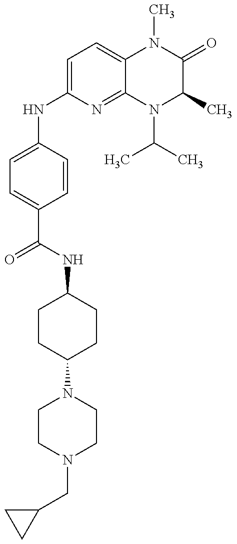 [SMILES: CC(C)N1c2nc(Nc3ccc(C(=O)N[C@H]4CC[C@H](N5CCN(CC6CC6)CC5)CC4)cc3)ccc2N(C)C(=O)[C@H]1C] | N-{trans-4-[4- (Cyclopropylmethyl)piperazin- 1-yl]cyclohexyl}-4-{[(3R)- 1,3-dimethyl-2-oxo-4- (propan-2-yl)-1,2,3,4- tetrahydropyrido[2,3- b]pyrazin-6- yl]amino}benzamide | analogously to Ex. 1, Intermediate 21; Amine No. 7 | UPLC-MS: RT = 0.75 min (M⁺ + 1 = 574) |
| 97 | 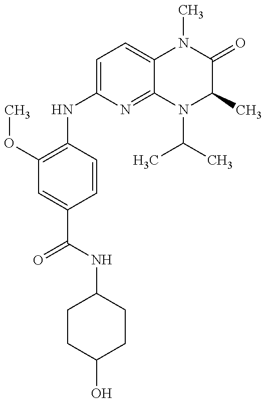 [SMILES: COc1cc(C(=O)NC2CCC(O)CC2)ccc1Nc1ccc2c(n1)N(C(C)C)[C@H](C)C(=O)N2C] | 4-{[(3R)-1,3-Dimethyl-2-oxo- 4-(propan-2-yl)-1,2,3,4- tetrahydropyrido[2,3- b]pyrazin-6-yl]amino}-N-(4- hydroxycyclohexyl)-3- methoxybenzamide | Analogously to Ex. 1, Intermediate 17; Amine No. 23 | ¹H-NMR (400 MHz, DMSO-d6, sel. signals): δ = 1.10 (d, 3H); 1.16-1.46 (m + 2d, 10H); 1.83 (bt, 4H); 3.20 (s, 3H); 3.64-3.79 (m, 1H); 3.91 (s, 3H); 4.26 (q, 1H); 4.53- 4.66 (m, 2H); 6.56 (d, 1H); 7.26 (d, 1H); 7.40-7.48 (m, 2H); 7.94 (d, 1H); 8.05 (s, 1H); 8.41 (d, 1H). |
| 98 | 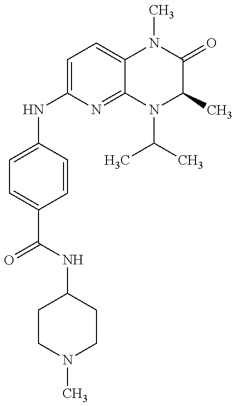 [SMILES: CC(C)N1c2nc(Nc3ccc(C(=O)NC4CCN(C)CC4)cc3)ccc2N(C)C(=O)[C@H]1C] | 4-{[(3R)-1,3-Dimethyl-2-oxo- 4-(propan-2-yl)-1,2,3,4- tetrahydropyrido[2,3- b]pyrazin-6-yl]amino}-N-(1- methylpiperidin-4- yl)benzamide | Analogously to Ex. 1, Intermediate 21; Amine No. 1 | ¹H-NMR (400 MHz, DMSO-d6, sel. signals): δ = 1.10 (d, 3H); 1.27 (d, 3H); 1.36 (d, 3H); 1.67- 1.80 (m, 2H); 1.89- 1.99 (m, 2H); 2.59- 2.69 (m, 3H); 3.21 (s, 3H); 3.89-4.00 (m, 1H); 4.27 (q, 1H); 4.62 (sp, 1H); 6.28 (d, 1H); 7.27 (d, 1H); 7.68 (d, 2H); 7.76 (d, 2H); 8.08 (d, 1H); 9.10 (s, 1H). |
| 99 | 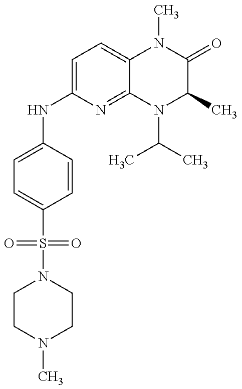 [SMILES: CC(C)N1c2nc(Nc3ccc(S(=O)(=O)N4CCN(C)CC4)cc3)ccc2N(C)C(=O)[C@H]1C] | (3R)-1,3-Dimethyl-6-({4-[(4- methylpiperazin-1- yl)sulphonyl]phenyl}amino)- 4-(propan-2-yl)-3,4- dihydropyrido[2,3-b]pyrazin- 2(1H)-one | Analogously to Ex. 15, Intermediate 15; Amine No. 11 | ¹H NMR (300 MHz, DMSO-d6): δ = 1.10 (d, 3H); 1.27 (d, 3H); 1.35 (d, 3H); 2.13 (s, 3H); 2.29-2.40 (m, 4H); 2.76-2.93 (m, 4H); 3.21 (s, 3H); 4.28 (q, 1H); 4.59 (h, 1H); 6.32 (d, 1H); 7.29 (d, 1H); 7.56 (d, 2H); 7.84 (d, 2H); 9.41 (s, 1H). |
| 100 | 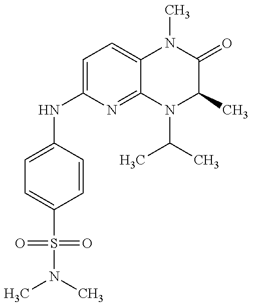 [SMILES: CC(C)N1c2nc(Nc3ccc(S(=O)(=O)N(C)C)cc3)ccc2N(C)C(=O)[C@H]1C] | 4-{[(3R)-1,3-Dimethyl-2-oxo- 4-(propan-2-yl)-1,2,3,4- tetrahydropyrido[2,3- b]pyrazin-6-yl]amino}-N,N- dimethylbenzenesulphon- amide | analogously to Ex. 15, Intermediate 15; Amine No. 9 | ¹H NMR (400 MHz, DMSO-d6): δ = 1.10 (d, 3H); 1.27 (d, 3H); 1.35 (d, 3H); 2.57 (s, 6H); 3.21 (s, 3H); 4.27 (q, 1H); 4.60 (sp, 1H); 6.32 (d, 1H); 7.29 (d, 1H); 7.58 (d, 2H); 7.84 (d, 2H); 9.37 (s, 1H). |
| 101 | 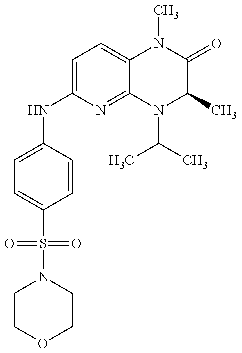 [SMILES: CC(C)N1c2nc(Nc3ccc(S(=O)(=O)N4CCOCC4)cc3)ccc2N(C)C(=O)[C@H]1C] | (3R)-1,3-Dimethyl-6-{[4- (morpholin-4- ylsulphonyl)phenyl]amino}-4- (propan-2-yl)-3,4- dihydropyrido[2,3-b]pyrazin- 2(1H)-one | analogously to Ex. 15, Intermediate 15; Amine No. 10 | ¹H NMR (300 MHz, DMSO-d6): δ = 1.11 (d, 3H); 1.28 (d, 3H); 1.36 (d, 3H); 2.77- 2.89 (m, 4H); 3.22 (s, 3H); 3.56-3.70 (m, 4H); 4.28 (q, 1H); 4.60 h, 1H); 6.33 (d, 1H); 7.30 (d, 1H); 7.57 (d, 2H); 7.86 (d, 2H); 9.44 (s, 1H). |
| 102 | 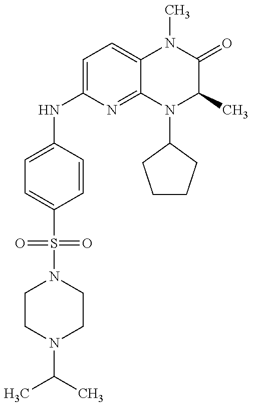 [SMILES: CC(C)N1CCN(S(=O)(=O)c2ccc(Nc3ccc4c(n3)N(C3CCCC3)[C@H](C)C(=O)N4C)cc2)CC1] | (3R)-4-Cyclopentyl-1,3- dimethyl-6-[(4-{[4-(propan-2- yl)piperazin-1-yl]sulphonyl} phenyl)amino]-3,4- dihydropyrido[2,3-b]pyrazin- 2(1H)-one | analogously to Ex. 15, Intermediate 5; Amine No. 8 | ¹H NMR (300 MHz, DMSO-d6): δ = 0.89 (d, 6H), 1.08 (d, 3H), 1.52-1.80 (m, 6H), 1.92-2.09 (m, 2H), 2.42-2.48 (m, 4H, superimposed by DMSO peak), 2.55- 2.68 (m, 1H), 2.76- 2.89 (m, 4H), 3.22 (s, 3H), 4.21 (q, 1H), 4.37 (h, 1H), 6.35 (d, 1H), 7.30 (d, 1H), 7.53 (d, 2H), 7.79 (d, 2H), 9.40 (s, 1H). |
| 103 | 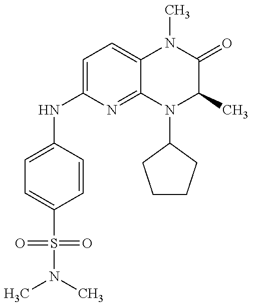 [SMILES: C[C@@H]1C(=O)N(C)c2ccc(Nc3ccc(S(=O)(=O)N(C)C)cc3)nc2N1C1CCCC1] | 4-{[(3R)-4-Cyclopentyl-1,3- dimethyl-2-oxo-1,2,3,4- tetrahydropyrido[2,3- b]pyrazin-6-yl]amino}-N,N- dimethylbenzenesulphonamide | analogously to Ex. 15, Intermediate 5; Amine No. 9 | ¹H NMR (400 MHz, DMSO-d6): δ = 1.08 (d, 3H); 1.56-1.78 (m, 6H); 1.94-2.07 (m, 2H); 2.56 (s, 6H); 3.22 (s, 3H); 4.21 (q, 1H); 4.38 (qi, 1H); 6.35 (d, 1H); 7.30 (d, 1H); 7.56 (d, 2H); 7.80 (d, 2H); 9.36 (s, 1H). |

### RP-HPLC Method:

Instrument: Waters Acquity UPLC-MS SQD; column: Acquity UPLC BEH C18 1.7 50×2.1 mm; mobile phase A: water+0.1% by volume of formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Example 104

### 4-{[4-(2-Methoxyethyl)-1,3-dimethyl-2-oxo-1,2,3,4-tetrahydropyrido[2,3-b]pyrazin-6-yl]amino}-N-(1-methylpiperidin-4-yl)benzamide

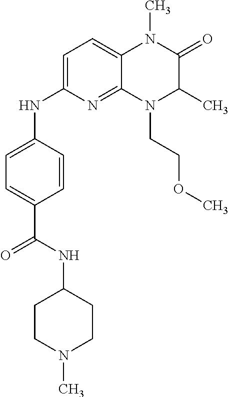 [SMILES: COCCN1c2nc(Nc3ccc(C(=O)NC4CCN(C)CC4)cc3)ccc2N(C)C(=O)C1C]

In analogy to the preparation of Example 1, 4-{[4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide was prepared from 70 mg of Intermediate 53, 74 mg of 4-amino-1-methylpiperidine (Amine No. 1), 108 mg of HATU and 77 mg of triethylamine in 1.5 ml of DMF. Purification by RP-HPLC (column: X-Bridge C18 5 μm 100×30 mm, mobile phase: acetonitrile/water (0.2% by volume of ammonia) gradient) gave 50 mg of 4-{[4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide.

¹H NMR (400 MHz, DMSO-d6, selected signals): δ=1.14 (d, 3H); 1.57 (dq, 1H); 1.73 (bd, 1H); 1.92 (dt, 2H); 2.16 (s, 3H); 2.75 (bd, 2H); 3.21 (s, 3H); 3.28 (s, 3H); 3.54-3.65 (m, 2H); 3.65-3.77 (m, 1H); 4.08 (dt, 1H); 4.19 (q, 1H); 6.26 (d, 1H); 7.24 (d, 1H); 7.61 (d, 2H); 7.74 (d, 2H); 7.94 (d, 1H); 9.06 (s, 1H).

### Example 105

### N-{trans-4-[4-(Cyclopropylmethyl)piperazin-1-yl]cyclohexyl}-4-{[4-(2-methoxyethyl)-1,3-dimethyl-2-oxo-1,2,3,4-tetrahydropyrido[2,3-b]pyrazin-6-yl]amino}benzamide

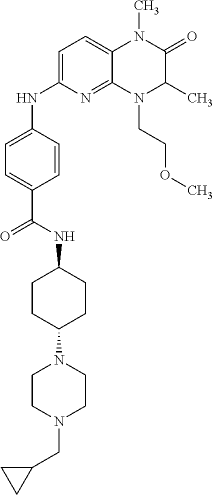 [SMILES: COCCN1c2nc(Nc3ccc(C(=O)N[C@H]4CC[C@H](N5CCN(CC6CC6)CC5)CC4)cc3)ccc2N(C)C(=O)C1C]

Analogously to the preparation of Example 1, N-{trans-4-[4-(cyclopropylmethyl)piperazin-1-yl]cyclohexyl}-4-{[4-(2-methoxyethyl)-1,3-dimethyl-2-oxo-1,2,3,4-tetrahydropyrido[2,3-b]pyrazin-6-yl]amino}benzamide was prepared from 70 mg of Intermediate 53, 89 mg of trans-4-[4-(cyclopropylmethyl)piperazin-1-yl]cyclohexanamine (Amine No. 7), 108 mg of HATU and 77 mg of triethylamine in 1.5 ml of DMF. Purification by RP-HPLC (column: X-Bridge C18 5 μm 100×30 mm, mobile phase: acetonitrile/water (0.2% by volume of ammonia) gradient) gave 23 mg of N-{trans-4-[4-(cyclopropylmethyl)piperazin-1-yl]cyclohexyl}-4-{[4-(2-methoxyethyl)-1,3-dimethyl-2-oxo-1,2,3,4-tetrahydropyrido[2,3-b]pyrazin-6-yl]amino}benzamide.

¹H NMR (400 MHz, DMSO-d6, selected signals): δ=0.01-0.08 (m, 2H); 0.40-0.48 (m, 2H); 0.74-0.85 (m, 1H); 1.14 (d, 3H); 1.21-1.41 (m, 4H); 1.77-1.92 (m, 4H); 2.13 (d, 2H); 2.14-2.24 (m, 1H); 3.21 (s, 3H); 3.28 (s, 3H); 2.54-3.60 (m, 2H); 3.60-3.75 (m, 1H); 4.08 (dt, 1H); 4.19 (q, 1H); 6.26 (d, 1H); 7.24 (d, 1H); 7.61 (d, 2H); 7.73 (d, 2H); 7.90 (d, 1H); 9.06 (s, 1H).

### Example 106

### tert-Butyl 4-[(3R)-6-{[4-(dimethylsulphamoyl)phenyl]amino}-1,3-dimethyl-2-oxo-2,3-dihydropyrido[2,3-b]pyrazin-4(1H)-yl]piperidine-1-carbonate

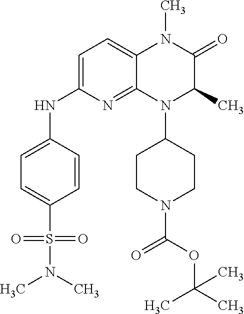 [SMILES: C[C@@H]1C(=O)N(C)c2ccc(Nc3ccc(S(=O)(=O)N(C)C)cc3)nc2N1C1CCN(C(=O)OC(C)(C)C)CC1]

Analogously to the preparation of Example 20, tert-butyl 4-[(3R)-6-{[4-(dimethylsulphamoyl)phenyl]amino}-1,3-dimethyl-2-oxo-2,3-dihydropyrido[2,3-b]pyrazin-4(1H)-yl]piperidine-1-carbonate was prepared from 74 mg of Intermediate 56, 56 mg of 4-amino-N,N-dimethylbenzenesulphonamide (Amine No. 9), 8.4 mg of palladium(II) acetate, 23 mg of (+)-BINAP and 305 mg of caesium carbonate in 2 ml of toluene and 0.2 ml of dioxane. Purification by RP-HPLC (column: Acquity BEH C18 1.7 50×2.1 mm, mobile phase: acetonitrile/water (0.2% by volume of ammonia) gradient) gave 12.4 mg of tert-butyl 4-[(3R)-6-{[4-(dimethylsulphamoyl)phenyl]amino}-1,3-dimethyl-2-oxo-2,3-dihydropyrido[2,3-b]pyrazin-4(1H)-yl]piperidine-1-carbonate.

¹H NMR (300 MHz, DMSO-d6): δ=1.07 (d, 3H); 1.41 (s, 9H); 1.49-1.84 (m, 3H); 2.07 (bd, 1H); 2.56 (s, 6H); 2.76-3.01 (m, 2H); 3.21 (s, 3H); 4.10 (bt, 2H); 4.21-4.36 (m, 2H); 6.33 (d, 1H); 7.31 (d, 1H); 7.59 (d, 2H); 7.79 (d, 2H); 9.42 (s, 1H).

### Example 107

### 4-[(1,3-Dimethyl-2-oxo-4-phenyl-1,2,3,4-tetrahydropyrido[2,3-b]pyrazin-6-yl)amino]-N-(1-methylpiperidin-4-yl)benzenesulphonamide

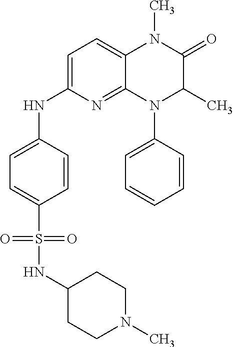 [SMILES: CC1C(=O)N(C)c2ccc(Nc3ccc(S(=O)(=O)NC4CCN(C)CC4)cc3)nc2N1c1ccccc1]

Analogously to the preparation of Example 20, 4-[(1,3-dimethyl-2-oxo-4-phenyl-1,2,3,4-tetrahydropyrido[2,3-b]pyrazin-6-yl)amino]-N-(1-methylpiperidin-4-yl)benzenesulphonamide was prepared from 100 mg of Intermediate 60, 56 mg of 4-amino-N-(1-methylpiperidin-4-yl)benzenesulphonamide (Amine No. 13; preparation: WO2008052847, Example 66, steps a+b), 4.5 mg of tris(dibenzylideneacetone)dipalladium, 8.4 mg of Xanthphos and 161 mg of caesium carbonate in 6.6 ml of dioxane. Purification by RP-HPLC (column: Acquity BEH C18 1.7 50×2.1 mm, mobile phase: acetonitrile/water (0.2% by volume of ammonia) gradient) gave 50 mg of 4-[(1,3-dimethyl-2-oxo-4-phenyl-1,2,3,4-tetrahydropyrido[2,3-b]pyrazin-6-yl)amino]-N-(1-methylpiperidin-4-yl)benzenesulphonamide.

¹H NMR (400 MHz, DMSO-d6): δ=1.27-1.39 (m+s, 2+3H); 1.43-1.52 (m, 2H); 1.77 (bt, 2H); 2.06 (s, 3H); 2.58 (bd, 2H); 2.69-2.81 (m, 1H); 3.31 (s, 3H); 4.57 (q, 1H); 6.44 (d, 1H); 7.29 (t, 1H); 7.31-7.49 (m, 10H); 9.31 (s, 1H).

### Example 108

### 4-{[(3R)-1,3-Dimethyl-2-oxo-4-phenyl-1,2,3,4-tetrahydropyrido[2,3-b]pyrazin-6-yl]amino}-N-(1-methylpiperidin-4-yl)benzenesulphonamide

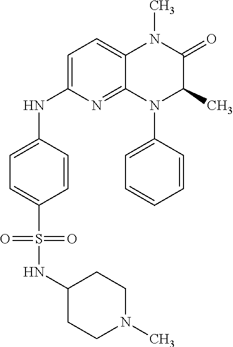 [SMILES: C[C@@H]1C(=O)N(C)c2ccc(Nc3ccc(S(=O)(=O)NC4CCN(C)CC4)cc3)nc2N1c1ccccc1]

36 mg of 4-[(1,3-dimethyl-2-oxo-4-phenyl-1,2,3,4-tetrahydropyrido[2,3-b]pyrazin-6-yl)amino]-N-(1-methylpiperidin-4-yl)benzenesulphonamide (Example 107) were separated into the enantiomers by chiral HPLC (Chiralpak IA 5 m 250×30 mm, hexane/2-propanol/diethylamine 70:30:0.1 (v/v)). This gave 9.2 mg of 4-{[(3R)-1,3-dimethyl-2-oxo-4-phenyl-1,2,3,4-tetrahydropyrido[2,3-b]pyrazin-6-yl]amino}-N-(1-methylpiperidin-4-yl)benzenesulphonamide.

Chiral HPLC: Rt=7.44 min

Instrument: Waters Alliance 2695; column: Chiralpak IA 3 μm 100×4.6 mm; mobile phase A: hexane/2-propanol/diethylamine 70:30:0.1; flow rate 1 ml/min; temperature: 25° C.; injection: 5 μl (1 mg/ml ethanol/methanol, 1:1); DAD 996 scan: 280 nm.

### Example 109

### 4-{[(3R)-4-Benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzenesulphonamide

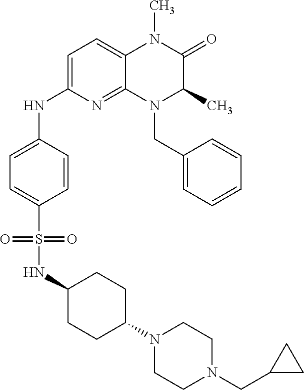 [SMILES: C[C@@H]1C(=O)N(C)c2ccc(Nc3ccc(S(=O)(=O)N[C@H]4CC[C@H](N5CCN(CC6CC6)CC5)CC4)cc3)nc2N1Cc1ccccc1]

A suspension of 100 mg of Intermediate 43, 260 mg of 4-amino-N-{trans-4-[4-(cyclopropylmethyl)piperazin-1-yl]cyclohexyl}benzenesulphonamide (Intermediate 28), 15 mg of palladium(II) acetate, 540 mg of caesium carbonate and 41 mg of (+)-BINAP in 10 ml of toluene was stirred at 120° C. under an argon atmosphere for 38 hours. The reaction solution was filtered off and concentrated under reduced pressure. The residue was purified by RP-HPLC chromatography (column: X-Bridge C18 5 μm 100×30 mm, mobile phase: acetonitrile/water (0.1% by volume of diethylamine) gradient). This gave 20 mg of N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide.

¹H NMR (400 MHz, DMSO-d6): δ=−0.02-0.06 (m, 2H); 0.37-0.46 (m, 2H); 0.69-0.83 (m, 1H); 1.10-1.2 (m+d, 7H); 1.57-1.72 (m, 4H); 2.00-2.13 (m+d, 3H); 2.28-2.45 (m, 8H); 2.69-2.82 (m, 1H); 3.25 (s, 3H); 4.11 (q, 1H); 4.38 (d, 1H); 5.12 (d, 1H); 6.31 (d, 1H); 7.20-7.39 (m, 7H); 7.48 (d, 2H); 7.54 (d, 2H); 9.26 (s, 1H).

Chiral HPLC: Rt=3.28 min

Instrument: Waters Alliance 2695; column: Chiralpak IC 3 μm 100×4.6 mm; mobile phase A: hexane/methanol/diethylamine 50:50:0.1; flow rate 1 ml/min; temperature: 25° C.; injection: 5 μl (1 mg/ml ethanol/methanol, 1:1); DAD 996 scan: 280 nm.

### Example 110

### 4-{[(3R)-1,3-Dimethyl-2-oxo-4-(tetrahydro-2H-pyran-4-yl)-1,2,3,4-tetrahydropyrido[2,3-b]pyrazin-6-yl]amino}-3-methoxy-N-(1-methylpiperidin-4-yl)benzenesulphonamide

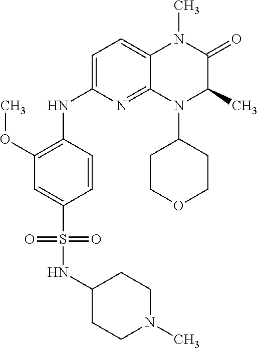 [SMILES: COc1cc(S(=O)(=O)NC2CCN(C)CC2)ccc1Nc1ccc2c(n1)N(C1CCOCC1)[C@H](C)C(=O)N2C]

A suspension of 107 mg of Intermediate 24, 162 mg of Intermediate 72, 5 mg of tris(dibenzylideneacetone)dipalladium(0) (CAS 51364-51-3), 177 mg of caesium carbonate and 9 mg of Xanthphos (CAS 161265-03-8) in 7 ml of dioxane was stirred under an argon atmosphere at 100° C. for 8 hours. The reaction solution was filtered off, water was added and the mixture was extracted with ethyl acetate. The organic phase was washed with saturated sodium chloride solution and dried over sodium sulphate, and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 37 mg of 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(1-methylpiperidin-4-yl)benzenesulphonamide.

¹H NMR (400 MHz, DMSO-d6): δ=1.09 (d, 3H); 1.37/qd, 1H); 1.54 (bd, 2H); 1.62 (bd, 1H); 1.72-1.89 (m, 3H(; 1.92-2.02 (m, 2H); 2.09 (s, 3H); 2.57-2.66 (m, 2H); 2.81-2.92 (m, 1H); 3.37-3.52 (m, 2H); 4.00 (dt, 2H); 4.25 (q, 1H); 4.36 (tt, 1H); 6.64 (d, 1H); 7.27-7.37 (m, 3H); 7.44 (d, 1H); 8.29 (s, 1H); 8.49 (d, 1H).

### Example 111

### N-{trans-4-[4-(Cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzenesulphonamide

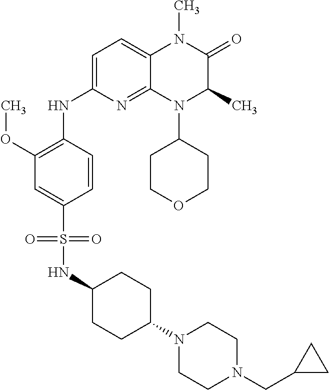 [SMILES: COc1cc(S(=O)(=O)N[C@H]2CC[C@H](N3CCN(CC4CC4)CC3)CC2)ccc1Nc1ccc2c(n1)N(C1CCOCC1)[C@H](C)C(=O)N2C]

A suspension of 110 mg of Intermediate 24, 224 mg of Intermediate 76, 5 mg of tris(dibenzylideneacetone)dipalladium(0) (CAS 51364-51-3), 173 mg of caesium carbonate and 9 mg of Xanthphos (CAS 161265-03-8) in 7 ml of dioxane was stirred under an argon atmosphere at 100° C. for 8 hours. The reaction solution was filtered off, water was added and the mixture was extracted with ethyl acetate. The organic phase was washed with saturated sodium chloride solution and dried over sodium sulphate, and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 58 mg of N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzenesulphonamide.

¹H NMR (400 MHz, DMSO-d6): δ=0.00-0.05 (m, 2H); 0.38-0.45 (m, 2H); 0.71-0.82 (m, 1H); 1.04-1.20 (m+d, 7H); 1.59-1.72 (m, 5H); 1.79 (qd, 1H); 1.89-2.01 (m, 2H); 2.04-2.15 (m+d, 3H); 2.31-2.46 (m, 7H); 2.83 (bs, 1H); 3.22 (s, 3H); 3.37-3.52 (m, 2H); 3.92 (s, 3H); 3.99 (dt, 2H); 4.25 (q, 1H); 4.37 (tt, 1H); 6.64 (d, 1H); 7.27-7.35 (m, 3H); 7.39 (d, 1H); 8.28 (s, 1H); 8.48 (d, 1H).

### Example 112

### (3R)-6-({2-Methoxy-4-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one

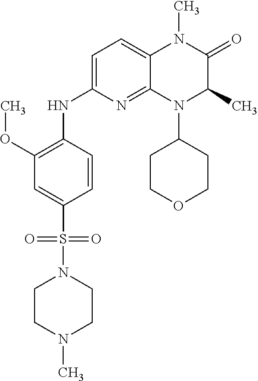 [SMILES: COc1cc(S(=O)(=O)N2CCN(C)CC2)ccc1Nc1ccc2c(n1)N(C1CCOCC1)[C@H](C)C(=O)N2C]

A suspension of 103 mg of Intermediate 24, 142 mg of Intermediate 74, 5 mg of tris(dibenzylideneacetone)dipalladium(0) (CAS 51364-51-3), 162 mg of caesium carbonate and 8.5 mg of Xanthphos (CAS 161265-03-8) in 6.6 ml of dioxane was stirred under argon at 100° C. for 16 hours and heated in a microwave oven at 150° C. for a further 16.5 hours. The reaction solution was filtered off, water was added and the mixture was extracted with ethyl acetate. The organic phase was washed with saturated sodium chloride solution and dried over sodium sulphate, and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 11.6 mg of (3R)-6-({2-methoxy-4-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one.

¹H NMR (400 MHz, DMSO-d6): δ=1.09 (d, 3H); 1.62 (bd, 1H); 1.79 (qd, 1H); 1.89-2.02 (m, 2H); 2.14 (s, 3H); 2.31-2.40 m, 4H); 2.85-2.95 (m, 4H); 3.22 (s, 3H); 3.42 (dt, 1H); 3.48 (dt, 1H); 3.92-4.03 (m+s, 5H); 4.26 (q, 1H); 4.35 (tt, 1H); 6.67 (d, 1H); 7.25 (d, 1H); 7.24 (dd, 1H); 7.31 (d, 1H); 8.39 (s, 1H); 8.55 (d, 1H).

### Biological Efficacy of the Compounds According to the Invention

Protein-Protein Interaction Assay: BRD4/Acetylated Peptide H4 Binding Assay

### 1. Assay Description for BRD4 Bromo Domain 1 [BRD4(1)]

To assess the BRD4(1) binding strength of the substances described in this application, the ability thereof to inhibit the interaction between BRD4(1) and acetylated histone H4 in a dose-dependent manner was quantified.

For this purpose, a time-resolved fluorescence resonance energy transfer (TR-FRET) assay was used, which measures the binding between N-terminally His6-tagged BRD4(1) (amino acids 67-152) and a synthetic acetylated histone H4 (Ac-H4) peptide with sequence GRGK(Ac)GGK(Ac)GLGK(Ac)GGAK(Ac)RHGSGSK-biotin. The recombinant BRD4(1) protein produced in-house according to Filippakopoulos et al., Cell, 2012, 149:214-231 was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4(1) solution (final concentration typically 10 nM in the 5 μl of reaction volume) in aqueous assay buffer [50 mM HEPES pH 7.5, 50 mM sodium chloride (NaCl), 0.25 mM CHAPS and 0.05% serum albumin (BSA)] to the substances in the test plate. This was followed by a 10-minute incubation step at 22° C. for the pre-equilibration of putative complexes between BRD4(1) and the substances. Subsequently, 3 μl of a 1.67-fold concentrated solution (in assay buffer) consisting of Ac-H4 peptide (83.5 nM) and TR-FRET detection reagents [16.7 nM anti-6His-XL665 and 3.34 nM streptavidin cryptate (both from Cisbio Bioassays, Codolet, France), and 668 mM potassium fluoride (KF)] were added.

The mixture was then incubated in the dark at 22° C. for one hour and then at 4° C. for at least 3 hours and for no longer than overnight. The formation of BRD4(1)/Ac-H4 complexes was determined by the measurement of the resonance energy transfer from the streptavidin-Eu cryptate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4(1)/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used. Inhibition of 100% corresponded to the mean from the measurements for a set of controls (typically 32 data points) in which all the reagents except BRD4(1) were present. The IC₅₀ was determined by regression analysis based on a 4-parameter equation (minimum, maximum, IC₅₀, Hill; Y=max+(min−max)/(1+(X/IC₅₀)Hill).

### 2. Assay Description for BRD4 Bromo Domain 2 [BRD4(2)]

To assess the BRD4(2) binding strength of the substances described in this application, the ability thereof to inhibit the interaction between BRD4(2) and acetylated histone H4 in a dose-dependent manner was quantified.

For this purpose, a time-resolved fluorescence resonance energy transfer (TR-FRET) assay was used, which measures the binding between N-terminally His6-tagged BRD4(2) (amino acids 357-445) and a synthetic acetylated histone H4 (Ac-H4) peptide with sequence SGRGK(Ac)GGK(Ac)GLGK(Ac)GGAK(Ac)RHRKVLRDNGSGSK-biotin. The recombinant BRD4(2) protein produced in-house according to Filippakopoulos et al., Cell, 2012, 149:214-231 was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4(2) solution (final concentration typically 100 nM in the 5 μl of reaction volume) in aqueous assay buffer [50 mM HEPES pH 7.5, 50 mM sodium chloride (NaCl); 50 mM potassium fluoride (KF); 0.25 mM CHAPS and 0.05% serum albumin (BSA)] to the substances in the test plate. This was followed by a 10-minute incubation step at 22° C. for the pre-equilibration of putative complexes between BRD4(2) and the substances. Subsequently, 3 μl of a 1.67-fold concentrated solution (in assay buffer) consisting of Ac-H4 peptide (83.5 nM) and TR-FRET detection reagents [83.5 nM anti-6His-XL665 (Cisbio Bioassays, Codolet, France) and 12.52 nM streptavidin-Eu), (Perkin Elmer, #W1024)] were added.

The mixture was then incubated in the dark at 22° C. for one hour and then at 4° C. for at least 3 hours and for no longer than overnight. The formation of BRD4(2)/Ac-H4 complexes was determined by the measurement of the resonance energy transfer from the streptavidin-Eu chelate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4(2)/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used. Inhibition of 100% corresponded to the mean from the measurements for a set of controls (typically 32 data points) in which all the reagents except BRD4(2) were present. The IC₅₀ was determined by regression analysis based on a 4-parameter equation (minimum, maximum, IC₅₀, Hill; Y=max+(min−max)/(1+(X/IC₅₀)Hill).

### 3. Cell Assay

### Cell Proliferation Assay

In accordance with the invention, the substances were tested for their ability to inhibit the proliferation of the MOLM-13 cell linie (Deutsche Sammlung für Mikroorganismen und Zellkulturen [German Collection of Microorganisms and Cell Cultures], ACC 554; acute myeloid leukaemia). Cell viability was determined by means of the alamarBlue® reagent (Invitrogen) in a Victor X3 Multilabel Reader (Perkin Elmer). The excitation wavelength was 530 nm and the emission wavelength 590 nM.

The MOLM-13 cells were sown at a density of 4000 cells/well in 100 μl of growth medium on 96-well microtitre plates. After overnight incubation at 37° C., the fluorescence values were determined (CI values). The plates were then treated with various substance dilutions and incubated at 37° C. for 96 hours. Subsequently, the fluorescence values were determined (CO values). For the data analysis, the CI values were subtracted from the CO values and the results were compared between cells which had been treated with various dilutions of the substance or only with buffer solution. The IC₅₀ values (substance concentration needed for 50% inhibition of cell proliferation) were calculated therefrom.

### 4. Results:

### 4.1 Binding Assay

Table 5 shows the results from the BRD4(1) binding assay.

TABLE 5

| | IC₅₀ [BRD4(1)] |
|---|---|
| Example | (nmol/l) |
| 1 | 260 |
| 2 | 501 |
| 3 | 131 |
| 4 | 374 |
| 5 | 93 |
| 6 | 255 |
| 7 | 91 |
| 8 | 141 |
| 9 | 150 |
| 10 | 165 |
| 11 | 63 |
| 12 | 104 |
| 13 | 114 |
| 14 | 149 |
| 15 | 237 |
| 16 | 286 |
| 17 | 308 |
| 18 | 56 |
| 19 | 219 |
| 20 | 154 |
| 21 | 106 |
| 22 | 52 |
| 23 | 46 |
| 24 | 81 |
| 25 | 50 |
| 26 | 70 |
| 27 | 32 |
| 28 | 195 |
| 29 | 242 |
| 30 | 119 |
| 31 | 241 |
| 32 | 376 |
| 33 | 329 |
| 34 | 256 |
| 35 | 119 |
| 36 | 257 |
| 37 | 347 |
| 38 | 194 |
| 39 | 329 |
| 40 | 295 |
| 41 | 122 |
| 42 | 156 |
| 43 | 218 |
| 44 | 368 |
| 45 | 77 |
| 46 | 52 |
| 47 | 201 |
| 48 | 103 |
| 49 | 114 |
| 50 | 145 |
| 51 | 138 |
| 52 | 166 |
| 53 | 90 |
| 54 | 357 |
| 55 | 215 |
| 56 | 139 |
| 57 | 139 |
| 58 | 111 |
| 59 | 533 |
| 60 | 339 |
| 61 | 172 |
| 62 | 187 |
| 63 | 262 |
| 64 | 179 |
| 65 | 234 |
| 66 | 140 |
| 67 | 178 |
| 68 | 263 |
| 69 | 164 |
| 70 | 239 |
| 71 | 961 |
| 72 | 131 |
| 73 | 273 |
| 74 | 300 |
| 75 | 149 |
| 76 | 840 |
| 77 | 55 |
| 78 | 153 |
| 79 | 120 |
| 80 | 137 |
| 81 | 89 |
| 82 | 40 |
| 83 | 133 |
| 84 | 448 |
| 85 | 186 |
| 86 | 180 |
| 87 | 216 |
| 88 | 339 |
| 89 | 185 |
| 90 | 341 |
| 91 | 154 |
| 92 | 325 |
| 93 | 317 |
| 94 | 156 |
| 95 | 589 |
| 96 | 438 |
| 97 | 261 |
| 98 | 281 |
| 99 | 145 |
| 100 | 156 |
| 101 | 134 |
| 102 | 100 |
| 103 | 78 |
| 104 | 621 |
| 105 | 452 |
| 106 | 632 |
| 107 | 201 |
| 108 | 63 |
| 109 | 36 |
| 110 | 38 |
| 111 | 29 |
| 112 | 106 |

Table 6 shows the results from the BRD4(2) binding assay.

TABLE 6

| | IC₅₀ [BRD4(2)] |
|---|---|
| Example | (nmol/l) |
| 1 | 138 |
| 2 | 622 |
| 3 | 472 |
| 4 | 234 |
| 8 | 83 |
| 9 | 130 |
| 10 | 230 |
| 11 | 83 |
| 12 | 111 |
| 13 | 113 |
| 14 | 108 |
| 15 | 51 |
| 16 | 212 |
| 17 | 111 |
| 18 | 40 |
| 19 | 185 |
| 20 | 96 |
| 21 | 87 |
| 22 | 53 |
| 23 | 65 |
| 24 | 76 |
| 25 | 94 |
| 26 | 67 |
| 27 | 38 |
| 28 | 116 |
| 29 | 149 |
| 30 | 140 |
| 31 | 108 |
| 32 | 77 |
| 33 | 80 |
| 34 | 104 |
| 35 | 237 |
| 36 | 245 |
| 37 | 306 |
| 38 | 79 |
| 39 | 145 |
| 40 | 161 |
| 41 | 109 |
| 42 | 99 |
| 43 | 102 |
| 44 | 283 |
| 45 | 86 |
| 47 | 112 |
| 48 | 167 |
| 49 | 317 |
| 50 | 111 |
| 51 | 219 |
| 52 | 299 |
| 53 | 94 |
| 54 | 354 |
| 55 | 128 |
| 56 | 242 |
| 57 | 156 |
| 58 | 134 |
| 59 | 323 |
| 60 | 212 |
| 61 | 110 |
| 62 | 146 |
| 63 | 206 |
| 64 | 81 |
| 65 | 120 |
| 66 | 154 |
| 67 | 110 |
| 68 | 310 |
| 69 | 89 |
| 70 | 120 |
| 71 | 445 |
| 72 | 118 |
| 73 | 196 |
| 74 | 156 |
| 75 | 82 |
| 76 | 273 |
| 77 | 103 |
| 78 | 111 |
| 79 | 51 |
| 80 | 138 |
| 81 | 23 |
| 82 | 48 |
| 83 | 61 |
| 84 | 165 |
| 85 | 98 |
| 86 | 100 |
| 87 | 75 |
| 88 | 146 |
| 89 | 93 |
| 90 | 208 |
| 91 | 135 |
| 92 | 209 |
| 93 | 90 |
| 94 | 111 |
| 95 | 126 |
| 96 | 344 |
| 97 | 117 |
| 98 | 98 |
| 99 | 104 |
| 100 | 71 |
| 101 | 105 |
| 102 | 90 |
| 103 | 51 |
| 104 | 217 |
| 105 | 187 |
| 106 | 463 |
| 107 | 241 |
| 108 | 239 |
| 109 | 78 |
| 110 | 55 |
| 111 | 130 |
| 112 | 86 |

### 4.2 Cell Proliferation Assay

Table 7 shows the results from the MOLM-13 cell proliferation assay.

TABLE 7
The ability of the compounds according to the invention to inhibit
the proliferation of the MOLM-13 cell line was determined

| | IC₅₀ (MOLM- |
|---|---|
| Example | 13) (nmol/l) |
| 1 | 176 |
| 2 | 2360 |
| 3 | 630 |
| 4 | 1110 |
| 5 | 503 |
| 6 | 762 |
| 7 | 377 |
| 8 | 362 |
| 9 | 698 |
| 10 | 623 |
| 11 | 444 |
| 13 | 212 |
| 14 | 233 |
| 15 | 62 |
| 18 | 305 |
| 20 | 445 |
| 21 | 397 |
| 22 | 203 |
| 23 | 358 |
| 24 | 554 |
| 25 | 388 |
| 26 | 296 |
| 27 | 202 |
| 30 | 293 |
| 34 | 338 |
| 37 | 859 |
| 38 | 51 |
| 41 | 191 |
| 42 | 326 |
| 45 | 539 |
| 46 | 461 |
| 48 | 583 |
| 50 | 576 |
| 51 | 507 |
| 53 | 249 |
| 55 | 668 |
| 57 | 366 |
| 58 | 339 |
| 61 | 407 |
| 62 | 444 |
| 64 | 310 |
| 66 | 270 |
| 67 | 265 |
| 68 | 548 |
| 69 | 358 |
| 70 | 233 |
| 72 | 254 |
| 75 | 350 |
| 77 | 242 |
| 78 | 243 |
| 79 | 282 |
| 80 | 170 |
| 81 | 96 |
| 82 | 131 |
| 83 | 122 |
| 91 | 353 |
| 99 | 316 |
| 100 | 336 |
| 101 | 226 |
| 102 | 214 |
| 103 | 177 |
| 107 | 255 |

1. A compound of the general formula (I) 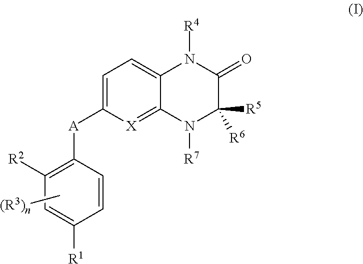
in which
A represents —NH— or —O—,
X represents —N—,
n represents 0 or 1,
R¹ represents —C(═O)NR⁸R⁹ or represents —S(═O)₂NR⁸R⁹,
or represents oxazolin-2-yl which may optionally be mono- or disubstituted by identical or different C₁-C₃-alkyl substituents,
R² represents hydrogen, halogen, cyano, C₁-C₄-alkyl, C₂-C₄-alkenyl, C₂-C₄-alkynyl, halo-C₁-C₄-alkyl-, C₁-C₄-alkoxy-, C₁-C₄-alkoxy-C₁-C₄-alkyl-, halo-C₁-C₄-alkoxy-, C₁-C₄-alkylthio-, halo-C₁-C₄-alkylthio- or —NR¹⁰R¹¹,
R³ represents halogen, C₁-C₃-alkyl, C₁-C₃-alkoxy-, C₁-C₄-alkoxy-C₁-C₄-alkyl-, trifluoromethyl- or cyano and may be attached to any of the still-unoccupied positions in the aromatic system,
R⁴ represents methyl or ethyl,
R⁵ represents hydrogen or C₁-C₃-alkyl,
R⁶ represents hydrogen or C₁-C₃-alkyl,
or
R⁵ and R⁶ together represent C₂-C₅-alkylene,
R⁷ represents C₁-C₆-alkyl, C₃-C₈-cycloalkyl, 4- to 8-membered heterocycloalkyl, phenyl or phenyl-C₁-C₃-alkyl,
in which C₁-C₆-alkyl may optionally be mono-, di- or trisubstituted by identical or different substituents from the group consisting of fluorine, oxo, cyano, hydroxy, C₁-C₃-alkoxy- and —NR¹⁰R¹¹,
and in which the phenyl radical may in each case optionally be mono-, di- or trisubstituted by identical or different substituents from the group consisting of halogen, cyano, C₁-C₄-alkyl, C₂-C₄-alkenyl, C₂-C₄-alkynyl, C₁-C₄-alkoxy-, halo-C₁-C₄-alkyl- and halo-C₁-C₄-alkoxy-,
and in which 4- to 8-membered heterocycloalkyl may optionally be mono- or disubstituted by identical or different substituents from the group consisting of oxo, fluorine, cyano, C₁-C₄-alkyl, C₁-C₄-alkoxy-, C₁-C₄-alkylcarbonyl- and C₁-C₄-alkoxycarbonyl-,
R⁸ represents C₁-C₆-alkyl which may optionally be mono-, di- or trisubstituted by identical or different substituents from the group consisting of hydroxy, oxo, fluorine, cyano, C₁-C₄-alkoxy-, halo-C₁-C₄-alkoxy-, —NR¹⁰R¹¹, C₃-C₈-cycloalkyl, C₄-C₈-cycloalkenyl, 4- to 8-membered heterocycloalkyl, 4- to 8-membered heterocycloalkenyl, C₅-C₁₁-spirocycloalkyl, C₅-C₁₁-heterospirocycloalkyl, bridged C₆-C₁₂-cycloalkyl, bridged C₆-C₁₂-heterocycloalkyl, C₆-C₁₂-bicycloalkyl, C₆-C₁₂-heterobicycloalkyl, phenyl or 5- to 6-membered heteroaryl,
in which C₃-C₈-cycloalkyl, C₄-C₈-cycloalkenyl, 4- to 8-membered heterocycloalkyl, 4- to 8-membered heterocycloalkenyl, C₅-C₁₁-spirocycloalkyl, C₅-C₁₁-heterospirocycloalkyl, bridged C₆-C₁₂-cycloalkyl, bridged C₆-C₁₂-heterocycloalkyl, C₆-C₁₂-bicycloalkyl, C₆-C₁₂-heterobicycloalkyl may in each case optionally be monosubstituted by oxo, C₁-C₄-alkyl or C₁-C₄-alkoxycarbonyl-,
and in which phenyl and 5- to 6-membered heteroaryl may optionally be mono- or disubstituted by identical or different substituents from the group consisting of halogen, cyano, trifluoromethyl-, C₁-C₃-alkyl and C₁-C₃-alkoxy-,
or represents C₃-C₆-alkenyl or C₃-C₆-alkynyl,
or represents C₃-C₈-cycloalkyl, C₄-C₈-cycloalkenyl, C₅-C₁₁-spirocycloalkyl-, bridged C₆-C₁₂-cycloalkyl- or C₆-C₁₂-bicycloalkyl-which may optionally be mono- or disubstituted by identical or different substituents from the group consisting of hydroxy, oxo, cyano, fluorine, C₁-C₃-alkyl-, C₁-C₃-alkoxy-, trifluoromethyl-, —NR¹⁰R¹¹ and 4- to 8-membered heterocycloalkyl,
or represents 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl which may optionally be mono- or disubstituted by identical or different substituents from the group consisting of hydroxy, oxo, cyano, fluorine, C₁-C₃-alkyl, C₁-C₃-alkoxy-, trifluoromethyl-, —NR₁₀R¹¹, C₁-C₄-alkylcarbonyl- and C₁-C₄-alkoxycarbonyl-,
or represents hydrogen,
R⁹ represents hydrogen or C₁-C₃-alkyl,
or
R⁸ and R⁹ together with the nitrogen atom to which they are attached represent 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl which may optionally be mono- or disubstituted by identical or different substituents from the group consisting of hydroxy, oxo, cyano, fluorine, C₁-C₃-alkyl, C₃-C₆-cycloalkyl, C₁-C₃-alkoxy-, trifluoromethyl-, —NR¹⁰R¹¹, C₁-C₄-alkylcarbonyl- or C₁-C₄-alkoxycarbonyl-,
R¹⁰ and R¹¹ independently of one another represent hydrogen or represent C₁-C₆-alkyl which is optionally mono-, di- or trisubstituted by identical or different substituents from the group consisting of hydroxy, oxo and fluorine,
or represent C₁-C₄-alkylcarbonyl- or C₁-C₄-alkoxycarbonyl-,
or
R¹⁰ and R¹¹ together with the nitrogen atom to which they are attached represent 4- to 8-membered heterocycloalkyl which may optionally be mono- or disubstituted by identical or different substituents from the group consisting of hydroxy, oxo, cyano, fluorine, C₁-C₃-alkyl, halo-C₁-C₄-alkyl-, C₃-C₆-cycloalkyl-, C₃-C₆-cycloalkyl-C₁-C₃-alkyl-, benzyl or C₁-C₄-alkoxycarbonyl-,
or a diastereomer or physiologically acceptable salt thereof.

2. A compound according to claim 1, in which
A represents —NH—,
X represents —N—,
n represents 0 or 1,
R¹ represents —C(═O)NR⁸R⁹ or represents —S(═O)₂NR⁸R⁹,
R² represents hydrogen, fluorine, chlorine, cyano, C₁-C₃-alkyl, fluoro-C₁-C₃-alkyl-, C₁-C₃-alkoxy-, fluoro-C₁-C₃-alkoxy-, C₁-C₃-alkylthio- or fluoro-C₁-C₃-alkylthio-,
R³ represents fluorine, chlorine, methoxy-, ethoxy- or cyano and may be attached to any of the still-unoccupied positions in the aromatic system,
R⁴ represents methyl or ethyl,
R⁵ represents C₁-C₃-alkyl,
R⁶ represents hydrogen,
R⁷ represents C₂-C₆-alkyl, C₃-C₇-cycloalkyl, 4- to 8-membered heterocycloalkyl, phenyl or phenyl-C₁-C₃-alkyl,
in which C₂-C₆-alkyl may optionally be mono-, di- or trisubstituted by identical or different substituents from the group consisting of fluorine, C₁-C₃-alkoxy- and —NR¹⁰R¹¹,
and in which the phenyl radical may in each case optionally be mono-, di- or trisubstituted by identical or different substituents from the group consisting of fluorine, chlorine, bromine, cyano, C₁-C₃-alkyl, C₁-C₃-alkoxy- and trifluoromethyl-,
and in which 4- to 8-membered heterocycloalkyl may optionally be mono- or disubstituted by identical or different substituents from the group consisting of oxo, fluorine, C₁-C₄-alkyl, C₁-C₄-alkylcarbonyl- and C₁-C₄-alkoxycarbonyl-,
R⁸ represents C₁-C₆-alkyl which may optionally be mono-, di- or trisubstituted by identical or different substituents from the group consisting of hydroxy, oxo, fluorine, cyano, C₁-C₃-alkoxy, fluoro-C₁-C₃-alkoxy, —NR¹⁰R¹¹, 4- to 8-membered heterocycloalkyl, phenyl and 5- to 6-membered heteroaryl,
in which the 4- to 8-membered heterocycloalkyl may optionally be monosubstituted by oxo, C₁-C₄-alkyl or C₁-C₄-alkoxycarbonyl-,
and in which phenyl and 5- to 6-membered heteroaryl may optionally be mono- or disubstituted by identical or different substituents from the group consisting of fluorine, chlorine, cyano, trifluoromethyl-, methyl or methoxy-,
or represents C₃-C₈-cycloalkyl which may optionally be mono- or disubstituted by identical or different substituents from the group consisting of hydroxy, oxo, cyano, fluorine, —NR¹⁰R¹¹ and 4- to 8-membered heterocycloalkyl,
or represents 4- to 8-membered heterocycloalkyl, C₆-C₈-heterospirocycloalkyl, bridged C₆-C₁₀-heterocycloalkyl or C₆-C₁₀-heterobicycloalkyl which may optionally be mono- or disubstituted by identical or different substituents from the group consisting of hydroxy, oxo, cyano, fluorine, C₁-C₃-alkyl, —NR¹⁰R¹¹, C₁-C₄-alkylcarbonyl- and C₁-C₄-alkoxycarbonyl-,
R⁹ represents hydrogen or C₁-C₃-alkyl,
or
R⁸ and R⁹ together with the nitrogen atom to which they are attached represent 4- to 8-membered heterocycloalkyl, C₆-C₈-heterospirocycloalkyl, bridged C₆-C₁₀-heterocycloalkyl or C₆-C₁₀-heterobicycloalkyl which may optionally be mono- or disubstituted by identical or different substituents from the group consisting of oxo, cyano, fluorine, C₁-C₃-alkyl, C₃-C₆-cycloalkyl, —NR¹⁰R¹¹, C₁-C₄-alkylcarbonyl- and C₁-C₄-alkoxycarbonyl-,
R¹⁰ and R¹¹ independently of one another represent hydrogen or represent C₁-C₄-alkyl which is optionally mono-, di- or trisubstituted by identical or different substituents from the group consisting of hydroxy, oxo and fluorine, or represent C₁-C₄-alkylcarbonyl- or C₁-C₄-alkoxycarbonyl-,
or
R¹⁰ and R¹¹ together with the nitrogen atom to which they are attached represent 4- to 7-membered heterocycloalkyl which may optionally be mono- or disubstituted by identical or different substituents from the group consisting of hydroxy, oxo, cyano, fluorine, C₁-C₃-alkyl, fluoro-C₁-C₃-alkyl-, C₃-C₆-cycloalkyl-, C₃-C₆-cycloalkylmethyl-, benzyl and C₁-C₄-alkoxycarbonyl-,
or a diastereomer or physiologically acceptable salt thereof.

3. A compound according to claim 1, in which
A represents —NH—,
X represents —N—,
n represents 0 or 1,
R¹ represents —C(═O)NR⁸R⁹ or represents —S(═O)₂NR⁸R⁹,
R² represents hydrogen, fluorine, chlorine, methyl, ethyl, methoxy- or ethoxy-,
R³ represents methoxy- and may be attached to any of the still-unoccupied positions in the aromatic system,
R⁴ represents methyl,
R⁵ represents methyl or ethyl,
R⁶ represents hydrogen,
R⁷ represents C₂-C₅-alkyl, C₃-C₇-cycloalkyl, 5- to 6-membered heterocycloalkyl, phenyl or phenyl-C₁-C₃-alkyl,
in which C₂-C₅-alkyl may optionally be monosubstituted by C₁-C₃-alkoxy, and in which 5- to 6-membered heterocycloalkyl may optionally be monosubstituted by C₁-C₄-alkoxycarbonyl-,
R⁸ represents C₁-C₄-alkyl which may optionally be monosubstituted by —NR¹⁰R¹¹, 4- to 8-membered heterocycloalkyl, phenyl or 5- to 6-membered heteroaryl,
in which the 4- to 8-membered heterocycloalkyl may optionally be monosubstituted by oxo, C₁-C₄-alkyl or C₁-C₄-alkoxycarbonyl-,
and in which phenyl and 5- to 6-membered heteroaryl may optionally be mono- or disubstituted by identical or different substituents from the group consisting of fluorine, chlorine, cyano, trifluoromethyl-, methyl and methoxy-,
or represents C₃-C₈-cycloalkyl which may optionally be mono- or disubstituted by identical or different substituents from the group consisting of hydroxy, oxo, —NR¹⁰R¹¹ and 5- to 6-membered heterocycloalkyl,
or represents 4- to 8-membered heterocycloalkyl which may optionally be mono- or disubstituted by identical or different substituents from the group consisting of oxo, C₁-C₃-alkyl, —NR¹⁰R¹¹, C₁-C₄-alkylcarbonyl- and C₁-C₄-alkoxycarbonyl-,
R⁹ represents hydrogen or methyl,
or
R⁸ and R⁹ together with the nitrogen atom to which they are attached represent 5- to 6-membered heterocycloalkyl or C₆-C₈-heterospirocycloalkyl which may optionally be mono- or disubstituted by identical or different substituents from the group consisting of oxo, fluorine, C₁-C₃-alkyl, C₃-C₅-cycloalkyl, —NR¹⁰R¹¹, C₁-C₄-alkylcarbonyl- and C₁-C₄-alkoxycarbonyl-,
R¹⁰ and R¹¹ independently of one another represent hydrogen, C₁-C₄-alkyl or represent C₁-C₄-alkoxycarbonyl-,
or
R¹⁰ and R¹¹ together with the nitrogen atom to which they are attached represent 5- to 6-membered heterocycloalkyl which may optionally be mono- or disubstituted by identical or different substituents from the group consisting of oxo, fluorine, C₁-C₃-alkyl, fluoro-C₁-C₃-alkyl-, C₃-C₅-cycloalkyl-, C₃-C₅-cycloalkylmethyl- and C₁-C₄-alkoxycarbonyl-,
or a diastereomer or physiologically acceptable salt thereof.

4. A compound according to claim 1, in which
A represents —NH—,
X represents —N—,
n represents 0 or 1,
R¹ represents —C(═O)NR⁸R⁹ or represents —S(═O)₂NR⁸R⁹,
R² represents hydrogen, fluorine, methyl or methoxy-,
R³ represents methoxy- and may be attached to any of the still-unoccupied positions in the aromatic system,
R⁴ represents methyl,
R⁵ represents methyl or ethyl,
R⁶ represents hydrogen,
R⁷ represents C₂-C₄-alkyl, C₅-C₇-cycloalkyl, pyrrolidinyl, piperidinyl, tetrahydropyranyl, phenyl or benzyl,
in which C₂-C₄-alkyl may optionally be monosubstituted by methoxy-, and in which pyrrolidinyl and piperidinyl may optionally be monosubstituted by methoxycarbonyl, ethoxycarbonyl or tert-butoxycarbonyl-,
R⁸ represents C₁-C₂-alkyl which may optionally be monosubstituted by N,N-dimethylamino-, N-ethyl-N-methylamino-, N,N-diethylamino-, pyrrolidinyl, piperidinyl, piperazinyl, morpholinyl, phenyl or pyridinyl, in which pyrrolidinyl, piperidinyl, piperazinyl and morpholinyl may optionally be monosubstituted by methyl, ethyl or tert-butoxycarbonyl-, and in which phenyl and pyridinyl may optionally be monosubstituted by fluorine, chlorine, methyl or methoxy-,
or represents C₅-C₆-cycloalkyl which may optionally be monosubstituted by hydroxy, oxo, —NR¹⁰R¹¹, pyrrolidinyl, piperidinyl, piperazinyl, morpholinyl,
or represents oxetanyl, azetidinyl, pyrrolidinyl, tetrahydrofuranyl or piperidinyl which may optionally be monosubstituted by methyl, ethyl or acetyl-,
R⁹ represents hydrogen or methyl,
or
R⁸ and R⁹ together with the nitrogen atom to which they are attached represent pyrrolidinyl, piperidinyl, piperazinyl, morpholinyl, 1-thia-6-azaspiro[3.3]hept-6-yl- or 2-oxa-6-azaspiro[3.3]hept-6-yl- which may optionally be mono- or disubstituted by identical or different substituents from the group consisting of oxo, fluorine, C₁-C₃-alkyl, cyclopropyl, piperidin-1-yl and tert-butoxycarbonyl-,
R¹⁰ and R¹¹ independently of one another represent hydrogen, C₁-C₃-alkyl or tert-butoxycarbonyl-,
or
R¹⁰ and R¹¹ together with the nitrogen atom to which they are attached represent pyrrolidinyl, piperidinyl, piperazinyl or morpholinyl which may optionally be mono- or disubstituted by identical or different substituents from the group consisting of fluorine, 2,2,2-trifluoroethyl-, cyclopropyl, cyclopropylmethyl- and tert-butoxycarbonyl-,
or a diastereomer or physiologically acceptable salt thereof.

5. A compound according to claim 1, in which
A represents —NH—,
X represents —N—,
n represents 0 or 1,
R¹ represents —C(═O)NR⁸R⁹ or represents —S(═O)₂NR⁸R⁹,
R² represents hydrogen, fluorine, methyl or methoxy-,
R³ represents methoxy- and may be attached to any of the still-unoccupied positions in the aromatic system,
R⁴ represents methyl,
R⁵ represents methyl,
R⁶ represents hydrogen,
R⁷ represents isopropyl, 2-methoxyethyl-, C₅-C₇-cycloalkyl, tetrahydropyran-4-yl, piperidin-4-yl, phenyl or benzyl,
in which piperidin-4-yl may optionally be monosubstituted at its nitrogen atom by tert-butoxycarbonyl-,
R⁸ represents one of the groups below 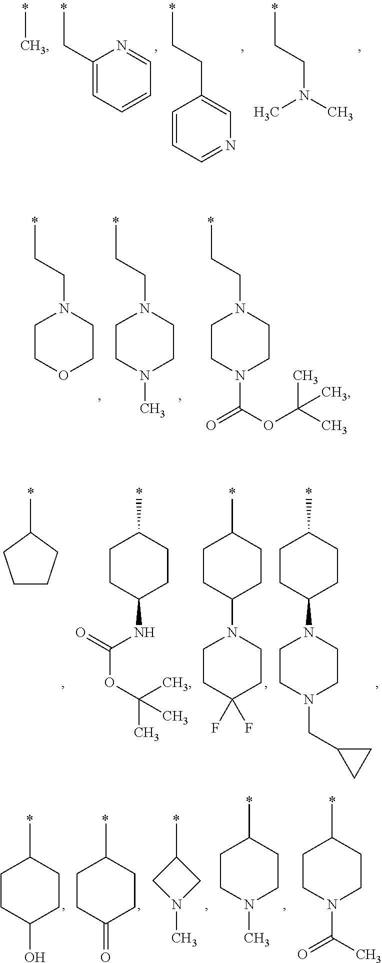
and in which “*” indicates the point of attachment to the nitrogen atom in —C(═O)NR⁸R⁹ and —S(═O)₂NR⁸R⁹, respectively,
R⁹ represents hydrogen or methyl,
or
R⁸ and R⁹ together with the nitrogen atom to which they are attached represent one of the groups below 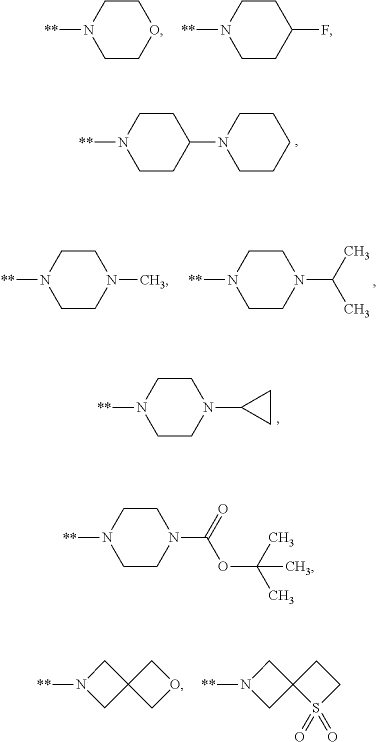
and in which “**” indicates the point of attachment to the carbonyl or sulphonyl group present in R¹,
or a diastereomer or physiologically acceptable salt thereof.

6. A compound according to claim 1 selected from
4-{[(3R)-4-cyclopentyl-1,3-dimethyl-2-oxo-1,2,3,4-tetrahydropyrido[2,3-b]pyrazin-6-yl]amino}-3-methoxy-N-(1-methylpiperidin-4-yl)benzamide;
4-{[(3R)-1,3-dimethyl-2-oxo-4-(propan-2-yl)-1,2,3,4-tetrahydropyrido[2,3-b]pyrazin-6-yl]amino}-3-methoxy-N-(1-methylpiperidin-4-yl)benzamide;
4-{[(3R)-1,3-dimethyl-2-oxo-4-(propan-2-yl)-1,2,3,4-tetrahydropyrido[2,3-b]pyrazin-6-yl]amino}-3-methoxy-N-[2-(morpholin-4-yl)ethyl]benzamide;
1-tert-butyl 4-{2-[(4-{[(3R)-1,3-dimethyl-2-oxo-4-(propan-2-yl)-1,2,3,4-tetrahydropyrido[2,3-b]pyrazin-6-yl]amino}-3-methoxybenzoyl)amino]ethyl}piperazinecarboxylate;
N-[2-(dimethylamino)ethyl]-4-{[(3R)-1,3-dimethyl-2-oxo-4-(propan-2-yl)-1,2,3,4-tetrahydropyrido[2,3-b]pyrazin-6-yl]amino}-3-methoxybenzamide;
N-cyclopentyl-4-{[(3R)-1,3-dimethyl-2-oxo-4-(propan-2-yl)-1,2,3,4-tetrahydropyrido[2,3-b]pyrazin-6-yl]amino}-3-methoxybenzamide;
(3R)-4-cyclopentyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one;
(3R)-6-({4-[(1,1-dioxido-1-thia-6-azaspiro[3.3]hept-6-yl)carbonyl]-2-methoxyphenyl}amino)-4-isopropyl-1,3-dimethyl-3,4-dihydropyrido[2,3-b]pyrazin-2(1H)-one;
(3R)-6-({4-[(1,1-dioxido-1-thia-6-azaspiro[3.3]hept-6-yl)carbonyl]phenyl}amino)-4-isopropyl-1,3-dimethyl-3,4-dihydropyrido[2,3-b]pyrazin-2(1H)-one;
(3R)-4-cyclopentyl-6-({4-[(1,1-dioxido-1-thia-6-azaspiro[3.3]hept-6-yl)carbonyl]-2-methoxyphenyl}amino)-1,3-dimethyl-3,4-dihydropyrido[2,3-b]pyrazin-2(1H)-one;
4-{[(3R)-4-cyclopentyl-1,3-dimethyl-2-oxo-1,2,3,4-tetrahydropyrido[2,3-b]pyrazin-6-yl]amino}-N-(1-methylpiperidin-4-yl)benzamide;
4-{[(3R)-4-cyclohexyl-1,3-dimethyl-2-oxo-1,2,3,4-tetrahydropyrido[2,3-b]pyrazin-6-yl]amino}-3-methoxy-N-(1-methylpiperidin-4-yl)benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(1-methylpiperidin-4-yl)benzamide;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzamide;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
(3R)-1,3-dimethyl-6-[(4-{[4-(propan-2-yl)piperazin-1-yl]sulphonyl}phenyl)amino]-4-(tetrahydro-2H-pyran-4-yl)-3,4-dihydropyrido[2,3-b]pyrazin-2(1H)-one;
4-{[(3R)-4-cyclohexyl-1,3-dimethyl-2-oxo-1,2,3,4-tetrahydropyrido[2,3-b]pyrazin-6-yl]amino}-N,N-dimethylbenzenesulphonamide;
(3R)-1,3-dimethyl-6-{[4-(morpholin-4-ylsulphonyl)phenyl]amino}-4-(tetrahydro-2H-pyran-4-yl)-3,4-dihydropyrido[2,3-b]pyrazin-2(1H)-one;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-[2-(pyridin-3-yl)ethyl]benzenesulphonamide;
(3R)-1,3-dimethyl-6-({4-[(4-methylpiperazin-1-yl)sulphonyl]phenyl}amino)-4-(tetrahydro-2H-pyran-4-yl)-3,4-dihydropyrido[2,3-b]pyrazin-2(1H)-one;
(3R)-6-({2-fluoro-4-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzenesulphonamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-[2-(4-methylpiperazin-1-yl)ethyl]benzenesulphonamide;
N-[2-(dimethylamino)ethyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(pyridin-2-ylmethyl)benzenesulphonamide;
(3R)-6-({3-methoxy-4-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N,N-dimethylbenzenesulphonamide;
(3R)-4-cyclohexyl-6-[(2-methoxy-4-{[4-(propan-2-yl)piperazin-1-yl]carbonyl}phenyl)amino]-1,3-dimethyl-3,4-dihydropyrido[2,3-b]pyrazin-2(1H)-one;
(3R)-4-cyclohexyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one;
4-{[(3R)-4-cyclohexyl-1,3-dimethyl-2-oxo-1,2,3,4-tetrahydropyrido[2,3-b]pyrazin-6-yl]amino}-N-(1-methylazetidin-3-yl)benzamide;
(3R)-4-cyclohexyl-1,3-dimethyl-6-[(4-{[4-(propan-2-yl)piperazin-1-yl]carbonyl}phenyl)amino]-3,4-dihydropyrido[2,3-b]pyrazin-2(1H)-one;
(3R)-4-cycloheptyl-6-{[2-methoxy-4-(2-oxa-6-azaspiro[3.3]hept-6-ylcarbonyl)phenyl]amino}-1,3-dimethyl-3,4-dihydropyrido[2,3-b]pyrazin-2(1H)-one;
(3R)-4-cycloheptyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one;
4-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}-3-methoxybenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(pyridin-2-ylmethyl)benzamide;
(3R)-1,3-dimethyl-6-({2-methyl-4-[(4-methylpiperazin-1-yl)carbonyl]phenyl}amino)-4-(tetrahydro-2H-pyran-4-yl)-3,4-dihydropyrido[2,3-b]pyrazin-2(1H)-one;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(4-oxocyclohexyl)benzamide;
N-(1-acetylpiperidin-4-yl)-4-{[(3R)-4-cyclohexyl-1,3-dimethyl-2-oxo-1,2,3,4-tetrahydropyrido[2,3-b]pyrazin-6-yl]amino}-3-methoxybenzamide;
(3R)-4-cycloheptyl-6-[(2-methoxy-4-{[4-(propan-2-yl)piperazin-1-yl]carbonyl}phenyl)amino]-1,3-dimethyl-3,4-dihydropyrido[2,3-b]pyrazin-2(1H)-one;
(3R)-4-benzyl-1,3-dimethyl-6-{[4-(2-oxa-6-azaspiro[3.3]hept-6-ylcarbonyl)phenyl]amino}-3,4-dihydropyrido[2,3-b]pyrazin-2(1H)-one;
4-{[(3R)-4-benzyl-1,3-dimethyl-2-oxo-1,2,3,4-tetrahydropyrido[2,3-b]pyrazin-6-yl]amino}-N-(4-hydroxycyclohexyl)benzamide;
(3R)-4-benzyl-6-({4-[(4-fluoropiperidin-1-yl)carbonyl]-2-methoxyphenyl}amino)-1,3-dimethyl-3,4-dihydropyrido[2,3-b]pyrazin-2(1H)-one;
4-{[(3R)-4-cycloheptyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide;
(3R)-6-({2-methoxy-4-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one;
N-[2-(dimethylamino)ethyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(pyridin-2-ylmethyl)benzamide;
N-[2-(dimethylamino)ethyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-[2-(pyridin-3-yl)ethyl]benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylazetidin-3-yl)benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-[2-(4-methylpiperazin-1-yl)ethyl]benzamide;
N-[4-(4,4-difluoropiperidin-1-yl)cyclohexyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}benzamide;
(3R)-6-{[4-(1,4′-bipiperidin-1′-ylcarbonyl)-2-methoxyphenyl]amino}-1,3-dimethyl-4-(tetrahydro-2H-pyran-4-yl)-3,4-dihydropyrido[2,3-b]pyrazin-2(1H)-one;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methyl-N-(1-methylpiperidin-4-yl)benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(1-methylazetidin-3-yl)benzamide;
(3R)-1,3-dimethyl-6-({4-[(4-methylpiperazin-1-yl)carbonyl]phenyl}amino)-4-(tetrahydro-2H-pyran-4-yl)-3,4-dihydropyrido[2,3-b]pyrazin-2(1H)-one;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}benzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzamide;
tert-butyl {trans-4-[(4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzoyl)amino]cyclohexyl}carbamate;
4-{[(3R)-1,3-dimethyl-2-oxo-4-(tetrahydro-2H-pyran-4-yl)-1,2,3,4-tetrahydropyrido[2,3-b]pyrazin-6-yl]amino}-N-(4-hydroxycyclohexyl)-3-methoxybenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-[2-(pyridin-3-yl)ethyl]benzamide;
N-[4-(4,4-difluoropiperidin-1-yl)cyclohexyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzamide;
N-[2-(dimethylamino)ethyl]-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-[2-(4-methylpiperazin-1-yl)ethyl]benzamide;
(3R)-6-({trans-4-[(4-cyclopropylpiperazin-1-yl)carbonyl]-2-methoxyphenyl}amino)-1,3-dimethyl-4-(tetrahydro-2H-pyran-4-yl)-3,4-dihydropyrido[2,3-b]pyrazin-2(1H)-one;
4-{[(3R)-4-cyclohexyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
4-{[(3R)-4-cyclohexyl-1,3-dimethyl-2-oxo-1,2,3,4-tetrahydropyrido[2,3-b]pyrazin-6-yl]amino}-3-methoxy-N-[2-(4-methylpiperazin-1-yl)ethyl]benzamide;
4-{[(3R)-4-cyclohexyl-1,3-dimethyl-2-oxo-1,2,3,4-tetrahydropyrido[2,3-b]pyrazin-6-yl]amino}-N-{trans-4-[4-(cyclopropylmethyl) piperazin-1-yl]cyclohexyl}benzamide;
(3R)-4-cyclohexyl-1,3-dimethyl-6-{[4-(2-oxa-6-azaspiro[3.3]hept-6-ylcarbonyl)phenyl]amino}-3,4-dihydropyrido[2,3-b]pyrazin-2(1H)-one;
4-{[(3R)-4-cyclohexyl-1,3-dimethyl-2-oxo-1,2,3,4-tetrahydropyrido[2,3-b]pyrazin-6-yl]amino}-N-{trans-4-[4-(cyclopropylmethyl) piperazin-1-yl]cyclohexyl}-3-methoxybenzamide;
4-{[(3R)-4-cyclohexyl-1,3-dimethyl-2-oxo-1,2,3,4-tetrahydropyrido[2,3-b]pyrazin-6-yl]amino}-N-[4-(4,4-difluoropiperidin-1-yl)cyclohexyl]benzamide;
4-{[(3R)-4-cyclohexyl-1,3-dimethyl-2-oxo-1,2,3,4-tetrahydropyrido[2,3-b]pyrazin-6-yl]amino}-3-methoxy-N-(1-methylazetidin-3-yl)benzamide;
(3R)-4-cyclohexyl-6-{[2-methoxy-4-(2-oxa-6-azaspiro[3.3]hept-6-ylcarbonyl)phenyl]amino}-1,3-dimethyl-3,4-dihydropyrido[2,3-b]pyrazin-2(1H)-one;
(3R)-4-cyclohexyl-6-({4-[(1,1-dioxido-1-thia-6-azaspiro[3.3]hept-6-yl)carbonyl]-2-methoxyphenyl}amino)-1,3-dimethyl-3,4-dihydropyrido[2,3-b]pyrazin-2(1H)-one;
(3R)-4-benzyl-6-({4-[(1,1-dioxido-1-thia-6-azaspiro[3.3]hept-6-yl)carbonyl]phenyl}amino)-1,3-dimethyl-3,4-dihydropyrido[2,3-b]pyrazin-2(1H)-one;
tert-butyl 4-(4-{[(3R)-4-benzyl-1,3-dimethyl-2-oxo-1,2,3,4-tetrahydropyrido[2,3-b]pyrazin-6-yl]amino}benzoyl)piperazine-1-carboxylate;
N-(1-acetylpiperidin-4-yl)-4-{[(3R)-4-benzyl-1,3-dimethyl-2-oxo-1,2,3,4-tetrahydropyrido[2,3-b]pyrazin-6-yl]amino}-3-methoxybenzamide;
N-(1-acetylpiperidin-4-yl)-4-{[(3R)-4-benzyl-1,3-dimethyl-2-oxo-1,2,3,4-tetrahydropyrido[2,3-b]pyrazin-6-yl]amino}benzamide;
4-{[(3R)-4-benzyl-1,3-dimethyl-2-oxo-1,2,3,4-tetrahydropyrido[2,3-b]pyrazin-6-yl]amino}-N-(4-hydroxycyclohexyl)-3-methoxybenzamide;
(3R)-4-benzyl-6-[(2-methoxy-4-{[4-(propan-2-yl)piperazin-1-yl]carbonyl}phenyl)amino]-1,3-dimethyl-3,4-dihydro pyrido[2,3-b]pyrazin-2(1H)-one;
4-{[(3R)-4-benzyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
4-{[(3R)-4-benzyl-1,3-dimethyl-2-oxo-1,2,3,4-tetrahydropyrido[2,3-b]pyrazin-6-yl]amino}-3-methoxy-N-[2-(4-methylpiperazin-1-yl)ethyl]benzamide;
(3R)-4-benzyl-6-{[2-methoxy-4-(2-oxa-6-azaspiro[3.3]hept-6-ylcarbonyl)phenyl]amino}-1,3-dimethyl-3,4-dihydropyrido[2,3-b]pyrazin-2(1H)-one;
(3R)-4-cycloheptyl-6-({4-[(1,1-dioxido-1-thia-6-azaspiro[3.3]hept-6-yl)carbonyl]-2-methoxyphenyl}amino)-1,3-dimethyl-3,4-dihydropyrido[2,3-b]pyrazin-2(1H)-one;
4-{[(3R)-4-cycloheptyl-1,3-dimethyl-2-oxo-1,2,3,4-tetrahydropyrido[2,3-b]pyrazin-6-yl]amino}-N-(1-methylazetidin-3-yl)benzamide;
N-(1-acetylpiperidin-4-yl)-4-{[(3R)-4-cycloheptyl-1,3-dimethyl-2-oxo-1,2,3,4-tetrahydropyrido[2,3-b]pyrazin-6-yl]amino}benzamide;
4-{[(3R)-4-cycloheptyl-1,3-dimethyl-2-oxo-1,2,3,4-tetrahydropyrido[2,3-b]pyrazin-6-yl]amino}-3-methoxy-N-(1-methylazetidin-3-yl)benzamide;
N-(1-acetylpiperidin-4-yl)-4-{[(3R)-4-cycloheptyl-1,3-dimethyl-2-oxo-1,2,3,4-tetrahydropyrido[2,3-b]pyrazin-6-yl]amino}-3-methoxybenzamide;
4-{[(3R)-4-cycloheptyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
4-{[(3R)-4-cycloheptyl-1,3-dimethyl-2-oxo-1,2,3,4-tetrahydropyrido[2,3-b]pyrazin-6-yl]amino}-N-(4-hydroxycyclohexyl)benzamide;
4-{[(3R)-4-cycloheptyl-1,3-dimethyl-2-oxo-1,2,3,4-tetrahydropyrido[2,3-b]pyrazin-6-yl]amino}-3-methoxy-N-[2-(4-methylpiperazin-1-yl)ethyl]benzamide;
(3R)-4-cycloheptyl-1,3-dimethyl-6-[(4-{[4-(propan-2-yl)piperazin-1-yl]carbonyl}phenyl)amino]-3,4-dihydropyrido[2,3-b]pyrazin-2(1H)-one;
(3R)-4-cycloheptyl-1,3-dimethyl-6-{[4-(2-oxa-6-azaspiro[3.3]hept-6-ylcarbonyl)phenyl]amino}-3,4-dihydropyrido[2,3-b]pyrazin-2(1H)-one;
4-{[(3R)-4-benzyl-1,3-dimethyl-2-oxo-1,2,3,4-tetrahydropyrido[2,3-b]pyrazin-6-yl]amino}-N,N-dimethylbenzenesulphonamide;
4-{[(3R)-4-cycloheptyl-1,3-dimethyl-2-oxo-1,2,3,4-tetrahydropyrido[2,3-b]pyrazin-6-yl]amino}-N,N-dimethylbenzenesulphonamide;
N-{trans-4-[4-(cyclopropylmethyl)piperazin-1-yl]cyclohexyl}-4-{[(3R)-1,3-dimethyl-2-oxo-4-(propan-2-yl)-1,2,3,4-tetrahydropyrido[2,3-b]pyrazin-6-yl]amino}benzamide;
4-{[(3R)-1,3-dimethyl-2-oxo-4-(propan-2-yl)-1,2,3,4-tetrahydropyrido[2,3-b]pyrazin-6-yl]amino}-N-(4-hydroxycyclohexyl)-3-methoxybenzamide;
4-{[(3R)-1,3-dimethyl-2-oxo-4-(propan-2-yl)-1,2,3,4-tetrahydropyrido[2,3-b]pyrazin-6-yl]amino}-N-(1-methylpiperidin-4-yl)benzamide;
(3R)-1,3-dimethyl-6-({4-[(4-methylpiperazin-1-yl)sulphonyl]phenyl}amino)-4-(propan-2-yl)-3,4-dihydropyrido[2,3-b]pyrazin-2(1H)-one;
4-{[(3R)-1,3-dimethyl-2-oxo-4-(propan-2-yl)-1,2,3,4-tetrahydropyrido[2,3-b]pyrazin-6-yl]amino}-N,N-dimethylbenzenesulphonamide;
(3R)-1,3-dimethyl-6-{[4-(morpholin-4-ylsulphonyl)phenyl]amino}-4-(propan-2-yl)-3,4-dihydropyrido[2,3-b]pyrazin-2(1H)-one;
(3R)-4-cyclopentyl-1,3-dimethyl-6-[(4-{[4-(propan-2-yl)piperazin-1-yl]sulphonyl}phenyl)amino]-3,4-dihydropyrido[2,3-b]pyrazin-2(1H)-one;
4-{[(3R)-4-cyclopentyl-1,3-dimethyl-2-oxo-1,2,3,4-tetrahydropyrido[2,3-b]pyrazin-6-yl]amino}-N,N-dimethylbenzenesulphonamide;
4-{[4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide;
N-{trans-4-[4-(cyclopropylmethyl)piperazin-1-yl]cyclohexyl}-4-{[4-(2-methoxyethyl)-1,3-dimethyl-2-oxo-1,2,3,4-tetrahydropyrido[2,3-b]pyrazin-6-yl]amino}benzamide;
tert-butyl 4-[(3R)-6-{[4-(dimethylsulphamoyl)phenyl]amino}-1,3-dimethyl-2-oxo-2,3-dihydropyrido[2,3-b]pyrazin-4(1H)-yl]piperidine-1-carbonate;
4-[(1,3-dimethyl-2-oxo-4-phenyl-1,2,3,4-tetrahydropyrido[2,3-b]pyrazin-6-yl)amino]-N-(1-methylpiperidin-4-yl)benzenesulphonamide;
4-{[(3R)-1,3-dimethyl-2-oxo-4-phenyl-1,2,3,4-tetrahydropyrido[2,3-b]pyrazin-6-yl]amino}-N-(1-methylpiperidin-4-yl)benzenesulphonamide;
4-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzenesulphonamide;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxy-N-(1-methylpiperidin-4-yl)benzenesulphonamide;
N-{trans-4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzenesulphonamide and
(3R)-6-({2-methoxy-4-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one,
or a diastereomer or physiologically acceptable salt thereof.

7. (canceled)

8. (canceled)

9. (canceled)

10. A method for the treatment of a neoplastic disorder comprising administering to a patient in need thereof a therapeutically effective amount of a compound according to claim 1.

11. A method for the treatment of a hyperproliferative disorder comprising administering to a patient in need thereof a therapeutically effective amount of a compound according to claim 1.

12. A method for the treatment of a viral infection, neurodegenerative disorder, inflammation disorder, atherosclerotic disorder or in male fertility control comprising administering to a patient in need thereof a therapeutically effective amount of a compound according to claim 1.

13. (canceled)

14. A pharmaceutical composition comprising a compound according to claim 1 in combination with one or more further pharmacologically active substances.

15. (canceled)

16. (canceled)

17. (canceled)

18. A compound of the general formula (VIII) 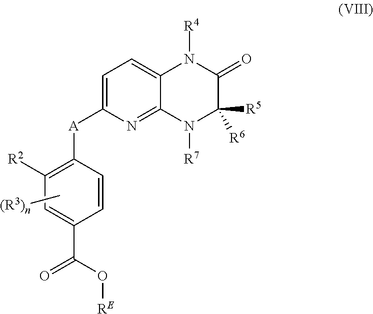
in which
A represents —NH— or —O—,
n represents 0 or 1,
R² represents hydrogen, halogen, cyano, C₁-C₄-alkyl, C₂-C₄-alkenyl, C₂-C₄-alkynyl, halo-C₁-C₄-alkyl-, C₁-C₄-alkoxy-, C₁-C₄-alkoxy-C₁-C₄-alkyl-, halo-C₁-C₄-alkoxy-, C₁-C₄-alkylthio-, halo-C₁-C₄-alkylthio- or —NR¹⁰R¹¹,
R³ represents halogen, C₁-C₃-alkyl, C₁-C₃-alkoxy-, C₁-C₄-alkoxy-C₁-C₄-alkyl-, trifluoromethyl- or cyano and may be attached to any of the still-unoccupied positions in the aromatic system,
R⁴ represents methyl or ethyl,
R⁵ represents hydrogen or C₁-C₃-alkyl,
R⁶ represents hydrogen or C₁-C₃-alkyl,
or
R⁵ and R⁶ together represent C₂-C₅-alkylene,
R⁷ represents C₁-C₆-alkyl, C₃-C₈-cycloalkyl, 4- to 8-membered heterocycloalkyl, phenyl or phenyl-C₁-C₃-alkyl,
in which C₁-C₆-alkyl may optionally be mono-, di- or trisubstituted by identical or different substituents from the group consisting of fluorine, oxo, cyano, hydroxy, C₁-C₃-alkoxy- and —NR¹⁰R¹¹,
and in which the phenyl radical may in each case optionally be mono-, di- or trisubstituted by identical or different substituents from the group consisting of halogen, cyano, C₁-C₄-alkyl, C₂-C₄-alkenyl, C₂-C₄-alkynyl, C₁-C₄-alkoxy-, halo-C₁-C₄-alkyl- and halo-C₁-C₄-alkoxy-,
and in which 4- to 8-membered heterocycloalkyl may optionally be mono- or disubstituted by identical or different substituents from the group consisting of oxo, fluorine, cyano, C₁-C₄-alkyl, C₁-C₄-alkoxy-, C₁-C₄-alkylcarbonyl- and C₁-C₄-alkoxycarbonyl-,
R¹⁰ and R¹¹ independently of one another represent hydrogen or represent C₁-C₆-alkyl which is optionally mono-, di- or trisubstituted by identical or different substituents from the group consisting of hydroxy, oxo and fluorine,
or represent C₁-C₄-alkylcarbonyl- or C₁-C₄-alkoxycarbonyl-,
or
R¹⁰ and R¹¹ together with the nitrogen atom to which they are attached represent 4- to 8-membered heterocycloalkyl which may optionally be mono- or disubstituted by identical or different substituents from the group consisting of hydroxy, oxo, cyano, fluorine, C₁-C₃-alkyl, halo-C₁-C₄-alkyl-, C₃-C₆-cycloalkyl-, C₃-C₆-cycloalkyl-C₁-C₃-alkyl-, benzyl or C₁-C₄-alkoxycarbonyl-,
and R^{E} represents C₁-C₆-alkyl.

19. A compound of the general formula (IX) 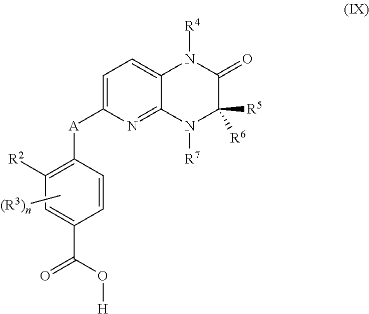
in which
A represents —NH— or —O—,
n represents 0 or 1,
R² represents hydrogen, halogen, cyano, C₁-C₄-alkyl, C₂-C₄-alkenyl, C₂-C₄-alkynyl, halo-C₁-C₄-alkyl-, C₁-C₄-alkoxy-, C₁-C₄-alkoxy-C₁-C₄-alkyl-, halo-C₁-C₄-alkoxy-, C₁-C₄-alkylthio-, halo-C₁-C₄-alkylthio- or —NR¹⁰R¹¹,
R³ represents halogen, C₁-C₃-alkyl, C₁-C₃-alkoxy-, C₁-C₄-alkoxy-C₁-C₄-alkyl-, trifluoromethyl- or cyano and may be attached to any of the still-unoccupied positions in the aromatic system,
R⁴ represents methyl or ethyl,
R⁵ represents hydrogen or C₁-C₃-alkyl,
R⁶ represents hydrogen or C₁-C₃-alkyl,
or
R⁵ and R⁶ together represent C₂-C₅-alkylene,
R⁷ represents C₁-C₆-alkyl, C₃-C₈-cycloalkyl, 4- to 8-membered heterocycloalkyl, phenyl or phenyl-C₁-C₃-alkyl,
in which C₁-C₆-alkyl may optionally be mono-, di- or trisubstituted by identical or different substituents from the group consisting of fluorine, oxo, cyano, hydroxy, C₁-C₃-alkoxy- and —NR¹⁰R¹¹,
and in which the phenyl radical may in each case optionally be mono-, di- or trisubstituted by identical or different substituents from the group consisting of halogen, cyano, C₁-C₄-alkyl, C₂-C₄-alkenyl, C₂-C₄-alkynyl, C₁-C₄-alkoxy-, halo-C₁-C₄-alkyl- and halo-C₁-C₄-alkoxy-,
and in which 4- to 8-membered heterocycloalkyl may optionally be mono- or disubstituted by identical or different substituents from the group consisting of oxo, fluorine, cyano, C₁-C₄-alkyl, C₁-C₄-alkoxy-, C₁-C₄-alkylcarbonyl- and C₁-C₄-alkoxycarbonyl-,
R¹⁰ and R¹¹ independently of one another represent hydrogen or represent C₁-C₆-alkyl which is optionally mono-, di- or trisubstituted by identical or different substituents from the group consisting of hydroxy, oxo and fluorine,
or represent C₁-C₄-alkylcarbonyl- or C₁-C₄-alkoxycarbonyl-,
or
R¹⁰ and R¹¹ together with the nitrogen atom to which they are attached represent 4- to 8-membered heterocycloalkyl which may optionally be mono- or disubstituted by identical or different substituents from the group consisting of hydroxy, oxo, cyano, fluorine, C₁-C₃-alkyl, halo-C₁-C₄-alkyl-, C₃-C₆-cycloalkyl-, C₃-C₆-cycloalkyl-C₁-C₃-alkyl-, benzyl or C₁-C₄-alkoxycarbonyl-.

20. A compound according to claim 18 selected from
methyl 4-{[(3R)-4-cyclopentyl-1,3-dimethyl-2-oxo-1,2,3,4-tetrahydropyrido[2,3-b]pyrazin-6-yl]amino}-3-methoxybenzoate;
methyl 4-{[(3R)-4-cyclohexyl-1,3-dimethyl-2-oxo-1,2,3,4-tetrahydropyrido[2,3-b]pyrazin-6-yl]amino}-3-methoxybenzoate;
methyl 4-{[(3R)-1,3-dimethyl-2-oxo-4-(propan-2-yl)-1,2,3,4-tetrahydropyrido[2,3-b]pyrazin-6-yl]amino}-3-methoxybenzoate;
methyl 4-{[(3R)-4-cyclopentyl-1,3-dimethyl-2-oxo-1,2,3,4-tetrahydropyrido[2,3-b]pyrazin-6-yl]amino}benzoate;
methyl 4-{[(3R)-4-isopropyl-1,3-dimethyl-2-oxo-1,2,3,4-tetrahydropyrido[2,3-b]pyrazin-6-yl]amino}benzoate;
methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzoate;
methyl 4-{[(3R)-4-cyclohexyl-1,3-dimethyl-2-oxo-1,2,3,4-tetrahydropyrido[2,3-b]pyrazin-6-yl]amino}benzoate;
methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}benzoate;
methyl 4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzoate;
methyl 4-{[(3R)-4-cycloheptyl-1,3-dimethyl-2-oxo-1,2,3,4-tetrahydropyrido[2,3-b]pyrazin-6-yl]amino}-3-methoxybenzoate;
methyl 4-{[(3R)-4-cycloheptyl-1,3-dimethyl-2-oxo-1,2,3,4-tetrahydropyrido[2,3-b]pyrazin-6-yl]amino}benzoate;
methyl 4-{[(3R)-4-benzyl-1,3-dimethyl-2-oxo-1,2,3,4-tetrahydropyrido[2,3-b]pyrazin-6-yl]amino}-3-methoxybenzoate;
methyl 4-{[(3R)-4-benzyl-1,3-dimethyl-2-oxo-1,2,3,4-tetrahydropyrido[2,3-b]pyrazin-6-yl]amino}benzoate;
ethyl 4-{[4-(2-methoxyethyl)-1,3-dimethyl-2-oxo-1,2,3,4-tetrahydropyrido[2,3-b]pyrazin-6-yl]amino}benzoate;
4-{[(3R)-4-cyclopentyl-1,3-dimethyl-2-oxo-1,2,3,4-tetrahydropyrido[2,3-b]pyrazin-6-yl]amino}-3-methoxybenzoic acid;
4-{[(3R)-4-cyclohexyl-1,3-dimethyl-2-oxo-1,2,3,4-tetrahydropyrido[2,3-b]pyrazin-6-yl]amino}-3-methoxybenzoic acid;
4-{[(3R)-1,3-dimethyl-2-oxo-4-(propan-2-yl)-1,2,3,4-tetrahydropyrido[2,3-b]pyrazin-6-yl]amino}-3-methoxybenzoic acid;
4-{[(3R)-4-cyclopentyl-1,3-dimethyl-2-oxo-1,2,3,4-tetrahydropyrido[2,3-b]pyrazin-6-yl]amino}benzoic acid;
4-{[(3R)-4-isopropyl-1,3-dimethyl-2-oxo-1,2,3,4-tetrahydropyrido[2,3-b]pyrazin-6-yl]amino}benzoic acid;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methoxybenzoic acid;
4-{[(3R)-4-cyclohexyl-1,3-dimethyl-2-oxo-1,2,3,4-tetrahydropyrido[2,3-b]pyrazin-6-yl]amino}benzoic acid;
4-{[(3R)-1,3-dimethyl-2-oxo-4-(tetrahydro-2H-pyran-4-yl)-1,2,3,4-tetrahydropyrido[2,3-b]pyrazin-6-yl]amino}benzoic acid;
4-{[(3R)-1,3-dimethyl-2-oxo-4-(tetrahydro-2H-pyran-4-yl)-1,2,3,4-tetrahydropyrido[2,3-b]pyrazin-6-yl]amino}-3-methylbenzoic acid;
4-{[(3R)-4-cycloheptyl-1,3-dimethyl-2-oxo-1,2,3,4-tetrahydropyrido[2,3-b]pyrazin-6-yl]amino}-3-methoxybenzoic acid;
4-{[(3R)-4-cycloheptyl-1,3-dimethyl-2-oxo-1,2,3,4-tetrahydropyrido[2,3-b]pyrazin-6-yl]amino}benzoic acid;
4-{[(3R)-4-benzyl-1,3-dimethyl-2-oxo-1,2,3,4-tetrahydropyrido[2,3-b]pyrazin-6-yl]amino}-3-methoxybenzoic acid;
4-{[(3R)-4-benzyl-1,3-dimethyl-2-oxo-1,2,3,4-tetrahydropyrido[2,3-b]pyrazin-6-yl]amino}benzoic acid and
4-{[4-(2-methoxyethyl)-1,3-dimethyl-2-oxo-1,2,3,4-tetrahydropyrido[2,3-b]pyrazin-6-yl]amino}benzoic acid.
